(19) United States
(12) Patent Application Publication
(10) Pub. No.: US 2021/0346356 A1
(43) Pub. Date: Nov. 11, 2021

(54) INHIBITING UBIQUITIN SPECIFIC PEPTIDASE 9X

(71) Applicant: FORMA THERAPEUTICS, INC., Watertown, MA (US)

(72) Inventors: Maureen LYNES, Watertown, MA (US); Weiping WANG, Lexington, MA (US); Bruce FOLLOWS, Littleton, MA (US); Katherine J. KAYSER-BRICKER, Branford, CT (US); Adam Charles TALBOT, Watertown, MA (US); Scot MENTE, Watertown, MA (US); Tatiana SHELEKHIN, Ridgefield, CT (US); Anna ERICSSON, Shrewsbury, MA (US)

(21) Appl. No.: 17/277,503

(22) PCT Filed: Sep. 19, 2019
(86) PCT No.: PCT/US2019/051828

§ 371 Date: Mar. 18, 2021

Related U.S. Application Data
(60) Provisional application No. 62/819,883, filed on Mar. 18, 2019.
(60) Provisional application No. 62/784,981, filed on Dec. 26, 2018.
(60) Provisional application No. 62/733,595, filed on Sep. 19, 2018.

(51) Int. Cl. A61K 31/436; A61K 31/538; A61K 31/407; A61K 31/497; A61K 45/06; A61P 35/00

(52) U.S. Cl. CPC A61K 31/436; A61K 31/538; A61P 35/00; A61K 31/497; A61K 45/06; A61K 31/407

(57) ABSTRACT

This disclosure provides methods of treating cancer with an inhibitor of ubiquitin specific peptidase 9X alone or in combination with an immune checkpoint pathway inhibitor.

### CROSS-REFERENCE TO RELATED APPLICATIONS

This application claims priority to and the benefit of U.S. Provisional Application No. 62/733,595, filed Sep. 19, 2018; U.S. Provisional Application No. 62/784,981, filed Dec. 26, 2018; and U.S. Provisional Application No. 62/819,883, filed Mar. 18, 2019; the entire contents of each of which are incorporated herein by reference.

### TECHNICAL FIELD

This disclosure relates to treating cancer with ubiquitin specific peptidase 9X (USP9X) inhibitors alone and/or in combination with one or more immune checkpoint pathway inhibitors.

### BACKGROUND

Currently 75-85% of patients who received cancer immunotherapy do not respond to it. Therefore, developing T cell-centric immunotherapy for cancer patients who fail to respond to cancer immunotherapies, such as anti-CTLA4 therapy and anti-PD1 therapy, is currently of high interest. T cell-centric immunotherapy has the potential to increase the capacity for the body's immune system to target and eliminate cancer cells. Hence, there is significant unmet need for immunomodulatory therapies that target dysfunctional T cells.

### SUMMARY

The present disclosure includes the recognition that the inhibition of USP9X has immune modulating function via activation of T cells and that this immune modulating function can reduce and/or prevent tumor growth. Accordingly, in some embodiments, USP9X inhibitors can be used to treat cancer.

The present disclosure provides methods of treating cancer in a patient in need thereof, comprising administering to the patient a USP9X Inhibitor. In some embodiments, a USP9X Inhibitor is a compound characterized in that it has an IC₅₀ value when tested in the Biochemical Assay of Example A of:
(i) ≤10 μM and >1 μM;
(ii) ≤2 μM and >0.2 μM;
(iii) ≤1 μM and >0.1 μM;
(iv) ≤0.2 μM and >0.05 μM;
(v) ≤0.1 μM and >0.001 μM; and/or
(vi) ≤0.05 μM and >0.001 μM.

Additionally, the present disclosure provides methods of treating cancer by administering a USP9X Inhibitor in combination with an immune checkpoint pathway inhibitor. In some embodiments, a method of treating cancer in a patient in need thereof comprises administering to the patient a USP9X Inhibitor, wherein the patient is receiving or has received an immune checkpoint pathway inhibitor. In some embodiments, a method of treating cancer in a patient in need thereof comprises administering to the patient an immune checkpoint pathway inhibitor, wherein the patient is receiving or has received a USP9X Inhibitor.

### BRIEF DESCRIPTION OF THE DRAWINGS

FIG. 1 is a graph of changes in IFNγ production in a human T cell activation assay.

FIG. 2 depicts Western blot analyses of changes in ITCH and Cbl-b proteins in SEB-exhausted human PBMCs treated with USP9X Inhibitors.

FIG. 3A is a graph of changes in basal IFNγ production in SEB-exhausted human PBMCs as a function of concentration of USP9X Inhibitor 3 and negative control compound 5.

FIG. 3B is a plot of changes in basal IFNγ production in SEB-exhausted human PBMCs of various USP9X Inhibitors at 1 μM concentration.

FIG. 4A is a graph of IFNγ production following SEB-restimulation in human PBMCs. FIG. 4B is a graph of IL2 production following SEB-restimulation in human PBMCs. FIG. 4A and FIG. 4B indicate that cytokine production in SEB-restimulated cells is attenuated.

FIG. 5 is a graph showing restoration of IFNγ in the presence of USP9X Inhibitor 3 (10 μM) and anti-PD-1 following SEB-restimulation. An asterisk (*) denotes p<0.05.

FIG. 6 is a graph. An asterisk (*) denotes p<0.05. “ns” indicates not significant. In an anti-CD3/CD28 T cell activation assay, several USP9X inhibitors demonstrated enhanced IFNγ production, with USP9X Inhibitor 3 yielding the most profound effect. Positive controls, anti-PD1 and anti-CTLA4 antibodies also increased IFNγ production, whereas negative control compound 5 showed weak to no activity.

FIG. 7 is a graph % increase of IFNγ production in the presence of various concentrations of USP9X Inhibitor 3 and negative control compound 5 in the MLR assay of Example 2.

FIG. 8A is a plot of fold change of IL-2 production in the presence of USP9X Inhibitors in allogenic CD4+ T cells in the MLR assay of Example 3. FIG. 8B is a plot of fold change of IFNγ production in the presence of USP9X Inhibitors in allogenic CD4+ T cells in the MLR assay of Example 3.

FIG. 9A is a plot of allogeneic T cell-mediated cell kill of A375 melanoma cells in the presence of USP9X Inhibitor 3, as measured in the caspase 3/7 assay of Example 4 (n=3).

FIG. 9B is a plot of allogeneic T cell-mediated cell kill of A375 melanoma cells in the presence of negative control compound 5, as measured in the caspase 3/7 assay of Example 4 (n=3). In FIG. 9A and FIG. 9B, *** indicates p<0.001, and **** indicates p<0.0001.

FIG. 10A is a plot of fold increase in immune cell kill of A-375 cells in the presence of USP9X Inhibitor 1 (i.e., “Compound 1”) in donor PBMCs as described in Example 5. FIG. 10B is a plot of fold increase in immune cell kill of A-375 cells in the presence of USP9X Inhibitor 2 (i.e., “Compound 2”) in donor PBMCs as described in Example 5. FIG. 10C is a plot of fold increase in immune cell kill of A-375 cells in the presence of negative control compound 5 in donor PBMCs as described in Example 5.

FIG. 11A is a plot of IFNγ production in one donor at various concentrations of USP9X Inhibitor 4 as measured in the assay of Example 6. “NC” indicates negative control compound 5. FIG. 11B is a plot of IFNγ production in a second donor at various concentrations of USP9X Inhibitor 4 as measured in the assay of Example 6. “NC” indicates negative control compound 5.

FIG. 12A is a plot of IFNγ production in one donor at various concentrations of USP9X Inhibitor 3 as measured in the assay of Example 6. “NC” indicates negative control compound 5. FIG. 12B is a plot of IFNγ production in a second donor at various concentrations of USP9X Inhibitor 3 as measured in the assay of Example 6. “NC” indicates negative control compound 5.

FIG. 13A is a graph of % specific cell kill of A-375 melanoma cells in the presence of USP9X Inhibitor 2, anti-CTLA-4, or a combination of both at various ratios of PBMCs. A-375 cells in one donor as described in Example 8. FIG. 13B is a graph of % specific cell kill of A-375 melanoma cells in the presence of USP9X Inhibitor 2, anti-CTLA-4, or a combination of both at various ratios of PBMCs. A-375 cells in another donor as described in Example 8.

### DETAILED DESCRIPTION

The present disclosure provides methods of treating cancer, comprising administering a USP9X Inhibitor to a patient in need thereof. The disclosure is based in part on the recognition that the inhibition of USP9X has immune modulating function via activation of T cells and that this immune modulating function can reduce and/or prevent tumor growth.

The present disclosure also provides methods of treating cancer, comprising administering a USP9X Inhibitor to a patient in need thereof, wherein the patient is receiving or has received an immune checkpoint pathway inhibitor. Additionally, the present disclosure also provides methods of treating cancer, comprising administering an immune checkpoint pathway inhibitor to a patient in need thereof, wherein the patient is receiving or has received a USP9X Inhibitor.

Without wishing to be bound by theory, USP9X Inhibitors and immune checkpoint pathway inhibitors may have separate mechanisms of action. USP9X Inhibitors, therefore, can be useful in treating cancer in a patient that is non-responsive to therapy with an immune checkpoint pathway inhibitor alone.

### Methods of Treating Cancer

Methods of treating cancer are provided herein. In some embodiments, a method of treating cancer comprises administering a USP9X Inhibitor to a patient in need thereof. In some embodiments, a method of treating cancer comprises administering a USP9X Inhibitor to a patient in need thereof, wherein the patient is receiving or has received an immune checkpoint pathway inhibitor. In some embodiments, a method of treating cancer comprises administering an immune checkpoint pathway inhibitor to a patient in need thereof, wherein the patient is receiving or has received a USP9X Inhibitor. In some embodiments, the cancer is refractory or resistant to treatment. In some embodiments, the cancer has progressed after one or more previous lines of chemotherapy. In some embodiments, the cancer has progressed after two or more previous lines of chemotherapy. In some embodiments, the cancer has progressed after three or more previous lines of chemotherapy.

In some embodiments, the cancer comprises a tumor that expresses PD-L1. PD-L1 expression can be detected by an FDA-approved test, such as PD-L1 TIC 22C3 pharmDx or PD-L1 (SP142). In some embodiments, the cancer comprises a tumor that expresses CTLA-4. In some embodiments, the cancer comprises a tumor in a patient that expresses CTLA-4 in the tumor environment or draining lymphoid tissues. CTLA-4 expression can be assessed by methods known to a person skilled in the art.

In some embodiments, the cancer is selected from unresectable or metastatic melanoma, cutaneous melanoma, advanced renal cell carcinoma, microsatellite instability-high (MSI-H) or mismatch repair deficient (dMMR) metastatic colorectal cancer, metastatic squamous non-small cell lung cancer, head and neck squamous cell cancer, classical Hodgkin lymphoma, primary mediastinal large B-cell lymphoma, urothelial carcinoma, microsatellite instability-high cancer, gastric cancer, cervical cancer, hepatocellular carcinoma, or Merkel cell carcinoma.

In some embodiments, a method of treating cancer comprises administering a USP9X Inhibitor to a patient in need thereof, wherein the patient is receiving or has received an immune checkpoint pathway inhibitor, and wherein the cancer is selected from unresectable or metastatic melanoma, cutaneous melanoma, advanced renal cell carcinoma, microsatellite instability-high (MSI-H) or mismatch repair deficient (dMMR) metastatic colorectal cancer, metastatic squamous non-small cell lung cancer, head and neck squamous cell cancer, classical Hodgkin lymphoma, primary mediastinal large B-cell lymphoma, urothelial carcinoma, microsatellite instability-high cancer, gastric cancer, cervical cancer, hepatocellular carcinoma, or Merkel cell carcinoma.

In some embodiments, a method of treating cancer comprises administering an immune checkpoint pathway inhibitor to a patient in need thereof, wherein the patient is receiving or has received a USP9X Inhibitor, and wherein the cancer is selected from unresectable or metastatic melanoma, cutaneous melanoma, advanced renal cell carcinoma, microsatellite instability-high (MSI-H) or mismatch repair deficient (dMMR) metastatic colorectal cancer, metastatic squamous non-small cell lung cancer, head and neck squamous cell cancer, classical Hodgkin lymphoma, primary mediastinal large B-cell lymphoma, urothelial carcinoma, microsatellite instability-high cancer, gastric cancer, cervical cancer, hepatocellular carcinoma, or Merkel cell carcinoma.

### Patient Selection

In addition, patients can be selected to receive treatment with a USP9X Inhibitor alone, and/or with a USP9X Inhibitor in combination with an immune checkpoint pathway inhibitor. For example, patients can be selected based on their prior treatment status and/or their status in a genetic risk panel analysis of the patient, such as PD-L1.

In some embodiments, methods provided herein are useful for treating patients who have not responded to previous cancer immunotherapy. In some embodiments, provided methods are useful for treating patients who have not responded to prior therapy with an immune checkpoint pathway inhibitor, such as ipilimumab, nivolumab, or pembrolizumab.

In some embodiments, methods provided herein are useful for treating patients who have not responded to previous chemotherapy. In some embodiments, the previous chemotherapy is selected from platinum-based chemotherapy (e.g., oxaliplatin, cisplatin, or carboplatin), fluoropyrimidine therapy, irinotecan therapy, paclitaxel therapy, nab-paclitaxel therapy, HER2/neu-targeted therapy, or sorafenib therapy.

In some embodiments, methods provided herein are useful for treating patients who have received one or more prior lines of chemotherapy. In some embodiments, methods provided herein are useful for treating patients who have received two or more prior lines of chemotherapy. In some embodiments, methods provided herein are useful for treating patients who have received three or more prior lines of chemotherapy.

Patients with cancer comprising a tumor expressing PD-1 can be identified using a diagnostic test. In some embodiments, an FDA-approved diagnostic test, such as PD-L1 IHC 22C3 pharmDx (Dako North America, Inc.) is used in the detection of PD-L1 protein in cancer. Results of the test are used as an aid in the identification of cancer patients who may be considered for treatment with a therapeutic agent, such as an immune checkpoint pathway inhibitor, including pembrolizumab. In some embodiments, patients evaluated with a diagnostic test (e.g., PD-L1 IHC 22C3 pharmDx (Dako North America, Inc.)) that are determined to express PD-L1 in cancer are treated with a therapeutic agent (e.g., an immune checkpoint pathway inhibitor) in accordance with provided methods.

PD-L1 IHC 22C3 pharmDx is a qualitative immunohistochemical assay using Monoclonal Mouse Anti-PD-L1, Clone 22C3 intended for use in the detection of PD-L1 protein in formalin-fixed, paraffin-embedded (FFPE) non-small cell lung cancer (NSCLC), gastric or gastroesophageal junction (GEJ) adenocarcinoma, cervical cancer and urothelial carcinoma tissues using EnVision FLEX visualization system on Autostainer Link 48.

In some embodiments, a method of treating cancer comprises administering a USP9X Inhibitor to a patient in need thereof, wherein the patient is or has been selected for treatment using a diagnostic test, such as PD-L1 IHC 22C3 pharmDx. In some embodiments, the patient is or has been determined to have a cancer expressing PD-L1 using PD-L1 IHC 22C3 pharmDx.

### Combination Therapy

In some embodiments, methods of treating cancer comprise administering two or more therapeutic regimens to a patient in need thereof (e.g., a USP9X Inhibitor and an immune checkpoint pathway inhibitor). In some embodiments, the two or more therapeutic regimens may be administered simultaneously. In some embodiments, such regimens may be administered sequentially (e.g., all “doses” of a first regimen are administered prior to administration of any doses of a second regimen). In some embodiments, such agents are administered in overlapping dosing regimens. For clarity, combination therapy does not require that individual agents be administered together in a single composition (or even necessarily at the same time). In some embodiments, two or more therapeutic agents or regimens of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition.

### USP9X Inhibitors

Deubiquitylating enzymes control a number of cellular processes, including the stability of a variety of oncoproteins, by reversing ubiquitination. USP9X is a member of the USP family of DUBs and is a key regulator of protein homeostasis for protein substrates including several that are known to be oncogenic or protumorigenic. Overexpression and/or mutation of DUBs and their substrates are associated with cancer initiation and progression.

USP9X inhibition can promote antitumor T cell responses. Although USP9X is not required for T cell survival, it is required for normal T cell development and proliferation. Additionally, USP9X may have a role in T cell activation and tolerance as a regulator of the ubiquitylation and stability of ITCH, a known E3 ubiquitin ligase. ITCH, as well as Cbl-b and GRAIL, are critical for T cell activation and T cell tolerance induction, which act in part by attenuating the T cell receptor (TCR) signal. Interestingly, the co-inhibitory receptor CTLA-4, a key mediator of T cell tolerance, may exert its inhibitory T cell function, at least in part, by activating ITCH. Thus, enhanced degradation of ITCH and consequent loss of T cell tolerance could explain the spontaneous autoimmunity and lymphoproliferative diseases manifested in T cell-specific USP9X knockout (KO) mice.

By way of non-limiting example, USP9X Inhibitors that may be used in accordance with the present disclosure include those described in WO2014/172638, WO2015/054555, and WO2015/187427, each of which is hereby incorporated by reference.

In some embodiments, a USP9X Inhibitor is a compound of Formula I:

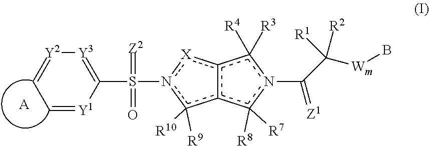

or a pharmaceutically acceptable salt thereof, wherein:
X is CR⁵R⁶, CR⁵, NR⁵, or N, as valency permits;
dashed bonds are each independently a single or a double bond, as valency permits;
Y¹, Y², and Y³ are each independently N or CR^{a};
each R^{a} is independently —H, halogen, or —CN;
Ring A is a 5- to 6-membered aryl, 5- to 6-membered heteroaryl containing 1-4 heteroatoms independently selected from the group consisting of O, N, and S, 5- to 7-membered heterocyclyl containing 1-4 heteroatoms independently selected from the group consisting of O, N, and S, or 5- to 7-membered cycloalkyl,
   wherein each aryl, heteroaryl, heterocyclyl, or cycloalkyl is optionally substituted with one or more halogen, —C₁-C₆alkyl, —C₂-C₆alkenyl, —C₂-C₆alkynyl, oxo, or —C(O)R′;
Z¹ is O, S, or NR;
Z² is O or NR;
W is CR^{1′}R^{2′}, O, S, or NR;
m is 0 or 1;
R¹ and R² are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each cycloalkyl, cycloalkenyl, heterocyclyl, aryl, or heteroaryl is optionally substituted with one or more R^{e},
   wherein each alkyl, alkenyl, or alkynyl is optionally substituted with one or more halogen,
   wherein each heterocyclyl is 3- to 14-membered and contains 1-4 heteroatoms independently selected from the group consisting of O, N, and S, and wherein the heterocyclyl does not contain an O in the γ-position relative to C(═Z¹), and
   wherein each heteroaryl is 5- to 14-membered and contains 1-4 heteroatoms independently selected from the group consisting of O, N, and S;
or R¹ and R² combine with the carbon to which they are attached to form oxo, a C₃-C₈cycloalkyl, or a 3- to 8-membered heterocyclyl containing 1-4 heteroatoms independently selected from the group consisting of O, N, and S, and wherein the heterocyclyl does not contain an O in the γ-position relative to C(═Z¹), and
   wherein each cycloalkyl or heterocyclyl is optionally substituted with one or more R^{e};
R^{1′} and R^{2′} are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —(CR^{b}R^{c})ₙNR², —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each alkyl, alkenyl, alkynyl, cycloalkyl, cycloalkenyl, heterocyclyl, aryl, or heteroaryl is optionally substituted with one or more R^{e},
   wherein each heterocyclyl is 3- to 14-membered and contains 1-4 heteroatoms independently selected from the group consisting of O, N, and S, and
   wherein each heteroaryl is 5- to 14-membered and contains 1-4 heteroatoms independently selected from the group consisting of O, N, and S;
or R^{1′} and R^{2′} combine with the carbon to which they are attached to form oxo, a C₃-C₈cycloalkyl, or a 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e}.
or R¹ and R^{1′} combine with the carbons to which they are attached to form a C₃-C₈cycloalkyl or 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e};
R^{b} and R^{c} are each independently selected from the group consisting of —H, halogen, and —C₁-C₆alkyl;
each n is independently 0, 1, 2, 3, or 4;
each R^{e} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S, and wherein —OR of R^{e} does not result in an O in the γ-position relative to C(═Z¹),
   wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
B is a monocyclic or bicyclic 3- to 14-membered ring,
   wherein the ring is saturated, fully or partially unsaturated, or aromatic, and
   wherein the ring contains 0-4 heteroatoms independently selected from the group consisting of O, N, and S,
   wherein the ring is optionally substituted with one or more R^{d}, and
   when m is 0 and the ring is saturated or partially unsaturated, then the ring does not contain an O in the γ-position relative to C(═Z¹);
each R^{d} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
each R³, R⁴, R⁵, R⁶, R⁷, R⁸, R⁹, and R¹⁰ is independently —H, —C₁-C₆alkyl, —C₃-C₈cycloalkyl, or 3- to 8-membered heterocyclyl containing 1-4 heteroatoms independently selected from the group consisting of O, N, and S,
   wherein each alkyl, cycloalkyl, or heterocyclyl is optionally substituted with one or more halogen, oxo, —C₁-C₆alkyl, —C₂-C₆alkenyl, —C₂-C₆alkynyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₃-C₈cycloalkyl, or 3- to 8-membered heterocyclyl containing 1-4 heteroatoms independently selected from the group consisting of O, N, and S, and
   wherein R³, R⁷, and R⁹ are each independently present or absent, as valency permits;
or R³ and R⁴, R⁵ and R⁶, R⁷ and R⁸, R⁹ and R¹⁰, or combinations thereof, combine with the carbon to which they are attached to form an oxo, C₃-C₈cycloalkyl, or 3- to 8-membered heterocyclyl containing 1-4 heteroatoms independently selected from the group consisting of O, N, and S;
each R is independently selected from the group consisting of —H, —OH, —O(C₁-C₆alkyl), —NH₂, —NH(C₁-C₆alkyl), —N(C₁-C₆alkyl)₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —O—C₁-C₆alkyl, —NH(C₁-C₆alkyl), —N(C₁-C₆alkyl)₂, —C₁-C₆alkyl optionally substituted with one or more oxo or —OH, —C₂-C₆alkenyl, —C₂-C₆alkynyl, —C₃-C₁₂cycloalkyl, or 3- to 8-membered heterocyclyl containing 1-4 heteroatoms independently selected from the group consisting of O, N, and S; and
each R′ is independently selected from the group consisting of —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —C₁-C₆alkyl optionally substituted with one or more oxo or —OH, —C₂-C₆alkenyl, —C₂-C₆alkynyl, —O—C₁-C₆alkyl, —NH(C₁-C₆alkyl), or —N(C₁-C₆alkyl)₂.

In some embodiments, a USP9X Inhibitor is a compound of Formula I-a:

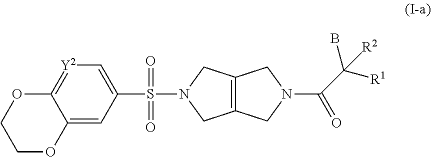

or a pharmaceutically acceptable salt thereof, wherein B, R¹, and R² are as defined above for Formula I, and wherein Y² is CH or N.

In some embodiments, a USP9X Inhibitor is a compound of Formula I-b:

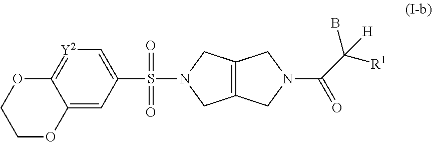

or a pharmaceutically acceptable salt thereof, wherein:
Y² is CH or N;
R¹ is —OH or —(CH₂)NHMe;
B is a phenyl ring or a bicyclic ring,
   wherein at least one of the rings in the bicyclic ring is a phenyl ring,
   wherein the phenyl ring or bicyclic ring contains 0-4 heteroatoms independently selected from the group consisting of O, N, and S, and
   wherein the phenyl ring or bicyclic ring is optionally substituted with one or more R^{d}.
each R^{d} is independently selected from the group consisting of halogen, —C₁-C₆alkyl, and —OR; and
each R is independently —H, —C₁-C₆alkyl, or 3- to 8-membered heterocyclyl optionally substituted with —C₁-C₆alkyl.

In some embodiments, a USP9X Inhibitor is a compound of Formula I-c:

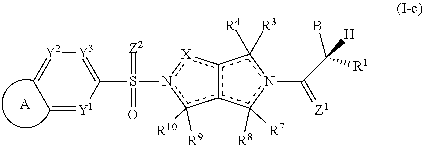

or a pharmaceutically acceptable salt thereof,
wherein X, dashed bonds, Y¹, Y², Y³, Ring A, B, Z¹, Z², R¹, R³, R⁴, R⁷, R⁸, R⁹, and R¹⁰ are as defined above for Formula I and described in classes and subclasses of Formula I herein, both singly and in combination.

In some embodiments, a USP9X Inhibitor is a compound of Formula I-d:

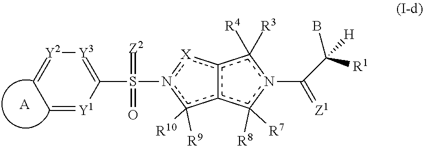

or a pharmaceutically acceptable salt thereof,
wherein X, dashed bonds, Y¹, Y², Y³, Ring A, B, Z¹, Z², R¹, R³, R⁴, R⁷, R⁸, R⁹, and R¹⁰ are as defined above for Formula I and described in classes and subclasses of Formula I herein, both singly and in combination.

In some embodiments, a USP9X Inhibitor is a compound of Formula I-e:

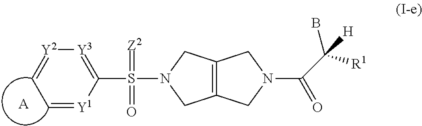

or a pharmaceutically acceptable salt thereof,
wherein Y¹, Y², Y³, Ring A, B, Z¹, Z², and R¹ are as defined above for Formula I and described in classes and subclasses of Formula I herein, both singly and in combination.

In some embodiments, a USP9X Inhibitor is a compound of Formula I-f:

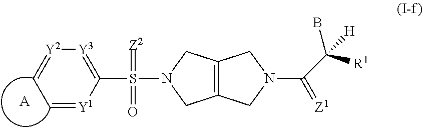

or a pharmaceutically acceptable salt thereof,
wherein Y¹, Y², Y³, Ring A, B, Z¹, Z², and R¹ are as defined above for Formula I and described in classes and subclasses of Formula I herein, both singly and in combination.

In some embodiments of Formulas I, I-a, I-b, I-c, I-d, I-e, and I-f, Y¹, Y², and Y³ are each independently CR^{a}. In some embodiments, Y¹, Y², and Y³ are each CH. In some embodiments, at least one of Y¹, Y², and Y³ is N. In some embodiments, at least one of Y¹ and Y² is N. In some embodiments, Y¹ is CR^{a}. In some embodiments, Y¹ is N. In some embodiments, Y² is CR^{a}. In some embodiments, Y² is N. In some embodiments, Y³ is CR^{a}. In some embodiments, Y³ is N.

In some embodiments of Formulas I, I-c, I-d, I-e, and I-f,

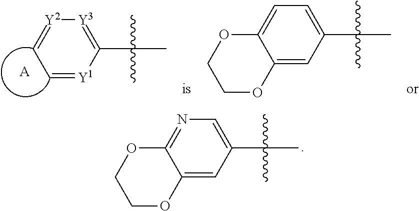

In some embodiments,

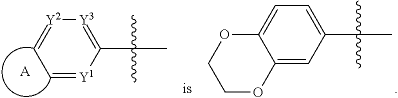

In some embodiments,

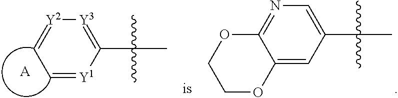

In some embodiments of Formulas I, I-c, I-d, I-e, and I-f, and V, Z¹ is O or S. In some embodiments, Z¹ is O. In some embodiments, Z¹ is S. In some embodiments, Z¹ is NR. In some embodiments, Z¹ is NH, NOH, or NNH₂.

In some embodiments of Formulas I, I-c, I-d, I-e, and I-f, Z² is O or NH. In some embodiments, Z² is O. In some embodiments, Z² is NR. In some embodiments, Z² is NH.

In some embodiments of Formulas I, I-a, I-b, I-c, I-d, I-e, and I-f, R¹ and R² are each independently selected from the group consisting of —H, halogen, —C₁-C₆alkyl, —(CR^{b}R^{c})ₙheterocyclyl, —OR, —(CR^{b}R^{c})ₙNR², —(CR^{b}R^{c})ₙNRC(O)R′, or —(CR^{b}R^{c})ₙNRC(O)NR₂, wherein each heterocyclyl is optionally substituted with one or more halogen, and wherein each heterocyclyl is 3- to 14-membered and contains 1-4 heteroatoms independently selected from the group consisting of O, N, and S, and wherein the heterocyclyl does not contain an O in the γ-position relative to C(═Z¹); or R¹ and R² combine with the carbon to which they are attached to form a 3- to 8-membered heterocyclyl containing 1-4 heteroatoms independently selected from O, N, and S, wherein the heterocyclyl does not contain an O in the γ-position relative to C(═Z¹).

In some embodiments, R¹ and R² are each independently —H, —OR, —(CR^{b}R^{c})ₙNR₂, or —(CR^{b}R^{c})ₙNRC(O)R′. In some embodiments, R¹ and R² are each independently —H, —OR, —CH₂NR₂, or —CH₂NRC(O)R′. In some embodiments, R¹ and R² are each independently —H, —OH, —CH₂NHMe, or —CH₂NHC(O)Me. In some embodiments, R¹ and R² are each independently —H, —OH, or —CH₂NHMe. In some embodiments, one of R¹ and R² is not —H. In some embodiments, R¹ is —OH or —(CH₂)NHMe. In some embodiments, R¹ is —OH. In some embodiments, R² is —H.

In some embodiments of Formulas I, I-a, I-b, I-c, I-d, I-e, and I-f, B is:
(i) a monocyclic 3- to 8-membered ring, comprising a C₃-C₈cycloalkyl, 3- to 8-membered heterocyclyl, phenyl, or 5- to 8-membered heteroaryl ring,
wherein the ring contains 0-4 heteroatoms independently selected from the group consisting of O, N, and S,
wherein the ring is optionally substituted with one or more R^{d}, and
wherein the ring comprising a 3- to 8-membered heterocyclyl does not contain an O in the γ-position relative to C(═Z); or
(ii) a bicyclic 6- to 14-membered ring, comprising a C₃-C₁₀cycloalkyl, 3- to 11-membered heterocyclyl, phenyl, or 5- to 11-membered heteroaryl ring,
wherein the ring is fused to an aromatic, saturated, or partially unsaturated 3- to 8-membered carbocyclic or heterocyclic ring,
wherein the ring contains 0-4 heteroatoms independently selected from the group consisting of O, N, and S,
wherein the ring is optionally substituted with one or more R^{d}, and
wherein the ring comprising a 3- to 11-membered heterocyclyl does not contain an O in the γ-position relative to C(═Z).

In some embodiments of Formulas I, I-a, I-b, I-c, I-d, I-e, and I-f, B is a phenyl ring or a bicyclic ring, wherein at least one of the rings in the bicyclic ring is a phenyl ring, wherein the phenyl ring or bicyclic ring contains 0-4 heteroatoms independently selected from the group consisting of O, N, and S, and wherein the phenyl ring or bicyclic ring is optionally substituted with one or more R^{d}. In some embodiments, B is a phenyl ring optionally substituted with one or more R^{d}. In some embodiments, B is a phenyl ring optionally substituted with one or more R^{d} and is fused to an aromatic, saturated, or partially unsaturated 5- to 8-membered carbocyclic or heterocyclic ring. In some embodiments, B is a phenyl ring optionally substituted with one or more R^{d} and is fused to a saturated or partially unsaturated 5- to 8-membered heterocyclic ring. In some embodiments, B is a monocyclic or bicyclic heteroaryl ring, wherein the ring contains 1-4 heteroatoms independently selected from the group consisting of O, N, and S, and wherein the ring is optionally substituted with one or more R^{d}.

In some embodiments, B is selected from:

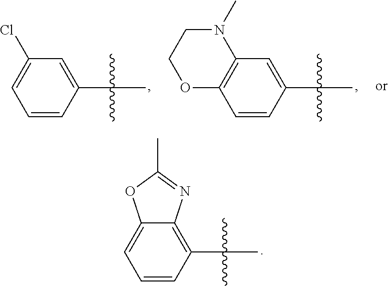 [SMILES: *c1cccc(Cl)c1 ; *c1ccc2c(c1)N(C)CCO2 ; *c1cccc2oc(C)nc12]

In some embodiments of Formulas I, I-a, I-b, I-c, I-d, I-e, and I-f, each R^{d} is independently selected from the group consisting of halogen, —OR, —NR₂ (e.g., —N(Me)(CH₂CH₂OMe)), —C(O)NR₂, —C₁-C₆alkyl, —C₃-C₁₂cycloalkyl, 3- to 14-membered heterocyclyl containing 1-4 heteroatoms independently selected from the group consisting of O, N, and S, and C₆-C₁₄aryl, wherein each alkyl, heterocyclyl, or aryl is optionally substituted with one or more substituents selected from the group consisting of halogen, —C₁-C₆alkyl optionally substituted with one or more halogen, or —C₃-C₁₂cycloalkyl. In some embodiments, each R^{d} is independently selected from the group consisting of halogen, —OR, —C₁-C₆alkyl (e.g., methyl, ethyl, —CHF₂, or —CF₃), —C₃-C₁₂cycloalkyl, and 3- to 14-membered heterocyclyl containing 1-4 heteroatoms independently selected from the group consisting of O, N, and S. In some embodiments, each R^{d} is independently selected from the group consisting of halogen, —C₁-C₆alkyl, and —OR.

In some embodiments of Formulas I, I-a, I-b, I-c, I-d, I-e, and I-f, each R is independently selected from the group consisting of —H, —C₁-C₆alkyl, —C₃-C₁₂cycloalkyl, and 3- to 14-membered heterocyclyl containing 1-4 heteroatoms independently selected from the group consisting of O, N, and S, wherein each alkyl or heterocyclyl is optionally substituted with one or more halogen, —O—C₁-C₆alkyl, —NH—C₁-C₆alkyl, —N(C₁-C₆alkyl)₂, —C₁-C₆alkyl optionally substituted with —OH, —C₃-C₁₂cycloalkyl, or 3- to 8-membered heterocyclyl containing 1-4 heteroatoms independently selected from the group consisting of O, N, and S. In some embodiments, each R is independently —H, —C₁-C₆alkyl, or 3- to 8-membered heterocyclyl optionally substituted with C₁-C₆alkyl. In some embodiments, each R is independently —H or methyl.

In some embodiments of Formulas I, I-a, I-b, I-c, I-d, I-e, and I-f, each R′ is independently —C₁-C₆alkyl, —C₃-C₁₂cycloalkyl, or 3- to 14-membered heterocyclyl containing 1-4 heteroatoms independently selected from the group consisting of O, N, and S. In some embodiments, each R′ is independently —C₁-C₆alkyl.

In some embodiments, a USP9X Inhibitor is a compound of Formula II:

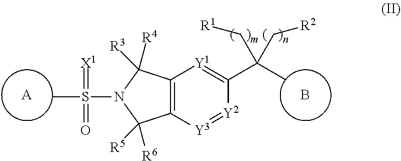

or a pharmaceutically acceptable salt thereof, wherein:
X¹ is NR or O;
Y¹ is CR⁷ or N;
Y² is CR⁸ or N;
Y³ is CR⁹ or N;
   wherein the heteroaryl formed when at least one of Y¹, Y², or Y³ is N may comprise an N-oxide;
Ring A is a monocyclic or bicyclic 3- to 12-membered ring,
   wherein the ring is saturated, fully or partially unsaturated, or aromatic, and
   wherein the ring contains 0-4 heteroatoms independently selected from the group consisting of N, O, and S, and
   wherein Ring A is optionally substituted with one or more R^{a};
each R^{a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R^{a} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
Ring B is a monocyclic or bicyclic 3- to 12-membered ring,
   wherein the ring is saturated, fully or partially unsaturated, or aromatic, and
   wherein the ring contains 0-4 heteroatoms independently selected from the group consisting of N, O, and S, and
   wherein Ring B is optionally substituted with one or more R^{b};
each R^{b} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R^{b} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R¹ and R² are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
or R¹ and R² combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
   wherein an optionally substituted R¹ and R² group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R³, R⁴, R⁵, and R⁶ are each independently selected from the group consisting of —H, optionally substituted C₁-C₆aliphatic, optionally substituted C₃-C₈cycloalkyl, and optionally substituted 3- to 8-membered heterocyclyl containing 1-4 heteroatoms independently selected from the group consisting of N, O, and S,
or R³ and R⁴, or R⁵ and R⁶, or a combination thereof, combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
   wherein an optionally substituted R³, R⁴, R⁵, and R⁶ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆ aliphatic;
R⁷, R⁸, and R⁹ are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, and optionally substituted C₁-C₆aliphatic,
   wherein an optionally substituted R⁷, R⁸, and R⁹ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
each R is independently selected from the group consisting of —H, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R group may be optionally substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
each R′ is independently selected from the group consisting of optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R′ group may be substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
m is 0, 1, or 2; and
n is 0, 1, or 2.

In some embodiments, a USP9X Inhibitor is a compound of formula II-a:

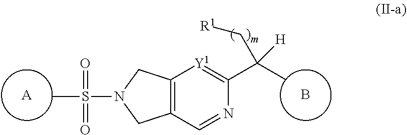

or a pharmaceutically acceptable salt thereof,
wherein Ring A, Ring B, Y¹, R¹, and m are as defined above for Formula II and described in classes and subclasses of Formula II herein, both singly and in combination.

In some embodiments, a USP9X Inhibitor is a compound of Formula II-b:

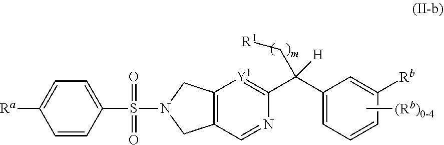

or a pharmaceutically acceptable salt thereof,
wherein Y¹, R¹, R^{a}, R^{b}, and m are as defined above for Formula II and described in classes and subclasses of Formula II herein, both singly and in combination.

In some embodiments, a USP9X Inhibitor is a compound of Formula II-c:

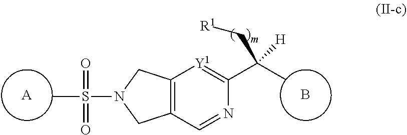

or a pharmaceutically acceptable salt thereof,
wherein Ring A, Ring B, Y¹, R¹, and m are as defined above for Formula II and described in classes and subclasses of Formula II herein, both singly and in combination.

In some embodiments, a USP9X Inhibitor is a compound of Formula II-d:

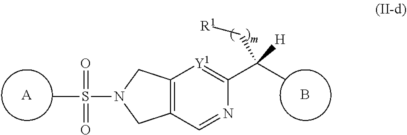

or a pharmaceutically acceptable salt thereof,
wherein Ring A, Ring B, Y¹, R¹, and m are as defined above for Formula II and described in classes and subclasses of Formula II herein, both singly and in combination.

In some embodiments, a USP9X Inhibitor is a compound of Formula II-e:

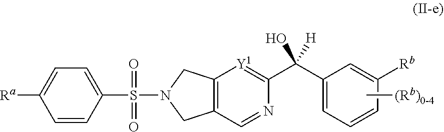

or a pharmaceutically acceptable salt thereof,
wherein Y¹, R^{a}, and R^{b} are as defined above for Formula II and described in classes and subclasses of Formula II herein, both singly and in combination.

In some embodiments, a USP9X Inhibitor is a compound of Formula II-f:

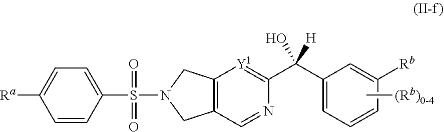

or a pharmaceutically acceptable salt thereof,
wherein Y¹, R^{a}, and R^{b} are as defined above for Formula II and described in classes and subclasses of Formula II herein, both singly and in combination.

In some embodiments of Formulas II, II-a, II-b, II-c, II-d, II-e, and II-f, Y¹ is CR⁷ or N. In some embodiments, Y¹ is CH or N. In some embodiments, Y¹ is CR⁷. In some embodiments, Y¹ is N. In some embodiments, Y¹ is CH.

In some embodiments of Formulas II, II-a, II-b, II-c, II-d, II-e, and II-f, each R^{a} is independently halogen, —OR, —NRC(O)R′, optionally substituted 3- to 10-membered heterocyclyl containing 1-4 heteroatoms independently selected from N, O, and S, or optionally substituted 5- to 10-membered heteroaryl containing 1-4 heteroatoms independently selected from N, O, and S, wherein an optionally substituted R^{a} group may be substituted with one or more halogen. In some embodiments, each R^{a} is independently halogen or optionally substituted 5- to 10-membered heteroaryl containing 1-4 heteroatoms independently selected from N, O, and S, wherein an optionally substituted R^{a} group may be substituted with one or more halogen. In some embodiments, each R^{a} is independently halogen or optionally substituted 5-membered heteroaryl containing 1-4 heteroatoms independently selected from N, O, and S, wherein an optionally substituted R^{a} group may be substituted with one or more halogen. In some embodiments, each R^{a} is

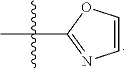 [SMILES: *c1ncco1]

In some embodiments of Formulas II, II-a, II-b, II-c, II-d, II-e, and II-f, each R^{b} is independently selected from the group consisting of halogen, —OR, optionally substituted C₁-C₆ aliphatic, and optionally substituted 3- to 10-membered heterocyclyl containing 1-4 heteroatoms independently selected from N, O, and S, wherein an optionally substituted R^{b} group may be substituted with one or more substituents independently selected from the group consisting of —NR₂ and C₁-C₆ aliphatic. In some embodiments, each R^{b} is independently selected from the group consisting of —OR, optionally substituted C₁-C₆ aliphatic, and optionally substituted 3- to 10-membered heterocyclyl containing 1-4 heteroatoms independently selected from N, O, and S, wherein an optionally substituted R^{b} group may be substituted with one or more substituents independently selected from the group consisting of —NR₂ and C₁-C₆ aliphatic. In some embodiments, each R^{b} is

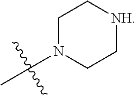 [SMILES: *N1CCNCC1]

In some embodiments of Formulas II, II-a, II-b, II-c, II-d, II-e, and II-f, R¹ is selected from the group consisting of —OR, —NR₂, —CN, —C(O)NR₂, and C₁-C₆aliphatic. In some embodiments, R¹ is selected from the group consisting of —H, —OR, —CN, and C₁-C₆aliphatic. In some embodiments, R¹ is —OR. In some embodiments, R¹ is —OR, and m is 0.

In some embodiments of Formulas II, II-a, II-b, II-c, II-d, II-e, and II-f, R⁷ is selected from the group consisting of —H, —OR, and C₁-C₆aliphatic. In some embodiments, R⁷ is —H.

In some embodiments of Formulas II, II-a, II-b, II-c, II-d, II-e, and II-f, each R is independently selected from the group consisting of —H, optionally substituted C₁-C₆aliphatic, and optionally substituted 3- to 10-membered heterocyclyl containing 1-4 heteroatoms independently selected from N, O, and S, wherein an optionally substituted R group may be optionally substituted with one or more C₁-C₆aliphatic. In some embodiments, each R is independently selected from the group consisting of —H, methyl, and 4- to 6-membered heterocyclyl containing 1-2 heteroatoms independently selected from N, O, and S optionally substituted with methyl. In some embodiments, each R is —H.

In some embodiments of Formulas II, II-a, II-b, II-c, II-d, II-e, and II-f, each R′ is independently C₁-C₆aliphatic or C₃-C₁₀cycloalkyl. In some embodiments, each R′ is independently C₃-C₁₀cycloalkyl. In some embodiments, each R′ is cyclopropyl.

In some embodiments of Formulas II, II-a, II-b, II-c, II-d, II-e, and II-f, m is 0, 1, or 2. In some embodiments, m is 0. In some embodiments, m is 0 or 1. In some embodiments, m is 0 or 2. In some embodiments, m is 1 or 2.

The compounds of the present disclosure may be made by a variety of methods, including standard chemistry. Suitable synthetic routes are described in the Examples given below.

Additionally, unless otherwise stated, structures depicted herein are also meant to include compounds that differ only in the presence of one or more isotopically enriched atoms. For example, compounds having the present structures including the replacement of hydrogen by deuterium or tritium (e.g., Examples 103-46 and 103-47), or the replacement of a carbon by a ¹³C- or ¹⁴C-enriched carbon are within the scope of this disclosure.

### Analytical Methods, Materials, and Instrumentation

Unless otherwise noted, reagents and solvents were used as received from commercial suppliers. Unless otherwise noted, reactions were conducted under an inert atmosphere of nitrogen. NMR instrument: Bruker BBFO ASCEND™400 AVANCE III 400 MHz and Bruker BBFO ULTRASHIELD™300 AVANCE III 300 MHz. Internal standard: Tetramethylsilane (TMS). MassSpec instruments and ionization method: Shimadzu LC-2020, electrospray ionization, ESI. Chromatography instruments (Reverse phase chromatography: Agela TechnologiesMP200. Preparatory HPLC (Prep-HPLC): Waters. Supercritical fluid chromatography (SFC): Shimadzu).

### Abbreviations

atm atmosphere
CbzCl Benzyl chloroformate
δ chemical shift
DCM Dichloromethane or methylene chloride
DCE 1,2-Dichloroethane
DEA Diethylamine
DIEA N,N-Diisopropylethylamine
DMAP 4-Dimethylaminopyridine
DMF N,N-Dimethylformamide
DMSO Dimethylsulfoxide
dppf 1,1′-Bis(diphenylphosphino)ferrocene
EA Ethyl acetate
EDCI N-(3-Dimethylaminopropyl)-N′-ethylcarbodiimide hydrochloride
EDTA Ethylenediaminetetraacetic acid
ee enantiomeric excess
h hour
¹H NMR proton nuclear magnetic resonance
HATU 2-(3H-[1,2,3]Triazolo[4,5-b]pyridin-3-yl)-1,1,3,3-tetramethylisouronium hexafluorophosphate
HOBT 1H-Benzo[d][1,2,3]triazol-1-ol hydrate
HPLC high performance liquid chromatography
Hz Hertz
IPA Isopropyl alcohol
LCMS liquid chromatography/mass spectrometry
m-CPBA m-Chloroperoxybenzoic acid
MeOH Methanol
min minutes
MS mass spectrometry
Pd₂(dba)₃.CH₂Cl₂ Tris(dibenzylideneacetone)dipalladium(O) chloroform adduct
Pd(dppf)Cl₂ [1,1′-Bis(diphenylphosphino)ferrocene]dichloropalladium(II)
Pd(PPh₃)₄ Tetrakis(triphenrylphosphirie)palladium (0)
PE Petroleum ether
rt room temperature
Rt retention time
RuPhos 2-Dicyclohexylphosphino-2′,6′-diisopropoxybiphenyl
RuPhos 2G Chloro(2-dicyclohexylphosphino-2′,6′-diisopropoxy-1,1′-biphenyl)[2-(2′-amino-1,1′-biphenyl)]palladium(II)
SPhos 2-Dicyclohexylphosphino-2′,6′-dimethoxybiphenyl
STAB Sodium triacetoxyborohydride
TEA Triethylamine
TFA Trifluoroacetic acid
THF Tetrahydrofuran
TLC thin layer chromatography
XPhos 2-Dicyclohexylphosphino-2′,4′,6′-triisopropylbiphenyl
XPhos 3G Methanesulfonato(2-dicyclohexylphosphino-2′,4′,6′-tri-i-propyl-1,1′-biphenyl)(2′-amino-1,1′-biphenyl-2-yl)palladium(II) dichloromethane adduct

In some embodiments, a USP9X Inhibitor has one or more of the following characteristics when tested in the Biochemical Assay of Example A:
(i) an IC₅₀ value of ≤10 μM and >1 μM;
(ii) an IC₅₀ value of ≤2 μM and >0.2 μM;
(iii) an IC₅₀ value of ≤1 μM and >0.1 μM;
(iv) an IC₅₀ value of ≤0.2 μM and >0.05 μM;
(v) an IC₅₀ value of ≤0.1 μM and >0.001 μM; and/or
(vi) an IC₅₀ value of ≤0.05 μM and >0.001 μM.

In some embodiments, a USP9X Inhibitor is a compound having an IC₅₀ value of ≤2 μM and >0.2 μM when tested in the Biochemical Assay of Example A. In some embodiments, a USP9X Inhibitor is a compound having an IC₅₀ value of ≤0.2 μM and >0.05 μM when tested in the Biochemical Assay of Example A. In some embodiments, a USP9X Inhibitor is a compound having an IC₅₀ value of ≤0.05 μM and >0.001 μM when tested in the Biochemical Assay of Example A. In some embodiments, a USP9X Inhibitor is a compound having an IC₅₀ value of ≤0.1 μM and >0.001 μM when tested in the Biochemical Assay of Example A. In some embodiments, a USP9X Inhibitor is a compound having an IC₅₀ value of ≤1 μM and >0.1 μM when tested in the Biochemical Assay of Example A. In some embodiments, a USP9X Inhibitor is a compound having an IC₅₀ value≤10 μM and >1 μM when tested in the Biochemical Assay of Example A.

In some embodiments, a USP9X Inhibitor is selected based on various characteristics of the USP9X Inhibitor, including but not limited to the IC₅₀ value in the Biochemical Assay of Example A.

In some embodiments, a USP9X Inhibitor is a compound, or pharmaceutically acceptable salt thereof, selected from:

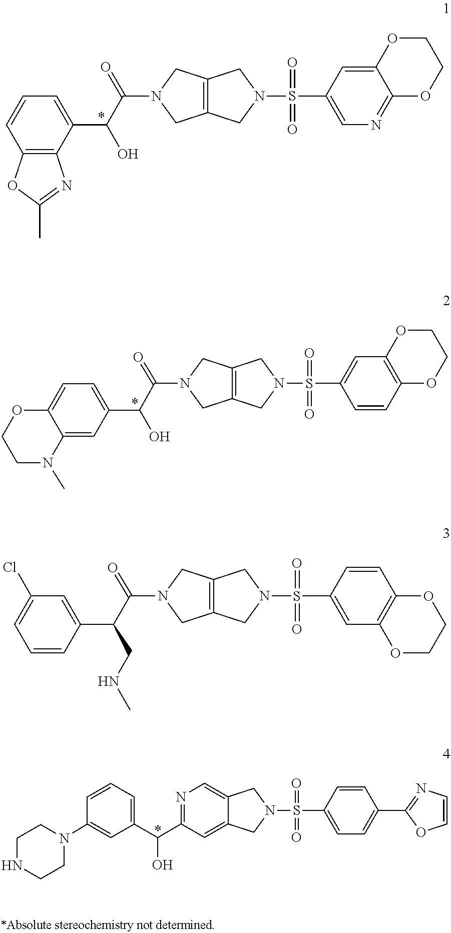 [SMILES: Cc1nc2c(C(O)C(=O)N3CC4=C(C3)CN(S(=O)(=O)c3cnc5c(c3)OCCO5)C4)cccc2o1 ; CN1CCOc2ccc(C(O)C(=O)N3CC4=C(C3)CN(S(=O)(=O)c3ccc5c(c3)OCCO5)C4)cc21 ; CNC[C@H](C(=O)N1CC2=C(C1)CN(S(=O)(=O)c1ccc3c(c1)OCCO3)C2)c1cccc(Cl)c1 ; O=S(=O)(c1ccc(-c2ncco2)cc1)N1Cc2cnc(C(O)c3cccc(N4CCNCC4)c3)cc2C1]

In some embodiments, the amount of USP9X Inhibitor administered in methods provided herein is a therapeutically effective amount.

### Immune Checkpoint Pathway Inhibitors

Checkpoint blockade therapies have produced durable clinical responses in a subset of cancers. For example, binding of the ligand PD-L1 and PD-L2 to the PD-1 receptor found on T-cells inhibits T-cell proliferation and cytokine production. Upregulation of PD-1 ligands occurs in some tumors and signaling through this pathway can contribute to inhibition of active T-cell surveillance of tumors. Therefore, therapies, such as an immune checkpoint pathway inhibitor, that bind to the PD-1 receptor and block its interaction with PD-L1 and PD-L2, prevents PD-1 pathway-mediated inhibition of the immune response and can result in decreased tumor growth.

An immune checkpoint pathway inhibitor can be selected from compounds that inhibit cytotoxic T-lymphocyte-associated protein 4 (CTLA-4) and/or programmed death 1 (PD-1). In some embodiments, the checkpoint pathway inhibitor is a CTLA-4 inhibitor. In some embodiments, the checkpoint pathway inhibitor is a PD-1 inhibitor. In some embodiments, the immune checkpoint pathway inhibitor is atezolizumab, durvalumab, ipilimumab, nivolumab, or pembrolizumab.

In some embodiments, the immune checkpoint pathway inhibitor is an antibody. In some embodiments, the immune checkpoint pathway inhibitor is anti-CTLA-4 antibody. In some embodiments, the anti-CTLA-4 antibody is imilimumab. In some embodiments, the immune checkpoint pathway inhibitor is anti-PD-1 antibody. In some embodiments, the anti-PD-1 antibody is nivolumab. In some embodiments, the anti-PD-1 antibody is pembrolizumab.

In some embodiments, the immune checkpoint pathway inhibitor is ipilimumab. Ipilimumab is a fully human IgGlK monoclonal antibody targeting CTLA-4 that inhibits the negative downstream signaling that occurs when CTLA-4 engages its ligands, CD80 and CD86, expressed on antigen presenting cells, thereby, blocking the negative down-regulation of the immune responses elicited by the interaction of these molecules. As a result, activated T cells are able to maintain their CD28 mediated signaling resulting in IL-2 secretion and proliferation of CD8 T cells in response to an antigen.

Ipilimumab is approved by the FDA for:
(a) treatment of unresectable or metastatic melanoma in adults and pediatric patients;
(b) adjuvant treatment of patients with cutaneous melanoma with pathologic involvement of regional lymph nodes of more than 1 mm who have undergone complete resection, including total lymphadenectomy;
(c) treatment of patients with intermediate or poor risk, previously untreated advanced renal cell carcinoma, in combination with nivolumab; and
(d) treatment of adult and pediatric patients 12 years of age and older with microsatellite instability-high (MSI-H) or mismatch repair deficient (dMMR) metastatic colorectal cancer that has progressed following treatment with a fluoropyrimidine, oxaliplatin, and irinotecan, in combination with nivolumab.
   Accordingly, a method of treating cancer in a patient in need thereof can comprise administering to the patient a USP9X Inhibitor, wherein the patient is receiving or has received ipilimumab, and wherein the cancer, treatment, and patient are selected from one of (a)-(d) above. Additionally, a method of treating cancer in a patient in need thereof can comprise administering to the patient ipilimumab, wherein the patient is receiving or has received a USP9X Inhibitor, and wherein the cancer, treatment, and patient are selected from one of (a)-(d) above.

In some embodiments, the immune checkpoint pathway inhibitor is nivolumab. Nivolumab is a fully human IgG4 programmed death 1 (PD-1) immune checkpoint pathway inhibitor antibody that selectively blocks the interaction of the PD-1 receptor with its two known programmed death ligands, PD-L1 and PD-L2, disrupting the negative signal that regulates T-cell activation and proliferation.

Nivolumab is approved by the FDA for:
(e) treatment of patients with unresectable or metastatic melanoma, as a single agent or in combination with ipilimumab;
(f) treatment of patients with melanoma with lymph node involvement or metastatic disease who have undergone complete resection, in the adjuvant setting;
(g) treatment of patients with metastatic non-small cell lung cancer and progression on or after platinum-based chemotherapy;
(h) treatment of patients with metastatic small cell lung cancer with progression after platinum-based chemotherapy and at least one other line of therapy;
(i) treatment of patients with advanced renal cell carcinoma who have received prior antiangiogenic therapy;
(j) treatment of patients with intermediate or poor risk, previously untreated advanced renal cell carcinoma, in combination with ipilimumab;
(k) treatment of adult patients with classical Hodgkin lymphoma that has relapsed or progressed after:
   (1) autologous hematopoietic stem cell transplantation (HSCT) and brentuximab vedotin, or
   (2) 3 or more lines of systemic therapy that includes autologous HSCT;
(l) treatment of patients with recurrent or metastatic squamous cell carcinoma of the head and neck with disease progression on or after a platinum-based therapy;
(m) treatment of patients with locally advanced or metastatic urothelial carcinoma who:
   (1) have disease progression during or following platinum-containing chemotherapy
   (2) have disease progression within 12 months of neoadjuvant or adjuvant treatment with platinum-containing chemotherapy
(n) treatment of adult and pediatric (12 years and older) patients with microsatellite instability-high (MSI-H) or mismatch repair deficient (dMMR) metastatic colorectal cancer that has progressed following treatment with a fluoropyrimidine, oxaliplatin, and irinotecan, as a single agent or in combination with ipilimumab; and
(o) treatment of patients with hepatocellular carcinoma who have been previously treated with sorafenib.
   Accordingly, a method of treating cancer in a patient in need thereof can comprise administering to the patient a USP9X Inhibitor, wherein the patient is receiving or has received nivolumab, and wherein the cancer, treatment, and patient are selected from one of (e)-(o) above. Additionally, a method of treating cancer in a patient in need thereof can comprise administering to the patient nivolumab, wherein the patient is receiving or has received a USP9X Inhibitor, and wherein the cancer, treatment, and patient are selected from one of (e)-(o) above.

In some embodiments, the immune checkpoint pathway inhibitor is pembrolizumab. Pembrolizumab is a humanized IgG4 monoclonal antibody against programmed death receptor-1 (PD-1).

Pembrolizumab is approved by the FDA for:
(p) treatment of patients with unresectable or metastatic melanoma;
(q) adjuvant treatment of patients with melanoma with involvement of lymph node(s) following complete resection;
(r) in combination with pemetrexed and platinum chemotherapy, as first-line treatment of patients with metastatic nonsquamous NSCLC, with no EGFR or ALK genomic tumor aberrations;
(s) in combination with carboplatin and either paclitaxel or nabpaclitaxel, as first-line treatment of patients with metastatic squamous NSCLC;
(t) as a single agent for first-line treatment of patients with metastatic NSCLC whose tumors have high PD-L1 expression [Tumor Proportion Score (TPS) 50%] as determined by an FDA-approved test, with no EGFR or ALK genomic tumor aberrations; and is
   (1) stage III where patients are not candidates for surgical resection or definitive chemoradiation, or
   (2) metastatic;
(u) as a single agent for treatment of patients with metastatic NSCLC whose tumors express PD-L1 (TPS≥1%) as determined by an FDA-approved test, with disease progression on or after platinum-containing chemotherapy;
(v) treatment of patients with metastatic small cell lung cancer with disease progression on or after platinum-based chemotherapy and at least one other prior line of therapy;
(w) in combination with platinum and FU for the first-line treatment of patients with metastatic or with unresectable, recurrent head and neck squamous cell cencer (HNSCC)
(x) as a single agent for the first-line treatment of patients with metastatic or with unresectable, recurrent HNSCC whose tumors express PD-L1 (Combined Positive Score (CPS)≥1) as determined by an FDA-approved test;
(y) treatment of patients with recurrent or metastatic HNSCC with disease progression on or after platinum-containing chemotherapy;
(z) treatment of adult and pediatric patients with refractory classical Hodgkin lymphoma (cHL), or who have relapsed after 3 or more prior lines of therapy;
(aa) treatment of adult and pediatric patients with refractory primary mediastinal large B-cell lymphoma (PMBCL), or who have relapsed after 2 or more prior lines of therapy;
(bb) treatment of patients with locally advanced or metastatic urothelial carcinoma who are not eligible for cisplatin-containing chemotherapy and whose tumors express PD-L1 [Combined Positive Score (CPS)≥10] as determined by an FDA-approved test, or in patients who are not eligible for any platinum-containing chemotherapy regardless of PD-L1 status;
(cc) treatment of patients with locally advanced or metastatic urothelial carcinoma who have disease progression during or following platinum-containing chemotherapy or within 12 months of neoadjuvant or adjuvant treatment with platinum-containing chemotherapy;
(dd) treatment of adult and pediatric patients with unresectable or metastatic, microsatellite instability-high (MSI-H) or mismatch repair deficient solid tumors that have progressed following prior treatment and who have no satisfactory alternative treatment options, or microsatellite instability-high (MSI-H) or mismatch repair deficient
   (1) solid tumors that have progressed following prior treatment and who have no satisfactory alternative treatment options, or
   (2) colorectal cancer that has progressed following treatment with a fluoropyrimidine, oxaliplatin, and irinotecan;
(ee) treatment of patients with recurrent locally advanced or metastatic gastric or gastroesophageal junction adenocarcinoma whose tumors express PD-L1 [Combined Positive Score (CPS)≥1] as determined by an FDA-approved test, with disease progression on or after two or more prior lines of therapy including fluoropyrimidine- and platinum-containing chemotherapy and if appropriate, HER2/neu-targeted therapy;
(t) treatment of patients with recurrent locally advanced or metastatic gastric or gastroesophageal junction adenocarcinoma whose tumors express PD-L1 (CPS≥1) as determined by an FDA-approved test, with disease progression after one or more prior lines of systemic therapy;
(ff) treatment of patients with recurrent locally advanced or metastatic squamous cell carcinoma of the esophagus whose tumors express PD-L1 (CPS≥10) as determined by an FDA-approved test, with disease progression after one or more prior lines of systemic therapy;
(gg) treatment of patients with recurrent or metastatic cervical cancer with disease progression on or after chemotherapy whose tumors express PD-L1 (CPS≥1) as determined by an FDA-approved test;
(hh) treatment of patients with hepatocellular carcinoma (HCC) who have been previously treated with sorafenib;
(ii) treatment of adult and pediatric patients with recurrent locally advanced or metastatic Merkel cell carcinoma; and
(jj) in combination with axitinib, for the first line treatment of patients with advanced renal cell carcinoma.
   Accordingly, a method of treating cancer in a patient in need thereof can comprise administering to the patient a USP9X Inhibitor, wherein the patient is receiving or has received pembrolizumab, and wherein the cancer, treatment, and patient are selected from one of (p)-(jj) above. Additionally, a method of treating cancer in a patient in need thereof can comprise administering to the patient pembrolizumab, wherein the patient is receiving or has received a USP9X Inhibitor, and wherein the cancer, treatment, and patient are selected from one of (p)-(jj) above.

In some embodiments, the immune checkpoint pathway inhibitor is atezolizumab. is a programmed cell death ligand 1 (PD-L1) blocking antibody. Atezolizumab is an Fc-engineered, humanized, non-glycosylated IgG1 kappa immunoglobulin that has a calculated molecular mass of 145 kDa.

Atezolizumab is approved by the FDA for:
(kk) treatment of adult patients with locally advanced or metastatic urothelial carcinoma who:
   (1) are not eligible for cisplatin-containing chemotherapy and whose tumors express PD-L1 (PD-L1 stained tumor-infiltrating immune cells [IC] covering ≥5% of the tumor area), as determined by an FDA-approved test, or
   (2) are not eligible for any platinum-containing chemotherapy regardless of PD-L1 status, or
   (3) have disease progression during or following any platinum-containing chemotherapy, or within 12 months of neoadjuvant or adjuvant chemotherapy;
(ll) in combination with bevacizumab, paclitaxel, and carboplatin, for the firstline treatment of adult patients with metastatic non-squamous NSCLC with no EGFR or ALK genomic tumor aberrations;
   (mm) treatment of adult patients with metastatic NSCLC who have disease progression during or following platinum-containing chemotherapy;
   (nn) in combination with paclitaxel protein-bound for the treatment of adult patients with unresectable locally advanced or metastatic TNBC whose tumors express PD-L1 (PD-L1 stained tumor-infiltrating immune cells [IC] of any intensity covering ≥1% of the tumor area), as determined by an FDA approved test; and
   (oo) in combination with carboplatin and etoposide, for the first-line treatment of adult patients with extensive-stage small cell lung cancer (ES-SCLC).
      Accordingly, a method of treating cancer in a patient in need thereof can comprise administering to the patient a USP9X Inhibitor, wherein the patient is receiving or has received pembrolizumab, and wherein the cancer, treatment, and patient are selected from one of (kk)-(oo) above. Additionally, a method of treating cancer in a patient in need thereof can comprise administering to the patient pembrolizumab, wherein the patient is receiving or has received a USP9X Inhibitor, and wherein the cancer, treatment, and patient are selected from one of (kk)-(oo) above.

In some embodiments, the immune checkpoint pathway inhibitor is durvalumab. Durvalumab is a programmed cell death ligand 1 (PD-L1) blocking antibody. Durvalumab is a human immunoglobulin G1 kappa (IgG1κ) monoclonal antibody that is produced by recombinant DNA technology in Chinese Hamster Ovary (CHO) cell suspension culture.

Durvalumab is approved by the FDA for:
(pp) treatment of patients with locally advanced or metastatic urothelial carcinoma who
   (1) have disease progression during or following platinum-containing chemotherapy
   (2) have disease progression within 12 months of neoadjuvant or adjuvant treatment with platinum-containing chemotherapy; and
(qq) treatment of patients who have unresectable, Stage III non-small cell lung cancer (NSCLC) whose disease has not progressed following concurrent platinum-based chemotherapy and radiation therapy. Accordingly, a method of treating cancer in a patient in need thereof can comprise administering to the patient a USP9X Inhibitor, wherein the patient is receiving or has received pembrolizumab, and wherein the cancer, treatment, and patient are selected from one of (pp)-(qq) above. Additionally, a method of treating cancer in a patient in need thereof can comprise administering to the patient pembrolizumab, wherein the patient is receiving or has received a USP9X Inhibitor, and wherein the cancer, treatment, and patient are selected from one of (pp)-(qq) above.

The dose of an immune checkpoint pathway inhibitor and the frequency of dosing can be selected based on various characteristics of the immune checkpoint pathway inhibitor, including the pharmacokinetic properties of the inhibitor (e.g., half-life), prior dosing regimens, and patient characteristics. In some embodiments, the amount of immune checkpoint pathway inhibitor administered in methods provided herein is a therapeutically effective amount.

In some embodiments, the method of treating cancer in a patient in need thereof comprises administering to the patient a USP9X Inhibitor, wherein the patient is receiving or has received ipilimumab. In some embodiments, a method of treating cancer in a patient in need thereof comprises administering to the patient a USP9X Inhibitor, wherein the patient is receiving or has received ipilimumab in a dose of 3 mg/kg over 90 minutes every 3 weeks for a total of 4 doses. In some embodiments, a method of treating cancer in a patient in need thereof comprises administering to the patient a USP9X Inhibitor, wherein the patient is receiving or has received ipilimumab in a dose of 10 mg/kg over 90 minutes every 3 weeks for a total of 4 doses. In some embodiments, a method of treating cancer in a patient in need thereof comprises administering to the patient a USP9X Inhibitor, wherein the patient is receiving or has received ipilimumab in a dose of 10 mg/kg over 90 minutes every 3 weeks for a total of 4 doses, followed by 10 mg/kg every 12 weeks for up to 3 years. In some embodiments, a method of treating cancer in a patient in need thereof comprises administering to the patient a USP9X Inhibitor, wherein the patient is receiving or has received ipilimumab in a dose of 1 mg/kg over 30 minutes every 3 weeks for a total of 4 doses.

In some embodiments, the method of treating cancer in a patient in need thereof comprises administering to the patient a ipilimumab, wherein the patient is receiving or has received USP9X Inhibitor. In some embodiments, a method of treating cancer in a patient in need thereof comprises administering to the patient ipilimumab in a dose of 3 mg/kg over 90 minutes every 3 weeks for a total of 4 doses, wherein the patient is receiving or has received a USP9X Inhibitor. In some embodiments, a method of treating cancer in a patient in need thereof comprises administering to the patient ipilimumab in a dose of 10 mg/kg over 90 minutes every 3 weeks for a total of 4 doses, wherein the patient is receiving or has received a USP9X Inhibitor. In some embodiments, a method of treating cancer in a patient in need thereof comprises administering to the patient ipilimumab in a dose of 10 mg/kg over 90 minutes every 3 weeks for a total of 4 doses, followed by 10 mg/kg every 12 weeks for up to 3 years, wherein the patient is receiving or has received a USP9X Inhibitor. In some embodiments, a method of treating cancer in a patient in need thereof comprises administering to the patient ipilimumab in a dose of 1 mg/kg over 30 minutes every 3 weeks for a total of 4 doses, wherein the patient is receiving or has received a USP9X Inhibitor.

In some embodiments, the method of treating cancer in a patient in need thereof comprises administering to the patient a USP9X Inhibitor, wherein the patient is receiving or has received nivolumab. In some embodiments, a method of treating cancer in a patient in need thereof comprises administering to the patient a USP9X Inhibitor, wherein the patient is receiving or has received nivolumab in a dose of 3 mg/kg over 60 minutes every 2 weeks. In some embodiments, the method of treating cancer in a patient in need thereof comprises administering to the patient a USP9X Inhibitor, wherein the patient is receiving or has received nivolumab in a dose of 3 mg/kg over 30 minutes.

In some embodiments, the method of treating cancer in a patient in need thereof comprises administering to the patient a nivolumab, wherein the patient is receiving or has received USP9X Inhibitor. In some embodiments, a method of treating cancer in a patient in need thereof comprises administering to the patient nivolumab in a dose of 3 mg/kg over 60 minutes every 2 weeks, wherein the patient is receiving or has received USP9X Inhibitor. In some embodiments, a method of treating cancer in a patient in need thereof comprises administering to the patient nivolumab in a dose of 3 mg/kg over 30 minutes, wherein the patient is receiving or has received USP9X Inhibitor.

In some embodiments, the method of treating cancer in a patient in need thereof comprises administering to the patient a USP9X Inhibitor, wherein the patient is receiving or has received ipilimumab and nivolumab. In some embodiments, the method of treating cancer in a patient in need thereof comprises administering to the patient a USP9X Inhibitor, wherein the patient is receiving or has received nivolumab in a dose of 3 mg/kg over 30 minutes followed by ipilimumab in a dose of 1 mg/kg over 30 minutes on the same day, every 3 weeks for a total of 4 doses. In some embodiments, the method of treating cancer in a patient in need thereof comprises administering to the patient a USP9X Inhibitor, wherein the patient is receiving or has received nivolumab in a dose of 3 mg/kg over 30 minutes followed by ipilimumab in a dose of 1 mg/kg over 30 minutes on the same day, every 3 weeks for a total of 4 doses, then nivolumab in a dose of 240 mg every 2 weeks over 30 minutes. In some embodiments, the method of treating cancer in a patient in need thereof comprises administering to the patient a USP9X Inhibitor, wherein the patient is receiving or has received nivolumab in a dose of 3 mg/kg over 30 minutes followed by ipilimumab in a dose of 1 mg/kg over 30 minutes on the same day, every 3 weeks for a total of 4 doses, then nivolumab in a dose of 480 mg every 4 weeks over 30 minutes.

In some embodiments, the method of treating cancer in a patient in need thereof comprises administering to the patient ipilimumab and nivolumab, wherein the patient is receiving or has received a USP9X Inhibitor. In some embodiments, a method of treating cancer in a patient in need thereof comprises administering to the patient nivolumab in a dose of 3 mg/kg over 30 minutes and (e.g., followed by) ipilimumab in a dose of 1 mg/kg over 30 minutes on the same day, every 3 weeks for a total of 4 doses, wherein the patient is receiving or has received a USP9X Inhibitor. In some embodiments, a method of treating cancer in a patient in need thereof comprises administering to the patient nivolumab in a dose of 3 mg/kg over 30 minutes and (e.g., followed by) ipilimumab in a dose of 1 mg/kg over 30 minutes on the same day, every 3 weeks for a total of 4 doses, then nivolumab in a dose of 240 mg every 2 weeks over 30 minutes, wherein the patient is receiving or has received a USP9X Inhibitor. In some embodiments, a method of treating cancer in a patient in need thereof comprises administering to the patient nivolumab in a dose of 3 mg/kg over 30 minutes and (e.g., followed by) ipilimumab in a dose of 1 mg/kg over 30 minutes on the same day, every 3 weeks for a total of 4 doses, then nivolumab in a dose of 480 mg every 4 weeks over 30 minutes, wherein the patient is receiving or has received a USP9X Inhibitor.

In some embodiments, the method of treating cancer in a patient in need thereof comprises administering to the patient a USP9X Inhibitor, wherein the patient is receiving or has received pembrolizumab. In some embodiments, the method of treating cancer in a patient in need thereof comprises administering to the patient a USP9X Inhibitor, wherein the patient is receiving or has received pembrolizumab in a dose of 200 mg every 3 weeks. In some embodiments, the method of treating cancer in a patient in need thereof comprises administering to the patient a USP9X Inhibitor, wherein the patient is receiving or has received pembrolizumab in a dose of 2 mg/kg over 30 minutes every 3 weeks.

In some embodiments, the method of treating cancer in a patient in need thereof comprises administering to the patient pembrolizumab, wherein the patient is receiving or has received a USP9X Inhibitor. In some embodiments, the method of treating cancer in a patient in need thereof comprises administering to the patient pembrolizumab in a dose of 200 mg every 3 weeks, wherein the patient is receiving or has received a USP9X Inhibitor. In some embodiments, the method of treating cancer in a patient in need thereof comprises administering to the patient pembrolizumab in a dose of 2 mg/kg over 30 minutes every 3 weeks, wherein the patient is receiving or has received a USP9X Inhibitor.

### EXAMPLES

### Example 1: USP9X Inhibition Promotes T Cell Cytokine Production by Reducing T Cell Tolerance in Restimulated PBMC Assay

In this Example, human peripheral blood mononuclear cells (PBMCs) were stimulated with Staphylococcal enterotoxin B (SEB) to induce inactive T cells in vitro. PBMCs were then washed to remove SEB and allowed to rest for 2 days in the presence or absence of an agent (e.g., a USP9X Inhibitor, anti-CTLA4 antibody, or anti-PD1 antibody). The supernatant was collected for IFN-γ measurement, and cell pellets were collected for Western blot analysis.

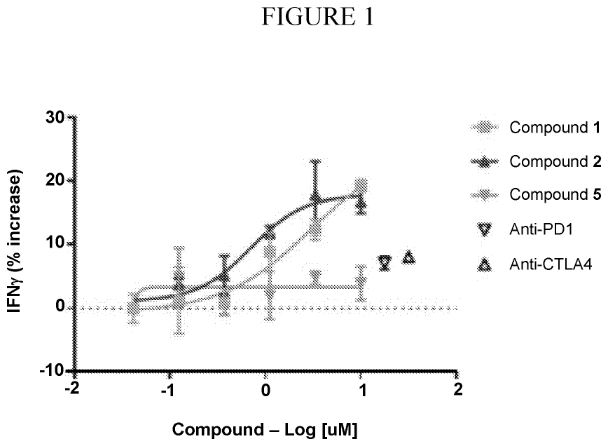

Production of IFNγ was restored in a concentration-dependent manner by USP9X Inhibitors 1 and 2 and was not restored by the negative control compound 5, anti-CTLA4 antibody, or anti-PD1 antibody (FIG. 1). The estimated EC₅₀ potencies for 1 and 2 were consistent with their known biochemical IC₅₀ values.

As used herein a “negative control compound” is a compound with an IC₅₀ value of >12 μM in the Biochemical Assay of Example A. In some embodiments, a negative control compound is compound 5.

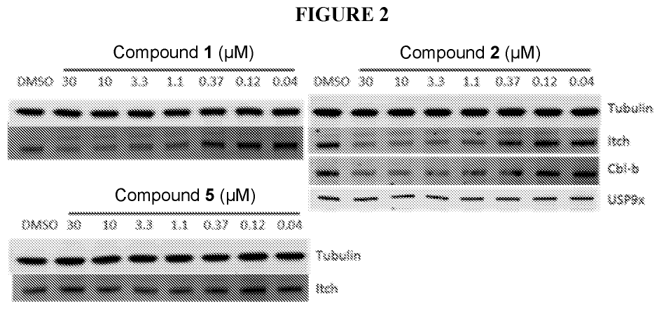

Further, concentration-dependent reductions of ITCH and Cbl-b protein levels were observed in the presence of USP9X Inhibitors 1 and 2 (FIG. 2). Both ITCH and Cbl-b are known key regulators of T cell tolerance. Importantly, these changes correlated with EC₅₀ values for IFNγ secretion, suggesting target engagement-dependent biology. In this experiment, USP9X Inhibitor 1 had an EC₅₀ value≤10 μM and >1 μM; USP9X Inhibitor 2 had an EC₅₀ value≤1 μM and >0.1 μM; and negative control compound 5 had an EC₅₀ value>30 μM.

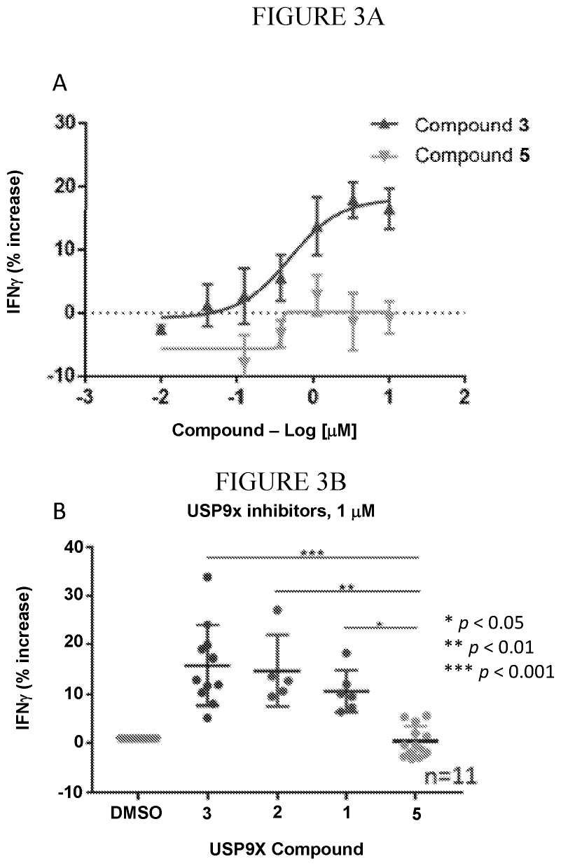

In addition, USP9X Inhibitor 3 also promoted IFNγ secretion in SEB-stimulated PBMCs in a concentration-dependent manner (EC₅₀≤1 μM and >0.1 μM), whereas negative control compound 5 did not have any significant effect (FIG. 3A). In a study of PBMCs from 11 donors, three USP9X Inhibitors (3, 2, and 1) were shown to significantly increase IFNγ production compared to DMSO or negative control compound 5 (FIG. 3B).

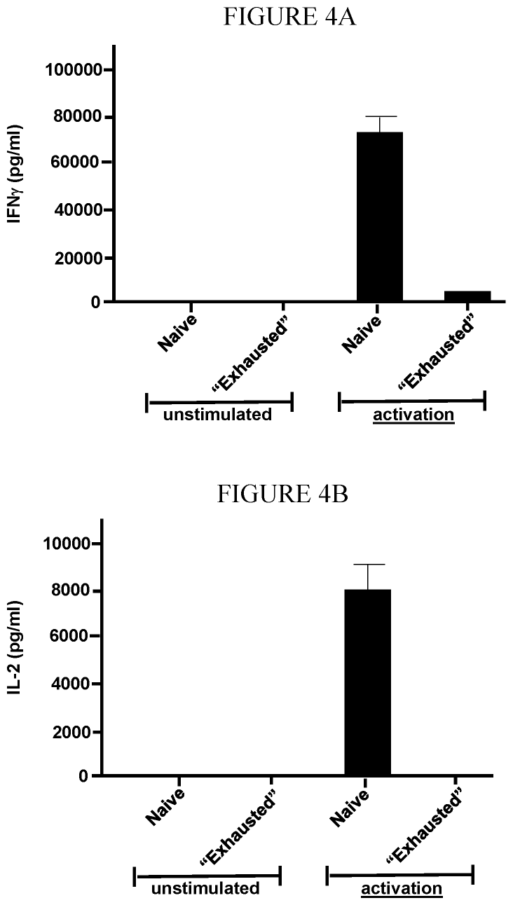

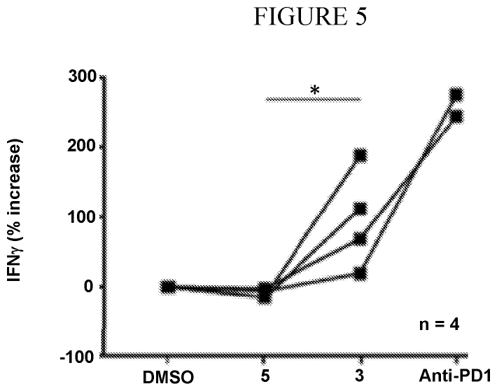

The effect of USP9X inhibition on PBMCs that have been re-stimulated with SEB was also investigated. Naïve PBMCs produced robust increases in IFNγ (FIG. 4A) or interleukin (IL)-2 (FIG. 4B), while SEB-restimulated PBMCs did not show cytokine induction. This is consistent with the notion that the SEB-restimulated T cells were in a state of exhaustion or rendered tolerant to SEB. Treatment of SEB-restimulated PBMCs with USP9X Inhibitor 3 or an anti-PD1 antibody restored IFNγ production to varying degrees depending on the donor (FIG. 5).

Taken together, these results suggest USP9X inhibition can rescue T cell restimulation by upregulating IFNγ secretion and by downregulating ITCH and Cbl-b levels.

### Example 2: USP9X Inhibition Promotes IFN-γ Production in CD3/CD28 Activated T Cells and MLR Assay

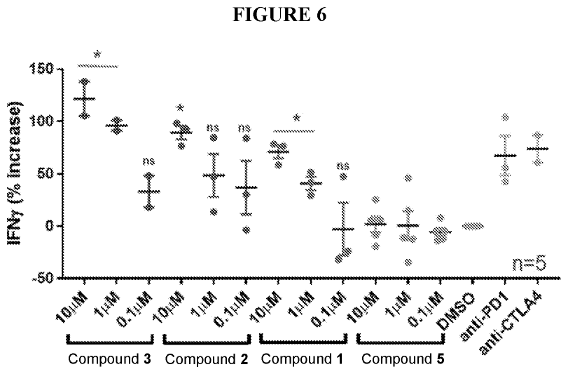

Given the reported role of ITCH and Cbl-b in T cell tolerance and as a negative regulator of T-cell receptor (TCR)/catalytic domain 28 (CD28) signaling, the effects of USP9X inhibition were evaluated in a CD3/CD28 activation assay and in a mixed lymphocyte reaction (MLR) assay. In an anti-CD3/CD28 T cell activation assay, several USP9X inhibitors demonstrated enhanced IFNγ production, with USP9X Inhibitor 3 yielding the most profound effect (FIG. 6). Positive controls, anti-PD1 and anti-CTLA4 antibodies also increased IFNγ production, whereas negative control compound 5 showed weak to no activity.

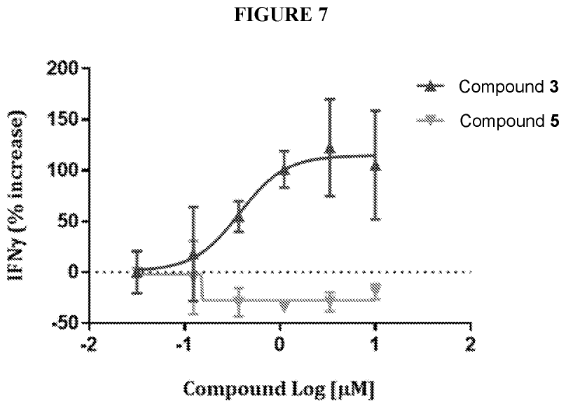

USP9X Inhibitor 3 also promoted IFNγ secretion in the MLR assay in a concentration-dependent manner (EC₅₀≤1 μM and >0.1 μM), whereas negative control compound 5 did not have any significant effect (FIG. 7). These results further support a negative regulatory role for USP9X in T cell activation.

### Example 3: Increased IFN-γ and IL-2 Production from Allogenic CD4+ T Cells in MLR Assay Following USP9X Inhibition

Activation of allogenic CD4+ T cells cultured with allogenic dendritic cells (DCs) in the presence or absence of a USP9X Inhibitor was determined in an MLR assay. Monocytes were first isolated from healthy human PBMCs using magnetic beads and plated in RPMI 1640 medium with 10% fetal bovine serum (FBS) for dendritic cell maturation. Monocytes were then cultured with granulocyte-macrophage colony-stimulating factor (GM-CSF) and interleukin (IL)-4 (20 ng/mL; RnD systems) in order to induce formation of immature dendritic cells. Cytokines were added every other day. After 5-6 days of culture, a maturation cocktail containing 100 ng/ml tumor necrosis factor alpha (TNFa), IL-6, and IL-1β, as well as 1 μg/mL prostaglandin E2 (PGE2) was added to the culture medium for 24 hours. 145,000 matured DCs per well were added to a 96 well plate. Allogeneic CD4+ T cells were then isolated and added to 70 μL of diluted compounds in a fresh 96 well plate. 70 μL of CD4+ cells were added at a concentration of 1.45 million cells per well, resulting in a 10:1 CD4+T:DC ratio in the final experiment. CD4+ T cells were pre-incubated with agent (e.g., USP9X Inhibitor, anti-PD-1 antibody, or anti-CTLA-4 antibody) for 1 hour at 37° C. After pre-incubation, 70 μL of pre-diluted DCs were added to the CD4/compound plate. The co-culture was incubated for 4 days. The supernatant was removed and analyzed for IFNγ and IL-2 using Meso Scale Discovery Immunoassay (Meso Scale Discovery).

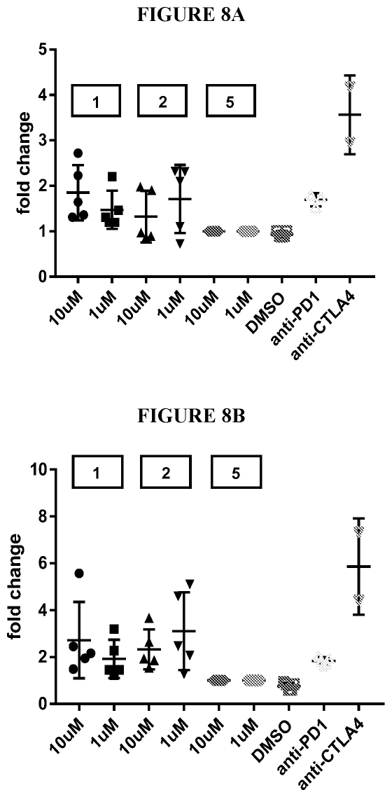

USP9X Inhibitors 1 and 2 enhanced IL-2 (FIG. 8A) and IFNγ production (FIG. 8B). Positive controls, i.e., anti-PD-1 and anti-CTLA-4 antibodies, also increased IFNγ and IL-2 production, whereas negative control compound 5 showed no activity.

### Example 4: USP9X Inhibition Enhances Allogeneic PBMC-Mediated Cytotoxicity of A375 Melanoma Cells

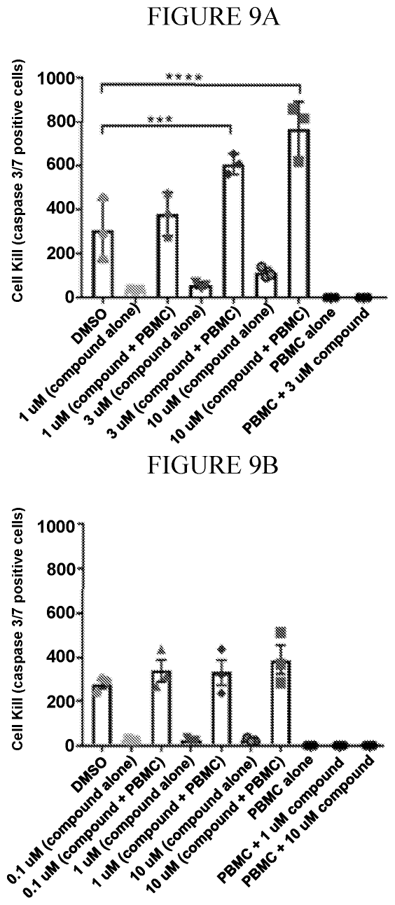

To determine if USP9X inhibition could potentiate immune-mediated cytotoxicity against tumor cells, human PBMCs were rendered inactive by SEB stimulation prior to incubation with A375 melanoma cells in the presence of DMSO, USP9X Inhibitor 3, or a negative control, and apoptosis of the A375 melanoma cells was evaluated. USP9X Inhibitor 3 enhanced A375 apoptosis in a concentration-dependent manner as indicated by caspase 3/7 activity (FIG. 9A), while no enhancement of apoptosis was observed with negative control compound 5 (FIG. 9B). A375 cells incubated alone, in the absence of SEB-stimulated PMBCs, with either compound had no significant effect on cellular apoptosis.

### Example 5: USP9X Inhibition Enhances Tumor Cell Killing in an Caspase 3/7 Apoptosis Assay

A-375 tumor cells were cultured in 96 well plates overnight in DMEM/10% FBS such that a density of 10,000 cells per well was reached. After 24 hours, media was removed and replaced with Dulbecco's Modified Eagle Medium (DMEM) with 10% FBS containing 1× IncuCyte® Caspase 3/7 Apoptosis Assay Reagent, agent, and PBMCs from healthy human donors. PBMCs were added in a 20:1 ratio relative to A-375 cell number. As a positive control, anti-CD3 (0.1 ug/mL) and IL-2 (10 ng/mL) were added to 2 wells per plate. Plates were incubated and imaged in an IncuCyte® Live Cell Imaging System (Essen Biosciences) for 4 days. Apoptotic A-375 cells were counted via an IncuCyte® image analysis algorithm.

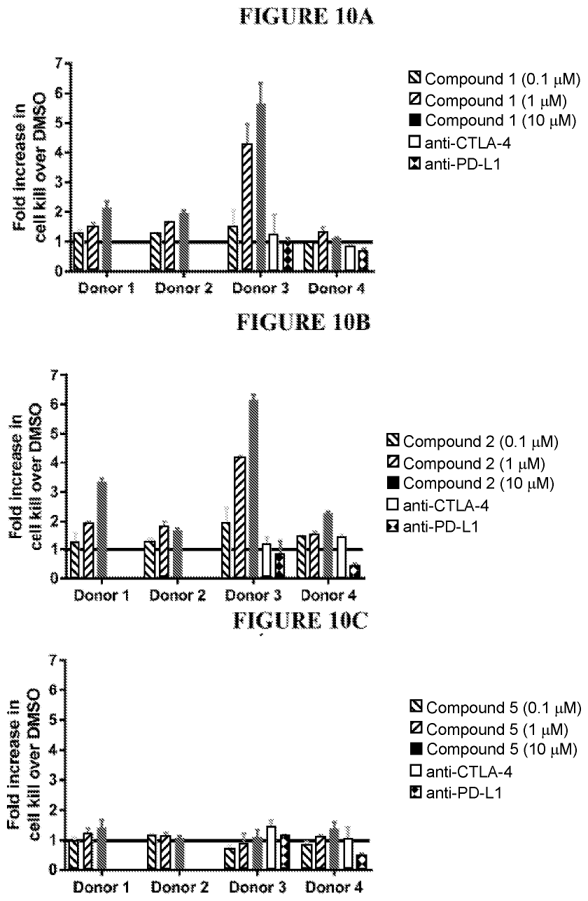

Both USP9X Inhibitor 1 (FIG. 10A, 3 out of 4 donors) and USP9X Inhibitor 2 (FIG. 10B, 4 out of 4 donors) showed an increased immune cell kill of A-375 cells in a dose-dependent manner, whereas negative control compound 5 showed weak or no activity (FIG. 10C). These results suggest that USP9X inhibition can effect immune-mediated cytotoxicity against tumor cells.

### Example 6: Human T Cell Activation with USP9X Inhibitors

Human total T cells (CD3+) were isolated using EasySep™ immunomagnetic beads (Stemcell Technologies) from healthy human PBMCs (Hemacare). Cells were stained with Cell Trace Violet proliferation dye (Invitrogen), and treated with agent or DMSO. Cells were also stimulated with a CD3/CD28 activator (Stemcell Technologies). After 4 days of incubation, cells and supernatant were collected; cells were washed with staining buffer (PBS with 2% FBS) twice and centrifuged (350 g for 5 min). Cells were stained with LIVE/DEAD stain (Invitrogen), FITC-CD4 (BD Pharmingen) and CD8 (BD Pharmingen) in 100 μL volume for 30 mins at 4° C. Cells were washed twice as described above, resuspended in 100 μL staining buffer and analyzed using a BD Canto II (BD Biosciences). Data was analyzed using FlowJo V10 (FlowJo LLC). Secreted IFNγ was measured by ELISA (Dakewe Biotech Co., Ltd). All data were analyzed by Graphpad Prism 6.0 (GraphPad Software).

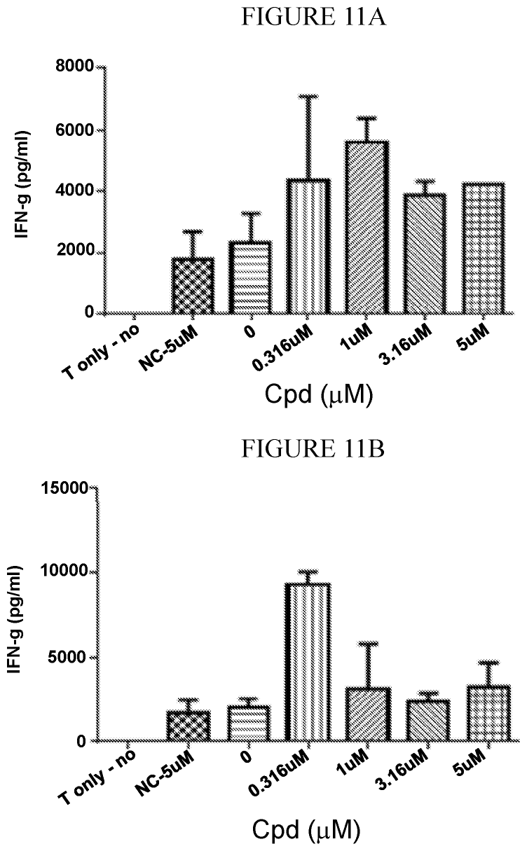

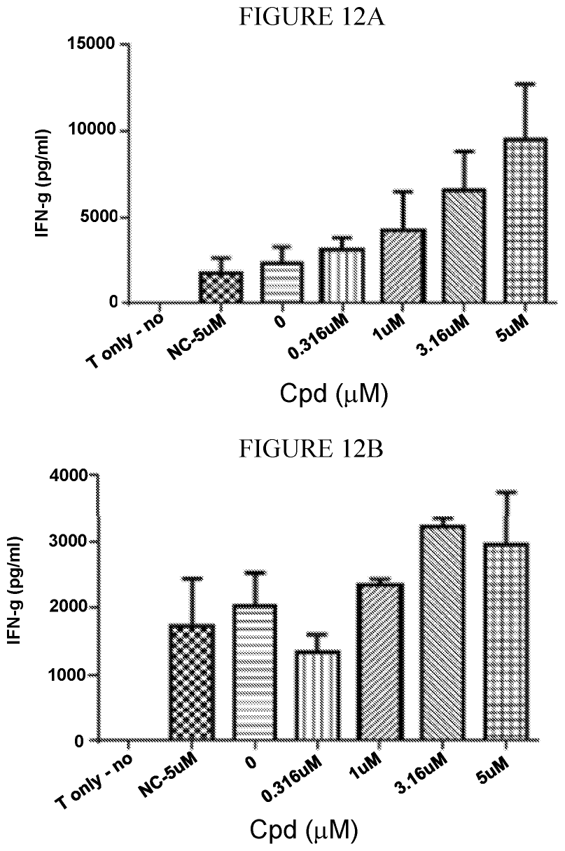

IFNγ production was increased in the presence of USP9X Inhibitors 4 (FIGS. 11A and 111B) and 3 (FIGS. 12A and 12B) across two donors, whereas little to no IFNγ secretion was observed with the negative control (NC) compound 5.

### Example 7: USP9X Inhibition Reactivated T Cells In Vivo

Peripheral tolerance/inactivation was induced in female BALB/c mice by a single intraperitoneal injection of 30 μg SEB. Mice were treated with a positive control (anti-CTLA4) or various doses of USP9X Inhibitor 3. Spleens and splenocytes were harvested at 24 hr after the last dose. Spleen protein levels of Cbl-b and ITCH were reduced 81% and 64%, respectively in the highest dose group, and there were no notable changes at the lower doses. No changes in IL-2 and IFNγ levels were detected in the supernatants from SEB-restimulated splenocytes. Splenocytes isolated from SEB+anti-CTLA4-treated mice showed a significant reduction of Cbl-b and ITCH and significant increase in IL-2 and IFNγ levels. These results suggest target engagement of USP9X occurs in vivo.

### Example 8: Combination Therapy of USP9X and Anti-CTLA-4 and Anti-PDL-1

The effect of a combination of USP9X Inhibitor and an immune checkpoint pathway inhibitor on tumor cells was evaluated. A-375 tumor cells were cultured in 96 well plates overnight in DMEM with 10% FBS such that a density of 10,000 cells per well was reached. After 24 hours, media was removed and replaced with DMEM with 10% FBS containing 1X IncuCyte® Caspase 3/7 Apoptosis Assay Reagent, agent, and PBMCs from healthy human donors. The agent was selected from USP9X Inhibitor 2 alone, anti-CTLA-4 alone, or a combination thereof. PBMCs were added in a 5:1, 10:1, or 20:1 ratio relative to A-375 cell number. As a positive control, anti-CD3 (0.1 ug/mL) and IL-2 (10 ng/mL) were added to 2 wells per plate. Plates were incubated and imaged in an IncuCyte® Live Cell Imaging System (Essen Biosciences) for 4 days. Apoptotic A-375 cells were counted via an IncuCyte® image analysis algorithm.

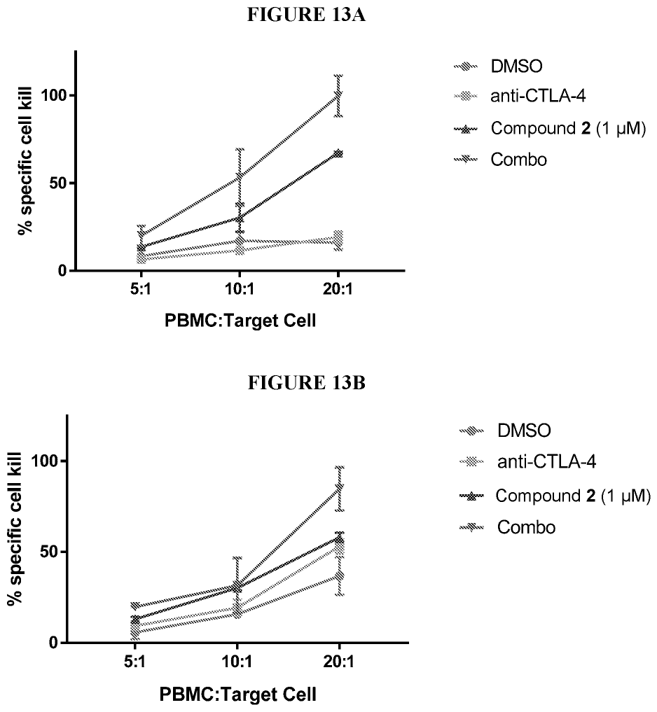

As can be seen in FIGS. 13A and 13B, a combination of anti-CTLA-4 and USP9X Inhibitor 2 is more effective than either single agent alone for at least one cell ratio point tested in each donor. Furthermore, USP9X Inhibitor 2 alone outperforms anti-CTLA-4 alone in all donors tested.

### Example 9. Synthesis of USP9X Inhibitor 1 and Negative Control Compound 5

### Synthesis of Intermediate A1

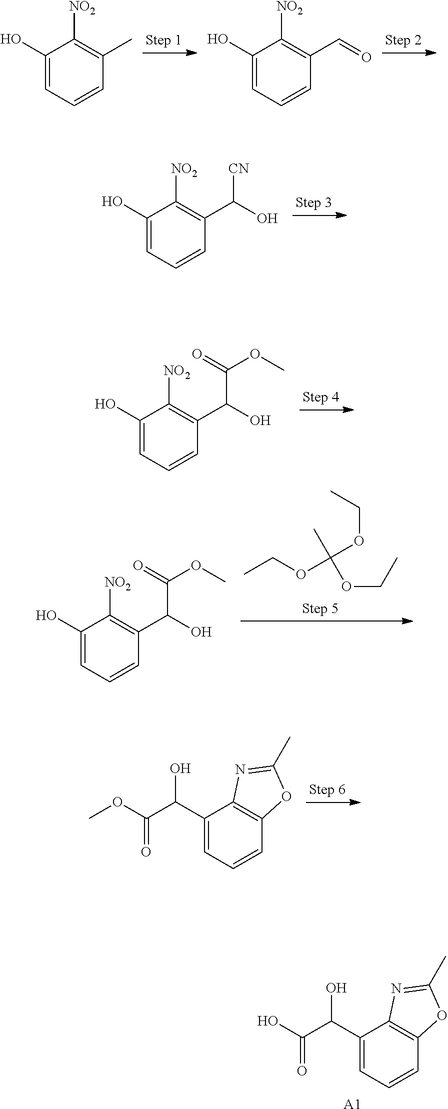

### Step 1. 3-hydroxy-2-nitrobenzaldehyde

To a solution of 3-methyl-2-nitrophenol (200 g, 1.29 mol) in acetic anhydride (1600 mL) was added sulfuric acid (240 mL) and acetic acid (1620 mL). This was followed by the addition of chromium trioxide (280 g, 2.77 mol) in several batches with stirring at 0° C. The resulting mixture was stirred for 2.5 h at 0° C. and then poured into ice/water (5000 mL). The solids were collected by filtration and then washed with water (3×1 L), saturated sodium carbonate solution (3×800 mL), and water (3×1 L). The solids were dissolved in ethanol (380 mL) and concentrated hydrochloric acid (617 mL). The resulting solution was stirred for 1.5 h at 110° C. and then cooled to room temperature. The reaction mixture was concentrated under vacuum to afford 3-hydroxy-2-nitrobenzaldehyde as a yellow solid (38.0 g, 18%). LCMS (ES, m/z): 166 [M−H]⁻.

### Step 2. 2-hydroxy-2-(3-hydroxy-2-nitrophenyl)acetonitrile

To a solution of 3-hydroxy-2-nitrobenzaldehyde (38.0 g, 204 mmol) in dichloromethane (500 mL) was added ZnI2 (14.5 g, 44.5 mmol). The reaction was treated with trimethylsilyl cyanide (100 mL, 708 mmol) added dropwise with stirring at 0° C. The resulting mixture was stirred for 2.5 h at 25° C. The reaction was poured into brine (200 mL) and then extracted with ethyl acetate (3×500 mL). The combined organic layers were dried over anhydrous sodium sulfate, filtered, and concentrated under vacuum to afford 2-hydroxy-2-(3-hydroxy-2-nitrophenyl)acetonitrile as a yellow solid (34.0 g, 73%). LCMS (ES, m/z): 195 [M+H]⁺.

### Step 3. Methyl 2-hydroxy-2-(3-hydroxy-2-nitrophenyl)acetate

To a solution of 2-hydroxy-2-(3-hydroxy-2-nitrophenyl)acetonitrile (34.0 g, 157 mmol) in methanol (80 mL) was added hydrochloric acid (80 mL, 4 N in 1,4-dioxane). The resulting solution was stirred for 45 min at 60° C. and cooled to room temperature. The reaction mixture was concentrated under vacuum and purified by silica gel chromatography (eluting with 0:100 to 35:65 ethyl acetate/petroleum ether) to afford methyl 2-hydroxy-2-(3-hydroxy-2-nitrophenyl)acetate as a yellow solid (23.0 g, 58%). LCMS (ES, m/z): 228 [M+H]⁺.

### Step 4. Methyl 2-(2-amino-3-hydroxyphenyl)-2-hydroxyacetate

To a solution of methyl 2-hydroxy-2-(3-hydroxy-2-nitrophenyl)acetate (23.0 g, 0.11 mol) in methanol (500 mL) was added anhydrous palladium carbon (2.3 g, 10 wt % Pd). The resulting mixture was stirred for 16 h at 25° C. under hydrogen atmosphere (3 atm). The reaction mixture was filtered and concentrated under vacuum to afford methyl 2-(2-amino-3-hydroxyphenyl)-2-hydroxyacetate as a yellow solid (14.0 g, 60%). LCMS (ES, m/z): 198 [M+H]⁺.

### Step 5. Methyl 2-hydroxy-2-(2-methyl-1,3-benzoxazol-4-yl)acetate

To a solution of methyl 2-(2-amino-3-hydroxyphenyl)-2-hydroxyacetate (9.0 g, 43.4 mmol) in 1,1,1-triethoxyethane (150 mL) was added bismuth (III) trifluoromethanesulfonate (800 mg, 1.18 mmol). The resulting mixture was stirred for 10 min at 85° C. After cooling to room temperature, the reaction mixture was concentrated under vacuum. The resulting crude product was purified by silica gel chromatography (eluting with 0:100 to 50:50 ethyl acetate/petroleum ether) to afford methyl 2-hydroxy-2-(2-methyl-1,3-benzoxazol-4-yl)acetate as a white solid (6.3 g, 63%). LCMS (ES, m/z): 222 [M+H]⁺.

### Step 6. 2-hydroxy-2-(2-methyl-1,3-benzoxazol-4-yl)acetic acid (A1)

To a solution of methyl 2-hydroxy-2-(2-methyl-1,3-benzoxazol-4-yl)acetate (500 mg, 2.26 mmol) in tetrahydrofuran (20 mL) and water (2 mL) was added lithium hydroxide (271 mg, 11.3 mmol). The resulting mixture was stirred for 1 h at room temperature. The reaction mixture was washed with diethyl ether (1×10 mL) and then acidified to pH=6 with hydrochloric acid (1 N). The resulting solution was extracted with ethyl acetate (2×20 mL). The combined organic layers were dried over anhydrous sodium sulfate, filtered, and concentrated under vacuum to afford 2-hydroxy-2-(2-methyl-1,3-benzoxazol-4-yl)acetic acid as a white solid (386 mg, 82%). LCMS (ES, m/z): 208 [M+H]⁺.

### Synthesis of Intermediate B1

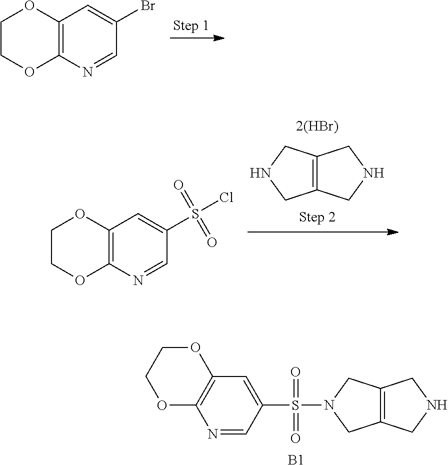

### Step 1. 2H,3H-[1,4]dioxino[2,3-b]pyridine-7-sulfonyl chloride

To a solution of n-BuLi (2.0 mL, 2.5 M in hexane) was added n-Bu₂Mg (4.8 mL, 1.0 M in heptane). The resulting mixture was stirred for 10 min at room temperature. The reaction was treated with 7-bromo-2H,3H-[1,4]dioxino[2,3-b]pyridine (2.0 g, 9.26 mmol) in tetrahydrofuran (16 mL) added dropwise with stirring at −10° C. over a period of 10 min. The mixture was stirred for 1 h at −10° C. and then slowly added to a solution of sulfuryl dichloride (16 mL, 0.20 mol) in toluene (16 mL) at −10° C. and stirred for an additional 1 h. The reaction was quenched by the careful addition of saturated aqueous ammonium chloride solution (30 mL) at 0° C. The product was extracted with dichloromethane (3×50 mL). The organic layers were combined, dried over anhydrous sodium sulfate, filtered and concentrated under vacuum. The resulting crude product was purified by silica gel column (eluted with 1:3 ethyl acetate/petroleum ether) to afford 2H,3H-[1,4]dioxino[2,3-b]pyridine-7-sulfonyl chloride as a white solid (1.3 g, 60%). LCMS: (ES, m/z): 236, 238 [M+H]⁺.

### Step 2. 7-(4,5-dihydropyrrolo[3,4-c]pyrrol-2(1H,3H,4H)-ylsulfonyl)-2,3-dihydro-[1,4]dioxino[2,3-b]pyridine (B1)

To a solution of 1H-imidazole (14.5 g, 212 mmol) in dichloromethane (140 mL) was added 2H,3H-[1,4]dioxino[2,3-b]pyridine-7-sulfonyl chloride (25.0 g, 96 mmol) in dichloromethane (250 mL) dropwise with stirring at 0° C. The resulting mixture was stirred for 2 h at room temperature and then filtered and concentrated under vacuum. The solids were dissolved in absolute ethanol (125 mL) and added dropwise to a solution of 1H,2H,3H,4H,5H,6H-pyrrolo[3,4-c]pyrrole dihydrobromide (86.8 g, 319 mmol) in water (125 mL). The reaction was stirred for 18 h at room temperature and then 48 h at 60° C. After cooling to room temperature, the mixture was rendered basic (pH=14) with aqueous sodium hydroxide (50 wt %). The product was extracted with dichloromethane (3×300 mL). The organic layers were combined, dried over anhydrous sodium sulfate, filtered and concentrated under vacuum to afford of 2-[2H,3H-[1,4]dioxino[2,3-b]pyridine-7-sulfonyl]-1H,2H,3H,4H,5H,6H-pyrrolo[3,4-c]pyrrole as a yellow solid (13 g, 39.5%). LCMS: (ES, m/z): 310 [M+H]⁺.

### Synthesis of Compounds 1 and 5

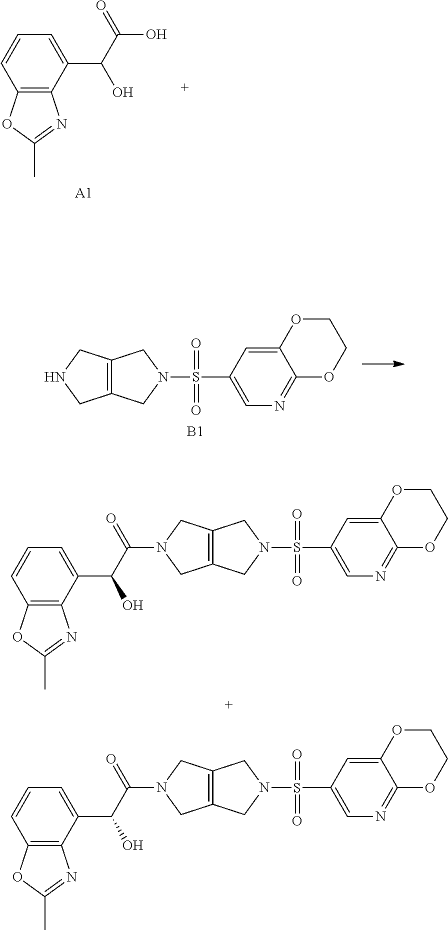

To a solution of 7-(4,5-dihydropyrrolo[3,4-c]pyrrol-2(1H,3H,4H)-ylsulfonyl)-2,3-dihydro-[1,4]dioxino[2,3-b]pyridine (B1) (1 equiv) in N,N-dimethylformamide was added 2-hydroxy-2-(2-methyl-1,3-benzoxazol-4-yl)acetic acid (A1) (1 equiv), DIEA (2 equiv), HOBt (1.1 equiv) and EDCI (1.1 equiv). The resulting mixture was stirred for 2 h at room temperature and poured into water. The resulting solution was extracted with EtOAc. The combined organic layers were dried over anhydrous sodium sulfate, filtered and concentrated under vacuum. The resulting crude product was purified by prep-TLC. ¹H-NMR: (DMSO-d₆, 400 MHz) δ (ppm): 8.16 (d, J=2.4 Hz, 1H), 7.63-7.58 (m, 2H), 7.33-7.32 (m, 2H), 5.70 (s, 2H), 4.52-4.50 (m, 2H), 4.34-4.32 (m, 3H), 4.09-4.05 (m, 7H), 2.63 (s, 3H). The two enantiomers were separated by Chiral Prep-HPLC (Column: CHIRALPAK IF, 5 μm, 20×250 mm; Mobile Phase, A: DCM and B: MeOH (keep 60% B over 18 min); Flow rate: 16 mL/min; Detector: UV 254/220 nm; Retention time: 1^{st} eluting isomer (1), 11.23 min; 2^{nd} eluting isomer (5), 15.39 min).

### Example 10. Synthesis of USP9X Inhibitor 2

### Synthesis of Intermediate A2

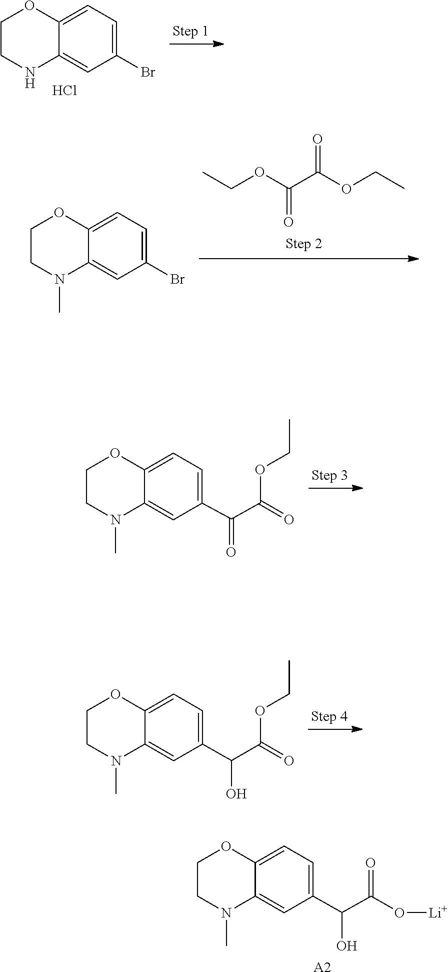

### Step 1. 6-bromo-4-methyl-3,4-dihydro-2H-1,4-benzoxazine

To a solution of 6-bromo-3,4-dihydro-2H-1,4-benzoxazine (5.00 g, 20.1 mmol) in CH₃CN (150 mL) was added paraformaldehyde (3.26 g, 40.2 mmol) and sodium cyanoborohydride (2.30 g, 36.49 mmol). The resulting mixture was stirred for 15 min at 0° C. The reaction was treated with acetic acid (5 mL) and stirred for 16 h at room temperature. The reaction was quenched with water (50 mL) and then extracted with ethyl acetate (3×150 mL). The combined organic layers were dried over anhydrous sodium sulfate, filtered, and concentrated under vacuum. The resulting crude product was purified by silica gel chromatography (eluting with 0:100 to 80:20 ethyl acetate/petroleum ether) to afford 6-bromo-4-methyl-3,4-dihydro-2H-1,4-benzoxazine as a red oil (2.50 g, 55%). LCMS (ES, m/z): 228, 230 [M+H]⁺.

### Step 2. Ethyl 2-(4-methyl-3,4-dihydro-2H-1,4-benzoxazin-6-yl)-2-oxoacetate

To a solution of 6-bromo-4-methyl-3,4-dihydro-2H-1,4-benzoxazine (2.50 g, 11.0 mmol) in THF (25 mL) was added n-BuLi (13.2 mL, 2.5 M in n-hexane) dropwise with stirring at −78° C. After 15 min diethyl oxalate (4.46 mL, 33.0 mmol) was added and stirring continued for 2 h. The reaction was poured into saturated aqueous ammonium chloride (10 mL). The product was extracted with ethyl acetate (3×25 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford ethyl 2-(4-methyl-3,4-dihydro-2H-1,4-benzoxazin-6-yl)-2-oxoacetate as a yellow oil (500 mg, 18%). LCMS (ES, m/z): 250 [M+H]⁺.

### Step 3. Ethyl 2-hydroxy-2-(4-methyl-3,4-dihydro-2H-1,4-benzoxazin-6-yl)acetate

To a solution of ethyl 2-(4-methyl-3,4-dihydro-2H-1,4-benzoxazin-6-yl)-2-oxoacetate (250 mg, 1.00 mmol) in tetrahydrofuran (10 mL) was added sodium borohydride (57 mg, 1.51 mmol). The resulting mixture was stirred for 10 min at 0° C. and then poured into water (10 mL). The product was extracted with ethyl acetate (3×10 mL). The combined organic layers were dried over anhydrous sodium sulfate, filtered, and concentrated under vacuum. The crude product was purified by silica gel chromatography (eluting with 0:100 to 20:80 ethyl acetate/petroleum ether) to afford ethyl 2-hydroxy-2-(4-methyl-3,4-dihydro-2H-1,4-benzoxazin-6-yl)acetate as a colorless oil (180 mg, 71%). LCMS (ES, m/z): 252 [M+H]⁺.

### Step 4 Lithium 2-hydroxy-2-(4-methyl-3,4-dihydro-2H-1,4-benzoxazin-6-yl)acetate (A2)

To a solution of ethyl 2-hydroxy-2-(4-methyl-3,4-dihydro-2H-1,4-benzoxazin-6-yl)acetate (180 mg, 0.72 mmol) in tetrahydrofuran (2 mL) and water (2 mL) was added lithium hydroxide (87 mg, 3.63 mmol). The resulting mixture was stirred for 16 h at room temperature. The reaction mixture was washed with diethyl ether (1×8 mL) and then concentrated under vacuum to afford lithium 2-hydroxy-2-(4-methyl-3,4-dihydro-2H-1,4-benzoxazin-6-yl)acetate as a yellow oil (100 mg, 16%). LCMS (ES, m/z): 224 [M+H]⁺.

### Synthesis of Intermediate B2

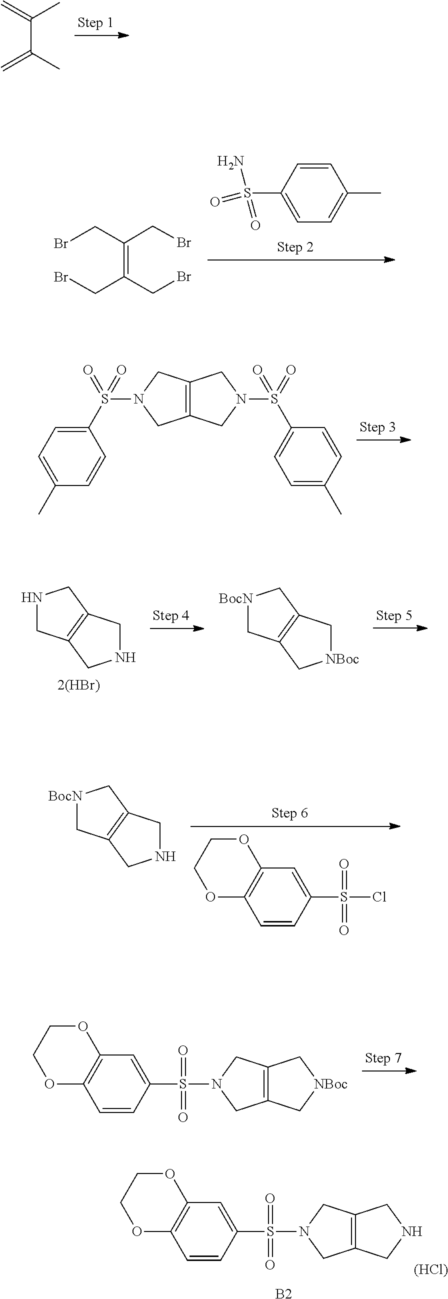

### Step 1. 1,4-dibromo-2,3-bis(bromomethyl)but-2-ene

To a solution of 2,3-dimethylbut-2-ene (1000 g, 11.9 mol) in DCM (1000 mL) in a 4 L 4-necked round bottom flask was added aqueous hydrogen bromide solution (150 mL, 48%) with stirring at 10-15° C. To the reaction was added bromine (9.90 kg, 62.0 mol) with stirring at 0° C. The resulting mixture was stirred for 2 days at 45° C. in an oil bath. After cooling to room temperature, the reaction mixture was carefully poured into saturated aqueous sodium hydrogen sulfite solution (10 L). The precipitate was collected by filtration and dried in oven to afford 1,4-dibromo-2,3-bis(bromomethyl)but-2-ene as a light yellow solid (3000 g, 44%). GCMS: (EI, m/z): 398, 400, 402 [M]⁺.

### Step 2. 2,5-ditosyl-1,2,3,4,5,6-hexahydropyrrolo[3,4-c]pyrrole

To a solution of 1,4-dibromo-2,3-bis(bromomethyl)but-2-ene (2000 g, 3.50 mol) in DMF (20 L) was added 4-methylbenzene-1-sulfonamide (2137 g, 12.5 mol), and potassium carbonate (5175 g, 37.4 mol). The resulting mixture was stirred for 2 days at room temperature. The reaction mixture was slowly poured into water/ice (20 L). The precipitate was collected by filtration, washed with ethanol and dried in an oven to afford 2,5-ditosyl-1,2,3,4,5,6-hexahydropyrrolo[3,4-c]pyrrole as a light yellow solid (1345 g, 78%). LCMS: (ES, m/z): 419 [M+H]⁺.

### Step 3. 1,2,3,4,5,6-hexahydropyrrolo[3,4-c]pyrrole hydro bromide salt

To a solution of 2,5-ditosyl-1,2,3,4,5,6-hexahydropyrrolo[3,4-c]pyrrole (1345 g, 2.73 mol) in aqueous hydrogen bromide solution (4500 mL, 48%) in 10 L 4-necked round-bottom flask, was added phenol (1270 g, 13.5 mol). The resulting mixture was stirred for 2 days at 120° C. After cooling to room temperature, the aqueous layer was collected and concentrated under vacuum. The resulting solids were washed with DCM/MeOH (v:v=10:1, 3×300 mL) and dried in an oven to afford 1,2,3,4,5,6-hexahydropyrrolo[3,4-c]pyrrole hydrogen bromide salt as a yellow solid (480 g, 61%). L CMS: (ES, m/z): 111 [M+H]⁺.

### Step 4. Di-tert-butyl pyrrolo[3,4-c]pyrrole-2,5(1H,3H,4H,6H)-dicarboxylate

To a suspension of 1,2,3,4,5,6-hexahydropyrrolo[3,4-c]pyrrole hydrogen bromide salt (458 g, 1.52 mol) in water (4 L) was added sodium bicarbonate (424 g, 5.05 mol) followed by dropwise addition of a solution of di-tert-butyl dicarbonate (807 g, 3.70 mol) in methanol (500 mL) with stirring at 0° C. The resulting solution was stirred for 16 h at 25° C. The precipitate was collected by filtration and dried in an oven to afford di-tert-butyl pyrrolo[3,4-c]pyrrole-2,5(1H,3H,4H,6H)-dicarboxylate as a white solid (300 g, 61%). LCMS (ES, m/z): 311[M+H]⁺.

### Step 5. Tert-butyl 4,5-dihydropyrrolo[3,4-c]pyrrole-2(1H,3H,4H)-carboxylate 4-methylbenzene-1-sulfonic acid salt

To a solution of di-tert-butyl pyrrolo[3,4-c]pyrrole-2,5(1H,3H,4H,6H)-dicarboxylate (200 g, 612 mmol) in propan-2-yl acetate (5 L) was added 4-methylbenzene-1-sulfonic acid (123 g, 647 mmol) in portions at 0° C. The resulting mixture was stirred for 16 h at 55° C. in an oil bath. After cooling to room temperature, the precipitate was collected by filtration and dried in an oven to afford tert-butyl 4,5-dihydropyrrolo[3,4-c]pyrrole-2(1H,3H,4H)-carboxylate 4-methylbenzene-1-sulfonic acid salt as a yellow solid (197 g, 80%). LCMS: (ES, m/z): 211[M+H]⁺.

### Step 6. Tert-butyl 5-(2,3-dihydrobenzo[b][1,4]dioxin-6-ylsulfonyl)-4,5-dihydropyrrolo[3,4-c]pyrrole-2(1H,3H,4H)-carboxylate

To a suspension of tert-butyl 4,5-dihydropyrrolo[3,4-c]pyrrole-2(1H,3H,4H)-carboxylate 4-methylbenzene-1-sulfonic acid salt (61 g, 142 mmol) in water (100 mL) and tetrahydrofuran (30 mL) was added sodium hydroxide (13 g, 325 mmol) followed by portion-wise addition of 2,3-dihydro-1,4-benzodioxine-6-sulfonyl chloride (25 g, 95.9 mmol) at 0° C. The resulting mixture was stirred for 2 h at 25° C. The product was extracted with ethyl acetate (3×200 mL). The organic layers were combined, dried over anhydrous sodium sulfate, filtered and concentrated under vacuum. The resulting material was purified by silica gel chromatography (eluting with 1:10 ethyl acetate/petroleum ether) to afford tert-butyl 5-(2,3-dihydrobenzo[b][1,4]dioxin-6-ylsulfonyl)-4,5-dihydropyrrolo[3,4-c]pyrrole-2(1H,3H,4H)-carboxylate as a white solid (30 g, 73%). LCMS: (ES, m/z): 409 [M+H]⁺.

### Step 7. 2-(2,3-dihydrobenzo[b][1,4]dioxin-6-ylsulfonyl)-1,2,3,4,5,6-hexahydropyrrolo[3,4-c]pyrrole hydrochloric salt (B2)

To a solution of tert-butyl 5-(2,3-dihydrobenzo[b][1,4]dioxin-6-ylsulfonyl)-4,5-dihydropyrrolo[3,4-c]pyrrole-2(1H,3H,4H)-carboxylate (30.0 g, 69.8 mmol) in 1,4-dioxane (100 mL) was added hydrochloric acid (200 mL, 4 M in 1,4-dioxane). The resulting solution was stirred for 2 h at 25° C. and then concentrated under vacuum to afford 2-(2,3-dihydrobenzo[b][1,4]dioxin-6-ylsulfonyl)-1,2,3,4,5,6-hexahydropyrrolo[3,4-c]pyrrole hydrochloric salt (B2) as a yellow solid (20 g, 79%). LCMS: (ES, m/z): 309 [M+H]⁺.

### Synthesis of Compound 2

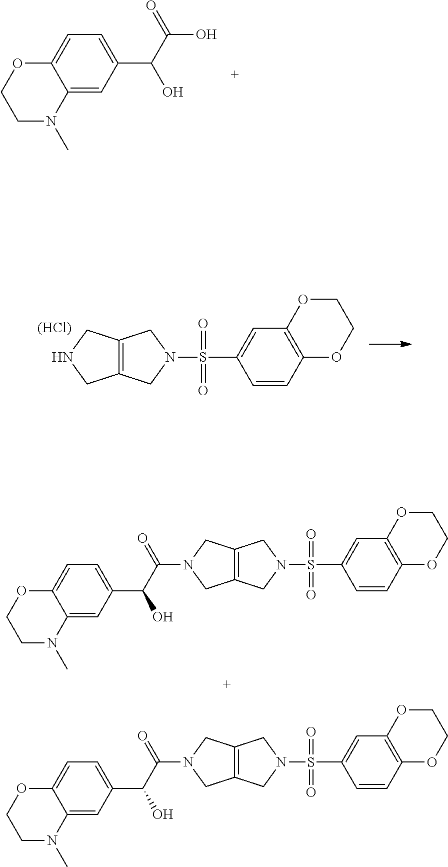

To a solution of lithium 2-hydroxy-2-(4-methyl-3,4-dihydro-2H-1,4-benzoxazin-6-yl)acetate (A2) (1 equiv) in DMF was added HATU (1.2 equiv), 2-(2,3-dihydrobenzo[b][1,4]dioxin-6-ylsulfonyl)-1,2,3,4,5,6-hexahydropyrrolo[3,4-c]pyrrole hydrochloric salt (1B2) (1 equiv) and DIEA (3 equiv). The resulting mixture was stirred for 1 h at room temperature. The reaction mixture was poured into water and then extracted with ethyl acetate. The combined organic layers were dried over anhydrous sodium sulfate, filtered, and concentrated under vacuum. The resulting crude product was purified by Prep-HPLC. The product fractions were concentrated under vacuum. ¹H-NMR: (CDCl₃, 400 MHz) δ (ppm): 7.37-7.27 (m, 2H), 7.02-6.96 (m, 1H), 6.73 (d, J=7.6 Hz, 1H), 6.63-6.61 (m, 2H), 4.89-4.87 (m, 1H), 4.33-4.25 (m, 7H), 4.15-4.01 (m, 6H), 3.73-3.71 (m, 1H), 3.29-3.27 (m, 2H), 2.90 (s, 3H). The two enantiomers were further separated by Chiral Prep-HPLC (Column: CHIRALPAK IF, 5 μm, 100×460 mm; Mobile Phase, A: DCM, and B: MeOH (containing 0.1% DEA) (keep 75% B over 16 min); Flow rate: 20 mL/min; Detector: UV 254/220 nm; Retention time: 1^{st} eluting isomer, 2.182 min; 2^{nd} eluting isomer (2), 2.988 min).

### Example 11. Synthesis of USP9X Inhibitor 3

### Synthesis of Intermediate A3

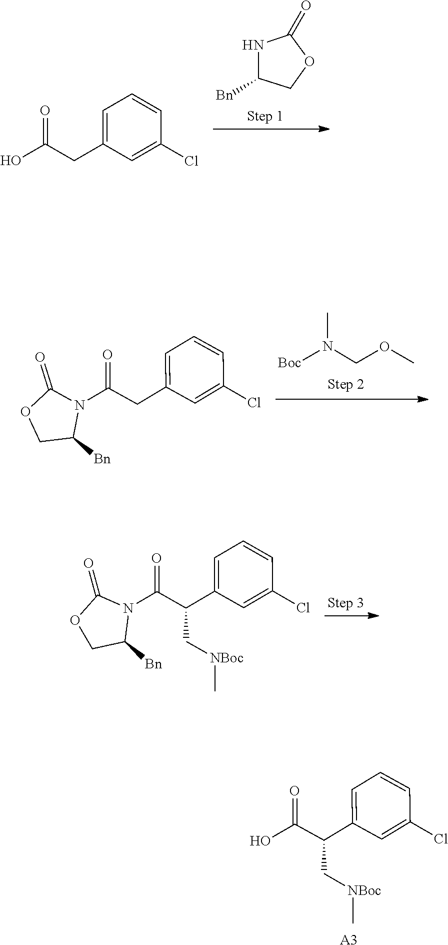

### Step 1. (4S)-4-benzyl-3-[2-(3-chlorophenyl)acetyl]-1,3-oxazolidin-2-one

To a solution of 2-(3-chlorophenyl)acetic acid (23.2 g, 0.14 mol) in toluene (300 mL) was added (4S)-4-benzyl-1,3-oxazolidin-2-one (20 g, 0.11 mol) followed by the slow addition of TEA (46 g, 0.45 mol) with stirring at 15° C., and then slow addition of 2,2-dimethylpropanoyl chloride (17.4 g, 0.14 mol) with stirring at 30° C. The resulting mixture was stirred for 3 h at 110° C., and then cooled to room temperature. The mixture was concentrated under vacuum and the resulting crude product was purified by silica gel chromatography (eluting with 0:100 to 5:95 ethyl acetate/petroleum ether) to afford (4S)-4-benzyl-3-[2-(3-chlorophenyl)acetyl]-1,3-Oxazolidin-2-one as a yellow solid (18 g, 40%). ¹H-NMR (CDCl₃, 400 MHz) δ (ppm): 7.43-7.12 (m, 9H), 4.76-4.64 (m, 1H), 4.41-4.15 (m, 4H), 3.35-3.23 (m, 1H), 2.87-2.74 (m, 1H). LCMS (ES, m/z) 330, 332 [M+H]⁺.

### Step 2. tert-butyl N-[(2R)-3-[(4S)-4-benzyl-2-oxo-1,3-oxazolidin-3-yl]-2-(3-chlorophenyl)-3-oxopropyl]-N-methylcarbamate

To a solution of (4S)-4-benzyl-3-[2-(3-chlorophenyl)acetyl]-1,3-oxazolidin-2-one (15 g, 0.41 mol) in dichloromethane (180 mL) was added a solution of titanium(IV) chloride (48.4 mL, 1 M in DCM) dropwise with stirring at −20° C. After stirring for 2 h at −20° C., a solution of DIEA (5.1 mL, 0.31 mol) in DCM (10 mL) was added slowly with stirring. After 1.5 h at −20° C., tert-butyl N-(methoxymethyl)-N-methylcarbamate (10.4 g, 0.59 mol) in dichloromethane (10 mL) was added dropwise. The reaction mixture was stirred for 2 h at −20° C. and then treated with saturated ammonium chloride solution (100 mL). The product was extracted with ethyl acetate (3×300 mL). The combined organic layers were dried over anhydrous sodium sulfate, filtered, and concentrated under vacuum to afford tert-butyl N-[(2R)-3-[(4S)-4-benzyl-2-oxo-1,3-oxazolidin-3-yl]-2-(3-chlorophenyl)-3-oxopropyl]-N-methylcarbamate as a yellow solid (18 g, 84%).

### Step 3. (2R)-3-{[(tert-butoxy)carbonyl](methyl)amino}-2-(3-chlorophenyl)propanoic acid

To a solution of lithium hydroxide (2.3 g, 0.09 mol) in water (125 mL) was added THF (170 mL) followed by the sequential addition of a solution of hydrogen peroxide (9.2 mL, 30% in water) and a solution of tert-butyl N-[(2R)-3-[(4S)-4-benzyl-2-oxo-1,3-oxazolidin-3-yl]-2-(3-chlorophenyl)-3-oxopropyl]-N-methylcarbamate (18 g, 0.04 mol) in tetrahydrofuran (10 mL) dropwise with stirring at 0° C. The resulting mixture was stirred for 3 h at 0° C. The reaction was carefully quenched with aqueous sodium sulfite solution (100 mL, 12.5 wt %) while maintaining reaction temperature<10° C. After stirring for 30 min at room temperature, the mixture (pH=14) was concentrated to remove organic solvent and then washed with diethyl ether (3×200 mL). The aqueous layer was then acidified to pH=2˜3 with aqueous potassium bisulfate solution (27 wt %) while maintaining temperature<15° C. The resulting solution was extracted with ethyl acetate (3×300 mL). The combined organic layers were dried over anhydrous sodium sulfate, filtered and concentrated under vacuum to afford (2R)-3-{[(tert-butoxy)carbonyl](methyl)amino}-2-(3-chlorophenyl)propanoic acid as a yellow oil (10 g, 84%). Note: the material contains about 20 wt % of (4S)-4-benzyl-1,3-oxazolidin-2-one based on HNMR determination, and its ee value is about 96%.

The crude material (4.5 g) was dissolved in MeCN (5 mL) and N-cyclohexylcyclohexanamine (3 g, 16.5 mmol) was added. The reaction was heated to 60° C. for 3 h and cooled to room temperature slowly over 16 h without stirring. The solids were collected by filtration and dried under vacuum to afford N-cyclohexylcyclohexanamine (2R)-3-{[(tert-butoxy)carbonyl](methyl)amino}-2-(3-chlorophenyl) propanoic acid complex as a white solid (5 g). The complex was then dissolved with aqueous solution of KHSO₄ (50 mL, 27 wt %) and EtOAc (50 mL). The resulting solution was stirred for 1.5 h at rt and extracted with EtOAc (3×50 mL). The combined organic layers were dried over anhydrous sodium sulfate, filtered and concentrated under vacuum to afford pure (2R)-3-{[(tert-butoxy)carbonyl](methyl)amino}-2-(3-chlorophenyl)propanoic acid as a white solid (2.30 g, 99% purity, >99% ee). ¹H-NMR (DMSO-d₆, 400 MHz) δ (ppm): 7.43-7.21 (m, 4H), 4.07-3.90 (m, 1H), 3.94-3.89 (m, 2H), 2.84-2.70 (m, 3H), 1.39 (s, 9H). LCMS (ES, m/z) 314,316 [M+H]⁺.

### Synthesis of Compound 3

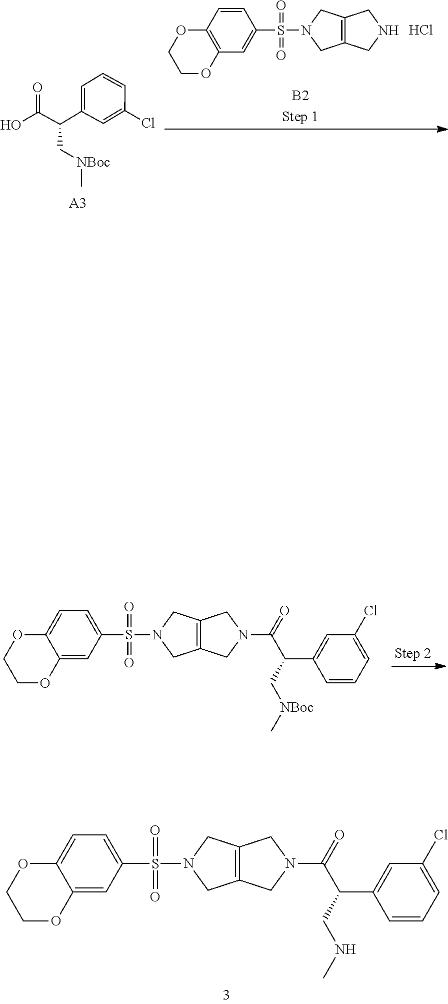

### Step 1. Tert-butyl N-[(2R)-2-(3-chlorophenyl)-3-[5-(2,3-dihydro-1,4-benzodioxine-6-sulfonyl)1H,2H,3H,4H,5H, 6H-pyrrolo[3,4-c]pyrrol-2-yl]-3-oxopropyl]-N-methylcarbamate

To a solution of (2R)-3-{[(tert-butoxy)carbonyl](methyl)amino}-2-(3-chlorophenyl)propanoic acid (A3) (2.30 g, 7.34 mmol) in N,N-dimethylformamide (20 mL) was added HATU (3.07 g, 8.08 mmol), 2-(2,3-dihydro-1,4-benzodioxine-6-sulfonyl)-1H,2H,3H,4H,5H,6H-pyrrolo[3,4-c]pyrrole hydrochloride (B2) (2.52 g, 7.34 mmol), and DIEA (3.82 mL, 22.1 mmol). The resulting solution was stirred for 2 h at rt. The reaction mixture was poured into water (100 mL) and then extracted with ethyl acetate (3×100 mL). The combined organic layers were dried over anhydrous sodium sulfate, filtered, and concentrated under vacuum. The crude product was purified by silica gel chromatography (eluting with 1:15 ethyl acetate/dichloromethane) to afford tert-butyl N-[(2R)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate as a white solid (3.5 g, 79%). ¹H-NMR (DMSO-d₆, 400 MHz) δ (ppm): 7.40-7.18 (m, 6H), 7.07-7.06 (m, 1H), 4.45-4.22 (m, 5H), 4.11-3.88 (m, 7H), 3.88-3.58 (m, 2H), 3.44-3.36 (m, 1H), 2.75-2.67 (m, 3H), 1.27-1.16 (m, 9H). LCMS (ES, m/z) 604, 606 [M+H]⁺.

### Step 2. (2R)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-3-(methylamino)propan-1-one (3)

To a solution of tert-butyl N-[(2R)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate (1.5 g, 2.49 mmol) in ethyl acetate (10 mL) was added a solution of hydrochloric acid (10 mL, 4 N in 1,4-dioxane). The resulting solution was stirred for 3 h at 25° C. The mixture was concentrated under vacuum to about ⅓ volume and the solids were collected by filtration. The solids were treated with EtOAc (10 mL) at 70° C., filtered at room temperature, and dissolved with saturated potassium carbonate solution/EA (1:1, 10 mL). The resulting solution was stirred for 3 h and then extracted with EtOAc (3×5 mL). The combined organic layers were dried over anhydrous sodium sulfate, filtered, and concentrated under vacuum to afford (2R)-2-(3-chlorophenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one (3) as a white solid (1 g, 80%). ¹H-NMR (DMSO-d₆, 400 MHz) δ (ppm): 7.36-7.25 (m, 6H), 7.07-7.06 (m, 1H), 4.40-4.29 (m, 5H), 4.06-3.88 (m, 8H), 3.06-3.01 (m, 1H), 2.60-2.50 (m, 1H), 2.23 (s, 3H), 1.66 (s, 1H). LCMS (ES, m/z) 504, 506 [M+H]⁺.

### Example 12. Synthesis of USP9X Inhibitor 4

### Synthesis of Intermediate C1

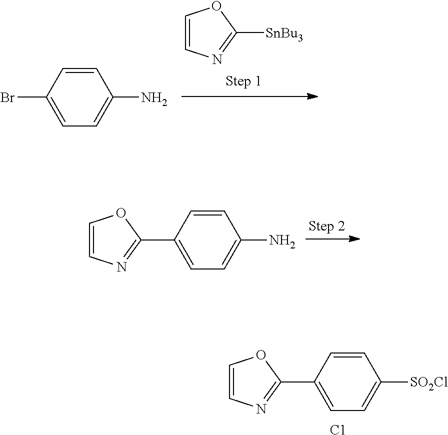

### Step 1. 4-(1,3-oxazol-2-yl)aniline

To a solution of 4-bromoaniline (1 equiv) in 1,4-dioxane was added Pd(dppf)Cl₂ (10 mol %) and 2-(tributylstannyl)-1,3-oxazole (1 equiv). The resulting mixture was stirred for 48 h at 100° C. and then cooled to room temperature. The reaction mixture was poured into water and then extracted with ethyl acetate. The combined organic layers were dried over anhydrous sodium sulfate, filtered, and concentrated under vacuum. The resulting crude product was purified by silica gel chromatography to afford 4-(1,3-oxazol-2-yl)aniline.

### Step 2. 4-(1,3-oxazol-2-yl)benzene-1-sulfonyl chloride (C1)

Into glacial acetic acid was bubbled in SO₂ gas for 1 h at room temperature. Then CuCl₂ (25 mol %) was added and SO₂ gas was bubbled in for additional 2 h to afford solution A. To a pre-cooled solution of 4-(1,3-oxazol-2-yl)aniline (1 equiv) in acetic acid and concentrated hydrochloric acid was added a solution of sodium nitrite (1.1 equiv) in distilled water dropwise with stirring at −10° C. After stirring for 15 min, solution A was added to this diazonium salt solution at −10° C. The resulting solution was allowed to warm to room temperature naturally and stirred for 16 h. The reaction mixture was treated with water and then extracted with EtOAc. The combined organic layers were dried over anhydrous sodium sulfate, filtered, and concentrated under vacuum. The resulting crude product was purified by silica gel chromatography to afford 4-(1,3-oxazol-2-yl)benzene-1-sulfonyl chloride (Cl). LCMS (ES, m/z) 244 [M+H]⁺.

### Synthesis of Intermediate D1

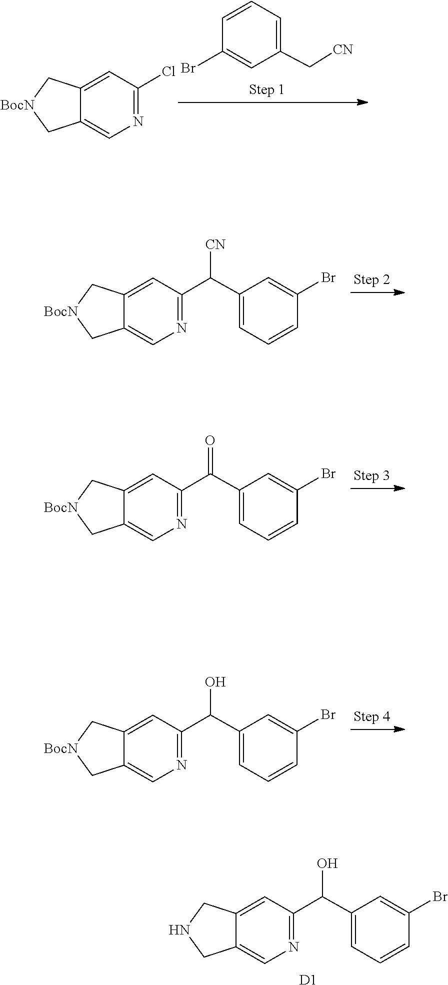

### Step 1. Tert-butyl 6-[(3-bromophenyl)(cyano)methyl]-1H,2H,3H-pyrrolo[3,4-c]pyridine-2-carboxylate

To a solution of tert-butyl 6-chloro-1H,2H,3H-pyrrolo[3,4-c]pyridine-2-carboxylate (2.0 g, 7.79 mmol) in tetrahydrofuran (20 mL) was added NaNH₂ (2.0 g, 7.87 mmol), and 2-(3-bromophenyl)acetonitrile (2.32 g, 11.8 mmol). The resulting mixture was stirred for 16 h at 50° C. and then cooled to room temperature. The reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford tert-butyl 6-[(3-bromophenyl)(cyano)methyl]-1H,2H,3H-pyrrolo[3,4-c]pyridine-2-carboxylate (1.1 g, 34%). LCMS (ES, m/z) 414, 416 [M+H]⁺.

### Step 2. Tert-butyl 6-(3-bromobenzoyl)-1H,2H,3H-pyrrolo[3,4-c]pyridine-2-carboxylate

To a solution of sodium hydroxide (128 mg, 0.012 mmol) in water (0.13 mL) was added DMSO (8 mL), benzyltriethylammonium chloride (27 mg, 0.12 mmol) and a solution of tert-butyl 6-[(3-bromophenyl)(cyano)methyl]-1H,2H,3H-pyrrolo[3,4-c]pyridine-2-carboxylate (1.1 g, 2.66 mmol) in DMSO (10 mL). The resulting solution was stirred for 3 h at room temperature while oxygen was bubbling in. The reaction mixture was poured into water (20 mL) and then extracted with EtOAc (3×20 mL). The combined organic layers were washed by brine, dried over anhydrous sodium sulfate, filtered and concentrated under vacuum. The resulting crude product was purified by silica gel chromatography (eluting with 0:100 to 20:80 ethyl acetate/petroleum ether) to afford tert-butyl 6-(3-bromobenzoyl)-1H,2H,3H-pyrrolo[3,4-c]pyridine-2-carboxylate (640 mg, 60%). LCMS (ES, m/z) 403, 405 [M+H]⁺.

### Step 3. Tert-butyl 6-[(3-bromophenyl)(hydroxy)methyl]-1H,2H,3H-pyrrolo[3,4-c]pyridine-2-carboxylate

To a solution of tert-butyl 6-(3-bromobenzoyl)-1H,2H,3H-pyrrolo[3,4-c]pyridine-2-carboxylate (500 mg, 1.24 mmol) in tetrahydrofuran (10 mL) was added NaBH₄ (95 mg, 2.49 mmol) at 0° C. The resulting mixture was stirred for 1 h at 0° C. The reaction mixture was poured into water (10 mL) and then extracted with EA (3×20 mL). The combined organic layers were washed with brine (3×20 mL), dried over anhydrous sodium sulfate, filtered, and concentrated under vacuum. The resulting crude product was purified by silica gel chromatography (eluting with 0:100 to 25:80 ethyl acetate/petroleum ether) to afford tert-butyl 6-[(3-bromophenyl)(hydroxy)methyl]-1H,2H,3H-pyrrolo[3,4-c]pyridine-2-carboxylate (400 mg, 80%). LCMS (ES, m/z) 405, 407 [M+H]⁺.

### Step 4. (3-bromophenyl)(2,3-dihydro-1H-pyrrolo[3,4-c]pyridin-6-yl)methanol (D1)

To a solution of tert-butyl 6-[(3-bromophenyl)(hydroxy)methyl]-1H,2H,3H-pyrrolo[3,4-c]pyridine-2-carboxylate (1 equiv) in DCM was added TFA. The resulting solution was stirred for 1 h at rt and then concentrated under vacuum. The resulting mixture was then basified to pH 8 with saturated aqueous potassium carbonate solution. The resulting mixture was extracted with DCM. The combined organic layers were dried over anhydrous sodium sulfate, filtered and concentrated under vacuum to afford (3-bromophenyl)(2,3-dihydro-1H-pyrrolo[3,4-c]pyridin-6-yl)methanol (D1). LCMS (ES, m/z) 305 [M+H]⁺.

### Synthesis of Compound 4

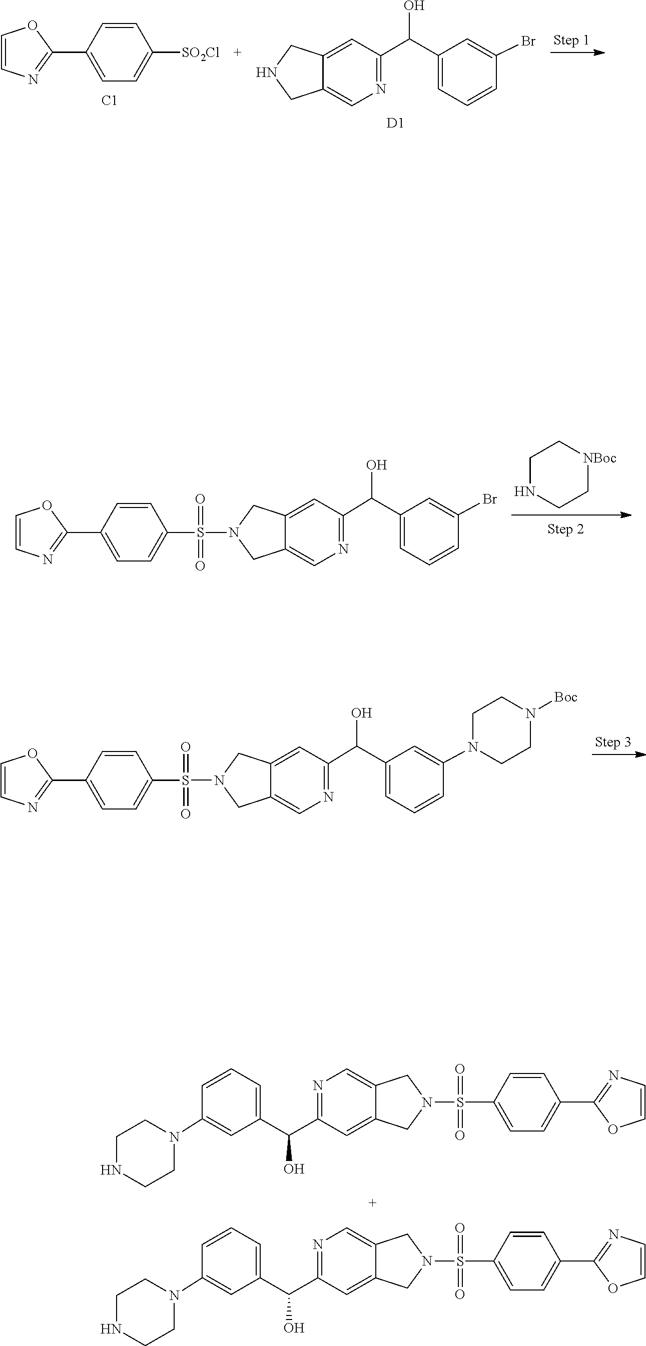

### Step 1. (3-bromophenyl)(2-((4-(oxazol-2-yl)phenyl)sulfonyl)-2,3-dihydro-1H-pyrrolo[3,4-c]pyridin-6-yl)methanol

To a solution of 4-(1,3-oxazol-2-yl)benzene-1-sulfonyl chloride (Cl) (1.5 equiv) in DCM was added (3-bromophenyl)(2,3-dihydro-1H-pyrrolo[3,4-c]pyridin-6-yl)methanol (D1) (1 equiv) and TEA (3.0 equiv). The resulting solution was stirred for 2 h at 25° C. The reaction mixture was poured into water and then extracted with ethyl acetate. The combined organic layers were dried over anhydrous sodium sulfate, filtered, and concentrated under vacuum. The resulting crude product was purified by silica gel chromatography to afford (3-bromophenyl)(2-((4-(oxazol-2-yl)phenyl)sulfonyl)-2,3-dihydro-1H-pyrrolo[3,4-c]pyridin-6-yl)methanol. LCMS (ES, m/z) 512, 514 [M+H]⁺.

### Step 2. Tert-butyl 4-(3-(hydroxy(2-((4-(oxazol-2-yl)phenyl)sulfonyl)-2,3-dihydro-1H-pyrrolo[3,4-c]pyridin-6-yl)methyl)phenyl)piperazine-1-carboxylate

To a solution of (3-bromophenyl)(2-((4-(oxazol-2-yl)phenyl)sulfonyl)-2,3-dihydro-1H-pyrrolo[3,4-c]pyridin-6-yl)methanol (1 equiv) in toluene was added N-Boc-piperidine (1.4 equiv), XPhos (25 mol %), Cs₂CO₃ (3.3 equiv) and Pd₂(dba)₃.CHCl₃ (12 mol %). The resulting mixture was stirred for 16 h at 100° C. and then cooled to room temperature. The reaction mixture was filtered and concentrated under vacuum. The resulting crude product was purified by silica gel chromatography to afford tert-butyl 4-(3-(hydroxy(2-((4-(oxazol-2-yl)phenyl)sulfonyl)-2,3-dihydro-1H-pyrrolo[3,4-c]pyridin-6-yl)methyl)phenyl)piperazine-1-carboxylate. LCMS (ES, m/z) 618 [M+H]⁺.

### Step 3. [2-[4-(1,3-oxazol-2-yl)benzenesulfonyl]-1H,2H,3H-pyrrolo[3,4-c]pyridin-6-yl][3-(piperazin-1-yl)phenyl] methanol (4)

To a solution of HCl in 1,4-dioxane (4 N) was added tert-butyl 4-(3-(hydroxy(2-((4-(oxazol-2-yl)phenyl)sulfonyl)-2,3-dihydro-1H-pyrrolo[3,4-c]pyridin-6-yl)methyl)phenyl)piperazine-1-carboxylate (1 equiv) and water. The resulting mixture was stirred for 4 h at room temperature. The reaction mixture was concentrated and lyophilized. The resulting crude product was purified by reverse phase chromatography. The product fractions were concentrated and lyophilized to afford tert-butyl 4-(3-(hydroxy(2-((4-(oxazol-2-yl)phenyl)sulfonyl)-2,3-dihydro-1H-pyrrolo[3,4-c]pyridin-6-yl)methyl)phenyl)piperazine-1-carboxylate as a white solid (79%). ¹H-NMR (DMSO-d₆, 400 MHz) δ (ppm): 9.09 (br s, 2H), 8.33 (d, J=2.8 Hz, 2H), 8.16 (d, J=8.4 Hz, 2H), 8.01 (d, J=8.4 Hz, 2H), 7.48 (d, J=3.2 Hz, 2H), 7.12-7.08 (m, 1H), 7.03-7.01 (m, 1H), 6.80-6.77 (m, 2H), 6.07 (br s, 1H), 5.62 (s, 1H), 4.69-4.58 (m, 4H), 3.52-3.35 (m, 4H), 3.30-3.19 (m, 4H). LCMS (ES, m/z) 518 [M+H]⁺. The two enantiomers were further separated by Chiral Prep-HPLC (Column: CHIRALPAK IF, 5 μm, 20×250 mm; Mobile Phase, A: MTBE (containing 0.2% IPA) and B: EtOH (keep 50% B over 25 min); Detector: UV 254/220 nm; Retention time: 1^{st} eluting isomer, 10.759 min; 2^{nd} eluting isomer (4), 17.742 min).

### Synthesis of Compounds of Formula I

In some embodiments, the USP9X inhibitor may be a compound of Table 1:

TABLE 1

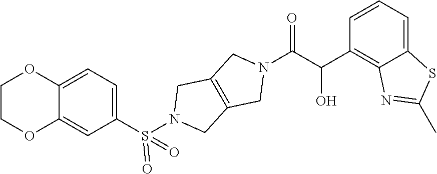 [SMILES: Cc1nc2c(C(O)C(=O)N3CC4=C(C3)CN(S(=O)(=O)c3ccc5c(c3)OCCO5)C4)cccc2s1]

Examples 94-31 and 94-32

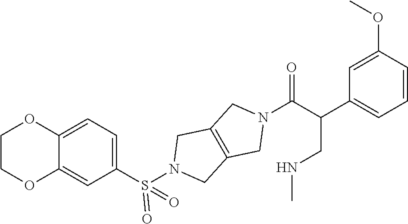 [SMILES: CNCC(C(=O)N1CC2=C(C1)CN(S(=O)(=O)c1ccc3c(c1)OCCO3)C2)c1cccc(OC)c1]

Examples 95-3 and 95-4

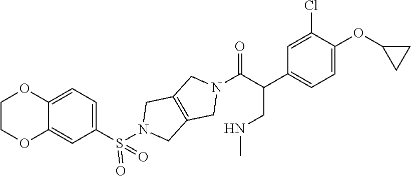 [SMILES: CNCC(C(=O)N1CC2=C(C1)CN(S(=O)(=O)c1ccc3c(c1)OCCO3)C2)c1ccc(OC2CC2)c(Cl)c1]

Examples 95-29 and 95-30

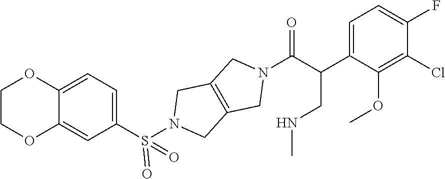 [SMILES: CNCC(C(=O)N1CC2=C(C1)CN(S(=O)(=O)c1ccc3c(c1)OCCO3)C2)c1ccc(F)c(Cl)c1OC]

Examples 95-31 and 95-32

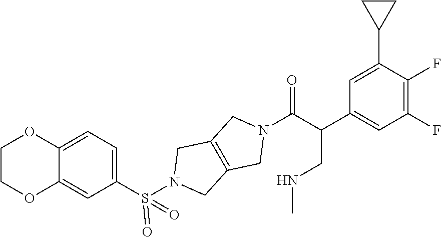 [SMILES: CNCC(C(=O)N1CC2=C(C1)CN(S(=O)(=O)c1ccc3c(c1)OCCO3)C2)c1cc(F)c(F)c(C2CC2)c1]

Examples 95-70 and 95-71

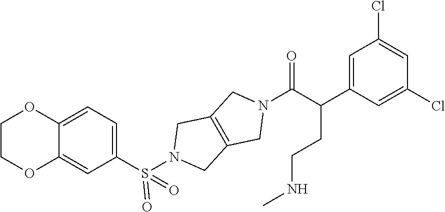 [SMILES: CNCCC(C(=O)N1CC2=C(C1)CN(S(=O)(=O)c1ccc3c(c1)OCCO3)C2)c1cc(Cl)cc(Cl)c1]

Examples 96-8 and 96-9

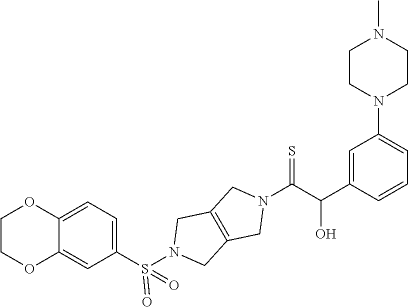 [SMILES: CN1CCN(c2cccc(C(O)C(=S)N3CC4=C(C3)CN(S(=O)(=O)c3ccc5c(c3)OCCO5)C4)c2)CC1]

Examples 97-15 and 97-16

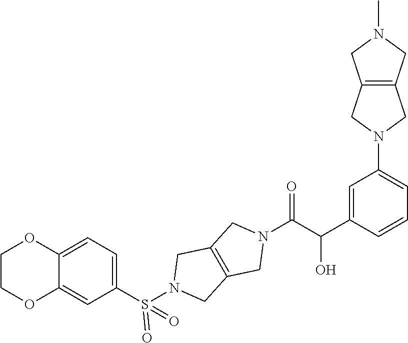 [SMILES: CN1CC2=C(C1)CN(c1cccc(C(O)C(=O)N3CC4=C(C3)CN(S(=O)(=O)c3ccc5c(c3)OCCO5)C4)c1)C2]

Examples 97-17 and 97-18

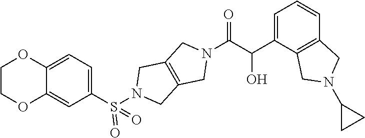 [SMILES: O=C(C(O)c1cccc2c1CN(C1CC1)C2)N1CC2=C(C1)CN(S(=O)(=O)c1ccc3c(c1)OCCO3)C2]

Examples 98-1 and 98-2

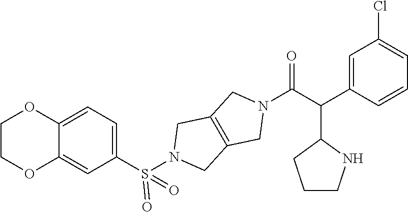 [SMILES: O=C(C(c1cccc(Cl)c1)C1CCCN1)N1CC2=C(C1)CN(S(=O)(=O)c1ccc3c(c1)OCCO3)C2]

Examples 99-8 and 99-9

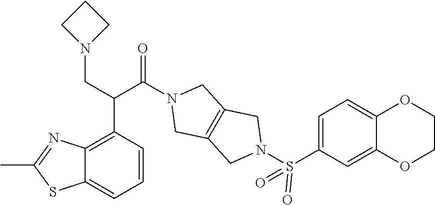 [SMILES: Cc1nc2c(C(CN3CCC3)C(=O)N3CC4=C(C3)CN(S(=O)(=O)c3ccc5c(c3)OCCO5)C4)cccc2s1]

Examples 103-7 and 103-8

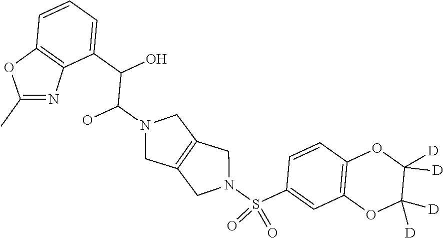 [SMILES: [2H]C1([2H])Oc2ccc(S(=O)(=O)N3CC4=C(CN(C(O)C(O)c5cccc6oc(C)nc56)C4)C3)cc2OC1([2H])[2H]]

Examples 103-46 and 103-47

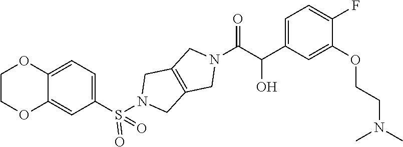 [SMILES: CN(C)CCOc1cc(C(O)C(=O)N2CC3=C(C2)CN(S(=O)(=O)c2ccc4c(c2)OCCO4)C3)ccc1F]

Examples 112-3 and 112-4

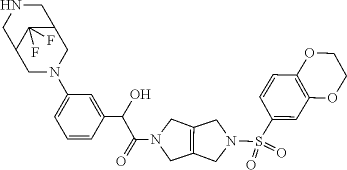 [SMILES: O=C(C(O)c1cccc(N2CC3CNCC(C2)C3(F)F)c1)N1CC2=C(C1)CN(S(=O)(=O)c1ccc3c(c1)OCCO3)C2]

Examples 114-1 and 114-2

### Intermediates for Compounds of Table 1

### Intermediate 2-2. 2-((2,3-dihydrobenzo[b][1,4]dioxin-6-yl)sulfonyl)-1,2,3,4,5,6-hexahydropyrrolo[3,4-c]pyrrole

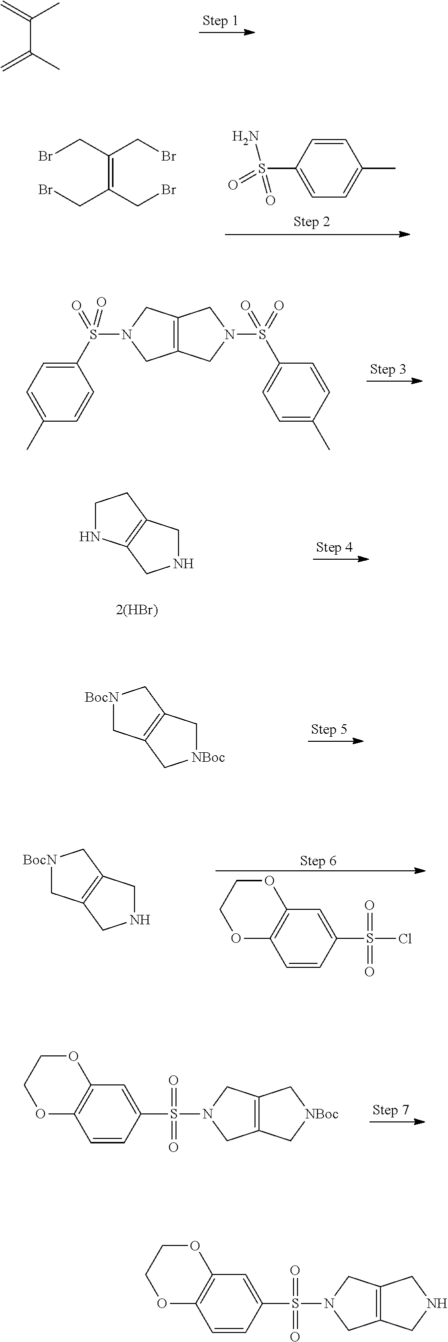

### Step 1. 1,4-dibromo-2,3-bis(bromomethyl)but-2-ene

To a solution of 2,3-dimethylbut-2-ene (1000 g, 11.9 mol) in DCM (1000 mL) in a 4 L 4-necked round bottom flask was added aqueous hydrogen bromide solution (150 mL, 48%) with stirring at 10-15° C. To the reaction was added bromine (9.90 kg, 62.0 mol) with stirring at 0° C. The resulting mixture was stirred for 2 days at 45° C. in an oil bath. After cooling to room temperature, the reaction mixture was carefully poured into saturated aqueous sodium hydrogen sulfite solution (10 L). The precipitate was collected by filtration and dried in oven to afford 1,4-dibromo-2,3-bis(bromomethyl)but-2-ene as a light yellow solid (3000 g, 44%). GCMS: (EI, m/z): 398, 400, 402 [M]⁺.

### Step 2. 2,5-ditosyl-1,2,3,4,5,6-hexahydropyrrolo[3,4-c]pyrrole

To a solution of 1,4-dibromo-2,3-bis(bromomethyl)but-2-ene (2000 g, 3.50 mol) in DMF (20 L) was added 4-methylbenzene-1-sulfonamide (2137 g, 12.5 mol), and potassium carbonate (5175 g, 37.4 mol). The resulting mixture was stirred for 2 days at room temperature. The reaction mixture was slowly poured into water/ice (20 L). The precipitate was collected by filtration, washed with ethanol and dried in an oven to afford 2,5-ditosyl-1,2,3,4,5,6-hexahydropyrrolo[3,4-c]pyrrole as a light yellow solid (1345 g, 78%). LCMS: (ES, m/z): 419 [M+H]⁺.

### Step 3. 1,2,3,4,5,6-hexahydropyrrolo[3,4-c]pyrrole hydro bromide salt

To a solution of 2,5-ditosyl-1,2,3,4,5,6-hexahydropyrrolo[3,4-c]pyrrole (1345 g, 2.73 mol) in aqueous hydrogen bromide solution (4500 mL, 48%) in 10 L 4-necked round-bottom flask, was added phenol (1270 g, 13.5 mol). The resulting mixture was stirred for 2 days at 120° C. After cooling to room temperature, the aqueous layer was collected and concentrated under vacuum. The resulting solids were washed with DCM/MeOH (v:v=10:1, 3×300 mL) and dried in an oven to afford 1,2,3,4,5,6-hexahydropyrrolo[3,4-c]pyrrole hydrogen bromide salt as a yellow solid (480 g, 61%). L CMS: (ES, m/z): 111 [M+H]⁺.

### Step 4. Di-tert-butyl pyrrolo[3,4-c]pyrrole-2,5(1H,3H,4H,6H)-dicarboxylate

To a suspension of 1,2,3,4,5,6-hexahydropyrrolo[3,4-c]pyrrole hydrogen bromide salt (458 g, 1.52 mol) in water (4 L) was added sodium bicarbonate (424 g, 5.05 mol) followed by dropwise addition of a solution of di-tert-butyl dicarbonate (807 g, 3.70 mol) in methanol (500 mL) with stirring at 0° C. The resulting solution was stirred for 16 h at 25° C. The precipitate was collected by filtration and dried in an oven to afford di-tert-butyl pyrrolo[3,4-c]pyrrole-2,5(1H,3H,4H,6H)-dicarboxylate as a white solid (300 g, 61%). LCMS (ES, m/z): 311[M+H]⁺.

### Step 5. Tert-butyl 4,5-dihydropyrrolo[3,4-c]pyrrole-2(1H,3H,4H)-carboxylate 4-methylbenzene-1-sulfonic acid salt

To a solution of di-tert-butyl pyrrolo[3,4-c]pyrrole-2,5(1H,3H,4H,6H)-dicarboxylate (200 g, 612 mmol) in propan-2-yl acetate (5 L) was added 4-methylbenzene-1-sulfonic acid (123 g, 647 mmol) in portions at 0° C. The resulting mixture was stirred for 16 h at 55° C. in an oil bath. After cooling to room temperature, the precipitate was collected by filtration and dried in an oven to afford tert-butyl 4,5-dihydropyrrolo[3,4-c]pyrrole-2(1H,3H,4H)-carboxylate 4-methylbenzene-1-sulfonic acid salt as a yellow solid (197 g, 80%). LCMS: (ES, m/z): 211[M+H]⁺.

### Step 6. Tert-butyl 5-(2,3-dihydrobenzo[b][1,4]dioxin-6-ylsulfonyl)-4,5-dihydropyrrolo[3,4-c]pyrrole-2(1H,3H,4H)-carboxylate

To a suspension of tert-butyl 4,5-dihydropyrrolo[3,4-c]pyrrole-2(1H,3H,4H)-carboxylate 4-methylbenzene-1-sulfonic acid salt (61 g, 142 mmol) in water (100 mL) and tetrahydrofuran (30 mL) was added sodium hydroxide (13 g, 325 mmol) followed by portion-wise addition of 2,3-dihydro-1,4-benzodioxine-6-sulfonyl chloride (25 g, 95.9 mmol) at 0° C. The resulting mixture was stirred for 2 h at 25° C. The product was extracted with ethyl acetate (3×200 mL). The organic layers were combined, dried over anhydrous sodium sulfate, filtered and concentrated under vacuum. The resulting material was purified by silica gel chromatography (eluting with 1:10 ethyl acetate/petroleum ether) to afford tert-butyl 5-(2,3-dihydrobenzo[b][1,4]dioxin-6-ylsulfonyl)-4,5-dihydropyrrolo[3,4-c]pyrrole-2(1H,3H,4H)-carboxylate as a white solid (30 g, 73%). LCMS: (ES, m/z): 409 [M+H]⁺.

### Step 7. 2-(2,3-dihydrobenzo[b][1,4]dioxin-6-ylsulfonyl)-1,2,3,4,5,6-hexahydropyrrolo[3,4-c]pyrrole hydrochloric salt

To a solution of tert-butyl 5-(2,3-dihydrobenzo[b][1,4]dioxin-6-ylsulfonyl)-4,5-dihydropyrrolo[3,4-c]pyrrole-2(1H,3H,4H)-carboxylate (30.0 g, 69.8 mmol) in 1,4-dioxane (100 mL) was added hydrochloric acid (200 mL, 4 M in 1,4-dioxane). The resulting solution was stirred for 2 h at 25° C. and then concentrated under vacuum to afford 2-(2,3-dihydrobenzo[b][1,4]dioxin-6-ylsulfonyl)-1,2,3,4,5,6-hexahydropyrrolo[3,4-c]pyrrole hydrochloric salt as a yellow solid (20 g, 79%). LCMS: (ES, m/z): 309 [M+H]⁺.

### Intermediate 2-4. Methyl 2-(2-methyl-1,3-benzothiazol-4-yl)prop-2-enoate

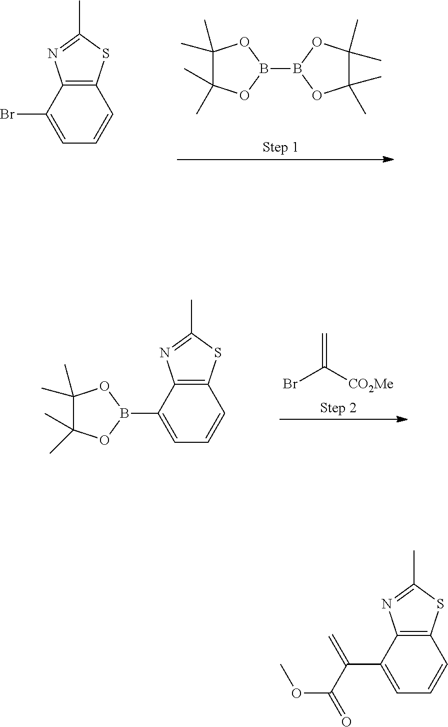

### Step 1. 2-Methyl-4-(4,4,5,5-tetramethyl-1,3,2-dioxaborolan-2-yl)-1,3-benzothiazole

To a solution of 4-bromo-2-methyl-1,3-benzothiazole (3.00 g, 12.9 mmol) in 1,4-dioxane (20 mL) was added 4,4,5,5-tetramethyl-2-(tetramethyl-1,3,2-dioxaborolan-2-yl)-1,3,2-dioxaborolane (4.01 g, 15.5 mmol), Pd(dppf)Cl₂ (960 mg, 1.29 mmol) and potassium acetate (2.58 g, 25.8 mmol). The resulting mixture was stirred for 16 h at 100° C. and then cooled to room temperature. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 2-methyl-4-(4,4,5,5-tetramethyl-1,3,2-dioxaborolan-2-yl)-1,3-benzothiazole as a light yellow oil (2.00 g, 46%). LCMS (ES, m/z) 276 [M+H]⁺.

### Step 2. Methyl 2-(2-methyl-1,3-benzothiazol-4-yl)prop-2-enoate

To a solution of 2-methyl-4-(4,4,5,5-tetramethyl-1,3,2-dioxaborolan-2-yl)-1,3-benzothiazole (600 mg, 1.86 mmol) in 1,4-dioxane (10 mL) was added methyl 2-bromoprop-2-enoate (447 mg, 2.66 mmol), XPhos 3G (80 mg, 0.11 mmol), potassium phosphate (1.4 g, 6.46 mmol) and water (1 mL). The resulting mixture was stirred for 16 h at 100° C. and then cooled to room temperature. The reaction mixture was poured into water (10 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford methyl 2-(2-methyl-1,3-benzothiazol-4-yl)prop-2-enoate as light yellow oil (280 mg, 55%). LCMS (ES, m/z) 234 [M+H]⁺.

The Intermediates in Table 2 were synthesized according to the procedure described for Intermediate 2-4 above.

TABLE 2

| | | LCMS: (ESI) |
|---|---|---|
| Intermediate | Structure and Name | m/z [M + H]⁺ |
| 2-8 | 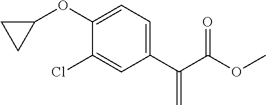 [SMILES: C=C(C(=O)OC)c1ccc(OC2CC2)c(Cl)c1] | 253 |
| | methyl 2-(3-chloro-4-cyclopropoxyphenyl) | |
| | acrylate | |
| 2-9 | 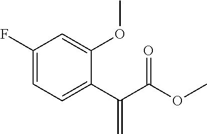 | 211 |
| | methyl 2-(4-fluoro-2-methoxyphenyl)prop-2- | |
| | enoate | |
| 2-27 | 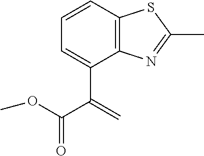 | 234 |
| | methyl 2-(2-methylbenzo[d]thiazol-4- | |
| | yl)acrylate | |

### Intermediate 3-1. methyl 2-(3-methoxyphenyl)acrylate

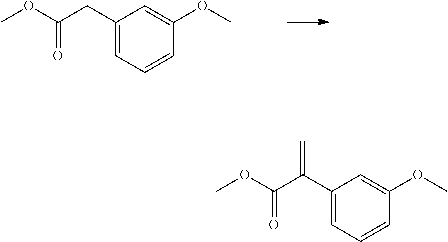

In a 250 mL round-bottom flask was placed methyl 2-(3-methoxyphenyl)acetate (5 g, 27.2 mmol), paraformaldehyde (3 g, 33.3 mmol), n-BU₄NI (1 g, 2.7 mmol), potassium carbonate (9.6 g, 69.5 mmol) and N,N-dimethylformamide (60 mL). The resulting solution was stirred for 10 min at 60° C. in an oil bath. After cooling to room temperature, the solution was diluted with 100 mL of water and extracted with ethyl acetate (3×100 mL). The organic layers were combined, dried over anhydrous sodium sulfate, filtered and concentrated under vacuum to afford 3 g (570%) of methyl 2-(3-methoxyphenyl)prop-2-enoate as a yellow oil. MS: (ESI, m/z): 193 [M+H]⁺.

The Intermediate in Table 3 was synthesized according to the procedure described for Intermediate 3-1 above.

TABLE 3

| | | LCMS: (ESI) |
|---|---|---|
| Intermediate | Structure and Name | m/z [M + H]⁺ |
| 3-16 | 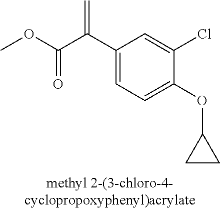 [SMILES: C=C(C(=O)OC)c1ccc(OC2CC2)c(Cl)c1] | 253 |

### Intermediate 5-1. 1-bromo-5-chloro-2-methoxy-4-methylbenzene

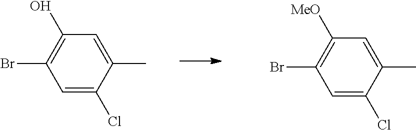

To a solution of 2-bromo-4-chloro-5-methylphenol (2.0 g, 8.1 mmol) in acetone (20 mL) was added potassium carbonate (2.5 g, 16 mmol), and iodomethane (0.66 mL, 9.5 mmol). The resulting mixture was stirred for 2 h at 25° C. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 1-bromo-5-chloro-2-methoxy-4-methylbenzene as a light yellow oil (1.87 g, 82%). GCMS (EI, m/z): 234, 236 [M]⁺.

The Intermediates in Table 4 were synthesized according to the procedure described for Intermediate 5-1 above.

TABLE 4

| | | LCMS: (ESI) |
|---|---|---|
| Intermediate | Structure and Name | m/z [M + H]⁺ |
| 5-2 | 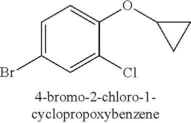 [SMILES: Clc1cc(Br)ccc1OC1CC1] | 247 |
| 5-4 | 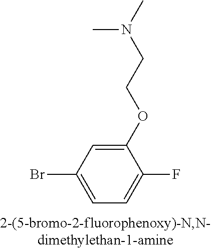 | |

### Intermediate 7-1. 2-{2-[(tert-butoxy)carbonyl]-2,3-dihydro-1H-isoindol-4-yl}-2-oxoacetic acid

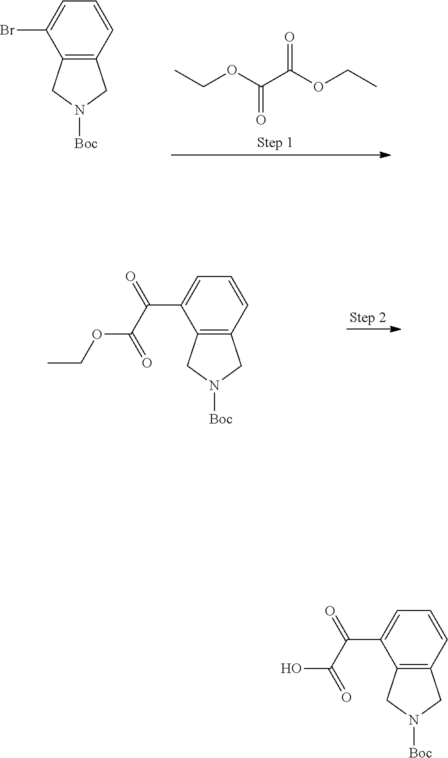

### Step 1. tert-butyl 4-(2-ethoxy-2-oxoacetyl)-2,3-dihydro-1H-isoindole-2-carboxylate

To a solution of tert-buty 4-bromo-2,3-dihydro-1H-isoindole-2-carboxylatein (2.0 g, 6.4 mmol) in THF (20 mL) was added a solution of n-BuLi (2.6 mL, 2.5 M in THF) dropwise with stirring at −78° C. After stirring for 15 min at −78° C., diethyl oxalate (3.1 mL, 32 mmol) was added in. The resulting mixture was stirred for 1 h at −60° C. The reaction mixture was poured into saturated ammonium chloride solution (20 mL) and then extracted with ethyl acetate (3×20 mL). The combined organic layers were dried over anhydrous sodium sulfate, filtered and concentrated under vacuum. The resulting crude product was purified by silica gel chromatography (eluting with 0:100 to 20:80 ethyl acetate/petroleum ether) to afford tert-butyl 4-(2-ethoxy-2-oxoacetyl)-2,3-dihydro-1H-isoindole-2-carboxylate as a light yellow solid (1.12 g, 47%). LCMS (ES, m/z) 320 [M+H]⁺.

### Step 2. 2-{2-[(tert-butoxy)carbonyl]-2,3-dihydro-1H-isoindol-4-yl}-2-oxoacetic acid

To a solution of tert-butyl 4-(2-ethoxy-2-oxoacetyl)-2,3-dihydro-1H-isoindole-2-carboxylate (1.12 g, 2.98 mmol) in tetrahydrofuran (6 mL) was added water (6 mL) and LiOH (421 mg, 16.70 mmol). The resulting mixture was stirred for 16 h at room temperature. The reaction mixture was washed with diethyl ether (1×10 mL) and then acidified to pH=5 with saturated citric acid. The resulting solution was extracted with ethyl acetate (2×10 mL). The combined organic layers were combined, dried over anhydrous sodium sulfate, filtered and concentrated under vacuum to afford 2-[2-[(tert-butoxy)carbonyl]-2,3-dihydro-1H-isoindol-4-yl]-2-oxoacetic acid as a light yellow solid (1.0 g, crude). LCMS (ES, m/z) 292 [M+H]⁺.

The Intermediate in Table 5 was synthesized according to the procedure described for Intermediate 7-1 above.

TABLE 5

| | | LCMS: (ESI) |
|---|---|---|
| Intermediate | Structure and Name | m/z [M + H]⁺ |
| 7-5 | 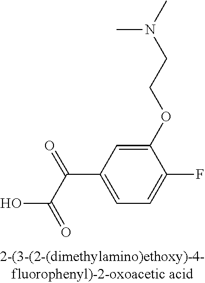 [SMILES: CN(C)CCOc1cc(C(=O)C(=O)O)ccc1F] | 256 |

### Intermediate 14-1. methyl 3-{[(tert-butoxy)carbonyl](methyl)amino}-2-(3-chloro-4-fluoro-2-methoxyphenyl)propanoate

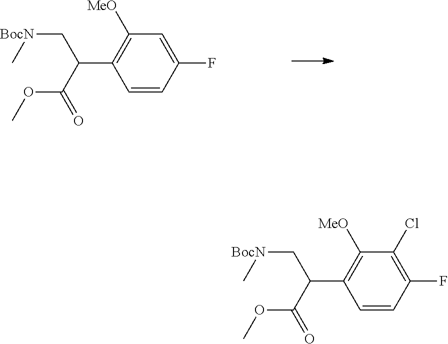 [SMILES: COC(=O)C(CN(C)C(=O)OC(C)(C)C)c1ccc(F)cc1OC ; COC(=O)C(CN(C)C(=O)OC(C)(C)C)c1ccc(F)c(Cl)c1OC]

To a solution of methyl 3-{[(tert-butoxy)carbonyl](methyl)amino}-2-(4-fluoro-2-methoxyphenyl)propanoate (180 mg, 0.53 mmol) in dichloromethane (3 mL) was added NCS (211 mg, 1.58 mmol). The resulting solution was stirred for 16 h at room temperature. The reaction mixture were filtered, and concentrated under vacuum. The resulting crude product was purified by prep-TLC (eluting with 1:5 ethyl acetate/petroleum ether) to afford methyl 3-{[(tert-butoxy)carbonyl](methyl)amino}-2-(3-chloro-4-fluoro-2-methoxyphenyl)propanoate as a light yellow oil (160 mg, 81%). LCMS (ES, m/z) 376, 378 [M+H]⁺.

### Intermediate 27-1. Methyl 2-(3-cyclopropyl-4,5-difluorophenyl)prop-2-enoate

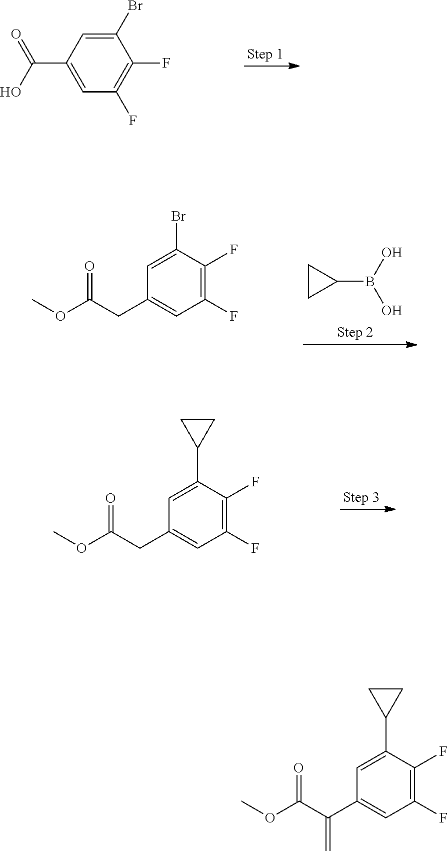 [SMILES: O=C(O)c1cc(F)c(F)c(Br)c1 ; COC(=O)Cc1cc(F)c(F)c(Br)c1 ; OB(O)C1CC1 ; COC(=O)Cc1cc(F)c(F)c(C2CC2)c1 ; C=C(C(=O)OC)c1cc(F)c(F)c(C2CC2)c1]

### Step 1. Methyl 2-(3-bromo-4,5-difluorophenyl)acetate

To a solution of 3-bromo-4,5-difluorobenzoic acid (2.50 g, 10.6 mmol) in toluene (15 mL), was added thionyl chloride (15 mL). The resulting solution was refluxed for 3 h, then cooled to room temperature and concentrated under vacuum. The resulting mixture was dissolved in THF (15 mL) and treated with triethylamine (2.47 mL, 17.9 mmol) and (diazomethyl)trimethylsilane (8.8 mL, 2.0 M in THF) at 0° C. The resulting mixture was stirred for 16 h at room temperature and then poured into saturated aqueous sodium bicarbonate (50 mL) and extracted with ethyl acetate (3×100 mL). The combined organic layers were dried over anhydrous sodium sulfate, filtered, and concentrated under vacuum. The resulting mixture was dissolved in methanol (40 mL) and treated with triethylamine (2.47 mL, 17.9 mmol) and silver (I) benzoate (1.40 g, 6.33 mmol) at 0° C. The mixture was stirred for 16 h at room temperature and then concentrated under vacuum. The crude product was purified by silica gel chromatography (eluting with 0:100 to 10:90 ethyl acetate/petroleum ether) to afford methyl 2-(3-bromo-4,5-difluorophenyl)acetate as a colorless oil (0.98 g, 35%). LCMS (ES, m/z): 265, 267 [M+H]⁺.

### Step 2. Methyl 2-(3-cyclopropyl-4,5-difluorophenyl)acetate

To a solution of methyl 2-(3-bromo-4,5-difluorophenyl)acetate (1.70 g, 6.72 mmol) in 1,4-dioxane (40 mL) was added cyclopropylboronic acid (865 mg, 10.1 mmol), potassium phosphate (4.20 g, 20.1 mmol), Pd(dppf)Cl₂ (246 mg, 0.34 mmol) and water (8 mL). The mixture was stirred for 16 h at 90° C. and cooled to room temperature. The reaction mixture was poured into water (50 mL) and then extracted with ethyl acetate (3×50 mL). The combined organic layers were dried over anhydrous sodium sulfate, filtered, and concentrated under vacuum. The crude product was purified by silica gel chromatography (eluting with 0:100 to 30:70 ethyl acetate/petroleum ether) to afford methyl 2-(3-cyclopropyl-4,5-difluorophenyl)acetate as a colorless oil (550 mg, 36%). LCMS (ES, m/z): 227 [M+H]⁺.

### Step 3. Methyl 2-(3-cyclopropyl-4,5-difluorophenyl)prop-2-enoate

To a solution of methyl 2-(3-cyclopropyl-4,5-difluorophenyl)acetate (550 mg, 2.43 mmol) in DMF (15 mL), was added potassium carbonate (840 mg, 6.08 mmol), tetrabutylammonium iodide (90 mg, 0.24 mmol) and paraformaldehyde (263 mg, 2.92 mmol). The resulting mixture was stirred for 10 min at 60° C. and then cooled to room temperature. The reaction mixture was poured into water (30 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford methyl 2-(3-cyclopropyl-4,5-difluorophenyl)prop-2-enoate as a colorless oil (159 mg, 27%). LCMS (ES, m/z): 239 [M+H]⁺.

### Intermediate 33-1. 2-{1-[(tert-butoxy)carbonyl]pyrrolidin-2-yl}-2-(3-chlorophenyl)acetic acid

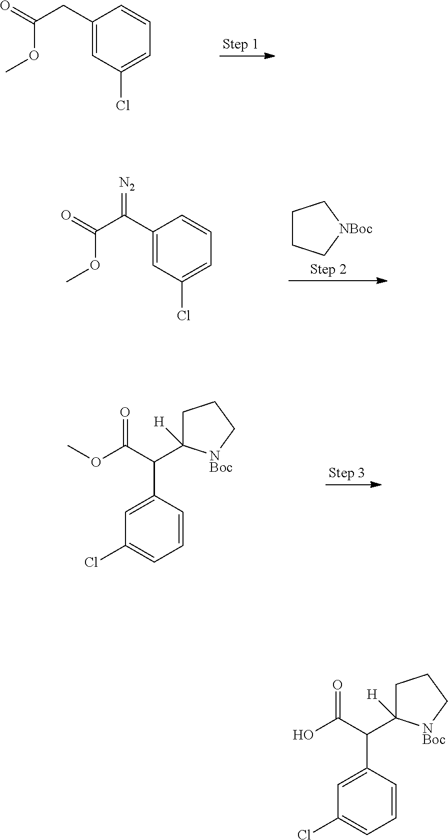

### Step 1. Methyl 2-(3-chlorophenyl)-2-diazoacetate

To a solution of methyl 2-(3-chlorophenyl)acetate (5.00 g, 25.7 mmol) in CH₃CN (50 mL) was added 1,8-diazabicyclo[5.4.0]undec-7-ene (4.87 mL, 32.6 mmol) dropwise with stirring at 0° C. followed by the addition of 4-methylbenzene-1-sulfonyl azide (6.40 g, 32.5 mmol) added dropwise with stirring at 0° C. The solution was stirred for 4 h at 25° C. The reaction mixture was treated with water (50 mL) and extracted with ethyl acetate (3×50 mL). The combined organic layers were dried over anhydrous sodium sulfate, filtered, and concentrated under vacuum. The resulting crude product was purified by silica gel chromatography (eluting with 0:100 to 10:90 ethyl acetate/dichloromethane) to afford methyl 2-(3-chlorophenyl)-2-diazoacetate as a yellow solid (5.00 g, 83%). LCMS (ES, m/z): 211, 213 [M+H]⁺.

### Step 2. Tert-butyl 2-[1-(3-chlorophenyl)-2-methoxy-2-oxoethyl]pyrrolidine-1-carboxylate

To a solution of tert-butyl pyrrolidine-1-carboxylate (894 mg, 5.22 mmol) in hexane (150 mL) was added tetrakis [(R)-(+)-N-(P-dodecylphenylsulfonyl)prolinato]dirhodium (II) (49 mg, 0.026 mmol) followed by treatment with methyl 2-(3-chlorophenyl)-2-diazoacetate (550 mg, 2.61 mmol) as a solution in hexane (100 mL) over 60 min with stirring at −50° C. The resulting solution was stirred for 10 h at −50° C. and then 16 h at room temperature. The reaction was poured into saturated ammonium chloride solution (100 mL) and then extracted with ethyl acetate (3×150 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 2-[1-(3-chlorophenyl)-2-methoxy-2-oxoethyl]pyrrolidine-1-carboxylate as a yellow solid (400 mg, 39%). LCMS (ES, m/z): 354, 356 [M+H]⁺.

### Step 3. 2-{1-[(tert-butoxy)carbonyl]pyrrolidin-2-yl}-2-(3-chlorophenyl)acetic acid

To a solution of tert-butyl 2-[1-(3-chlorophenyl)-2-methoxy-2-oxoethyl]pyrrolidine-1-carboxylate (400 mg, 1.13 mmol) in tetrahydrofuran (20 mL) and water (5 mL) was added lithium hydroxide (135 mg, 5.65 mmol). The resulting mixture was stirred for 18 h at room temperature. The reaction mixture was washed with diethyl ether (1×10 mL) and then acidified to pH=6 with saturated citric acid. The resulting solution was extracted with ethyl acetate (3×20 mL). The combined organic layers were dried over anhydrous sodium sulfate, filtered, and concentrated under vacuum to afford 2-{1-[(tert-butoxy)carbonyl]pyrrolidin-2-yl}-2-(3-chlorophenyl)acetic acid as yellow oil (300 mg, 78%). LCMS (ES, m/z): 340, 342 [M+H]⁺.

### Intermediate 38-1. 1,2,3,4,5,6-hexahydropyrrolo[3,4-c]pyrrole hydrogen bromide salt

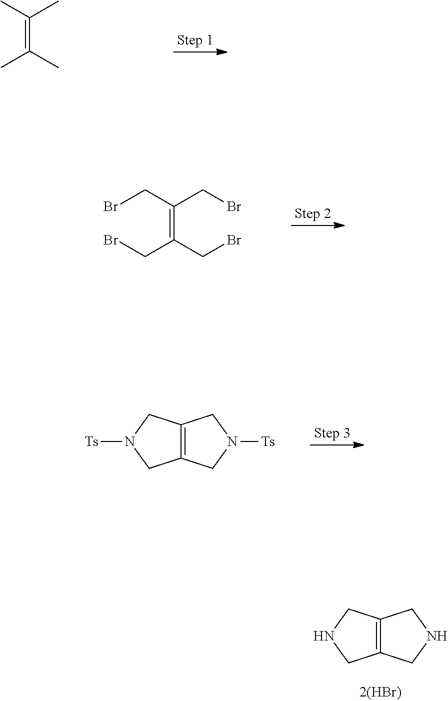

### Step 1. 1,4-dibromo-2,3-bis(bromomethyl)but-2-ene

To a solution of 2,3-dimethylbut-2-ene (1000 g, 11.9 mol) in DCM (500 mL) in 4 L 4-necked round bottom flask was added aqueous hydrogen bromide solution (150 mL, 48%). The reaction was treated with bromine (9.90 kg, 62.0 mol) while stirring at 0° C. and then heated to 45° C. in an oil bath and stirred for an additional 2 days. After cooling to room temperature, the reaction mixture was carefully poured into saturated sodium hydrogen sulfite solution (10 L). The precipitate was collected by filtration and dried in oven to afford 1,4-dibromo-2,3-bis(bromomethyl)but-2-ene as a light yellow solid (3345 g, 49%). GCMS: (EI, m/z): 398, 400, 402 [M]⁺.

### Step 2. 2,5-ditosyl-1,2,3,4,5,6-hexahydropyrrolo[3,4-c]pyrrole

To a solution of 1,4-dibromo-2,3-bis(bromomethyl)but-2-ene (2000 g, 3.50 mol) in DMF (20 L) was added 4-methylbenzene-1-sulfonamide (2137 g, 12.5 mol), and potassium carbonate (5175 g, 37.4 mol). The resulting mixture was stirred for 2 days at room temperature. The reaction mixture was then slowly poured into water/ice (20 L). The precipitate was collected by filtration, washed with ethanol and dried in oven to afford 2,5-ditosyl-1,2,3,4,5,6-hexahydropyrrolo[3,4-c]pyrrole as a light yellow solid (1345 g, 78%). LCMS: (ES, m/z): 419 [M+H]⁺.

### Step 3. 1,2,3,4,5,6-hexahydropyrrolo[3,4-c]pyrrole hydro bromide salt

To a solution of 2,5-ditosyl-1,2,3,4,5,6-hexahydropyrrolo[3,4-c]pyrrole (1345 g, 2.73 mol) in aqueous hydrogen bromide solution (4500 mL, 48%) in 10 L 4-necked round-bottom flask, was added phenol (1270 g, 13.5 mol). The resulting mixture was stirred for 2 days at 120° C. After cooling to room temperature, the aqueous layer was collected and concentrated under vacuum. The resulting solids were washed with DCM/MeOH (v:v=10:1, 3×300 mL) and dried in an oven to afford 1,2,3,4,5,6-hexahydropyrrolo[3,4-c]pyrrole hydrogen bromide salt as a yellow solid (480 g, 61%). L CMS: (ES, m/z): 111 [M+H]⁺.

### Intermediate 43-1. 2-hydroxy-2-(2-methyl-1,3-benzoxazol-4-yl)acetic acid

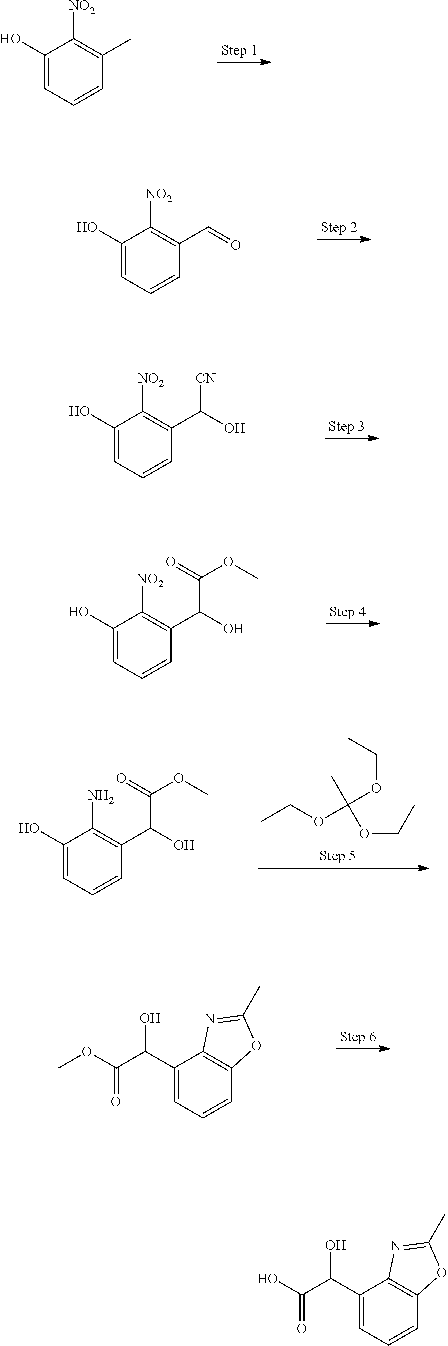

### Step 1. 3-hydroxy-2-nitrobenzaldehyde

To a solution of 3-methyl-2-nitrophenol (200 g, 1.29 mol) in acetic anhydride (1600 mL) was added sulfuric acid (240 mL) and acetic acid (1620 mL). This was followed by the addition of chromium trioxide (280 g, 2.77 mol) in several batches with stirring at 0° C. The resulting mixture was stirred for 2.5 h at 0° C. and then poured into ice/water (5000 mL). The solids were collected by filtration and then washed with water (3×1 L), saturated sodium carbonate solution (3×800 mL), and water (3×1 L). The solids were dissolved in ethanol (380 mL) and concentrated hydrochloric acid (617 mL). The resulting solution was stirred for 1.5 h at 110° C. and then cooled to room temperature. The reaction mixture was concentrated under vacuum to afford 3-hydroxy-2-nitrobenzaldehyde as a yellow solid (38.0 g, 18%). LCMS (ES, m/z): 166 [M−H]⁻.

### Step 2. 2-hydroxy-2-(3-hydroxy-2-nitrophenyl)acetonitrile

To a solution of 3-hydroxy-2-nitrobenzaldehyde (38.0 g, 204 mmol) in dichloromethane (500 mL) was added ZnI₂ (14.5 g, 44.5 mmol). The reaction was treated with trimethylsilyl cyanide (100 mL, 708 mmol) added dropwise with stirring at 0° C. The resulting mixture was stirred for 2.5 h at 25° C. The reaction was poured into brine (200 mL) and then extracted with ethyl acetate (3×500 mL). The combined organic layers were dried over anhydrous sodium sulfate, filtered, and concentrated under vacuum to afford 2-hydroxy-2-(3-hydroxy-2-nitrophenyl)acetonitrile as a yellow solid (34.0 g, 73%). LCMS (ES, m/z): 195 [M+H]⁺.

### Step 3. Methyl 2-hydroxy-2-(3-hydroxy-2-nitrophenyl)acetate

To a solution of 2-hydroxy-2-(3-hydroxy-2-nitrophenyl)acetonitrile (34.0 g, 157 mmol) in methanol (80 mL) was added hydrochloric acid (80 mL, 4 N in 1,4-dioxane). The resulting solution was stirred for 45 min at 60° C. and cooled to room temperature. The reaction mixture was concentrated under vacuum and purified by silica gel chromatography (eluting with 0:100 to 35:65 ethyl acetate/petroleum ether) to afford methyl 2-hydroxy-2-(3-hydroxy-2-nitrophenyl)acetate as a yellow solid (23.0 g, 58%). LCMS (ES, m/z): 228 [M+H]⁺.

### Step 4. Methyl 2-(2-amino-3-hydroxyphenyl)-2-hydroxyacetate

To a solution of methyl 2-hydroxy-2-(3-hydroxy-2-nitrophenyl)acetate (23.0 g, 0.11 mol) in methanol (500 mL) was added anhydrous palladium carbon (2.3 g, 10 wt % Pd). The resulting mixture was stirred for 16 h at 25° C. under hydrogen atmosphere (3 atm). The reaction mixture was filtered and concentrated under vacuum to afford methyl 2-(2-amino-3-hydroxyphenyl)-2-hydroxyacetate as a yellow solid (14.0 g, 60%). LCMS (ES, m/z): 198 [M+H]⁺.

### Step 5. Methyl 2-hydroxy-2-(2-methyl-1,3-benzoxazol-4-yl)acetate

To a solution of methyl 2-(2-amino-3-hydroxyphenyl)-2-hydroxyacetate (9.0 g, 43.4 mmol) in 1,1,1-triethoxyethane (150 mL) was added bismuth (III) trifluoromethanesulfonate (800 mg, 1.18 mmol). The resulting mixture was stirred for 10 min at 85° C. After cooling to room temperature, the reaction mixture was concentrated under vacuum. The resulting crude product was purified by silica gel chromatography (eluting with 0:100 to 50:50 ethyl acetate/petroleum ether) to afford methyl 2-hydroxy-2-(2-methyl-1,3-benzoxazol-4-yl)acetate as a white solid (6.3 g, 63%). LCMS (ES, m/z): 222 [M+H]⁺.

### Step 6. 2-hydroxy-2-(2-methyl-1,3-benzoxazol-4-yl)acetic acid

To a solution of methyl 2-hydroxy-2-(2-methyl-1,3-benzoxazol-4-yl)acetate (500 mg, 2.26 mmol) in tetrahydrofuran (20 mL) and water (2 mL) was added lithium hydroxide (271 mg, 11.3 mmol). The resulting mixture was stirred for 1 h at room temperature. The reaction mixture was washed with diethyl ether (1×10 mL) and then acidified to pH=6 with hydrochloric acid (1 N). The resulting solution was extracted with ethyl acetate (2×20 mL). The combined organic layers were dried over anhydrous sodium sulfate, filtered, and concentrated under vacuum to afford 2-hydroxy-2-(2-methyl-1,3-benzoxazol-4-yl)acetic acid as a white solid (386 mg, 82%). LCMS (ES, m/z): 208 [M+H]⁺.

### Intermediate 44-1. 2-methyl-1,3-benzothiazole-4-carbaldehyde

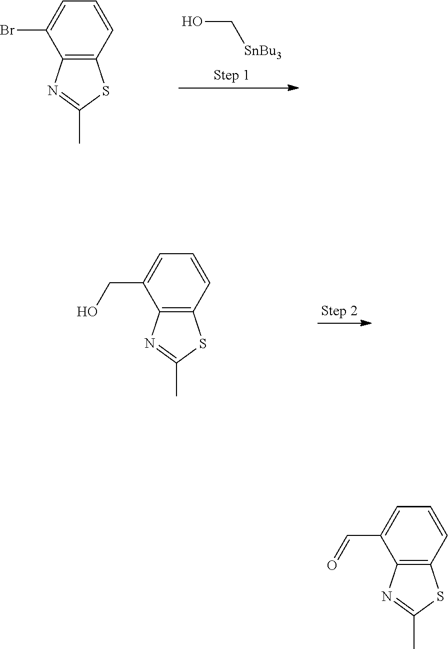

### Step 1. (2-methyl-1,3-benzothiazol-4-yl)methanol

To a solution of 4-bromo-2-methyl-1,3-benzothiazole (2.10 g, 9.21 mmol) in 1,4-dioxane (70 mL) was added (tributylstannyl)methanol (3.84 g, 12.0 mmol), and Pd(PPh₃)₄ (1.60 g, 1.38 mmol). The resulting mixture was stirred for 16 h at 100° C. and then cooled to room temperature. The reaction mixture was poured into water (50 mL) and then extracted with ethyl acetate (3×70 mL). The combined organic layers were dried over anhydrous sodium sulfate, filtered, and concentrated under vacuum. The crude product was purified by silica gel chromatography (eluting with 0:100 to 80:20 ethyl acetate/petroleum ether) to afford (2-methyl-1,3-benzothiazol-4-yl)methanol as a yellow oil (1.20 g, 73%). LCMS (ES, m/z): 180 [M+H]⁺.

### Step 2. 2-methyl-1,3-benzothiazole-4-carbaldehyde

To a solution of oxalyl chloride (1.39 mL, 13.39 mmol) in dichloromethane (30 mL) was added DMSO (1.43 mL, 20.1 mmol) dropwise with stirring at −78° C. The resulting mixture was stirred for 30 min at −78° C. The reaction was treated with (2-methyl-1,3-benzothiazol-4-yl)methanol (1.20 g, 6.69 mmol) in dichloromethane (10 mL) added slowly at −78° C. After 2 h TEA (5.58 mL, 40.1 mmol) was added and the mixture was warmed to room temperature and stirred for 2 h. The reaction was poured into brine (30 mL) and then extracted with ethyl acetate (3×50 mL). The combined organic layers were dried over anhydrous sodium sulfate, filtered, and concentrated under vacuum to afford 2-methyl-1,3-benzothiazole-4-carbaldehyde as a yellow oil (900 mg, 76%). LCMS (ES, m/z): 178 [M+H]⁺.

### Intermediate 63-1. tert-butyl (R)-(3-(5-((2,3-dihydrobenzo[b][1,4]dioxin-6-yl)sulfonyl)-3,4,5,6-tetrahydropyrrolo[3,4-c]pyrrol-2(1H)-yl)-3-oxo-2-phenylpropyl)carbamate

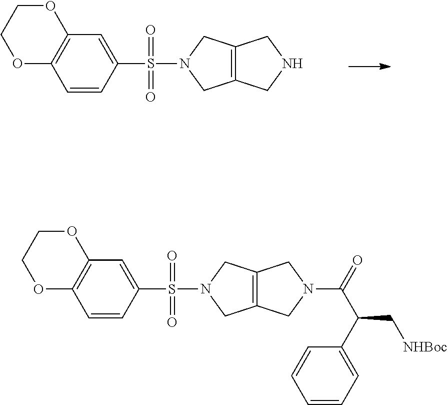 [SMILES: O=S(=O)(c1ccc2c(c1)OCCO2)N1CC2=C(CNC2)C1 ; CC(C)(C)OC(=O)NC[C@H](C(=O)N1CC2=C(C1)CN(S(=O)(=O)c1ccc3c(c1)OCCO3)C2)c1ccccc1]

In a dry 25 ml RBF under N₂ was added 2-((2,3-dihydrobenzo[b][1,4]dioxin-6-yl)sulfonyl)-1,2,3,4,5,6-hexahydropyrrolo[3,4-c]pyrrole (200 mg, 0.649 mmol), (R)-3-((tert-butoxycarbonyl)amino)-2-phenylpropanoic acid (189 mg, 0.713 mmol), DMF (1 mL), DIEA (170 μl, 0.973 mmol) and HATU (271 mg, 0.713 mmol). After 3 h, the reaction was diluted with 50 ml of saturated aqueous bicarbonate solution (50 mL) and extracted with EtOAc (4×20 mL). The extracts were dried over Na₂SO₄, filtered and the solvent was removed in vacuo to afford 524 mg of a brown gummy solid. The crude material was purified by a Biotage SNAP-25 Silica column and eluted with an EtOAc/Hexane gradient (0-100% EtOAc). The desired product was isolated affording 309 mg of a white solid.

The Intermediate in Table 6 was synthesized according to the procedure described for Intermediate 63-1 above.

TABLE 6

| | | LCMS: (ESI) |
|---|---|---|
| Intermediate | Structure and Name | m/z [M + H]⁺ |
| 63-9 | 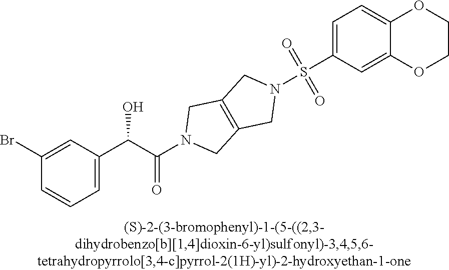 [SMILES: O=C([C@@H](O)c1cccc(Br)c1)N1CC2=C(C1)CN(S(=O)(=O)c1ccc3c(c1)OCCO3)C2] | 521 |

### Intermediate 66. tert-butyl 5-[3-[(1S)-2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]phenyl]-octahydropyrrolo[3,4-c]pyrrole-2-carboxylate

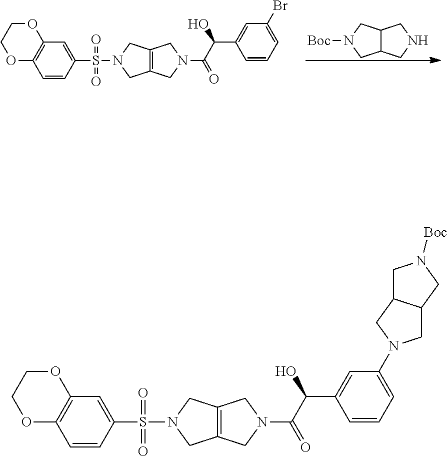 [SMILES: O=C([C@@H](O)c1cccc(Br)c1)N1CC2=C(C1)CN(S(=O)(=O)c1ccc3c(c1)OCCO3)C2 ; CC(C)(C)OC(=O)N1CC2CNCC2C1 ; CC(C)(C)OC(=O)N1CC2CN(c3cccc([C@H](O)C(=O)N4CC5=C(C4)CN(S(=O)(=O)c4ccc6c(c4)OCCO6)C5)c3)CC2C1]

To a solution of (2S)-2-(3-bromophenyl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (100 mg, 0.20 mmol) in toluene (5 mL) was added tert-butyl octahydropyrrolo[3,4-c]pyrrole-2-carboxylate (609 mg, 2.87 mmol), RuPhos 2G (15 mg, 0.02 mmol), RuPhos (18 mg, 0.04 mmol), and cesium carbonate (189 mg, 0.58 mmol). The resulting mixture was stirred overnight at 100° C. After cooling to room temperature, the reaction was concentrated under vacuum. The crude product was purified by silica gel chromatography (eluting with 1:25 MeOH/DCM) to afford tert-butyl 5-[3-[(1S)-2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]phenyl]-octahydropyrrolo-[3,4-c]pyrrole-2-carboxylate as a light yellow solid (100 mg, 80%). LCMS (ES, m/z): 653 [M+H]⁺.

The Intermediates in Table 7 were synthesized according to the procedure described for Intermediate 66 above.

TABLE 7

| | | LCMS: (ESI) |
|---|---|---|
| Intermediate | Structure and Name | m/z [M + H]⁺ |
| 66-2 | 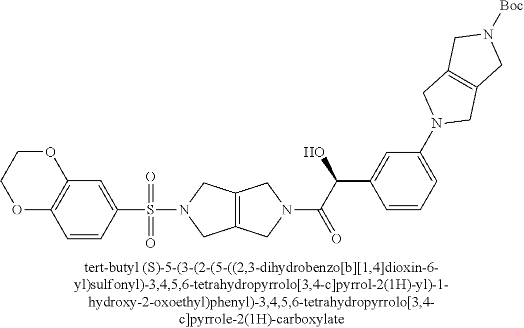 [SMILES: CC(C)(C)OC(=O)N1CC2=C(C1)CN(c1cccc([C@H](O)C(=O)N3CC4=C(C3)CN(S(=O)(=O)c3ccc5c(c3)OCCO5)C4)c1)C2] | 651 |
| 66-3 | 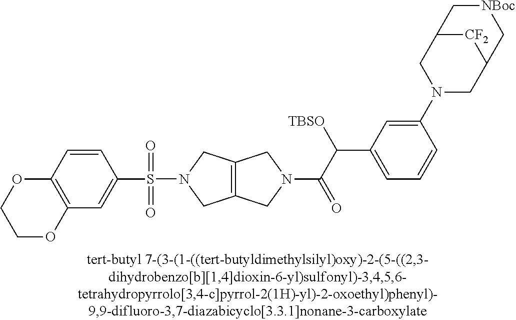 [SMILES: CC(C)(C)OC(=O)N1CC2CN(c3cccc(C(O[Si](C)(C)C(C)(C)C)C(=O)N4CC5=C(C4)CN(S(=O)(=O)c4ccc6c(c4)OCCO6)C5)c3)CC(C1)C2(F)F] | 817 |
| 66-4 | 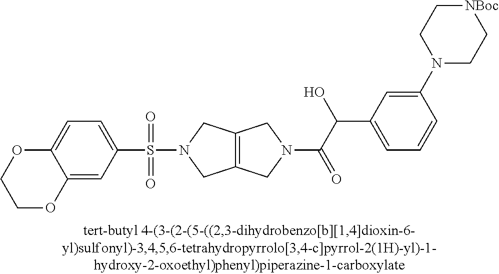 [SMILES: CC(C)(C)OC(=O)N1CCN(c2cccc(C(O)C(=O)N3CC4=C(C3)CN(S(=O)(=O)c3ccc5c(c3)OCCO5)C4)c2)CC1] | 627 |

### Intermediate 75. 4-[[(tert-butoxy)carbonyl]amino]-2-(3,5-dichlorophenyl)butanoic acid

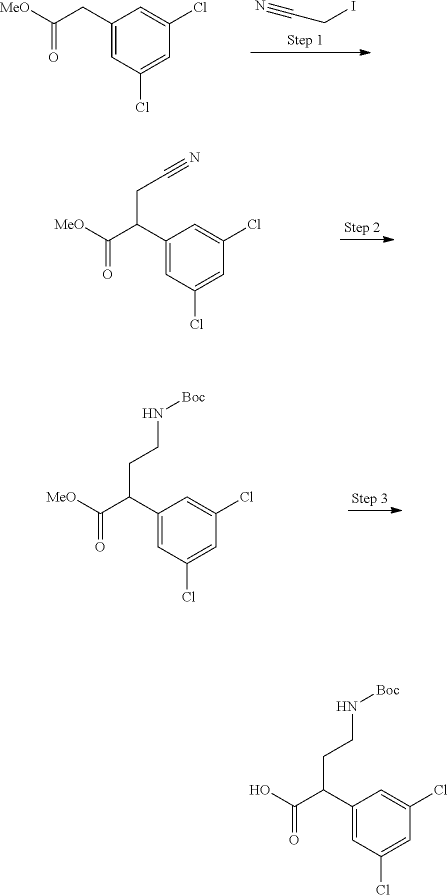

### Step 1. Methyl 3-cyano-2-(3,5-dichlorophenyl)propanoate

To a solution of methyl 2-(3,5-dichlorophenyl)acetate (2.00 g, 8.67 mmol) in tetrahydrofuran (20 mL) was added LDA (5.5 mL, 2 M in THF) dropwise with stirring at −78° C. The solution was stirred for 30 min at −78° C. The reaction was treated with 2-iodoacetonitrile (2.30 g, 13.8 mmol) and stirred for 1 h at −78° C. The contents were poured into saturated aqueous ammonium chloride solution (50 mL) and then extracted with ethyl acetate (3×50 mL). The combined organic layers were dried over anhydrous sodium sulfate, filtered, and concentrated under vacuum. The crude product was purified by silica gel chromatography (eluting with 0:100 to 20:80 ethyl acetate/petroleum ether) to afford methyl 3-cyano-2-(3,5-dichlorophenyl)propanoate as a yellow oil (1.30 g, 58%). LCMS (ES, m/z): 258, 260 [M+H]⁺.

### Step 2. Methyl 4-[[(tert-butoxy)carbonyl]amino]-2-(3,5-dichlorophenyl)butanoate

To a solution of methyl 3-cyano-2-(3,5-dichlorophenyl)propanoate (1.50 g, 5.52 mmol) in methanol (20 mL) was added Raney Ni (946 mg, 11.0 mmol), and di-tert-butyl dicarbonate (6.03 g, 27.6 mmol). The resulting mixture was stirred for 4 h at room temperature under hydrogen (2-3 atm). The reaction mixture was filtered and concentrated under vacuum. The resulting crude product was purified by silica gel chromatography (eluting with 0:100 to 20:80 ethyl acetate/petroleum ether) to afford methyl 4-[[(tert-butoxy)carbonyl]amino]-2-(3,5-dichlorophenyl)butanoate as a yellow oil (1.80 g, 90%). LCMS (ES, m/z): 362, 364 [M+H]⁺.

### Step 3. 4-[[(tert-butoxy)carbonyl]amino]-2-(3,5-dichlorophenyl)butanoic acid

To a solution of methyl 4-[[(tert-butoxy)carbonyl]amino]-2-(3,5-dichlorophenyl)butanoate (300 mg, 0.79 mmol) in tetrahydrofuran (2 mL) and water (2 mL) was added lithium hydroxide (94 mg, 3.93 mmol). The resulting mixture was stirred for 16 h at room temperature. The reaction mixture was washed with diethyl ether (1×10 mL) and then acidified to pH=7 with saturated aqueous citric acid. The product was extracted with ethyl acetate (3×20 mL). The combined organic layers were dried over anhydrous sodium sulfate, filtered, and concentrated under vacuum to afford 4-[[(tert-butoxy)carbonyl]amino]-2-(3,5-dichlorophenyl)butanoic acid as a yellow oil (180 mg, 66%). LCMS (ES, m/z): 348, 350 [M+H]⁺.

### Intermediate 80. Methyl 3-(azetidin-1-yl)-2-(2-methyl-1,3-benzothiazol-4-yl)propanoate

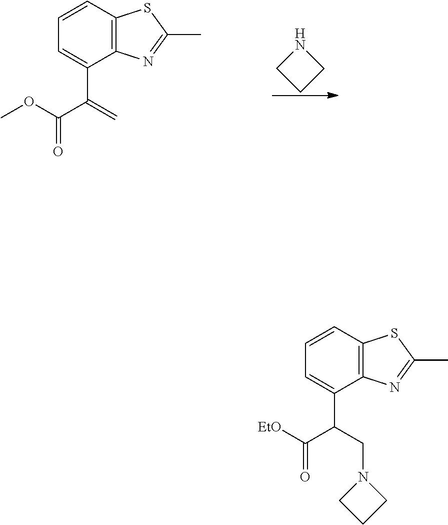

To a solution of methyl 2-(2-methyl-1,3-benzothiazol-4-yl)prop-2-enoate (200 mg, 0.86 mmol) in tetrahydrofuran (2 mL) was added azetidine (98 mg, 1.72 mmol). The resulting mixture was stirred for 2 h at room temperature. The reaction mixture was concentrated under vacuum. The resulting crude product was purified by prep-TLC (eluting with 20:1 dichloromethane/methanol) to afford methyl 3-(azetidin-1-yl)-2-(2-methyl-1,3-benzothiazol-4-yl)propanoate as a brown oil (170 mg, 68%). LCMS (ES, m/z): 305 [M+H]⁺.

### Intermediate 83. 2-(3-bromophenyl)-2-[(tert-butyldimethylsilyl)oxy]-1-[5-(2,3-dihydro-1,4-benzodioxine-6-sulfonyl)-1H,2H,3H,4H,5H,6H-pyrrolo[3,4-c]pyrrol-2-yl]ethan-1-one

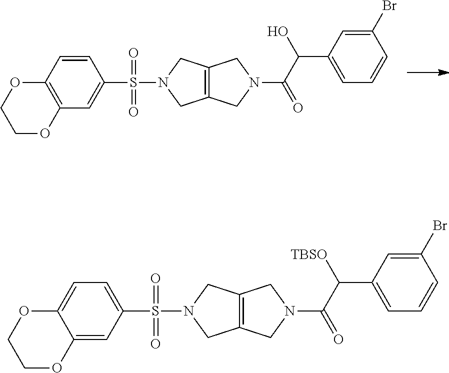 [SMILES: O=C(C(O)c1cccc(Br)c1)N1CC2=C(C1)CN(S(=O)(=O)c1ccc3c(c1)OCCO3)C2 ; CC(C)(C)[Si](C)(C)OC(C(=O)N1CC2=C(C1)CN(S(=O)(=O)c1ccc3c(c1)OCCO3)C2)c1cccc(Br)c1]

To a solution of 2-(3-bromophenyl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one (800 mg, 1.53 mmol) in DMF (10 mL) was added imidazole (314 mg, 4.61 mmol), tert-butyl(chloro)dimethylsilane (0.43 mL, 2.30 mmol) and DMAP (20 mg, 0.164 mmol). The resulting solution was stirred for 2 h at 70° C. and then cooled to room temperature. The reaction mixture was poured into water (10 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 2-(3-bromophenyl)-2-[(tert-butyldimethylsilyl)oxy]-1-[5-(2,3-dihydro-1,4-benzodioxine-6-sulfonyl)-1H,2H,3H,4H,5H,6H-pyrrolo[3,4-c]pyrrol-2-yl]ethan-1-one as a light yellow oil (750 mg, 69%). LCMS (ES, m/z): 635, 637 [M+H]⁺.

The Intermediates in Table 8 were synthesized according to the procedure described for Intermediate 83 above.

TABLE 8

| | | LCMS: (ESI) |
|---|---|---|
| Intermediate | Structure and Name | m/z [M + H]⁺ |
| 83-2 | 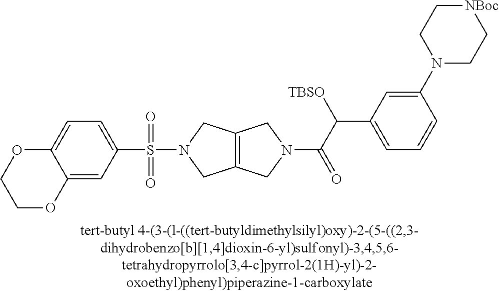 [SMILES: CC(C)(C)OC(=O)N1CCN(c2cccc(C(O[Si](C)(C)C(C)(C)C)C(=O)N3CC4=C(C3)CN(S(=O)(=O)c3ccc5c(c3)OCCO5)C4)c2)CC1] | 741 |

### Intermediate 85. Tert-butyl 7-(3-{2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl}phenyl)-9,9-difluoro-3,7-diazabicyclo[3.3.1]nonane-3-carboxylate

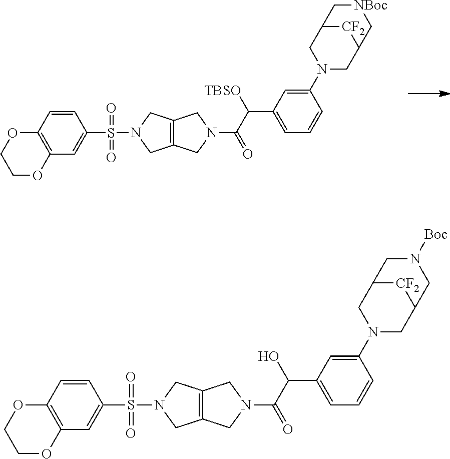 [SMILES: CC(C)(C)OC(=O)N1CC2CN(c3cccc(C(O[Si](C)(C)C(C)(C)C)C(=O)N4CC5=C(C4)CN(S(=O)(=O)c4ccc6c(c4)OCCO6)C5)c3)CC(C1)C2(F)F ; CC(C)(C)OC(=O)N1CC2CN(c3cccc(C(O)C(=O)N4CC5=C(C4)CN(S(=O)(=O)c4ccc6c(c4)OCCO6)C5)c3)CC(C1)C2(F)F]

To 2-[(tert-butyldimethylsilyl)oxy]-2-(3-{9,9-difluoro-3,7-diazabicyclo[3.3.1]nonan-3-yl}phenyl)-1-[5-(2,3-dihydro-1,4-benzodioxine-6-sulfonyl)-1H,2H,3H,4H,5H,6H-pyrrolo[3,4-c]pyrrol-2-yl]ethan-1-one (280 mg, 0.34 mmol) was added TBAF (5 mL, 1 M in TIF). The resulting solution was stirred for 30 min at room temperature. The reaction mixture was poured into water (5 mL) and then extracted with ethyl acetate (3×10 mL). The combined organic layers were dried over anhydrous sodium sulfate, filtered, and concentrated under vacuum. The resulting crude product was purified by prep-TLC (eluting with 3:1 ethyl acetate/petroleum ether) to afford tert-butyl 7-(3-[2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]phenyl)-9,9-difluoro-3,7-diazabicyclo[3.3.1]nonane-3-carboxylate as a yellow oil (220 mg, 82%). LCMS (ES, m/z): 703 [M+H]⁺.

### Intermediate 87. Tert-butyl 4-(3-{1-[(tert-butyldimethylsilyl)oxy]-2-[5-(2,3-dihydro-1,4-benzodioxine-6-sulfonyl)-1H,2H,3H,4H,5H,6H-pyrrolo[3,4-c]pyrrol-2-yl]-2-sulfanylideneethyl}phenyl)piperazine-1-carboxylate

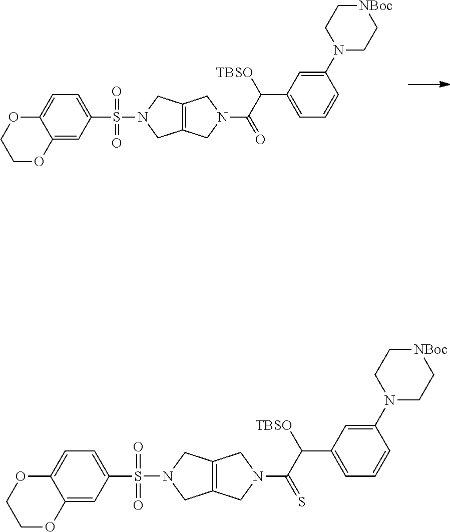 [SMILES: CC(C)(C)OC(=O)N1CCN(c2cccc(C(O[Si](C)(C)C(C)(C)C)C(=O)N3CC4=C(C3)CN(S(=O)(=O)c3ccc5c(c3)OCCO5)C4)c2)CC1 ; CC(C)(C)OC(=O)N1CCN(c2cccc(C(O[Si](C)(C)C(C)(C)C)C(=S)N3CC4=C(C3)CN(S(=O)(=O)c3ccc5c(c3)OCCO5)C4)c2)CC1]

To a solution of tert-butyl 4-(3-[1-[(tert-butyldimethylsilyl)oxy]-2-[5-(2,3-dihydro-1,4-benzodioxine-6-sulfonyl)-1H,2H,3H,4H,5H,6H-pyrrolo[3,4-c]pyrrol-2-yl]-2-oxoethyl]phenyl)piperazine-1-carboxylate (320 mg, 0.43 mmol) in dichloromethane (5 mL), was added Lawesson Reagent (88 mg, 0.22 mmol). The resulting mixture was stirred for 16 h at room temperature. The reaction mixture was concentrated under vacuum. The resulting crude product was purified by Prep-TLC eluting with 1:1 ethyl acetate/petroleum ether to give the product as a light yellow oil (130 mg, 36%). LCMS (ES, m/z) 757 [M+H]⁺.

### Intermediate 89. 2-[2,3-dihydro(2,2,3,3-2D4)-1,4-benzodioxine-6-sulfonyl]-1H,2H,3H,4H,5H,6H-pyrrolo[3,4-c]pyrrole

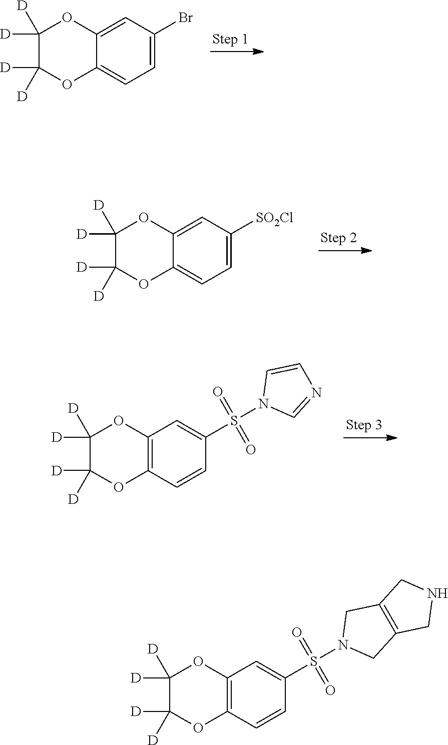 [SMILES: [2H]C1([2H])Oc2ccc(Br)cc2OC1([2H])[2H] ; [2H]C1([2H])Oc2ccc(S(=O)(=O)Cl)cc2OC1([2H])[2H] ; [2H]C1([2H])Oc2ccc(S(=O)(=O)n3ccnc3)cc2OC1([2H])[2H] ; [2H]C1([2H])Oc2ccc(S(=O)(=O)N3CC4=C(CNC4)C3)cc2OC1([2H])[2H]]

### Step 1. 2,3-dihydro(2,2,3,3-2H4)-1,4-benzodioxine-6-sulfonyl chloride

To a solution of n-BuLi (5.6 mL, 2.5 M in THF) was added n-Bu₂Mg (14 mL, 1 M in THF) at room temperature. The resulting mixture was stirred for 10 min at room temperature and treated with 6-bromo-2,3-dihydro(2,2,3,3-2H4)-1,4-benzodioxine (2.0 g, 9.04 mmol) in tetrahydrofuran (10 mL) added dropwise with stirring at −10° C. The resulting mixture was stirred for 1 h and then added to a solution of sulfuryl chloride (16 mL) in toluene (8 mL) with stirring at −10° C. The resulting mixture was stirred for 0.5 h and quenched with saturated aqueous ammonium chloride solution (30 mL). The product was extracted with ethyl acetate (3×30 mL). The organic layers were combined, dried over anhydrous sodium sulfate, filtered and concentrated under vacuum. The resulting crude product was purified by silica gel chromatography (eluting with 0 to 50% ethyl acetate/petroleum ether) to afford 2,3-dihydro(2,2,3,3-2H4)-1,4-benzodioxine-6-sulfonyl chloride as a yellow oil (1.3 g, 60%).

### Step 2. 1-[2,3-dihydro(2,2,3,3-2H4)-1,4-benzodioxine-6-sulfonyl]-1H-imidazole

To a solution of 2,3-dihydro(2,2,3,3-H)-1,4-benzodioxine-6-sulfonyl chloride (1.3 g, 5.01 mmol) in DCM (20 mL) was added 1H-imidazole (742 mg, 10.9 mmol). The resulting solution was stirred for 2 h at room temperature. Then the reaction mixture was filtered and concentrated under vacuum to afford 1-[2,3-dihydro(2,2,3,3-2H4)-1,4-benzodioxine-6-sulfonyl]-1H-imidazole as a white solid (1.2 g, 89%). LCMS (ES, m/z): 271 [M+H]⁺.

### Step 3. 2-[2,3-dihydro(2,2,3,3-2H4)-1,4-benzodioxine-6-sulfonyl]-1H,2H,3H,4H,5H,6H-pyrrolo[3,4-c]pyrrole

To a solution of 1H,2H,3H,4H,5H,6H-pyrrolo[3,4-c]pyrrole dihydrobromide (3.6 g, 13.2 mmol) in water (15 mL) and ethanol (15 mL) was added 1-[2,3-dihydro(2,2,3,3-2H4)-1,4-benzodioxine-6-sulfonyl]-1H-imidazole (1.2 g, 4.08 mmol). The resulting solution was stirred for 18 h at room temperature and then 48 h at 60° C. After cooling to room temperature, the solution was basified to pH=14 with sodium hydroxide and then extracted with DCM (3×20 mL). The organic layers were combined, dried over anhydrous sodium sulfate, filtered and concentrated under vacuum to afford 2-[2,3-dihydro(2,2,3,3-2H4)-1,4-benzodioxine-6-sulfonyl]-1H,2H,3H,4H,5H,6H-pyrrolo[3,4-c]pyrrole as a yellow solid (500 mg, 39%). LCMS (ES, m/z): 313 [M+H]⁺.

### Methods for Preparing Final Compounds

### Method A

### (2S)-1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one and (2R)-1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one

### Example 94-1. 1^{st} Eluting Isomer; Example 94-2. 2^{nd} Eluting Isomer

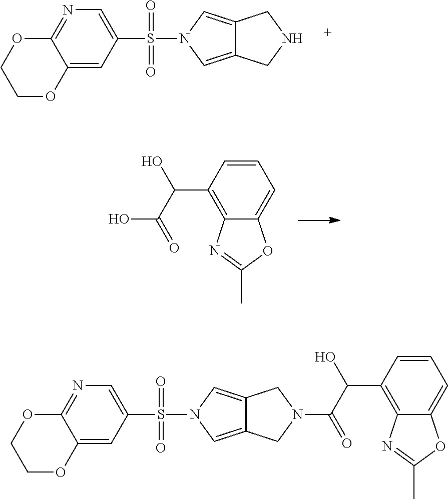 [SMILES: O=S(=O)(c1cnc2c(c1)OCCO2)n1cc2c(c1)CNC2 ; Cc1nc2c(C(O)C(=O)O)cccc2o1 ; Cc1nc2c(C(O)C(=O)N3Cc4cn(S(=O)(=O)c5cnc6c(c5)OCCO6)cc4C3)cccc2o1]

To a solution of 5-[2H,3H-[1,4]dioxino[2,3-b]pyridine-7-sulfonyl]-1H,2H,3H,5H-pyrrolo[3,4-c]pyrrole (180 mg, 0.43 mmol) in N,N-dimethylformamide (10 mL) was added 2-hydroxy-2-(2-methyl-1,3-benzoxazol-4-yl)acetic acid (89 mg, 0.43 mmol), DIEA (110.4 mg, 0.86 mmol), HOBt (63.5 mg, 0.47 mmol) and EDCI (90 mg, 0.47 mmol). The resulting mixture was stirred for 2 h at room temperature and poured into water (50 mL). The resulting solution was extracted with EtOAc (3×30 mL). The combined organic layers were dried over anhydrous sodium sulfate, filtered and concentrated under vacuum. The resulting crude product was purified by prep-TLC (eluting with 1:10 MeOH/DCM). The two enantiomers were separated by Chiral Prep-HPLC (Column: CHIRALPARK IC, 5 μm, 20×250 mm; Mobile Phase, A: DCM and B: MeOH (hold 85% B for 25 min); flow rate: 20 mL/min; Detector: UV 254/220 nm; RT: A (1^{st}), 16.24 min; B (2^{nd}), 21.61 min). The fractions of A were concentrated and lyophilized to afford 1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one, 1^{st} eluting isomer, as a white solid (39.8 mg, 19%). The fractions of B were concentrated and lyophilized to afford 1-(5-[2H,3H-[1,4]dioxino[2,3-b]pyridine-7-sulfonyl]-1H,2H,3H,5H-pyrrolo[3,4-c]pyrrol-2-yl)-2-hydroxy-2-(2-methyl-1,3-benzoxazol-4-yl)ethan-1-one, 2^{nd} eluting isomer, as a white solid (31.7 mg, 15%).

### Method B

### (2R)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one) and (2S)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one)

### Example 95-1. 1st Eluting Isomer; Example 95-2. 2^{nd} Eluting Isomer

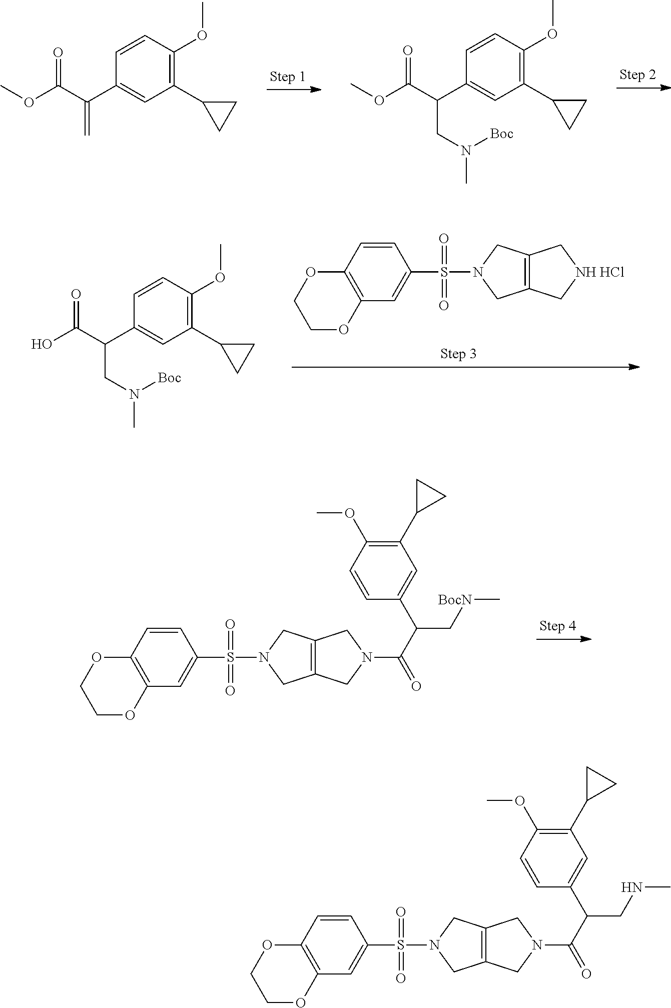

### Step 1. methyl 3-{[(tert-butoxy)carbonyl](methyl)amino}-2-(3-cyclopropyl-4-methoxyphenyl)propanoate

To a solution of methyl 2-(3-cyclopropyl-4-methoxyphenyl)prop-2-enoate (300 mg, 1.29 mmol) in tetrahydrofuran (10 mL) was added methylamine (2 mL). The resulting mixture was stirred for 30 min at room temperature and concentrated under vacuum to remove excess methylamine. The residue was dissolved in tetrahydrofuran (5 mL) and treated with di-tert-butyl dicarbonate (423 mg, 1.94 mmol). The reaction stirred for 16 h and was concentrated under vacuum. The resulting crude product was purified by silica gel chromatography (eluting with 0:100 to 5:95 ethyl acetate/petroleum ether) to afford methyl 3-{[(tert-butoxy)carbonyl](methyl)amino}-2-(3-cyclopropyl-4-methoxyphenyl)propanoate as a light yellow oil (200 mg, 43%). LCMS (ES, m/z) 364 [M+H]⁺.

### Step 2. 3-{[(tert-butoxy)carbonyl](methyl)amino}-2-(3-cyclopropyl-4-methoxyphenyl)propanoic acid

To a solution of methyl 3-{[(tert-butoxy)carbonyl](methyl)amino}-2-(3-cyclopropyl-4-methoxyphenyl)propanoate (200 mg, 0.55 mmol) in tetrahydrofuran (5 mL) was added water (5 mL), and lithium hydroxide (66 mg, 2.75 mmol). The resulting mixture was stirred for 16 h at room temperature. The reaction mixture was washed with diethyl ether (1×5 mL) and then acidified to pH=5 with saturated citric acid. The resulting solution was extracted with ethyl acetate (3×5 mL). The combined organic layers were dried over anhydrous sodium sulfate, filtered, and concentrated under vacuum to afford 3-{[(tert-butoxy)carbonyl](methyl)amino}-2-(3-cyclopropyl-4-methoxyphenyl)propanoic acid as a light yellow oil (150 mg, 87%). LCMS (ES, m/z) 350 [M+H]⁺.

### Step 3. tert-butyl N-[2-(3-cyclopropyl-4-methoxyphenyl)-3-[5-(2,3-dihydro-1,4-benzodioxine-6-sulfonyl)-1H,2H,3H,4H,5H,6H-pyrrolo[3,4-c]pyrrol-2-yl]-3-oxopropyl]-N-methylcarbamate

To a solution of 3-{[(tert-butoxy)carbonyl](methyl)amino}-2-(3-cyclopropyl-4-methoxyphenyl)propanoic acid (150 mg, 0.43 mmol) in DMF (5 mL) was added 2-(2,3-dihydro-1,4-benzodioxine-6-sulfonyl)-1H,2H,3H,4H,5H,6H-pyrrolo[3,4-c]pyrrole (177 mg, 0.51 mmol), DIEA (0.23 mL, 1.29 mmol) and HATU (196 mg, 0.51 mmol). The resulting solution was stirred for 1 h and poured into water (5 mL). The product was extracted with ethyl acetate (3×5 mL). The combined organic layers were dried over anhydrous sodium sulfate, filtered, and concentrated under vacuum. The resulting crude product was purified by prep-TLC (eluting with 1:1 ethyl acetate/petroleum ether) to afford tert-butyl N-[2-(3-cyclopropyl-4-methoxyphenyl)-3-[5-(2,3-dihydro-1,4-benzodioxine-6-sulfonyl)-1H,2H,3H,4H,5H,6H-pyrrolo[3,4-c]pyrrol-2-yl]-3-oxopropyl]-N-methylcarbamate as alight yellow oil (150 mg, 55%). LCMS (ES, m/z) 640 [M+H]⁺.

### Step 4. (2R)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one) and (2S)-2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one)

To a solution of tert-butyl N-[2-(3-cyclopropyl-4-methoxyphenyl)-3-[5-(2,3-dihydro-1,4-benzodioxine-6-sulfonyl)-1H,2H,3H,4H,5H,6H-pyrrolo[3,4-c]pyrrol-2-yl]-3-oxopropyl]-N-methylcarbamate (150 mg, 0.16 mmol) in dichloromethane (4 mL) was added trifluoroacetic acid (1 mL). The resulting solution was stirred for 2 h at room temperature and concentrated under vacuum. The reaction was quenched with saturated potassium carbonate solution (5 mL) and then extracted with dichloromethane (3×5 mL). The combined organic layers were dried over anhydrous sodium sulfate, filtered, and concentrated under vacuum. The crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (30% to 58% over 7 min); Flow rate: 20 mL/min; Detector: UV 254 nm). The product fractions were concentrated under vacuum. The two enantiomers were further separated by (Column: CHIRALPAK IC, 5 μm, 20×250 mm; Mobile Phase, A: MTBE (containing 0.1% DEA) and B: EtOH (keep 50% B over 18 min); Detector: UV 254/220 nm; Retention time: A (1^{st}), 9.54 min; B (2^{nd}), 12.96 min). The product fractions were concentrated and lyophilized to afford 2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one), 1^{st} eluting isomer, as a white solid (44.3 mg, 70%), and 2-(3-cyclopropyl-4-methoxyphenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one), 2^{nd} eluting isomer, as a white solid (30.2 mg 48%).

### Method C

### (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one and (2R)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one

### Example 96-1. 1^{st} Eluting Isomer; Example 96-2. 2^{nd} Eluting Isomer

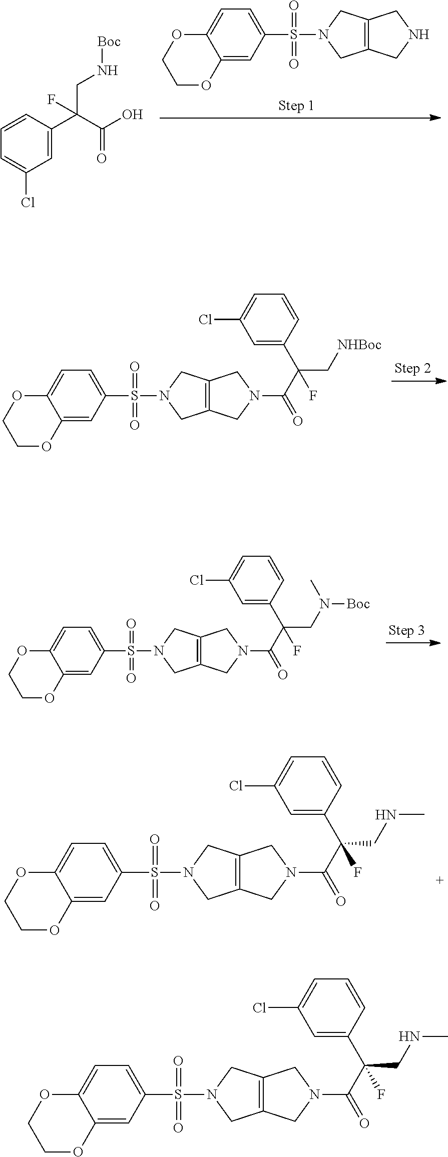 [SMILES: CC(C)(C)OC(=O)NCC(F)(C(=O)O)c1cccc(Cl)c1 ; O=S(=O)(c1ccc2c(c1)OCCO2)N1CC2=C(CNC2)C1 ; CC(C)(C)OC(=O)NCC(F)(C(=O)N1CC2=C(C1)CN(S(=O)(=O)c1ccc3c(c1)OCCO3)C2)c1cccc(Cl)c1 ; CN(CC(F)(C(=O)N1CC2=C(C1)CN(S(=O)(=O)c1ccc3c(c1)OCCO3)C2)c1cccc(Cl)c1)C(=O)OC(C)(C)C ; CNC[C@](F)(C(=O)N1CC2=C(C1)CN(S(=O)(=O)c1ccc3c(c1)OCCO3)C2)c1cccc(Cl)c1 ; CNC[C@@](F)(C(=O)N1CC2=C(C1)CN(S(=O)(=O)c1ccc3c(c1)OCCO3)C2)c1cccc(Cl)c1]

### Step 1. Tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]carbamate

To a solution of 3-[[(tert-butoxy)carbonyl]amino]-2-(3-chlorophenyl)-2-fluoropropanoic acid (120 mg, 0.34 mmol) in DMF (2 mL) was added HATU (155 mg, 0.41 mmol), 2-(2,3-dihydro-1,4-benzodioxine-6-sulfonyl)-1H,2H,3H,4H,5H,6H-pyrrolo[3,4-c]pyrrole (110 mg, 0.34 mmol) and DIEA (132 mg, 1.02 mmol). The resulting solution was stirred for 1 h at room temperature. The reaction mixture was poured into water (10 mL) and then extracted with ethyl acetate (3×20 mL). The combined organic layers were dried over anhydrous sodium sulfate, filtered, and concentrated under vacuum. The resulting crude product was purified by prep-TLC (eluting with 2:1 ethyl acetate/petroleum ether) to afford tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]carbamate as a yellow oil (120 mg, 58%). LCMS (ES, m/z) 608, 610 [M+H]⁺.

### Step 2. Tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]-N-methylcarbamate

To a solution of tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]carbamate (120 mg, 0.20 mmol) in THF (2 mL) was added sodium hydride (10 mg, 0.25 mmol, 60% dispersion in mineral oil) at 0° C. The resulting solution was stirred for 30 min at 0° C. and then treated with iodomethane (28 mg, 0.20 mmol). The resulting mixture was stirred for 6 h at room temperature. The reaction mixture was poured into aqueous ammonium chloride solution (10 mL) and then extracted with ethyl acetate (3×20 mL). The combined organic layers were dried over anhydrous sodium sulfate, filtered, and concentrated under vacuum to afford tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]-N-methylcarbamate as a yellow oil (120 mg, 98%). LCMS (ES, m/z) 622, 624 [M+H]⁺.

### Step 3. (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one and (2R)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-2-fluoro-3-(methylamino)propan-1-one

To a solution of tert-butyl N-[2-(3-chlorophenyl)-3-[5-(2,3-dihydro-1,4-benzodioxine-6-sulfonyl)-1H,2H,3H,4H,5H,6H-pyrrolo[3,4-c]pyrrol-2-yl]-2-fluoro-3-oxopropyl]-N-methyl carbamate (120 mg, 0.18 mmol) in dichloromethane (2 mL) was added TFA (0.4 mL). The resulting solution was stirred for 1 h at room temperature and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (28% to 50% over 15 min); Flow rate: 20 mL/min; Detector: UV 254 nm). The two enantiomers were further separated by (Column: CHIRALPAK IF, 5 μm, 20×250 mm; Mobile Phase, A: methanol (containing 0.1% DEA) and B: DCM (hold 50% B over 15 min); Detector: UV 254/220 nm; Retention time: A (1^{st}), 8.817 min; B (2^{nd}), 11.059 min). The product fractions of A were concentrated and lyophilized to afford 2-(3-chlorophenyl)-1-[5-(2,3-dihydro-1,4-benzodioxine-6-sulfonyl)-1H,2H,3H,4H,5H,6H-pyrrolo[3,4-c]pyrrol-2-yl]-2-fluoro-3-(methylamino)propan-1-one, 1^{st} eluting isomer, as a white solid (24.4 mg, 26%). The product fractions of B were concentrated and lyophilized to afford 2-(3-chloro-4-cyclopropoxyphenyl)-1-[5-(2,3-Dihydro-1,4-benzodioxine-6-sulfonyl)-1H,2H,3H,4H,5H,6H-pyrrolo[3,4-c]pyrrol-2-yl]-3-(methylamino)propan-1-one, 2^{nd} eluting isomer, as a white solid (11.9 mg, 12%).

### Method D

### (2S)-1-[5-(2,3-dihydro-1,4-benzodioxine-6-sulfonyl)-1H,2H,3H,4H,5H,6H-pyrrolo[3,4-c]pyrrol-2-yl]-2-(7-fluoro-2-methyl-2,3-dihydro-1H-isoindol-4-yl)-2-hydroxyethan-1-one and (2R)-1-[5-(2,3-dihydro-1,4-benzodioxine-6-sulfonyl)-1H,2H,3H,4H,5H,6H-pyrrolo[3,4-c]pyrrol-2-yl]-2-(7-fluoro-2-methyl-2,3-dihydro-1H-isoindol-4-yl)-2-hydroxyethan-1-one

### Example 97-1. 1^{st} Eluting Isomer; Example 97-2. 2^{nd} Eluting Isomer

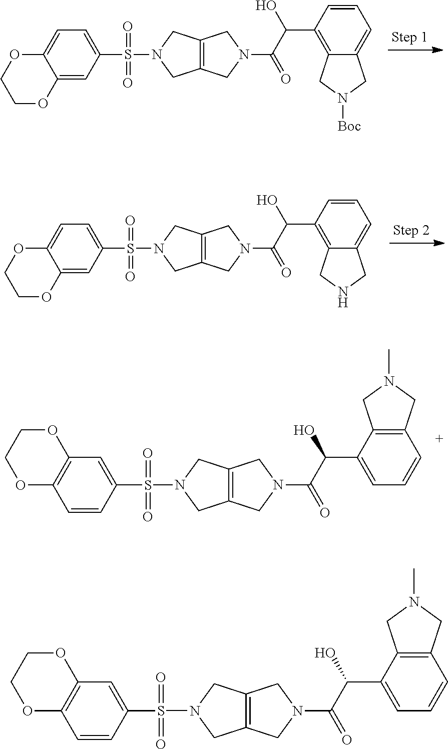 [SMILES: CC(C)(C)OC(=O)N1Cc2cccc(C(O)C(=O)N3CC4=C(C3)CN(S(=O)(=O)c3ccc5c(c3)OCCO5)C4)c2C1 ; O=C(C(O)c1cccc2c1CNC2)N1CC2=C(C1)CN(S(=O)(=O)c1ccc3c(c1)OCCO3)C2 ; CN1Cc2cccc([C@H](O)C(=O)N3CC4=C(C3)CN(S(=O)(=O)c3ccc5c(c3)OCCO5)C4)c2C1 ; CN1Cc2cccc([C@@H](O)C(=O)N3CC4=C(C3)CN(S(=O)(=O)c3ccc5c(c3)OCCO5)C4)c2C1]

### Step 1. 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)-2-hydroxyethan-1-one

To a solution of tert-butyl 4-[2-[5-(2,3-dihydro-1,4-benzodioxine-6-sulfonyl)-1H,2H,3H,4H,5H,6H-pyrrolo[3,4-c]pyrrol-2-yl]-1-hydroxy-2-oxoethyl]-2,3-dihydro-1H-isoindole-2-carboxylate (780 mg, 1.34 mmol) in dichloromethane (6 mL) was added hydrochloric acid (6 mL, 4 N in 1,4-dioxane). The resulting solution was stirred for 2 h at room temperature and concentrated under vacuum to afford 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)-2-hydroxyethan-1-one HCl salt as a dark red solid (680 mg, 62%). LCMS (ES, m/z) 484 [M+H]⁺.

### Step 2. (2R)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-2,3-dihydro-1H-isoindol-4-yl)ethan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-2,3-dihydro-1H-isoindol-4-yl)ethan-1-one

To a solution of 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)-2-hydroxyethan-1-one (680 mg, 1.40 mmol) in methanol (7 mL) was added formaldehyde (7 mL, 40 wt % in water). The resulting solution was stirred for 2 h at room temperature and then treated with sodium triacetoxyborohydride (893 mg, 4.21 mmol). The resulting mixture was stirred for 16 h at room temperature and concentrated under vacuum. The crude product was purified by prep-TLC (eluting with 1:10 MeOH/DCM). The enantiomers were separated by prep-Chiral HPLC (Column: CHIRAL ART Cellulose-SB, 5 μm, 20×250 mm; Mobile Phase, A: DCM and B: EtOH (0.1% DEA) (keep 40% B over 10 min); Detector: UV 254/220 nm; Retention time: 1^{st}, 6.63 min; 2^{nd}, 8.63 min. The product fractions were concentrated and lyophilized to afford 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-(2-methyl-2,3-dihydro-1H-isoindol-4-yl)ethan-1-one, 1^{st} eluting isomer, as a white solid (41.2 mg, 6%), and 1-[5-(2,3-dihydro-1,4-benzodioxine-6-sulfonyl)-1H,2H,3H,4H,5H,6H-pyrrolo[3,4-c]pyrrol-2-yl]-2-hydroxy-2-methyl-2,3-dihydro-1H-isoindol-4-yl)ethan-1-one, 2^{nd} eluting isomer, as a white solid (42.4 mg, 5%).

### Method E

### (2S)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one and (2R)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one

### Example 98-1. 1^{st} Eluting Isomer; Example 98-2. 2^{nd} Eluting Isomer

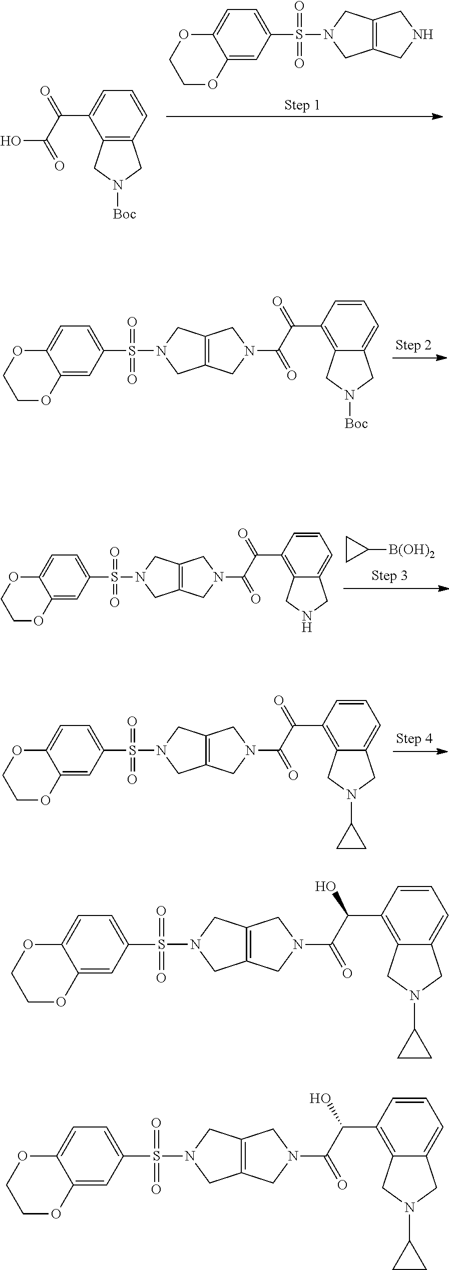

### Step 1. Tert-butyl4-{2-[5-(2,3-dihydro-1,4-benzodioxine-6-sulfonyl)-1H,2H,3H,4H,5H,6H-pyrrolo[3,4-c]pyrrol-2-yl]-2-oxoacetyl}-2,3-dihydro-1H-isoindole-2-carboxylate

To a solution of 2-(2,3-dihydro-1,4-benzodioxine-6-sulfonyl)-1H,2H,3H,4H,5H,6H-pyrrolo[3,4-c]pyrrole (529 mg, 1.63 mmol) in DMF (3 mL) was added 2-[2-[(tert-butoxy)carbonyl]-2,3-dihydro-1H-isoindol-4-yl]-2-oxoacetic acid (500 mg, 1.46 mmol), DIEA (665 mg, 4.89 mmol) and HATU (783 mg, 1.96 mmol). The resulting solution was stirred for 1 h at room temperature. The reaction mixture was poured into water (5 mL) and then extracted with EtOAc (3×5 mL). The combined organic layers were dried over anhydrous sodium sulfate, filtered, and concentrated under vacuum. The resulting crude product was purified by prep-TLC (eluting with 3:1 ethyl acetate/petroleum ether) to afford tert-butyl 4-[2-[5-(2,3-dihydro-1,4-benzodioxine-6-sulfonyl)-1H,2H,3H,4H,5H,6H-pyrrolo[3,4-c]pyrrol-2-yl]-2-oxoacetyl]-2,3-dihydro-1H-isoindole-2-carboxylate as a yellow oil (300 mg, 30%). LCMS (ES, m/z) 582 [M+H]⁺.

### Step 2. 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)ethane-1,2-dione

To a solution of tert-butyl 4-[2-[5-(2,3-dihydro-1,4-benzodioxine-6-sulfonyl)-1H,2H,3H,4H,5H,6H-pyrrolo[3,4-c]pyrrol-2-yl]-2-oxoacetyl]-2,3-dihydro-1H-isoindole-2-carboxylate (300 mg, 0.45 mmol) in DCM (6 mL) was added TFA (1.5 mL). The resulting mixture was stirred for 2 h at room temperature and concentrated under vacuum. The resulting mixture was basified to pH=8 with saturated potassium carbonate solution and then extracted with dichloromethane (2×5 mL). The combined organic layers were dried over anhydrous sodium sulfate, filtered and concentrated under vacuum to afford 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)ethane-1,2-dione as a yellow solid (200 mg, crude). LCMS (ES, m/z) 482 [M+H]⁺.

### Step 3. 1-(2-cyclopropyl-2,3-dihydro-1H-isoindol-4-yl)-2-[5-(2,3-dihydro-1,4-benzodioxine-6-sulfonyl)-1H,2H,3H,4H,5H,6H-pyrrolo[3,4-c]pyrrol-2-yl]ethane-1,2-dione

To a solution of 1-[5-(2,3-dihydro-1,4-benzodioxine-6-sulfonyl)-1H,2H,3H,4H,5H,6H-pyrrolo[3,4-c]pyrrol-2-yl]-2-(2,3-dihydro-1H-isoindol-4-yl)ethane-1,2-dione (217 mg, 0.38 mmol) in 1,2-dichloroethane (15 ml) was added copper (II) acetate (90 mg, 0.43 mmol), 2,2′-bipyridine (70 mg, 0.43 mmol), cyclopropylboronic acid (77 mg, 0.85 mmol) and sodium carbonate (95 mg, 0.86 mmol). The resulting mixture was stirred for 16 h at 70° C. under air atmosphere and cooled to room temperature. The reaction mixture was filtered and poured into water (10 mL). The resulting solution was extracted with ethyl acetate (3×10 mL). The combined organic layers were dried over anhydrous sodium sulfate, filtered and concentrated under vacuum. The resulting crude product was purified by prep-TLC (eluting with 3:1 ethyl acetate/petroleum ether) to afford 1-(2-cyclopropyl-2,3-dihydro-1H-isoindol-4-yl)-2-[5-(2,3-dihydro-1,4-benzodioxine-6-sulfonyl)-1H,2H,3H,4H,5H,6H-pyrrolo[3,4-c]pyrrol-2-yl]ethane-1,2-dione as a white solid (60 mg, 26%). LCMS (ES, m/z) 522 [M+H]⁺.

### Step 4. (2S)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one and (2R)-2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one

To a solution of 1-(2-cyclopropyl-2,3-dihydro-1H-isoindol-4-yl)-2-[5-(2,3-dihydro-1,4-benzodioxine-6-sulfonyl)-1H,2H,3H,4H,5H,6H-pyrrolo[3,4-c]pyrrol-2-yl]ethane-1,2-dione (60 mg, 0.10 mmol) in methanol (1.5 mL) was added sodium borohydride (9 mg, 0.23 mmol). The resulting solution was stirred for 2 h at room temperature. The reaction mixture was poured into water (5 mL) and then extracted with EtOAc (3×5 mL). The combined organic layers were dried over anhydrous sodium sulfate, filtered and concentrated under vacuum. The resulting crude product was purified by silica gel chromatography (eluting with 10:1 DCM/MeOH) and further purified by Prep-HPLC (Column: XBridge Prep C18 OBD Column (19×150 mm) 5 um; Mobile Phase A: Water (10 mmoL/L NH₄HCO₃), Mobile Phase B: MeCN (30% B to 55% B over 7 min); Flow rate: 20 mL/min; Detector: 254/220 nm). The two enantiomers were further separated by Chiral-Prep-HPLC (Column: CHIRALPAK IE, 2×25 cm, 5 μm; Mobile Phase A: MeOH (containing 0.1% DEA), Mobile Phase B: DCM (Hold 35% B over 14 min); Flow rate: 19 mL/min; Detector: 220/254 nm; A: 9.39 min; B: 12.4 min). The fractions of A were concentrated and lyophilized to afford 2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one, 1^{st} eluting isomer, as a white solid (5.0 mg, 10%). The fractions of B were concentrated and lyophilized to afford 2-(2-cyclopropyl-2,3-dihydro-1H-isoindol-4-yl)-1-[5-(2,3-dihydro-1,4-benzodioxine-6-sulfonyl)-1H,2H,3H,4H,5H,6H-pyrrolo[3,4-c]pyrrol-2-yl]-2-hydroxyethan-1-one, 2^{nd} eluting isomer, as a white solid (5.3 mg, 10%).

### Method F

### Example 99-1. (2S)-2-(3-chlorophenyl)-1-[5-(2,3-dihydro-1,4-benzodioxine-6-sulfonyl)-1H,2H,3H,4H,5H,6H-pyrrolo[3,4-c]pyrrol-2-yl]-3-(methylamino)propan-1-one

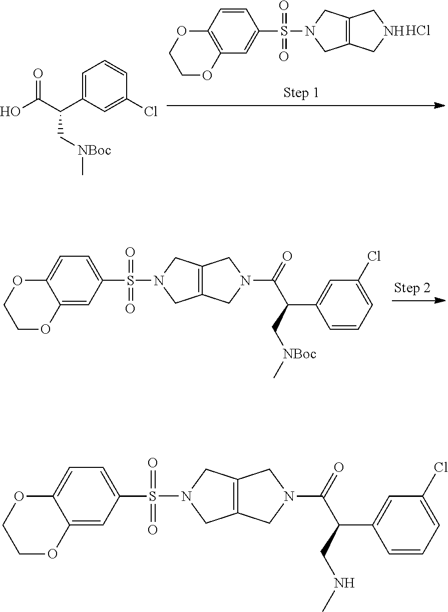 [SMILES: CN(C[C@H](C(=O)O)c1cccc(Cl)c1)C(=O)OC(C)(C)C ; O=S(=O)(c1ccc2c(c1)OCCO2)N1CC2=C(CNC2)C1 ; Cl ; CN(C[C@@H](C(=O)N1CC2=C(C1)CN(S(=O)(=O)c1ccc3c(c1)OCCO3)C2)c1cccc(Cl)c1)C(=O)OC(C)(C)C ; CNC[C@@H](C(=O)N1CC2=C(C1)CN(S(=O)(=O)c1ccc3c(c1)OCCO3)C2)c1cccc(Cl)c1]

### Step 1. Tert-butyl N-[(2S)-2-(3-chlorophenyl)-3-[5-(2,3-Dihydro-1,4-benzodioxine-6-sulfonyl)1H,2H,3H,4H,5H, 6H-pyrrolo[3,4-c]pyrrol-2-yl]-3-oxopropyl]-N-methylcarbamate

To a solution of (2R)-3-{[(tert-butoxy)carbonyl](methyl)amino}-2-(3-chlorophenyl)propanoic acid (2.30 g, 7.34 mmol) in N,N-dimethylformamide (20 mL) was added HATU (3.07 g, 8.08 mmol), 2-(2,3-dihydro-1,4-benzodioxine-6-sulfonyl)-1H,2H,3H,4H,5H,6H-pyrrolo[3,4-c]pyrrole hydrochloride (2.52 g, 7.34 mmol), and DIEA (3.82 mL, 22.1 mmol). The resulting solution was stirred for 2 h at rt. The reaction mixture was poured into water (100 mL) and then extracted with ethyl acetate (3×100 mL). The combined organic layers were dried over anhydrous sodium sulfate, filtered, and concentrated under vacuum. The crude product was purified by silica gel chromatography (eluting with 1:15 ethyl acetate/dichloromethane) to afford tert-butyl N-[(2S)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate as a white solid (3.5 g, 79%). ¹H-NMR (DMSO-d₆, 400 MHz) δ (ppm): 7.40-7.18 (m, 6H), 7.07-7.06 (m, 1H), 4.45-4.22 (m, 5H), 4.11-3.88 (m, 7H), 3.88-3.58 (m, 2H), 3.44-3.36 (m, 1H), 2.75-2.67 (m, 3H), 1.27-1.16 (m, 9H). LCMS (ES, m/z) 604, 606 [M+H]⁺.

### Step 2. (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-3-(methylamino)propan-1-one

To a solution of tert-butyl N-[(2S)-2-(3-chlorophenyl)-3-[5-(2,3-Dihydro-1,4-benzodioxine-6-sulfonyl)-1H,2H,3H,4H,5H,6H-pyrrolo[3,4-c]pyrrol-2-yl]-3-oxopropyl]-N-methylcarbamate (1.5 g, 2.49 mmol) in ethyl acetate (10 mL) was added a solution of hydrochloric acid (10 mL, 4 N in 1,4-dioxane). The resulting solution was stirred for 3 h at 25° C. The mixture was concentrated under vacuum to about ⅓ volume and the solids were collected by filtration. The solids were treated with EtOAc (10 mL) at 70° C., filtered at room temperature, and dissolved with saturated potassium carbonate solution/EA (1:1, 10 mL). The resulting solution was stirred for 3 h and then extracted with EA (3×5 mL). The combined organic layers were dried over anhydrous sodium sulfate, filtered, and concentrated under vacuum to afford (2S)-2-(3-chlorophenyl)-1-[5-(2,3-dihydro-1,4-Benzodioxine-6-sulfonyl)-1H,2H,3H,4H,5H,6H-pyrrolo[3,4-c]pyrrol-2-yl]-3-(methylamino)propan-1-one as a white solid (1 g, 80%). ¹H-NMR (DMSO-d₆, 400 MHz) δ (ppm): 7.36-7.25 (m, 6H), 7.07-7.06 (m, 1H), 4.40-4.29 (m, 5H), 4.06-3.88 (m, 8H), 3.06-3.01 (m, 1H), 2.60-2.50 (m, 1H), 2.23 (s, 3H), 1.66 (s, 1H). LCMS (ES, m/z) 504, 506 [M+H]⁺.

### Method J

### (2R)-1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one and (2S)-1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one

### Example 103-1. 1^{st} Eluting Isomer; Example 103-2. 2^{nd} Eluting Isomer

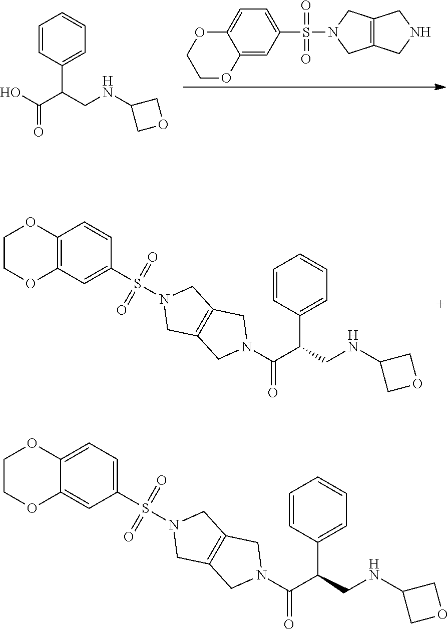 [SMILES: O=C(O)C(CNC1COC1)c1ccccc1 ; O=S(=O)(c1ccc2c(c1)OCCO2)N1CC2=C(CNC2)C1 ; O=C([C@@H](CNC1COC1)c1ccccc1)N1CC2=C(C1)CN(S(=O)(=O)c1ccc3c(c1)OCCO3)C2 ; O=C([C@H](CNC1COC1)c1ccccc1)N1CC2=C(C1)CN(S(=O)(=O)c1ccc3c(c1)OCCO3)C2]

To a solution of 3-[(oxetan-3-yl)amino]-2-phenylpropanoic acid (60 mg, 0.27 mmol) in DMF (10 mL) was added HATU (123 mg, 0.32 mmol), 2-(2,3-dihydro-1,4-benzodioxine-6-sulfonyl)-1H,2H,3H,4H,5H,6H-pyrrolo[3,4-c]pyrrole hydrochloride (93 mg, 0.27 mmol) and DIEA (0.13 mL, 0.81 mmol). The resulting mixture was stirred for 1 h at room temperature. The reaction mixture was poured into water (5 mL) and then extracted with ethyl acetate (3×10 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 30×150 mm; Mobile Phase, A: water (containing 10 mmol/L NH₄HCO₃) and B: CH₃CN (25% to 45% over 7 min); Flow rate: 60 mL/min; Detector: UV 254 nm). The product fractions were concentrated under vacuum. The two enantiomers were further separated by Chiral Prep-HPLC (Column: CHIRALPAK IF, 5 μm, 20×250 mm; Mobile Phase, A: MeOH (containing 0.1% DEA) and B: DCM (keep 10% B over 16 min); Flow rate: 20 mL/min; Detector: UV 254/220 nm; Retention time: 1^{st}, 17.285 min; 2^{nd}, 21.532 min). The product fractions were concentrated and lyophilized to afford 1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one, 1^{st} eluting isomer, as a white solid (1 mg, 1.4%), and 1-[5-(2,3-dihydro-1,4-benzodioxine-6-sulfonyl)-1H,2H,3H,4H,5H,6H-pyrrolo[3,4-c]pyrrol-2-yl]-3-[(oxetan-3-yl)amino]-2-phenylpropan-1-one, 2^{nd} eluting isomer, as a white solid (1 mg, 1.4%).

### Method R

### (2S)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one and (2R)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one

### Example 112-1. 1^{st} Eluting Isomer; Example 112-2. 2^{nd} Eluting Isomer

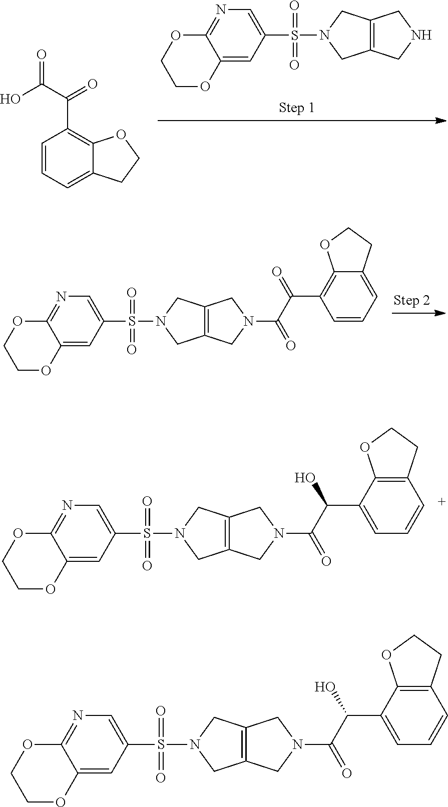 [SMILES: O=C(O)C(=O)c1cccc2c1OCC2 ; O=S(=O)(c1cnc2c(c1)OCCO2)N1CC2=C(CNC2)C1 ; O=C(C(=O)N1CC2=C(C1)CN(S(=O)(=O)c1cnc3c(c1)OCCO3)C2)c1cccc2c1OCC2 ; O=C([C@@H](O)c1cccc2c1OCC2)N1CC2=C(C1)CN(S(=O)(=O)c1cnc3c(c1)OCCO3)C2 ; O=C([C@H](O)c1cccc2c1OCC2)N1CC2=C(C1)CN(S(=O)(=O)c1cnc3c(c1)OCCO3)C2]

### Step 1. 1-(2,3-dihydro-1-benzofuran-7-yl)-2-(5-{2H,3H-[1,4]dioxino[2,3-b]pyridine-7-sulfonyl}-1H,2H,3H,4H,5H,6H-pyrrolo[3,4-c]pyrrol-2-yl)ethane-1,2-dione

To a solution of 2-(2,3-dihydro-1-benzofuran-7-yl)-2-oxoacetic acid (250 mg, 1.30 mmol) in DMF (4 mL) was added 2-[2H,3H-[1,4]dioxino[2,3-b]pyridine-7-sulfonyl]-1H,2H,3H,4H,5H,6H-pyrrolo[3,4-c]pyrrole (450 mg, 1.30 mmol), DIEA (0.43 mL, 2.60 mmol), and HATU (544 mg, 1.43 mmol). The resulting solution was stirred for 1 h at room temperature. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by prep-TLC (eluting with 1:1 ethyl acetate/petroleum ether) to afford 1-(2,3-dihydro-1-benzofuran-7-yl)-2-(5-{2H,3H-[1,4]dioxino[2,3-b]pyridine-7-sulfonyl}-1H,2H,3H,4H,5H,6H-pyrrolo[3,4-c]pyrrol-2-yl)ethane-1,2-dione as a light yellow solid (200 mg, 32%). LCMS (ES, m/z): 484 [M+H]⁺.

### Step 2. (2S)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one and (2R)-2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one

To a solution of 1-(2,3-dihydro-1-benzofuran-7-yl)-2-(5-{2H,3H-[1,4]dioxino[2,3-b]pyridine-7-sulfonyl}-1H,2H,3H,4H,5H,6H-pyrrolo[3,4-c]pyrrol-2-yl)ethane-1,2-dione (200 mg, 0.41 mmol) in tetrahydrofuran (2 mL) was added sodium borohydride (8 mg, 0.21 mmol). The resulting solution was stirred for 30 min at 0° C. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: MeCN (15% to 45% over 10 min); Flow rate: 20 mL/min; Detector: UV 254 nm). The product fractions were concentrated under vacuum. The two enantiomers were separated by Chiral Prep-HPLC (Column: CHIRALPAK IF, 5 μm, 20×250 mm; Mobile Phase, A: MeOH (containing 0.1% DEA) and B: DCM (keep 40% B over 50 min); Flow rate: 15 mL/min; Detector: UV 254/220 nm; Retention time: 1^{st}, 19.223 min; 2^{nd}, 29.404 min). The product fractions were concentrated and lyophilized to afford 2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one, 1^{st} eluting isomer, as a white solid (30.5 mg, 15%), and 2-(2,3-dihydro-1-benzofuran-7-yl)-1-(5-{2H,3H-[1,4]dioxino[2,3-b]pyridine-7-sulfonyl}-1H,2H,3H,4H,5H,6H-pyrrolo[3,4-c]pyrrol-2-yl)-2-hydroxyethan-1-one, 2′ eluting isomer, as a white solid (33.5 mg, 17%).

### Method S

### Example 114-1. (R)-3-((cyclopentylmethyl)amino)-1-(5-((2,3-dihydrobenzo[b][1,4]dioxin-6-yl)sulfonyl)-3,4,5,6-tetrahydropyrrolo[3,4-c]pyrrol-2(1H)-yl)-2-phenylpropan-1-one

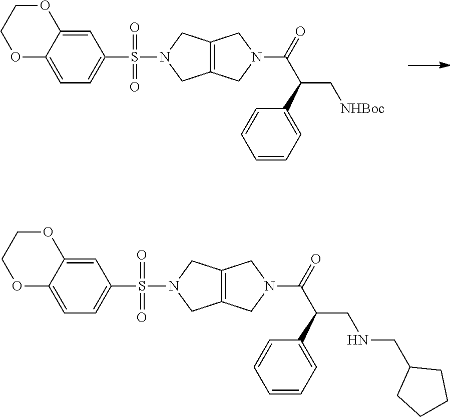 [SMILES: CC(C)(C)OC(=O)NC[C@H](C(=O)N1CC2=C(C1)CN(S(=O)(=O)c1ccc3c(c1)OCCO3)C2)c1ccccc1 ; O=C([C@@H](CNCC1CCCC1)c1ccccc1)N1CC2=C(C1)CN(S(=O)(=O)c1ccc3c(c1)OCCO3)C2]

To tert-butyl (R)-(3-(5-((2,3-dihydrobenzo[b][1,4]dioxin-6-yl)sulfonyl)-3,4,5,6-tetrahydropyrrolo[3,4-c]pyrrol-2(1H)-yl)-3-oxo-2-phenylpropyl)carbamate (180 μl, 36.0 μmol; 0.2M in dioxane) was added acetic acid (150 μl, 30.0 μmol; 0.2M in dioxane) and DCE, cyclopentanecarbaldehyde (180 μl, 36.0 μmol; 0.2M in dioxane) and sodium triacetoxyborohydride (300 μl, 60.0 μmol; 0.2M in dioxane). The reaction was heated at 50° C. for 4 h. The reaction was run through an SCX-SPE cartridge and eluted with 2 ml of 10% MeOH/EtOAc (ETW) followed by 2 ml of 2M Ammonia/MeOH (ETC). The basic eluent was dried under a stream of N₂ and the product was purified by reverse phase HPLC.

TABLE 9

| Example(s) | Purification Method |
|---|---|
| 94-31 and 94-32 | Chiral Prep-HPLC (Column: CHIRALPAK IF, 5 μm, 10 460 mm; Mobile Phase, A: DCM |
| | and B: MeOH (containing 0.1% DEA) (keep 50% B over 16 min); Flow rate: 20 mL/min; |
| | Detector: UV 254/220 nm; Retention time: 1^{st}, 17.97 min; 2^{nd}, 21.34 min) |
| 95-3 and 95-4 | Chiral Prep-HPLC (Column: CHIRALPAK IF, 5 μm, 20 × 250 mm; Mobile Phase, A: |
| | MeOH (containing 0.1% DEA) and B: DCM (keep 50% B over 15 min); Detector: UV |
| | 254/220 nm; Retention time: 1^{st}, 4.88 min; 2^{nd}, 6.81 min) |
| 95-29 and 95-30 | CHIRALPAK IF, 5 μm, 20 × 250 mm; Mobile Phase, A: methanol (containing 0.1% DEA) |
| | and B: DCM (keep 20% B over 13 min); Detector: UV 254/220 nm; Retention time: 1^{st}, |
| | 8.649 min; 0B, 9.625 min). |
| 95-31 and 95-32 | Chiral Prep-HPLC (Column: CHIRALPAK IF, 5 μm, 20 × 250 mm; Mobile Phase, A: |
| | methanol (containing 0.1% DEA) and B: DCM (keep 20% B over 13 min); Detector: UV |
| | 254/220 nm; Retention time: 1^{st}, 7.96 min; 2^{nd}, 8.97 min) |
| 95-70 and 95-71 | CHIRAL ART Cellulose-SB, 5 μm, 20 × 250 mm; Mobile Phase, A: MeOH (containing |
| | 0.1% NH3) and B: DCM (keep 0% B over 13 min); Flow rate: 20 mL/min; Detector: UV |
| | 254/220 nm; Retention time: 1^{st}, 9.16 min; 2^{nd}, 10.4 min). |
| 96-8 and 96-9 | Chiral Prep-HPLC (Column: CHIRALPAK IF, 5 μm, 20 × 250 mm; Mobile Phase, A: |
| | methanol (containing 0.1% DEA) and B: DCM (keep 20% B over 20 min); Detector: UV |
| | 254/220 nm; Retention time: 1^{st}, 11.312 min; 2^{nd}, 14.721 min) |
| 97-15 and 97-16 | Chiral Prep-HPLC (Column: CHIRALPAK IC, 2 × 25 cm, 5 μm; Mobile Phase A: MeCN, |
| | Mobile Phase B: MeOH (0.1% IPA); Flow rate: 20 mL/min; Gradient: keep 10% B over 16 |
| | min; Detector: UV 254/220 nm; Retention time: 1^{st}, 8.794 min; 2^{nd}, 13.19 min) |
| 97-17 and 97-18 | Chiral Prep-HPLC (Column: CHIRALPAK IF, 5 μm, 20 × 250 mm; Mobile Phase, A: |
| | MeOH (containing 0.1% DEA) and B: DCM; Gradient: keep 55% B over 11 min; |
| | Detector: UV 254/220 nm; Retention time: 1^{st}, 6.057 min; 2^{nd}, 8.405 min) |
| 98-1 and 98-2 | Prep-HPLC (Column: CHIRAL ART Cellulose-SB, 2 × 25 cm, 5 μm; Mobile Phase A: |
| | MTBE (containing 2 mM NH3), Mobile Phase B: EtOH (Hold 30% B over 10 min); Flow |
| | rate: 20 mL/min; Detector: 220/254 nm; RT: 1^{st}: 6.953 min; 2^{nd}: 8.329 min) |
| 99-8 and 99-9 | Chiral Prep-HPLC (Column: CHIRALPAK IF, 5 μm, 20 × 250 mm; Mobile Phase, A: |
| | MTBE (containing 0.1% DEA) and B: MeOH (keep 0% B over 17 min); Flow rate: 15 |
| | mL/min; Detector: UV 254/220 nm; Retention time: 1^{st}, 11.326 min; 2^{nd}, 13.972 min) |
| 103-7 and 103-8 | Chiral Prep-HPLC (Column: CHIRALPAK IF, 5 μm, 20 × 250 mm; Mobile Phase A: |
| | MTBE (0.1% DEA), Mobile Phase B: MeOH (keep 50% B over 20 min); Detector: UV |
| | 254/220 nm; Retention time: 1^{st}, 12.327 min; 2^{nd}, 17.189 min) |
| 103-46 and 103-47 | Chiral Prep-HPLC (Column: CHIRALPAK IG, 2 × 25 cm, 5 μm; Mobile Phase A: MeOH, |
| | Mobile Phase B: DCM; Flow rate: 18 mL/min; Gradient: keep 40% B over 15 min; |
| | Detector: UV 254/220 nm; Retention time: 1^{st}, 7.253 min; 2^{nd}, 10.19 min). |
| 112-3 and 112-4 | Chiral Prep-HPLC (Column: CHIRALPAK IG, 5 μm, 20 × 250 mm; Mobile Phase, A: |
| | MeOH (containing 0.1% DEA) and B: DCM; Gradient: keep 30% B in 18 min; Flow rate: |
| | 20 mL/min; Detector: UV 254/220 nm; Retention time: 1^{st}, 11.142 min; 2^{nd}, 14.7 min) |
| 114-1 and 114-2 | Prep-HPLC (Column: CHIRALPAK IE, 2 × 25 cm, 5 μm; Mobile Phase A: MTBE (0.2% |
| | IPA), Mobile Phase B: MeOH; Flow rate: 14 mL/min; Gradient: 50% B to 50% B over 40 |
| | min; Detector: UV 254/220 nm; Retention time: 1^{st}, 22.759 min; 2^{nd}, 34.952 min) |

As set forth in Table 10, IC₅₀ values are defined as follows. ≤25 μM and >2 μM (+); ≤2 μM and >0.2 μM (++); ≤0.2 μM and >0.05 μM (+++); ≤0.05 μM and >0.001 μM (++++); and not tested (−−), based upon the Biochemical Assay of Example A.

In Tables 1 and 10, absolute stereochemistry has not been determined for some Examples. Accordingly, assignment of any Examples as the “R” or “S” stereoisomer is arbitrary, unless otherwise noted. In some cases, Examples are labeled with “1^{st} eluting isomer”, “2^{nd} eluting isomer”, etc. based on the purification method used to separate the stereoisomers (see Table 9).

TABLE 10

| | IC₅₀ | | Synth. | | MS m/z | |
|---|---|---|---|---|---|---|
| Ex. | (μM) | Intermediates | Method | Chemical Name | [M + H]⁺ | ¹H NMR |
| 94-31 | ++++ | 2-2, | A | (2S)-1-[5-(2,3-dihydro-1,4- | 514.2 | (CDCl₃, 400 MHz) δ |
| (1^{st} | | 44-1 | | benzodioxine-6-sulfonyl)- | | (ppm): 7.81 (d, J = 5.6 |
| eluting | | | | 1H,2H,3H,4H,5H,6H-pyrrolo[3,4- | | Hz, 1H), 7.39-7.31 (m, |
| isomer) | | | | c]pyrrol-2-yl]-2-hydroxy-2-(2- | | 2H), 7.35-7.25 (m, |
| 94-32 | ++ | | | methyl-1,3-benzothiazol-4- | | 2H), 6.95 (d, J = 8.4 |
| (2^{nd} | | | | yl)ethan-1-one; | | Hz, 1H), 6.10 (s, 1H), |
| eluting | | | | (2R)-1-[5-(2,3-dihydro-1,4- | | 4.38-4.25 (m, 6H), |
| isomer) | | | | benzodioxine-6-sulfonyl)- | | 4.22-4.19 (m, 1H), |
| | | | | 1H,2H,3H,4H,5H,6H-pyrrolo[3,4- | | 4.16-4.02 (m, 3H), 4.01 |
| | | | | c]pyrrol-2-yl]-2-hydroxy-2-(2- | | (s, 1H), 3.94 (s, 1H), |
| | | | | methyl-1,3-benzothiazol-4- | | 3.55-3.53 (m, 1H), 2.89 |
| | | | | yl)ethan-1-one | | (s, 3H). |
| 95-3 | + | 3-1, | B | (2R)-1-[5-(2,3-dihydro-1,4- | 500.5 | (CD₃OD, 400 MHz) δ |
| (1^{st} | | 2-2 | | benzodioxine-6-sulfonyl)- | | (ppm): 7.32-7.24 (m, |
| eluting | | | | 1H,2H,3H,4H,5H,6H-pyrrolo[3,4- | | 3H), 7.07-6.99 (m, |
| isomer) | | | | c]pyrrol-2-yl]-2-(3- | | 1H), 6.88-6.85 (m, |
| 95-4 | ++++ | | | methoxyphenyl)-3- | | 3H), 4.38-4.31 (m, |
| (2^{nd} | | | | (methylamino)propan-1-one; | | 5H), 4.20-4.17 (m, |
| eluting | | | | (2S)-1-[5-(2,3-dihydro-1,4- | | 1H), 4.08-4.04 (m, |
| isomer) | | | | benzodioxine-6-sulfonyl)- | | 5H), 3.89-3.86 (m, |
| | | | | 1H,2H,3H,4H,5H,6H-pyrrolo[3,4- | | 2H), 3.78 (s, 3H), 3.32- |
| | | | | c]pyrrol-2-yl]-2-(3- | | 3.23 (m, 1H), 2.74-2.69 |
| | | | | methoxyphenyl)-3- | | (m, 1H), 2.39 (s, 3H). |
| | | | | (methylamino)propan-1-one | | |
| 95-29 | ++++ | 2-8, | B | (2R)-2-(3-chloro-4- | 560.4 | (DMSO-d₆, 400 MHz) |
| (1^{st} | | 2-2, | | cyclopropoxyphenyl)-1-[5-(2,3- | | δ (ppm): 7.41-7.35 (m, |
| eluting | | 3-16, | | dihydro-1,4-benzodioxine-6- | | 2H), 7.29-7.22 (m, |
| isomer) | | 5-2 | | sulfonyl)-1H,2H,3H,4H,5H,6H- | | 3H), 7.06 (d, J = 8.4 |
| 95-30 | ++ | | | pyrrolo[3,4-c]pyrrol-2-yl]-3- | | Hz, 1H), 4.33-4.29 (m, |
| (2^{nd} | | | | (methylamino)propan-1-one; | | 5H), 4.08-3.76 (m, |
| eluting | | | | (2S)-2-(3-chloro-4- | | 9H), 3.35 (d, J = 12.0 |
| isomer) | | | | cyclopropoxyphenyl)-1-[5-(2,3- | | Hz, 1H), 2.91-3.12 (m, |
| | | | | dihydro-1,4-benzodioxine-6- | | 1H), 2.51-2.48 (m, |
| | | | | sulfonyl)-1H,2H,3H,4H,5H,6H- | | 3H), 1.23 (s, 1H), 0.82 |
| | | | | pyrrolo[3,4-c]pyrrol-2-yl]-3- | | (d, J = 6.8 Hz, 2H), |
| | | | | (methylamino)propan-1-one | | 0.68 (s, 2H). |
| 95-31 | ++++ | 2-9, | B | (2R)-2-(3-chloro-4-fluoro-2- | 552.4 | (CD₃OD, 400 MHz) δ |
| (1^{st} | | 14-1, | | methoxyphenyl)-1-[5-(2,3- | | (ppm): 7.30-7.24 (m, |
| eluting | | 2-2 | | dihydro-1,4-benzodioxine-6- | | 3H), 6.99-6.97 (m, |
| isomer) | | | | sulfonyl)-1H,2H,3H,4H,5H,6H- | | 2H), 4.36-4.28 (m, |
| 95-32 | ++ | | | pyrrolo[3,4-c]pyrrol-2-yl]-3- | | 6H), 4.27-4.18 (m, |
| (2^{nd} | | | | (methylamino)propan-1-one; | | 1H), 4.15-4.04 (m, |
| eluting | | | | (2S)-2-(3-chloro-4-fluoro-2- | | 5H), 3.87 (s, 3H), 3.82- |
| isomer) | | | | methoxyphenyl)-1-[5-(2,3- | | 3.78 (m, 1H), 3.20-3.14 |
| | | | | dihydro-1,4-benzodioxine-6- | | (m, 1H), 2.71-2.67 (m, |
| | | | | sulfonyl)-1H,2H,3H,4H,5H,6H- | | 1H), 2.43 (s, 3H). |
| | | | | pyrrolo[3,4-c]pyrrol-2-yl]-3- | | |
| | | | | (methylamino)propan-1-one | | |
| 95-70 | ++++ | 27-1, | B | (2R)-2-(3-cyclopropyl-4,5- | 546.4 | (DMSO-d₆, 400 MHz) |
| (1^{st} | | 2-2 | | difluorophenyl)-1-[5-(2,3-dihydro- | | δ (ppm): 7.29-7.24 (m, |
| eluting | | | | 1,4-benzodioxine-6-sulfonyl)- | | 2H), 7.13-7.04 (m, |
| isomer) | | | | 1H,2H,3H,4H,5H,6H-pyrrolo[3,4- | | 2H), 6.74 (d, J = 6.4 |
| 95-71 | ++ | | | c]pyrrol-2-yl]-3- | | Hz, 1H), 4.36-4.30 (m, |
| (2^{nd} | | | | (methylamino)propan-1-one; | | 5H), 4.01-3.83 (m, |
| eluting | | | | (2S)-2-(3-cyclopropyl-4,5- | | 7H), 3.82-3.80 (m, |
| isomer) | | | | difluorophenyl)-1-[5-(2,3-dihydro- | | 1H), 3.00-2.95 (m, |
| | | | | 1,4-benzodioxine-6-sulfonyl)- | | 1H), 2.59-2.52 (m, |
| | | | | 1H,2H,3H,4H,5H,6H-pyrrolo[3,4- | | 1H), 2.22 (s, 3H), 2.05- |
| | | | | c]pyrrol-2-yl]-3- | | 2.00 (m, 1H), 1.02-1.00 |
| | | | | (methylamino)propan-1-one | | (m, 2H), 0.73-0.69 (m, |
| | | | | | | 2H). |
| 96-8 | ++++ | 2-2, | C | (2S)-2-(3,5-dichlorophenyl)-1-[5- | 552.3 | (DMSO-d₆, 400 MHz) |
| (1^{st} | | 75 | | (2,3-dihydro-1,4-benzodioxine-6- | | δ (ppm): 7.49-7.48 (m, |
| eluting | | | | sulfonyl)-1H,2H,3H,4H,5H,6H- | | 1H), 7.37 (d, J = 1.6 |
| isomer) | | | | pyrrolo[3,4-c]pyrrol-2-yl]-4- | | Hz, 2H), 7.30-7.25 (m, |
| 96-9 | ++ | | | (methylamino)butan-1-one; | | 2H), 7.07 (d, J = 8.4 |
| (2^{nd} | | | | (2R)-2-(3,5-dichlorophenyl)-1-[5- | | Hz, 1H), 4.45-4.34 (m, |
| eluting | | | | (2,3-dihydro-1,4-benzodioxine-6- | | 1H), 4.32-4.29 (m, |
| isomer) | | | | sulfonyl)-1H,2H,3H,4H,5H,6H- | | 4H), 4.14-3.89 (m, |
| | | | | pyrrolo[3,4-c]pyrrol-2-yl]-4- | | 9H), 2.33-2.31 (m, |
| | | | | (methylamino)butan-1-one | | 2H), 2.20 (s, 3H), 2.10- |
| | | | | | | 1.98 (m, 1H), 1.74-1.65 |
| | | | | | | (m, 1H). |
| 97-15 | ++++ | 63-9, | D | (2S)-1-[5-(2,3-dihydro-1,4- | 557.0 | (CDCl₃, 400 MHz) δ |
| (1^{st} | | 66-4, | | benzodioxine-6-sulfonyl)- | | (ppm): 7.36-7.31 (m, |
| eluting | | 83-2, | | 1H,2H,3H,4H,5H,6H-pyrrolo[3,4- | | 2H), 7.28-7.23 (m, |
| isomer) | | 87 | | c]pyrrol-2-yl]-2-hydroxy-2-[3-(4- | | 1H), 7.01-6.97 (m, |
| 97-16 | ++ | | | methylpiperazin-1- | | 2H), 6.90-6.88 (m, |
| (2^{nd} | | | | yl)phenyl]ethane-1-thione; | | 2H), 5.18-5.15 (m, |
| eluting | | | | (2R)-1-[5-(2,3-dihydro-1,4- | | 1H), 5.05-5.03 (m, |
| isomer) | | | | benzodioxine-6-sulfonyl)- | | 1H), 4.66-4.61 (m, |
| | | | | 1H,2H,3H,4H,5H,6H-pyrrolo[3,4- | | 1H), 4.49-4.44 (m, |
| | | | | c]pyrrol-2-yl]-2-hydroxy-2-[3-(4- | | 1H), 4.37-4.30 (m, |
| | | | | methylpiperazin-1- | | 5H), 4.20-4.11 (m, |
| | | | | yl)phenyl]ethane-1-thione | | 3H), 4.04-4.01 (m, |
| | | | | | | 1H), 3.92-3.87 (m, |
| | | | | | | 1H), 3.31 (s, 4H), 2.81- |
| | | | | | | 2.71 (m, 4H), 2.55-2.46 |
| | | | | | | (m, 3H). |
| 97-17 | −− | 66-2, | D | (2S)-1-[5-(2,3-dihydro-1,4- | 565.0 | (DMSO-d6, 400 MHz) |
| (1^{st} | | 2-2, | | benzodioxine-6-sulfonyl)- | | δ (ppm): 7.30-7.21 (m, |
| eluting | | 63-9 | | 1H,2H,3H,4H,5H,6H-pyrrolo[3,4- | | 2H), 7.13 (d, J = 7.6 |
| isomer) | | | | c]pyrrol-2-yl]-2-hydroxy-2-(3-{5- | | Hz, 1H), 7.06 (d, J = |
| 97-18 | ++++ | | | methyl-1H,2H,3H,4H,5H,6H- | | 8.4 Hz, 1H), 6.58 (d, J = |
| (2^{nd} | | | | pyrrolo[3,4-c]pyrrol-2- | | 7.6 Hz, 1H), 6.50 (d, |
| eluting | | | | yl}phenyl)ethan-1-one; | | J = 2.0 Hz, 1H), 6.42 |
| isomer) | | | | (2R)-1-[5-(2,3-dihydro-1,4- | | (d, J = 8.0 Hz, 1H), |
| | | | | benzodioxine-6-sulfonyl)- | | 5.47 (d, J = 6.4 Hz, |
| | | | | 1H,2H,3H,4H,5H,6H-pyrrolo[3,4- | | 1H), 5.06 (d, J = 5.6 |
| | | | | c]pyrrol-2-yl]-2-hydroxy-2-(3-{5- | | Hz, 1H), 4.33-4.29 (m, |
| | | | | methyl-1H,2H,3H,4H,5H,6H- | | 4H), 4.25-4.20 (m, |
| | | | | pyrrolo[3,4-c]pyrrol-2- | | 1H), 4.11-4.07 (m, |
| | | | | yl}phenyl)ethan-1-one | | 1H), 3.98-3.87 (m, |
| | | | | | | 10H), 3.48 (s, 4H), |
| | | | | | | 2.48 (s, 3H). |
| 98-1 | ++++ | 7-1, | E | (2S)-2-(2-cyclopropyl-2,3- | 524.5 | (CDCl₃, 400 MHz) δ |
| (1^{st} | | 2-2 | | dihydro-1H-isoindol-4-yl)-1-[5- | | (ppm): 7.35-7.28 (m, |
| eluting | | | | (2,3-dihydro-1,4-benzodioxine-6- | | 2H), 7.25-7.22 (m, |
| isomer) | | | | sulfonyl)-1H,2H,3H,4H,5H,6H- | | 2H), 7.14-7.12 (m, |
| | | | | pyrrolo[3,4-c]pyrrol-2-yl]-2- | | 1H), 7.03-6.97 (m, |
| | | | | hydroxyethan-1-one; | | 1H), 5.02 (s, 1H), 4.37- |
| 98-2 | + | | | (2R)-2-(2-cyclopropyl-2,3- | | 4.32 (m, 6H), 4.32-4.04 |
| (2^{nd} | | | | dihydro-1H-isoindol-4-yl)-1-[5- | | (m, 8H), 3.97-3.85 (m, |
| eluting | | | | (2,3-dihydro-1,4-benzodioxine-6- | | 1H), 3.52-3.48 (m, |
| isomer) | | | | sulfonyl)-1H,2H,3H,4H,5H,6H- | | 1H), 2.16-2.03 (s, 1H), |
| | | | | pyrrolo[3,4-c]pyrrol-2-yl]-2- | | 0.61-0.57 (br s, 4H). |
| | | | | hydroxyethan-1-one | | |
| 99-8 | ++++ | 33-1, | | (2S)-2-(3-chlorophenyl)-1-[5-(2,3- | 530.3 | (DMSO-d₆, 400 MHz) |
| (1^{st} | | 2-2 | | dihydro-1,4-benzodioxine-6- | | δ (ppm): 7.38 (s, 1H), |
| eluting | | | | sulfonyl)-1H,2H,3H,4H,5H,6H- | | 7.33-7.23 (m, 5H), |
| isomer) | | | | pyrrolo[3,4-c]pyrrol-2-yl]-2-[(2S)- | | 7.07-7.05 (m, 1H), |
| 99-9 | ++ | | F | pyrrolidin-2-yl]ethan-1-one; | | 4.42-4.31 (m, 1H), |
| (2^{nd} | | | | (2R)-2-(3-chlorophenyl)-1-[5-(2,3- | | 4.30-4.29 (m, 4H), |
| eluting | | | | dihydro-1,4-benzodioxine-6- | | 4.03-3.95 (m, 7H), |
| isomer) | | | | sulfonyl)-1H,2H,3H,4H,5H,6H- | | 3.53-3.43 (m, 2H), |
| | | | | pyrrolo[3,4-c]pyrrol-2-yl]-2-[(2R)- | | 2.77-2.72 (m, 1H), |
| | | | | pyrrolidin-2-yl]ethan-1-one | | 2.66-2.59 (m, 1H), 1.85 |
| | | | | | | (s, 1H), 1.84-1.81 (m, |
| | | | | | | 1H), 1.66-1.57 (m, |
| | | | | | | 2H), 1.28-1.19 (m, |
| | | | | | | 1H). |
| 103-7 | ++++ | 2-2, | J | (2S)-3-(azetidin-1-yl)-1-[5-(2,3- | 567.4 | (DMSO-d₆, 400 MHz) |
| (1^{st} | | 2-27, | | dihydro-1,4-benzodioxine-6- | | δ (ppm): 7.89-7.87 (m, |
| eluting | | 80 | | sulfonyl)-1H,2H,3H,4H,5H,6H- | | 1H), 7.38-7.36 (m, |
| isomer) | | | | pyrrolo[3,4-c]pyrrol-2-yl]-2-(2- | | 1H), 7.33-7.25 (m, |
| 103-8 | ++ | | | methyl-1,3-benzothiazol-4- | | 2H), 7.24-7.22 (m, |
| (2^{nd} | | | | yl)propan-1-one; | | 1H), 7.05 (d, J = 8.4 |
| eluting | | | | (2R)-3-(azetidin-1-yl)-1-[5-(2,3- | | Hz, 1H), 4.68-4.64 (m, |
| isomer) | | | | dihydro-1,4-benzodioxine-6- | | 1H), 4.42-4.23 (m, |
| | | | | sulfonyl)-1H,2H,3H,4H,5H,6H- | | 5H), 4.17-4.15 (m, |
| | | | | pyrrolo[3,4-c]pyrrol-2-yl]-2-(2- | | 1H), 4.06-3.88 (m, 6H), |
| | | | | methyl-1,3-benzothiazol-4- | | 3.15-3.05 (m, 5H), |
| | | | | yl)propan-1-one | | 2.81 (s, 3H), 2.56-2.51 |
| | | | | | | (m, 1H), 1.91-1.84 (m, |
| | | | | | | 2H). |
| 103-46 | + | 38-1, | | (2S)-1-{5-[2,3-dihydro(2,2,3,3- | | (DMSO-d₆, 400 MHz) |
| (1^{st} | | 43-1, | | ²H₄)-1,4-benzodioxine-6- | | δ (ppm): 7.58 (s, 1H), |
| eluting | | 89 | | sulfonyl]-1H,2H,3H,4H,5H,6H- | | 7.37-7.21 (m, 4H), 7.06 |
| isomer) | | | | pyrrolo[3,4-c]pyrrol-2-yl}-2- | | (d, J = 8.4 Hz, 1H), |
| 103-47 | ++++ | | J | hydroxy-2-(2-methyl-1,3- | 502.0 | 5.71 (q, J = 6.8 Hz, |
| (2^{nd} | | | | benzoxazol-4-yl)ethan-1-one; | | 2H), 4.38-4.35 (m, |
| eluting | | | | (2R)-1-{5-[2,3-dihydro(2,2,3,3- | | 1H), 4.11-3.86 (m, |
| isomer) | | | | ²H₄)-1,4-benzodioxine-6- | | 7H), 2.62 (s, 3H). |
| | | | | sulfonyl]-1H,2H,3H,4H,5H,6H- | | |
| | | | | pyrrolo[3,4-c]pyrrol-2-yl}-2- | | |
| | | | | hydroxy-2-(2-methyl-1,3- | | |
| | | | | benzoxazol-4-yl)ethan-1-one | | |
| 112-3 | ++ | 7-5, | R | (2S)-1-[5-(2,3-dihydro-1,4- | 548.0 | (DMSO-d₆, 400 MHz) |
| (1^{st} | | 2-2, | | benzodioxine-6-sulfonyl)- | | δ (ppm): 7.29-7.25 (m, |
| eluting | | 5-4 | | 1H,2H,3H,4H,5H,6H-pyrrolo[3,4- | | 2H), 7.18-7.13 (m, |
| isomer) | | | | c]pyrrol-2-yl]-2-{3-[2- | | 2H), 7.06 (d, J = 8.4 |
| 112-4 | ++++ | | | (dimethylamino)ethoxy]-4- | | Hz, 1H), 6.91 (s, 1H), |
| (2^{nd} | | | | fluorophenyl}-2-hydroxyethan-1- | | 5.71 (d, J = 6.4 Hz, |
| eluting | | | | one; | | 1H), 5.14 (d, J = 6.4 |
| isomer) | | | | (2R)-1-[5-(2,3-dihydro-1,4- | | Hz, 1H), 4.32-4.25 (m, |
| | | | | benzodioxine-6-sulfonyl)- | | 5H), 4.10-3.96 (m, |
| | | | | 1H,2H,3H,4H,5H,6H-pyrrolo[3,4- | | 9H), 2.64-2.62 (m, |
| | | | | c]pyrrol-2-yl]-2-{3-[2- | | 2H), 2.21 (s, 6H). |
| | | | | (dimethylamino)ethoxy]-4- | | |
| | | | | fluorophenyl}-2-hydroxyethan-1- | | |
| | | | | one | | |
| 114-1 | ++++ | 2-2, | S | (2S)-2-(3-{9,9-difluoro-3,7- | 603.0 | (CD₃OD, 400 MHz) δ |
| (1^{st} | | 83, | | diazabicyclo[3.3.1]nonan-3- | | (ppm): 7.33-7.26 (m, |
| eluting | | 84, | | yl}phenyl)-1-[5-(2,3-dihydro-l,4- | | 3H), 7.10-7.00 (m, |
| isomer) | | 66-3, | | benzodioxine-6-sulfonyl)- | | 3H), 6.93-6.91 (m, |
| | | 85 | | 1H,2H,3H,4H,5H,6H-pyrrolo[3,4- | | 1H), 5.20 (s, 1H), 4.33- |
| 114-2 | ++ | | | c]pyrrol-2-yl]-2-hydroxyethan-1- | | 4.20 (m, 6H), 4.09-4.05 |
| (2^{nd} | | | | one; | | (m, 5H), 3.93-3.85 (m, |
| eluting | | | | (2R)-2-(3-{9,9-difluoro-3,7- | | 3H), 3.36-3.33 (m, |
| isomer) | | | | diazabicyclo[3.3.1]nonan-3- | | 2H), 3.31-3.25 (m, |
| | | | | yl}phenyl)-1-[5-(2,3-dihydro-1,4- | | 2H), 3.17-3.12 (m, |
| | | | | benzodioxine-6-sulfonyl)- | | 2H), 2.22 (br s, 2H). |
| | | | | 1H,2H,3H,4H,5H,6H-pyrrolo[3,4- | | |
| | | | | c]pyrrol-2-yl]-2-hydroxyethan-1- | | |
| | | | | one | | |

### Synthesis of Compounds of Formula I

In some embodiments, the USP9X inhibitor may be a compound of Table 11:

TABLE 11

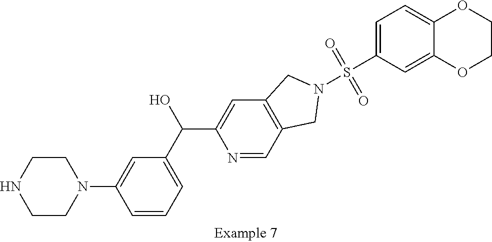 [SMILES: O=S(=O)(c1ccc2c(c1)OCCO2)N1Cc2cnc(C(O)c3cccc(N4CCNCC4)c3)cc2C1]

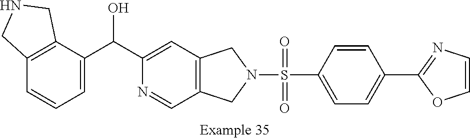 [SMILES: O=S(=O)(c1ccc(-c2ncco2)cc1)N1Cc2cnc(C(O)c3cccc4c3CNC4)cc2C1]

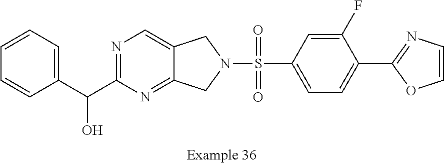 [SMILES: O=S(=O)(c1ccc(-c2ncco2)c(F)c1)N1Cc2cnc(C(O)c3ccccc3)nc2C1]

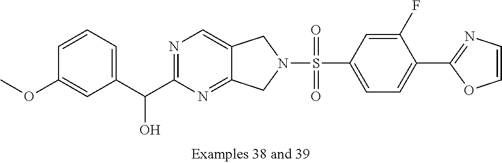 [SMILES: COc1cccc(C(O)c2ncc3c(n2)CN(S(=O)(=O)c2ccc(-c4ncco4)c(F)c2)C3)c1]

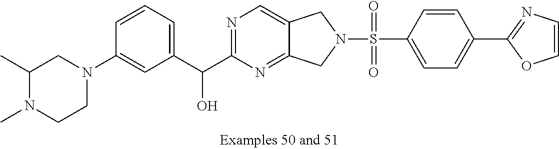 [SMILES: CC1CN(c2cccc(C(O)c3ncc4c(n3)CN(S(=O)(=O)c3ccc(-c5ncco5)cc3)C4)c2)CCN1C]

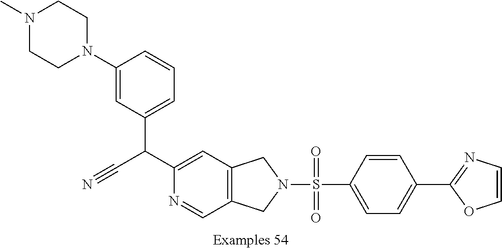 [SMILES: CN1CCN(c2cccc(C(C#N)c3cc4c(cn3)CN(S(=O)(=O)c3ccc(-c5ncco5)cc3)C4)c2)CC1]

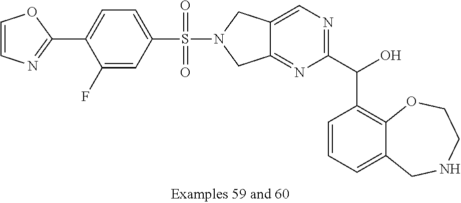 [SMILES: O=S(=O)(c1ccc(-c2ncco2)c(F)c1)N1Cc2cnc(C(O)c3cccc4c3OCCNC4)nc2C1]

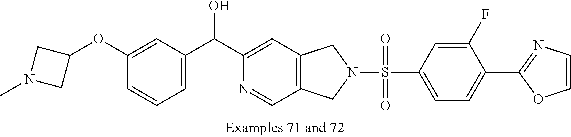 [SMILES: CN1CC(Oc2cccc(C(O)c3cc4c(cn3)CN(S(=O)(=O)c3ccc(-c5ncco5)c(F)c3)C4)c2)C1]

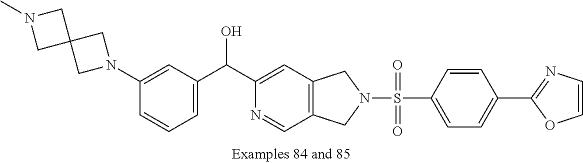 [SMILES: CN1CC2(C1)CN(c1cccc(C(O)c3cc4c(cn3)CN(S(=O)(=O)c3ccc(-c5ncco5)cc3)C4)c1)C2]

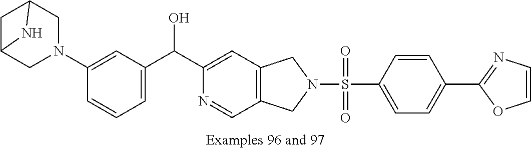 [SMILES: O=S(=O)(c1ccc(-c2ncco2)cc1)N1Cc2cnc(C(O)c3cccc(N4CC5CC(C4)N5)c3)cc2C1]

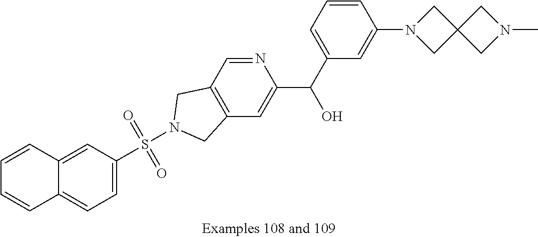 [SMILES: CN1CC2(C1)CN(c1cccc(C(O)c3cc4c(cn3)CN(S(=O)(=O)c3ccc5ccccc5c3)C4)c1)C2]

### Intermediates for Compounds of Table 11

### Intermediate 2′-1. (3-bromophenyl)([2-[3-fluoro-4-(1,3-oxazol-2-yl)benzenesulfonyl]-1H,2H,3H-pyrrolo[3,4-c]pyridin-6-yl])methanol

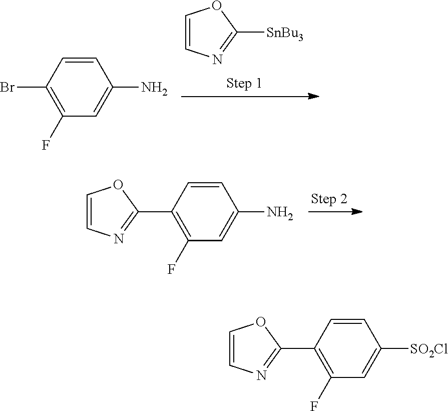

### Step 1. 3-fluoro-4-(1,3-oxazol-2-yl)aniline

To a solution of 4-bromo-3-fluoroaniline (474 mg, 2.51 mmol) in 1,4-dioxane (10 mL) was added Pd(dppf)Cl₂ (183 mg, 0.25 mmol) and 2-(tributylstannyl)-1,3-oxazole (900 mg, 2.52 mmol). The resulting mixture was stirred for 48 h at 100° C. and then cooled to room temperature. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 3-fluoro-4-(1,3-oxazol-2-yl)aniline (180 mg, 41%). LCMS (ES, m/z) 179 [M+H]⁺.

### Step 2. 3-fluoro-4-(1,3-oxazol-2-yl)benzene-1-sulfonyl chloride

Into glacial acetic acid (10 mL) was bubbled in SO₂ gas for 1 h at room temperature. Then CuCl₂ (34 mg, 0.25 mmol) was added and SO₂ gas was bubbled in for additional 2 h to afford solution A. To a pre-cooled solution of 3-fluoro-4-(1,3-oxazol-2-yl)aniline (180 mg, 1.01 mmol) in acetic acid (2 mL) and concentrated hydrochloric acid (6 mL) was added a solution of sodium nitrite (77 mg, 1.11 mmol) in distilled water (0.5 mL) dropwise with stirring at −10° C. After stirring for 15 min, solution A was added to this diazonium salt solution at −10° C. The resulting solution was allowed to warm to room temperature naturally and stirred for 16 h. The reaction mixture was treated with water (10 mL) and then extracted with EA (3×10 mL). The combined organic layers were dried over anhydrous sodium sulfate, filtered, and concentrated under vacuum. The resulting crude product was purified by silica gel chromatography (eluting with 0:100 to 12:88 ethyl acetate/petroleum ether) to afford 3-fluoro-4-(1,3-oxazol-2-yl)benzene-1-sulfonyl chloride (120 mg, 45%). LCMS (ES, m/z) 262, 264 [M+H]⁺.

The Intermediate in Table 12 was synthesized according to the procedure described for Intermediate 2′-1 above.

TABLE 12

| | | LCMS: (ESI) |
|---|---|---|
| Intermediate | Compound | m/z [M + H]⁺ |
| 2’-2 | 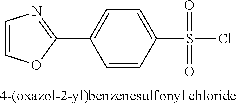 [SMILES: O=S(=O)(Cl)c1ccc(-c2ncco2)cc1] | 244 |

### Intermediate 3′-1. 1-tert-butyl-6-(3-[[2-(2,3-dihydro-1,4-benzodioxine-6-sulfonyl)-1H,2H,3H-pyrrolo[3,4-c]pyridin-6-yl]carbonyl]phenyl)-1lambda3,3,6-oxadiazocan-2-one

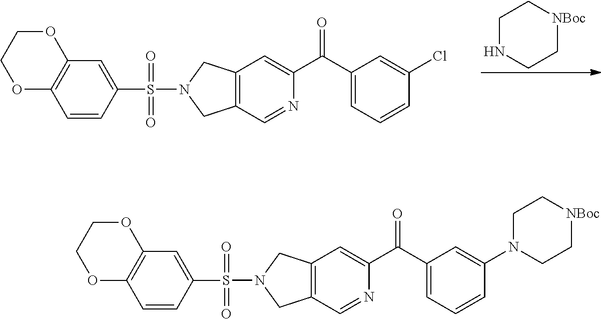 [SMILES: O=C(c1cccc(Cl)c1)c1cc2c(cn1)CN(S(=O)(=O)c1ccc3c(c1)OCCO3)C2 ; CC(C)(C)OC(=O)N1CCNCC1 ; CC(C)(C)OC(=O)N1CCN(c2cccc(C(=O)c3cc4c(cn3)CN(S(=O)(=O)c3ccc5c(c3)OCCO5)C4)c2)CC1]

To a solution of 6-[(3-chlorophenyl)carbonyl]-2-(2,3-dihydro-1,4-benzodioxine-6-sulfonyl)-1H,2H,3H-pyrrolo[3,4-c]pyridine (80 mg, 0.16 mmol) in toluene (8 mL) was added 1-tert-butyl-1lambda3,3,6-oxadiazocan-2-one (42 mg, 0.22 mmol), XPhos (19 mg, 0.04 mmol), Cs₂CO₃ (171 mg, 0.52 mmol) and Pd₂(dba)₃.CHCl₃ (18 mg, 0.02 mmol). The resulting mixture was stirred for 16 h at 100° C. and then cooled to room temperature. The reaction mixture was filtered and concentrated under vacuum. The resulting crude product was purified by silica gel chromatography (eluting with 1:10 EA/PE) to afford 1-tert-butyl-6-(3-[[2-(2,3-dihydro-1,4-benzodioxine-6-sulfonyl)-1H,2H,3H-pyrrolo[3,4-c]pyridin-6-yl]carbonyl]phenyl)-1lambda3,3,6-oxadiazocan-2-one as a yellow solid (80 mg, 84%). LCMS (ES, m/z) 607 [M+H]⁺.

The Intermediate in Table 13 was synthesized according to the procedure described for Intermediate 3′-1 above.

TABLE 13

| | | LCMS: (ESI) |
|---|---|---|
| Intermediate | Compound | m/z [M + H]⁺ |
| 3’-5^{a} | 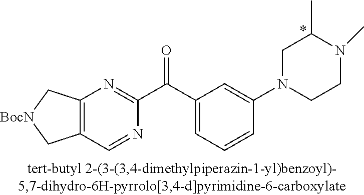 [SMILES: CC1CN(c2cccc(C(=O)c3ncc4c(n3)CN(C(=O)OC(C)(C)C)C4)c2)CCN1C] | 438 |

^{a}Ruphos 3G, Ruphos, K₃PO₄, dioxane, 100° C., 16 h;
*Absolute stereochemistry not determined.

### Intermediate 4′-1. 1-(3-[[2-(2,3-dihydro-1,4-benzodioxine-6-sulfonyl)-1H,2H,3H-pyrrolo[3,4-c]pyridin-6-yl]carbonyl]phenyl) piperazine

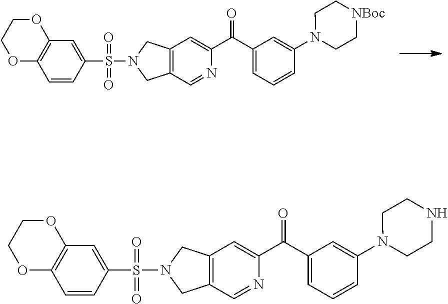 [SMILES: CC(C)(C)OC(=O)N1CCN(c2cccc(C(=O)c3cc4c(cn3)CN(S(=O)(=O)c3ccc5c(c3)OCCO5)C4)c2)CC1 ; O=C(c1cccc(N2CCNCC2)c1)c1cc2c(cn1)CN(S(=O)(=O)c1ccc3c(c1)OCCO3)C2]

To a solution of tert-butyl 4-(3-(2-((2,3-dihydrobenzo[b][1,4]dioxin-6-yl)sulfonyl)-2,3-dihydro-1H-pyrrolo[3,4-c]pyridine-6-carbonyl)phenyl)piperazine-1-carboxylate (80 mg, 0.12 mmol) in DCM (3 mL) was added TFA (1 mL). The resulting solution was stirred for 1 h at rt and then concentrated under vacuum. The resulting mixture was then basified to pH 8 with saturated aqueous potassium carbonate solution. The resulting mixture was extracted with DCM (3×5 mL). The combined organic layers were dried over anhydrous sodium sulfate, filtered and concentrated under vacuum to afford 1-(3-[[2-(2,3-dihydro-1,4-benzodioxine-6-sulfonyl)-1H,2H,3H-pyrrolo[3,4-c]pyridin-6-yl]carbonyl]phenyl)piperazine as a yellow solid (50 mg, 83%). LCMS (ES, m/z) 507 [M+H]⁺.

The Intermediates in Table 14 were synthesized according to the procedure described for Intermediate 4′-1 above.

TABLE 14

| | | LCMS: (ESI) |
|---|---|---|
| Intermediate | Compound | m/z [M + H]⁺ |
| 4’-11 | 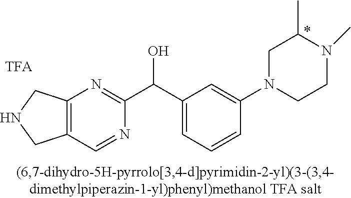 [SMILES: CC1CN(c2cccc(C(O)c3ncc4c(n3)CNC4)c2)CCN1C] | 340 |
| 4’-13 | 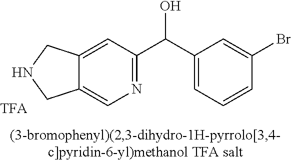 [SMILES: OC(c1cccc(Br)c1)c1cc2c(cn1)CNC2] | 305 |
| 4’-15 | 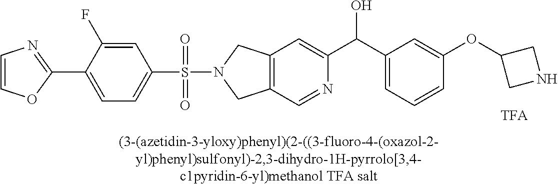 [SMILES: O=S(=O)(c1ccc(-c2ncco2)c(F)c1)N1Cc2cnc(C(O)c3cccc(OC4CNC4)c3)cc2C1] | 537 |

*Absolute stereochemistry not determined.

### Intermediate 8′-1. 1-[4-({6-benzoyl-1H,2H,3H-pyrrolo[3,4-c]pyridinyl}sulfonyl)phenyl]-1H-imidazole

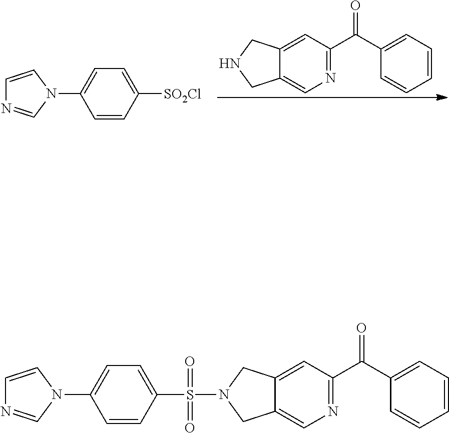 [SMILES: O=S(=O)(Cl)c1ccc(-n2ccnc2)cc1 ; O=C(c1ccccc1)c1cc2c(cn1)CNC2 ; O=C(c1ccccc1)c1cc2c(cn1)CN(S(=O)(=O)c1ccc(-n3ccnc3)cc1)C2]

To a solution of 4-(1H-imidazol-1-yl)benzene-1-sulfonyl chloride (200 mg, 0.82 mmol) in DCM (2 mL) was added 6-benzoyl-1H,2H,3H-pyrrolo[3,4-c]pyridine (124 mg, 0.55 mmol) and TEA (0.22 mL, 1.60 mmol). The resulting solution was stirred for 2 h at 25° C. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 10:90 ethyl acetate/petroleum ether) to afford 1-[4-([6-benzoyl-1H,2H,3H-pyrrolo[3,4-c]pyridin-2-yl]sulfonyl)phenyl]-1H-imidazole as a white solid (95 mg, 220%). LCMS (ES, m/z) 431 [M+H]⁺.

The Intermediates in Table 15 were synthesized according to the procedure described for Intermediate 8′-1 above.

TABLE 15

| | | LCMS: (ESI) |
|---|---|---|
| Intermediate | Compound | m/z [M + H]⁺ |
| 8’-11 | 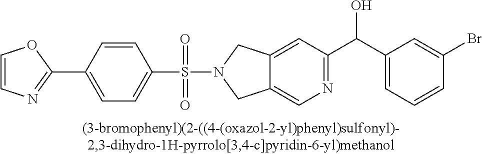 [SMILES: O=S(=O)(c1ccc(-c2ncco2)cc1)N1Cc2cnc(C(O)c3cccc(Br)c3)cc2C1] | 512, 514 |
| 8’-15 | 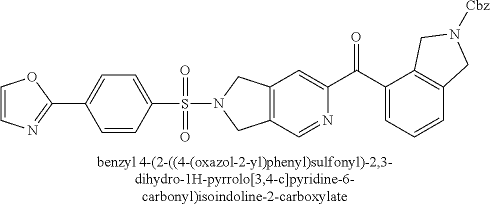 [SMILES: O=C(c1cc2c(cn1)CN(S(=O)(=O)c1ccc(-c3ncco3)cc1)C2)c1cccc2c1CN(C(=O)OCc1ccccc1)C2] | 607 |
| 8’-22 | 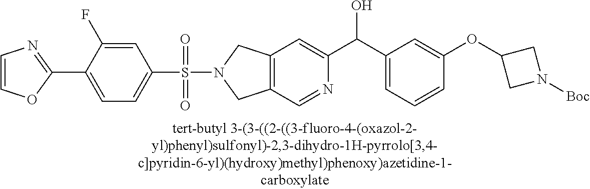 [SMILES: CC(C)(C)OC(=O)N1CC(Oc2cccc(C(O)c3cc4c(cn3)CN(S(=O)(=O)c3ccc(-c5ncco5)c(F)c3)C4)c2)C1] | 623 |

### Intermediate 10′-1. 6-[(3-chlorophenyl)carbonyl]-1H,2H,3H-pyrrolo[3,4-c]pyridine (TFA Salt)

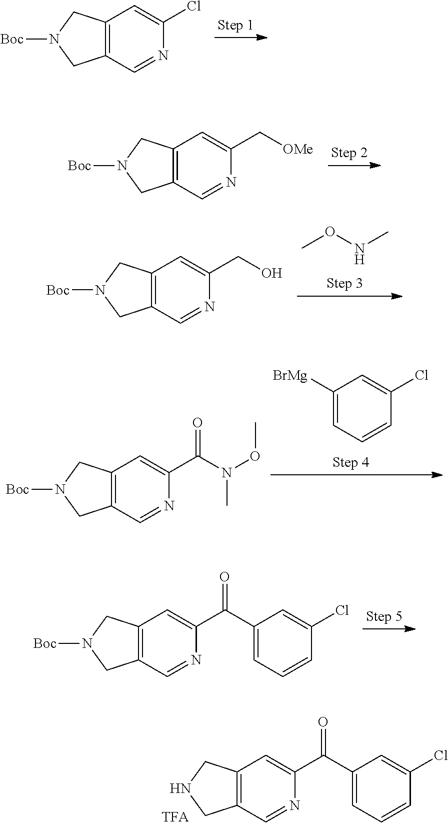

### Step 1. 2-tert-butyl 6-methyl 1H,2H,3H-pyrrolo[3,4-c]pyridine-2,6-dicarboxylate

Into a high pressure tank was placed a solution of tert-butyl 6-chloro-1H,2H,3H-pyrrolo[3,4-c]pyridine-2-carboxylate (2 g, 6.99 mmol) in MeOH (30 mL), Pd(dppf)Cl₂.CH₂Cl₂ (640 mg, 0.78 mmol) and TEA (3.28 mL, 23.7 mmol). Then CO (30 atm) was introduced. The resulting mixture was stirred for 16 h at 120° C. and cooled to room temperature. The reaction mixture was filtered and concentrated under vacuum. The resulting crude product was purified by silica gel chromatography (eluting with 1:5 EA/PE) to afford 2-tert-butyl 6-methyl 1H,2H,3H-pyrrolo[3,4-c]pyridine-2,6-dicarboxylate as a yellow solid (1.2 g, 56%). LCMS (ES, m/z) 279 [M+H]⁺.

### Step 2. 2-(tert-butoxycarbonyl)-2,3-dihydro-1H-pyrrolo[3,4-c]pyridine-6-carboxylic acid

To a solution of 2-tert-butyl 6-methyl 1H,2H,3H-pyrrolo[3,4-c]pyridine-2,6-dicarboxylate (2 g, 6.47 mmol) in THF (20 mL) was added water (15 mL) and LiOH (863 mg, 36.0 mmol). The resulting solution was stirred for 16 h at rt. The resulting mixture was washed with Et₂O (1×10 mL) and then acidified to pH 5 with hydrochloric acid solution (2 N). The resulting mixture was extracted with EA (3×25 mL). The combined organic layers were dried over anhydrous sodium sulfate, filtered and concentrated under vacuum. The resulting crude product was purified by reversed phase chromatography (eluting with 1:1 water/MeCN). The collected fractions were combined and concentrated under vacuum to afford 2-(tert-butoxycarbonyl)-2,3-dihydro-1H-pyrrolo[3,4-c]pyridine-6-carboxylic acid as yellow oil (1.0 g, 53%). LCMS (ES, m/z) 265 [M+H]⁺.

### Step 3. Tert-butyl 6-[methoxy(methyl)carbamoyl]-1H,2H,3H-pyrrolo[3,4-c]pyridine-2-carboxylate

To a solution of 2-[(tert-butoxy)carbonyl]-1H,2H,3H-pyrrolo[3,4-c]pyridine-6-carboxylic acid (1 g, 3.41 mmol) in DMF (15 mL) was added methoxy(methyl)amine hydrochloride (441 mg, 4.52 mmol), HATU (2.88 g, 7.57 mmol) and DIEA (1.98 mL, 11.37 mmol). The resulting solution was stirred for 1 h at rt. The reaction mixture was poured into water (15 mL) and then extracted with EA (3×15 mL). The combined organic layers were dried over anhydrous sodium sulfate, filtered and concentrate under vacuum. The resulting crude product was purified by silica gel chromatography (eluting with 1:10 EA/PE) to afford tert-butyl 6-[methoxy(methyl)carbamoyl]-1H,2H,3H-pyrrolo[3,4-c]pyridine-2-carboxylate as light yellow oil (700 mg, 67%). LCMS (ES, m/z) 308 [M+H]⁺.

### Step 4. Tert-butyl 6-[(3-chlorophenyl)carbonyl]-1H,2H,3H-pyrrolo[3,4-c]pyridine-2-carboxylate

To a solution of tert-butyl 6-[methoxy(methyl)carbamoyl]-1H,2H,3H-pyrrolo[3,4-c]pyridine-2-carboxylate (100 mg, 0.29 mmol) in THF (1 mL) was added a solution of bromo(3-chlorophenyl)magnesium (0.78 mL, 0.5 M in THF) dropwise with stirring at 0° C. The resulting solution was stirred for 1 h at rt and then poured into saturated ammonium chloride solution (5 mL). The resulting mixture was extracted with EA (3×5 mL). The combined organic layers were dried over anhydrous sodium sulfate, filtered and concentrated under vacuum. The resulting crude product was purified by silica gel chromatography (eluting with 1:3 EA/PE) to afford tert-butyl 6-[(3-chlorophenyl)carbonyl]-1H,2H,3H-pyrrolo[3,4-c]pyridine-2-carboxylate as yellow oil (80 mg, 76%). LCMS (ES, m/z) 359, 361 [M+H]⁺.

### Step 5. 6-[(3-chlorophenyl)carbonyl]-1H,2H,3H-pyrrolo[3,4-c]pyridine TFA salt

To a solution of tert-butyl 6-[(3-chlorophenyl)carbonyl]-1H,2H,3H-pyrrolo[3,4-c]pyridine-2-carboxylate (500 mg, 1.25 mmol) in dichloromethane (8 mL) was added TFA (2 mL). The resulting solution was stirred for 1 h at rt. The resulting mixture was concentrated under vacuum to afford 6-[(3-chlorophenyl)carbonyl]-1H,2H,3H-pyrrolo[3,4-c]pyridine (TFA salt) as brown oil (500 mg, crude). LCMS (ES, m/z) 259, 261 [M+H]⁺.

### Intermediate 20′-1. Phenyl({5H,6H,7H-pyrrolo[3,4-d]pyrimidin-2-yl})methanol

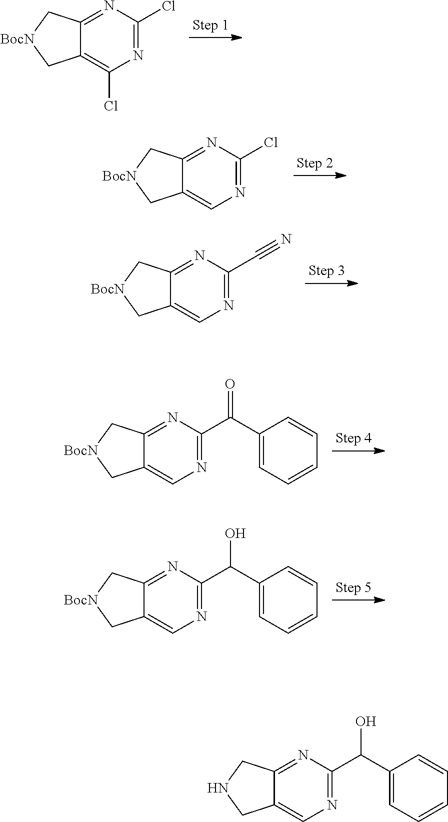 [SMILES: CC(C)(C)OC(=O)N1Cc2nc(Cl)nc(Cl)c2C1 ; CC(C)(C)OC(=O)N1Cc2cnc(Cl)nc2C1 ; CC(C)(C)OC(=O)N1Cc2cnc(C#N)nc2C1 ; CC(C)(C)OC(=O)N1Cc2cnc(C(=O)c3ccccc3)nc2C1 ; CC(C)(C)OC(=O)N1Cc2cnc(C(O)c3ccccc3)nc2C1 ; OC(c1ccccc1)c1ncc2c(n1)CNC2]

### Step 1. tert-Butyl 2-chloro-5H,6H,7H-pyrrolo[3,4-d]pyrimidine-6-carboxylate

To a solution of tert-butyl 2,4-dichloro-5H,6H,7H-pyrrolo[3,4-d]pyrimidine-6-carboxylate (6.00 g, 17.6 mmol) in methanol (48 mL) was added zinc powder (1.80 g, 26.4 mmol) and acetic acid (10.6 mL, 176 mmol). The resulting mixture was stirred for 16 h at 50° C. and cooled to room temperature. The resulting mixture was concentrated under vacuum. The resulting crude product was purified by silica gel chromatography (eluting with 1:1 ethyl acetate/petroleum ether) to afford tert-butyl 2-chloro-5H,6H,7H-pyrrolo[3,4-d]pyrimidine-6-carboxylate as a white solid (2.90 g, 54%). LCMS (ES, m/z): 256, 258 [M+H]⁺.

### Step 2. tert-Butyl 2-cyano-5H,6H,7H-pyrrolo[3,4-d]pyrimidine-6-carboxylate

To a solution of tert-butyl 2-chloro-5H,6H,7H-pyrrolo[3,4-d]pyrimidine-6-carboxylate (1.50 g, 4.99 mmol) in DMF (15 mL) was added Zn(CN)₂ (868 mg, 7.48 mmol) and Pd(dppf)Cl₂ (364 mg, 0.50 mmol). The resulting mixture was irradiated with microwave for 3 h at 140° C. After cooling to rt, the reaction mixture was poured into water (10 mL) and then extracted with ethyl acetate (3×15 mL). The combined organic layers were dried over anhydrous sodium sulfate, filtered, and concentrated under vacuum. The resulting crude product was purified by silica gel chromatography (eluting with 1:1 ethyl acetate/petroleum ether) to afford tert-butyl 2-cyano-5H,6H,7H-pyrrolo[3,4-d]pyrimidine-6-carboxylate as a yellow oil (500 mg, 34%). LCMS (ES, m/z): 247 [M+H]⁺.

### Step 3. tert-Butyl 2-benzoyl-5H,6H,7H-pyrrolo[3,4-d]pyrimidine-6-carboxylate

To a solution of tert-butyl 2-cyano-5H,6H,7H-pyrrolo[3,4-d]pyrimidine-6-carboxylate (200 mg, 0.69 mmol) in THF (2 mL) was added bromo(phenyl)magnesium (1.38 mL, 1 M in THF) dropwise at 0° C. The resulting mixture was stirred for 1 h at rt. Then 1 N hydrochloric acid (2 mL) was added. The resulting mixture was stirred for 30 min at rt and then extracted with EA (3×5 mL). The combined organic layers were dried over anhydrous sodium sulfate, filtered, and concentrated under vacuum. The resulting crude product was purified by silica gel chromatography (eluting with 4:5 ethyl acetate/petroleum ether) to afford tert-butyl 2-benzoyl-5H,6H,7H-pyrrolo[3,4-d]pyrimidine-6-carboxylate as a yellow oil (90 mg, 34%). LCMS (ES, m/z): 326 [M+H]⁺.

### Step 4. tert-Butyl 2-[hydroxy(phenyl)methyl]-5H,6H,7H-pyrrolo[3,4-d]pyrimidine-6-carboxylate

To a solution of tert-butyl 2-benzoyl-5H,6H,7H-pyrrolo[3,4-d]pyrimidine-6-carboxylate (90 mg, 0.24 mmol) in methanol (1 mL) was added sodium borohydride (18.7 mg, 0.47 mmol). The resulting mixture stirred for 1 h at rt. The reaction mixture was poured into water (5 mL) and then extracted with EA (3×5 mL). The combined organic layers were dried over anhydrous sodium sulfate, filtered, and concentrated under vacuum. The resulting crude product was purified by Prep-TLC (eluting with 2:5 ethyl acetate/petroleum ether) to afford tert-butyl 2-[hydroxy(phenyl)methyl]-5H,6H,7H-pyrrolo[3,4-d]pyrimidine-6-carboxylate as a yellow solid (60 mg, 77%). LCMS (ES, m/z): 328 [M+H]⁺.

### Step 5. Phenyl({5H,6H,7H-pyrrolo[3,4-d]pyrimidin-2-yl})methanol

To a solution of tert-butyl 2-[hydroxy(phenyl)methyl]-5H,6H,7H-pyrrolo[3,4-d]pyrimidine-6-carboxylate (60 mg, 0.18 mmol) in DCM (6 mL) was added trifluoroacetic acid (2 mL). The resulting mixture was stirred for 1 h at rt and concentrated under vacuum. The resulting mixture was basified to pH 8 with saturated potassium carbonate solution and extracted with DCM (3×5 mL). The combined organic layers were dried over anhydrous sodium sulfate, filtered, and concentrated under vacuum to afford phenyl({5H,6H,7H-pyrrolo[3,4-d]pyrimidin-2-yl})methanol as a yellow solid (35 mg, 85%). LCMS (ES, m/z): 228 [M+H]⁺.

The Intermediate in Table 16 was synthesized according to the procedure described for Intermediate 20′-1 above.

TABLE 16

| Inter- | | LCMS: (ESI) |
|---|---|---|
| mediate | Compound | m/z [M + H]⁺ |
| 20’-2 | 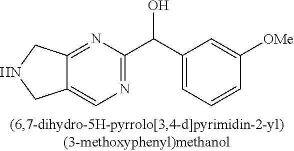 [SMILES: COc1cccc(C(O)c2ncc3c(n2)CNC3)c1] | 258 |

### Intermediate 21′-1. Benzyl 4-[1H,2H,3H-pyrrolo[3,4-c]pyridine-6-carbonyl]-2,3-dihydro-1H-isoindole-2-carboxylate

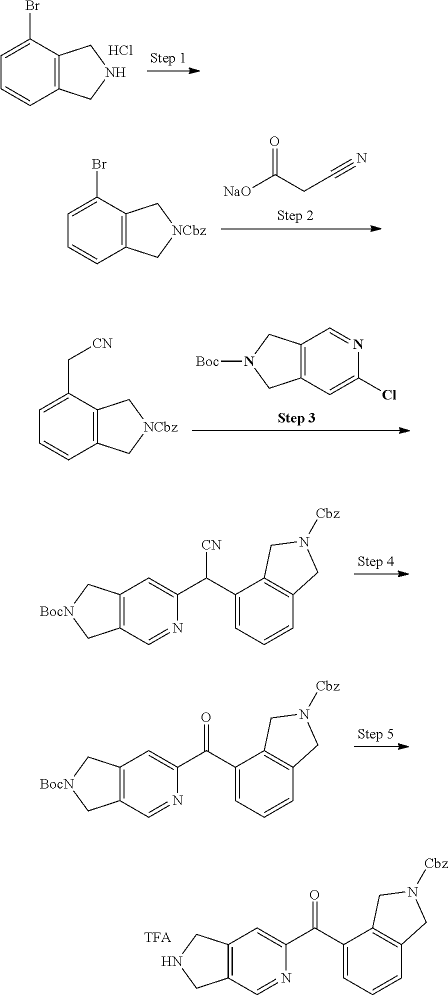

### Step 1. Benzyl 4-bromo-2,3-dihydro-1H-isoindole-2-carboxylate

To a solution of 4-bromo-2,3-dihydro-1H-isoindole hydrochloride (3.00 g, 12.2 mmol) and TEA (5.10 mL, 36.5 mmol) in dichloromethane (50 mL) was added CbzCl (4.10 g, 24.3 mmol) in portions at 0° C. The resulting solution was stirred for 5 h at room temperature. The reaction mixture was poured into water (50 mL) and then extracted with ethyl acetate (3×50 mL). The combined organic layers were dried over anhydrous sodium sulfate, filtered and concentrated under vacuum. The resulting crude product was purified by silica gel chromatography (eluting with 0:100 to 20:80 ethyl acetate/petroleum ether) to afford benzyl 4-bromo-2,3-dihydro-1H-isoindole-2-carboxylate as a pink solid (3.50 g, 87%). LCMS (ES, m/z): 332, 334[M+H]⁺.

### Step 2. tert-Butyl 4-(cyanomethyl)-2,3-dihydro-1H-isoindole-2-carboxylate

To a solution of tert-butyl 4-bromo-2,3-dihydro-1H-isoindole-2-carboxylate (1.50 g, 4.78 mmol) in mesitylene (20 mL) was added Pd(allyl)₂Cl₂ (46 mg, 0.10 mmol), SPhos (118 mg, 0.29 mmol) and sodium 2-cyanoacetate (808 mg, 7.17 mmol). The resulting mixture was stirred for 5 h at 140° C. After cooling to room temperature, the reaction mixture was filtered and concentrated under vacuum. The resulting crude product was purified by silica gel chromatography (eluting with 0:100 to 50:50 ethyl acetate/petroleum ether) to afford tert-butyl 4-(cyanomethyl)-2,3-dihydro-1H-isoindole-2-carboxylate as a brown solid (1.00 g, 81%). LCMS (ES, m/z): 293[M+H]⁺.

### Step 3. Benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate

To a solution of tert-butyl 6-chloro-1H,2H,3H-pyrrolo[3,4-c]pyridine-2-carboxylate (500 mg, 1.86 mmo) in THF (10 mL) was added benzyl 4-(cyanomethyl)-2,3-dihydro-1H-isoindole-2-carboxylate (861 mg, 2.80 mmol) and sodium amide (146 mg, 3.74 mmol). The resulting solution was stirred for 4 h at 50° C. After cooling to room temperature, the reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 30:60 ethyl acetate/petroleum ether) to afford benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate as yellow oil (300 mg, 32%). LCMS (ES, m/z): 511[M+H]⁺.

### Step 4. Benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate

To a solution of benzyl 4-([2-[(tert-butoxy)carbonyl]-1H,2H,3H-pyrrolo[3,4-c]pyridin-6-yl](cyano)methyl)-2,3-dihydro-1H-isoindole-2-carboxylate (300 mg, 0.56 mmol) in DMSO (5 mL) was added benzyltriethylammonium chloride (6 mg, 0.03 mmol) and sodium hydroxide (0.2 mL, 4 N in water). Then oxygen was bubbled in. The resulting mixture was stirred for 2 h at room temperature. The reaction mixture was poured into water (20 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford benzyl 4-[2-[(tert-butoxy)carbonyl]-1H,2H,3H-pyrrolo[3,4-c]pyridine-6-carbonyl]-2,3-dihydro-1H-isoindole-2-carboxylate as yellow oil (200 mg, 72%). LCMS (ES, m/z): 500 [M+H]⁺.

### Step 5. Benzyl 4-[1H,2H,3H-pyrrolo[3,4-c]pyridine-6-carbonyl]-2,3-dihydro-1H-isoindole-2-carboxylate (TFA Salt)

To a solution of benzyl 4-[2-[(tert-butoxy)carbonyl]-1H,2H,3H-pyrrolo[3,4-c]pyridine-6-carbonyl]-2,3-dihydro-1H-isoindole-2-carboxylate (200 mg, 0.38 mmol) in dichloromethane (5 mL) was added trifluoroacetic acid (1 mL). The resulting solution was stirred for 5 h at room temperature. The resulting mixture was concentrated under vacuum to afford benzyl 4-[1H,2H,3H-pyrrolo[3,4-c]pyridine-6-carbonyl]-2,3-dihydro-1H-isoindole-2-carboxylate (TFA salt) as yellow oil (180 mg, crude). LCMS (ES, m/z): 400[M+H]⁺.

### Intermediate 25′-1. Tert-butyl 6-[hydroxy[2-(4-methylpiperazin-1-yl)phenyl]methyl]-1H,2H,3H-pyrrolo[3,4-c]pyridine-2-carboxylate

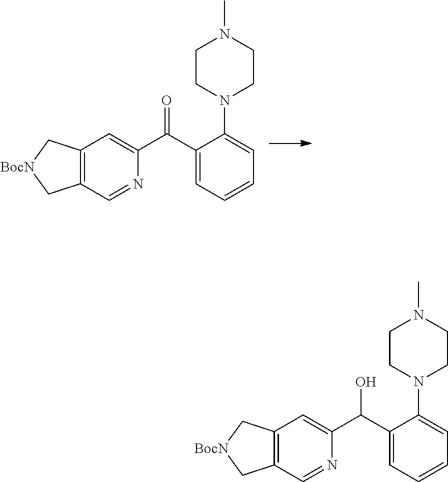 [SMILES: CN1CCN(c2ccccc2C(=O)c2cc3c(cn2)CN(C(=O)OC(C)(C)C)C3)CC1 ; CN1CCN(c2ccccc2C(O)c2cc3c(cn2)CN(C(=O)OC(C)(C)C)C3)CC1]

To a solution of tert-butyl 6-[2-(4-methylpiperazin-1-yl)benzoyl]-1H, 2H, 3H-pyrrolo[3,4-c]pyridine-2-carboxylate (200 mg, 0.43 mmol) in MeOH (10 mL) was added sodium borohydride (8 mg, 0.21 mmol). The resulting mixture was stirred for 1 h at 25° C. The reaction mixture was concentrated under vacuum. The resulting crude product was purified by silica gel chromatography (eluting with 1:10 MeOH/DCM) to afford tert-butyl 6-[hydroxy[2-(4-methylpiperazin-1-yl)phenyl]methyl]-1H,2H,3H-pyrrolo[3,4-c]pyridine-2-carboxylate as yellow oil (170 mg, 85%). LCMS (ES, m/z): 425 [M+H]⁺.

The Intermediate in Table 17 was synthesized according to the procedure described for Intermediate 25′-1 above.

TABLE 17

| | | LCMS: (ESI) |
|---|---|---|
| Intermediate | Compound | m/z [M + H]⁺ |
| 25’-3 | 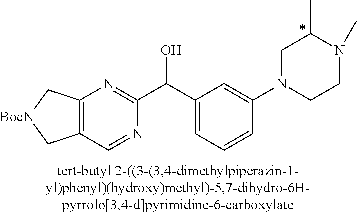 [SMILES: CC1CN(c2cccc(C(O)c3ncc4c(n3)CN(C(=O)OC(C)(C)C)C4)c2)CCN1C] | 440 |

*Absolute stereochemistry not determined.

### Intermediate 27′-1. Tert-butyl 2-(3-bromobenzoyl)-5H,6H,7H-pyrrolo[3,4-d]pyrimidine-6-carboxylate

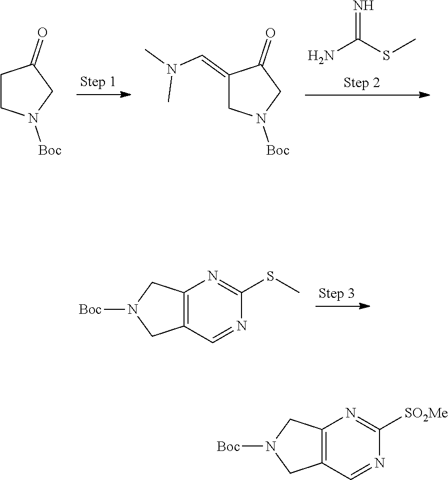

### Step 1. Tert-butyl (3E)-3-[(dimethylamino)methylidene]-4-oxopyrrolidine-1-carboxylate

To tert-butyl 3-oxopyrrolidine-1-carboxylate (20 g, 102 mmol) was added dimethylformamide dimethyl acetal (200 mL). The resulting solution was stirred for 12 h at 140° C. After cooling to room temperature, the resulting mixture was concentrated. The residue was dissolved with a minimum amount of DCM and then treated with hexane (100 mL). The resulting solids were collected by filtration and dried under vacuum to afford tert-butyl (3E)-3-[(dimethylamino)methylidene]-4-oxopyrrolidine-1-carboxylate as a yellow solid (15 g, 58%). LCMS (ES, m/z): 241 [M+H]⁺.

### Step 2. Tert-butyl 2-(methylsulfanyl)-5H,6H,7H-pyrrolo[3,4-d]pyrimidine-6-carboxylate

To a solution of (methylsulfanyl)methanimidamide (17 g, 183 mmol) in EtOH (200 mL) was added sodium ethoxide (13 g, 183 mmol) at 0° C. After stirring for 10 min, to the above solution was added tert-butyl (3E)-3-[(dimethylamino)methylidene]-4-oxopyrrolidine-1-carboxylate (15 g, 61.2 mmol). The resulting mixture was stirred for 4 h at 80° C. After cooling to room temperature, the reaction mixture was concentrated under vacuum. The residue was dissolved with water (100 mL) and then extracted with ethyl acetate (3×100 mL). The organic layers were combined, dried over anhydrous sodium sulfate, filtered and concentrated under vacuum. The resulting crude product was purified by silica gel chromatography (eluting with 1:2 ethyl acetate/petroleum ether) to afford tert-butyl 2-(methylsulfanyl)-5H,6H,7H-pyrrolo[3,4-d]pyrimidine-6-carboxylate as a yellow solid (4 g, 41%). LCMS (ES, m/z): 268 [M+H]⁺.

### Step 3. Tert-butyl 2-methanesulfonyl-5H,6H,7H-pyrrolo[3,4-d]pyrimidine-6-carboxylate

To a solution of tert-butyl 2-(methylsulfanyl)-5H,6H,7H-pyrrolo[3,4-d]pyrimidine-6-carboxylate (4 g, 14.1 mmol) in DCM (80 mL) was added m-CPBA (7.5 g, 42.6 mmol). The resulting mixture was stirred for 5 h at 0° C. The resulting mixture was washed with saturated sodium bicarbonate solution (5×100 mL), dried over anhydrous sodium sulfate, filtered and concentrated. The resulting crude product was purified by silica gel chromatography (eluting with 2:1 ethyl acetate/petroleum ether) to afford tert-butyl 2-methanesulfonyl-5H,6H,7H-pyrrolo[3,4-d]pyrimidine-6-carboxylate as a yellow solid (4 g, 89%). LCMS (ES, m/z): 300 [M+H]⁺.

### Intermediate 32′-1. 4-({6-[hydroxy(phenyl)methyl]-1H,2H,3H-pyrrolo[3,4-c]pyridin-2-yl}sulfonyl)benzonitrile

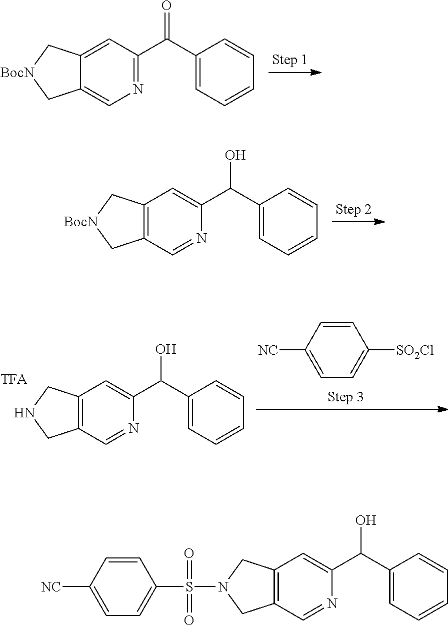 [SMILES: CC(C)(C)OC(=O)N1Cc2cnc(C(=O)c3ccccc3)cc2C1 ; CC(C)(C)OC(=O)N1Cc2cnc(C(O)c3ccccc3)cc2C1 ; OC(c1ccccc1)c1cc2c(cn1)CNC2 ; N#Cc1ccc(S(=O)(=O)Cl)cc1 ; N#Cc1ccc(S(=O)(=O)N2Cc3cnc(C(O)c4ccccc4)cc3C2)cc1]

### Step 1. Tert-butyl 6-[hydroxy(phenyl)methyl]-1H,2H,3H-pyrrolo[3,4-c]pyridine-2-carboxylate

To a solution of tert-butyl 6-benzoyl-1H,2H,3H-pyrrolo[3,4-c]pyridine-2-carboxylate (340 mg, 1.05 mmol) in MeOH (10 mL), was added sodium borohydride (12 mg, 0.32 mmol). The resulting mixture was stirred for 1 h at 25° C. The reaction mixture was poured into water (15 mL) and then extracted with ethyl acetate (3×10 mL). The organic layers were combined, dried over anhydrous sodium sulfate, filtered, and concentrated under vacuum. The resulting crude product was purified by silica gel chromatography (eluting with 0:100 to 10:90 ethyl acetate/petroleum ether) to afford tert-butyl 6-[hydroxy(phenyl)methyl]-1H,2H,3H-pyrrolo[3,4-c]pyridine-2-carboxylate as a yellow solid (300 mg, 88%). LCMS (ES, m/z): 327 [M+H]⁺.

### Step 2. Phenyl({1H,2H,3H-pyrrolo[3,4-c]pyridin-6-yl})methanol (TFA Salt)

To a solution of tert-butyl 6-[hydroxy(phenyl)methyl]-1H,2H,3H-pyrrolo[3,4-c]pyridine-2-carboxylate (280 mg, 0.86 mmol) in dichloromethane (4 mL) was added TFA (1 mL). The resulting mixture was stirred for 1 h at 25° C. The reaction mixture was concentrated under vacuum to afford phenyl({1H,2H,3H-pyrrolo[3,4-c]pyridin-6-yl})methanol (TFA salt) as light yellow oil (200 mg, crude). LCMS (ES, m/z): 227 [M+H]⁺.

### Step 3. 4-({6-[hydroxy(phenyl)methyl]-1H,2H,3H-pyrrolo[3,4-c]pyridin-2-yl}sulfonyl)benzonitrile

To a solution of phenyl({1H,2H,3H-pyrrolo[3,4-c]pyridin-6-yl})methanol (TFA salt) (225 mg, 0.99 mmol) in dichloromethane (7 mL) was added TEA (0.55 mL, 3.98 mmol) and 4-cyanobenzene-1-sulfonyl chloride (200 mg, 0.99 mmol). The resulting mixture was stirred for 2 h at 25° C. and concentrated under vacuum. The resulting crude product was purified by silica gel chromatography (eluting with 0:100 to 10:90 ethyl acetate/petroleum ether) to afford 4-({6-[hydroxy(phenyl)methyl]-1H,2H,3H-pyrrolo[3,4-c]pyridin-2-yl}sulfonyl)benzonitrile as a white solid (200 mg, 51%). LCMS (ES, m/z): 392 [M+H]⁺.

The Intermediate in Table 18 was synthesized according to the procedure described for Intermediate 32′-1 above.

TABLE 18

| | | LCMS: (ESI) |
|---|---|---|
| Intermediate | Compound | m/z [M + H]⁺ |
| 32’-2 | 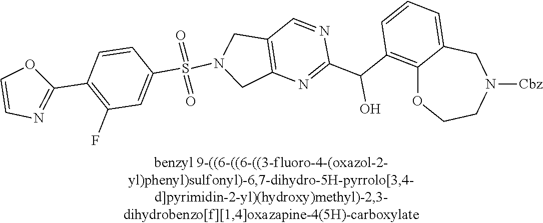 [SMILES: O=C(OCc1ccccc1)N1CCOc2c(cccc2C(O)c2ncc3c(n2)CN(S(=O)(=O)c2ccc(-c4ncco4)c(F)c2)C3)C1] | 658 |

### Intermediate 33′-1. Tert-butyl 2-(3-bromobenzoyl)-5H,6H,7H-pyrrolo[3,4-d]pyrimidine-6-carboxylate

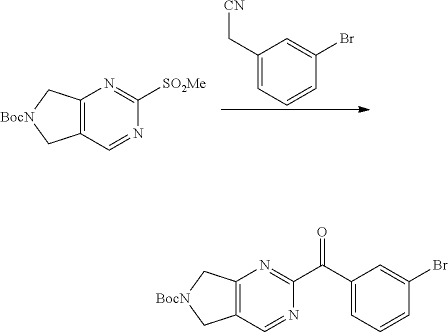 [SMILES: CC(C)(C)OC(=O)N1Cc2cnc(S(C)(=O)=O)nc2C1 ; N#CCc1cccc(Br)c1 ; CC(C)(C)OC(=O)N1Cc2cnc(C(=O)c3cccc(Br)c3)nc2C1]

To a solution of 2-(3-bromophenyl)acetonitrile (1.96 g, 9.52 mmol) in THF (50 mL) was added tert-butyl 2-methanesulfonyl-5H,6H,7H-pyrrolo[3,4-d]pyrimidine-6-carboxylate (2 g, 6.35 mmol) and potassium bis(trimethylsilyl)amide solution (10 mL, 1 M in THF). The resulting mixture was stirred for 12 h at room temperature while oxygen was kept bubbling in. The reaction mixture was poured into water (50 mL) and then extracted with ethyl acetate (3×50 mL). The organic layers were combined, dried over anhydrous sodium sulfate, filtered, and concentrated under vacuum. The resulting crude product was purified by silica gel chromatography (eluting with 0:100 to 30:70 ethyl acetate/petroleum ether) to afford tert-butyl 2-(3-bromobenzoyl)-5H,6H,7H-pyrrolo[3,4-d]pyrimidine-6-carboxylate as a yellow solid (1.20 g, 44%). LCMS (ES, m/z): 404, 406 [M+H]⁺.

The Intermediate in Table 19 was synthesized according to the procedure described for Intermediate 33′-1 above.

TABLE 19

| | | LCMS: (ESI) |
|---|---|---|
| Intermediate | Compound | m/z [M + H]⁺ |
| 33-2 | 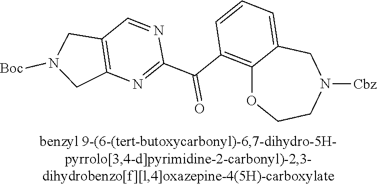 [SMILES: CC(C)(C)OC(=O)N1Cc2cnc(C(=O)c3cccc4c3OCCN(C(=O)OCc3ccccc3)C4)nc2C1] | 531 |

### Methods for Preparing Final Compounds

### Method A′

### [(2S)-2-[2-(2,3-dihydro-1,4-benzodioxine-6-sulfonyl)-1H,2H,3H-pyrrolo[3,4-c]pyridin-6-yl]-2-phenylethyl] (methyl)amine; [(2R)-2-[2-(2,3-dihydro-1,4-benzodioxine-6-sulfonyl)-1H,2H,3H-pyrrolo[3,4-c]pyridin-6-yl]-2-phenylethyl](methyl) amine

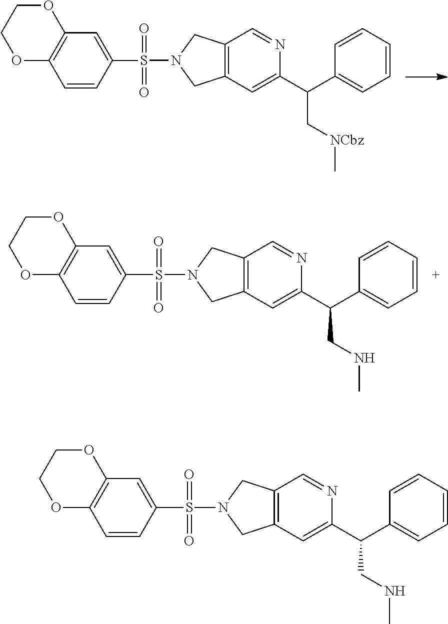 [SMILES: CN(CC(c1ccccc1)c1cc2c(cn1)CN(S(=O)(=O)c1ccc3c(c1)OCCO3)C2)C(=O)OCc1ccccc1 ; CNC[C@H](c1ccccc1)c1cc2c(cn1)CN(S(=O)(=O)c1ccc3c(c1)OCCO3)C2 ; CNC[C@@H](c1ccccc1)c1cc2c(cn1)CN(S(=O)(=O)c1ccc3c(c1)OCCO3)C2]

To a solution of (3-[2-[2-(2,3-dihydro-1,4-benzodioxine-6-sulfonyl)-1H,2H,3H-pyrrolo[3,4-c]pyridin-6-yl]-2-phenylethyl]phenyl)methyl N-methylcarbamate (100 mg, 0.17 mmol) in methanol (2 mL) was added palladium carbon (10 mg, 10 wt % palladium on charcoal). Then hydrogen was introduced with hydrogen balloon. The resulting mixture was stirred for 16 h at room temperature. The reaction mixture was filtered and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (5% to 30% over 25 min); Flow rate: 20 mL/min; Detector: UV 254 nm). The two enantiomers were further separated by Chiral-Pre-HPLC (Column: CHIRALPAK IG, 5 μm, 20×250 mm; Mobile Phase, A: methanol (containing 0.1% DEA) and B: DCM (hold 50% B over 10 min); Detector: UV 254/220 nm; Retention time: 1^{st} eluting isomer, 3.965 min; 2^{nd} eluting isomer, 5.955 min). The product fractions of 1^{st} eluting isomer were concentrated and lyophilized to afford a white solid (10.1 mg, 26%). ¹H-NMR (Methanol-d₄, 400 MHz) δ (ppm): 8.40 (s, 1H), 7.36-7.16 (m, 7H), 7.17 (s, 1H), 6.99-6.92 (m, 1H), 4.60 (s, 2H), 4.54 (s, 2H), 4.35-4.33 (m, 1H), 4.26-4.22 (m, 4H), 3.53-3.50 (m, 1H), 3.19-3.14 (m, 1H), 2.44 (s, 3H). LCMS (ES, m/z) 452 [M+H]⁺. The product fractions of 2^{nd} eluting isomer were concentrated and lyophilized to a white solid (11.5 mg, 30%). ¹H-NMR (Methanol-d₄, 400 MHz) δ (ppm): 8.40 (s, 1H), 7.36-7.16 (m, 7H), 7.17 (s, 1H), 6.99-6.92 (m, 1H), 4.61 (s, 2H), 4.56 (s, 2H), 4.42-4.39 (m, 1H), 4.26-4.22 (m, 4H), 3.66-3.59 (m, 1H), 3.19-3.14 (m, 1H), 2.55 (s, 3H). LCMS (ES, m/z) 452 [M+H]⁺.

### Method B′

### [2-[3-fluoro-4-(1,3-oxazol-2-yl)benzenesulfonyl]-1H,2H,3H-pyrrolo[3,4-c]pyridin-6-yl][3-(piperazin-1-yl)phenyl]methanol

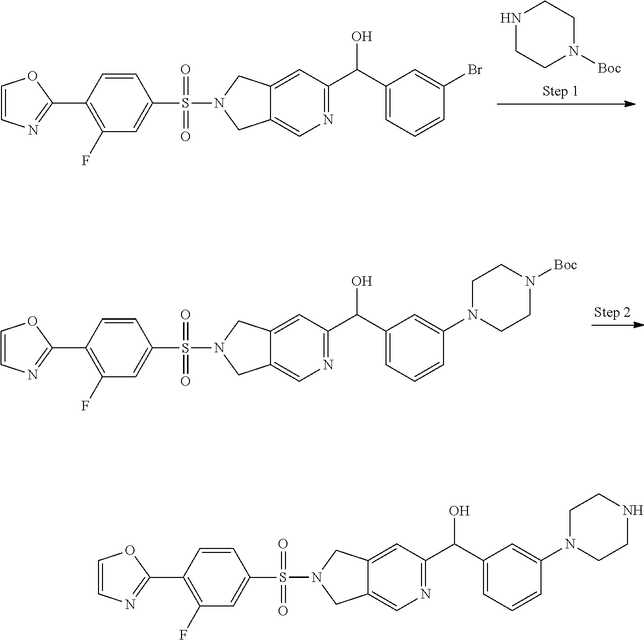 [SMILES: O=S(=O)(c1ccc(-c2ncco2)c(F)c1)N1Cc2cnc(C(O)c3cccc(Br)c3)cc2C1 ; CC(C)(C)OC(=O)N1CCNCC1 ; CC(C)(C)OC(=O)N1CCN(c2cccc(C(O)c3cc4c(cn3)CN(S(=O)(=O)c3ccc(-c5ncco5)c(F)c3)C4)c2)CC1 ; O=S(=O)(c1ccc(-c2ncco2)c(F)c1)N1Cc2cnc(C(O)c3cccc(N4CCNCC4)c3)cc2C1]

### Step 1. Tert-butyl 4-[3-[[2-[3-fluoro-4-(1,3-oxazol-2-yl)benzenesulfonyl]-1H,2H,3H-pyrrolo[3,4-c]pyridin-6-yl](hydroxy)methyl]phenyl]piperazine-1-carboxylate

To a solution of (3-bromophenyl)([2-[3-fluoro-4-(1,3-oxazol-2-yl)benzenesulfonyl]-1H,2H,3H-pyrrolo[3,4-c]pyridin-6-yl])methanol (50 mg, 0.09 mmol) in 1,4-dioxane (2 mL) was added K₃PO₄ (61 mg, 0.29 mmol), tert-butyl piperazine-1-carboxylate (88 mg, 0.47 mmol), RuPhos 3G (16 mg, 0.02 mmol), and RuPhos (9 mg, 0.02 mmol). The resulting mixture was stirred for 2 h at 100° C. and then cooled to room temperature. The reaction mixture was poured into water (3 mL) and then extracted with ethyl acetate (3×10 mL). The combined organic layers were dried over anhydrous sodium sulfate, filtered, and concentrated under vacuum. The resulting crude product was purified by prep-TLC (eluting with 1:1 ethyl acetate/petroleum ether) to afford tert-butyl 4-[3-[[2-[3-fluoro-4-(1,3-oxazol-2-yl)benzenesulfonyl]-1H,2H,3H-pyrrolo[3,4-c]pyridin-6-yl](hydroxy)methyl]phenyl}piperazine-1-carboxylate (40 mg, 67%). LCMS (ES, m/z) 636 [M+H]⁺.

### Step 2. [2-[3-fluoro-4-(1,3-oxazol-2-yl)benzenesulfonyl]-1H,2H,3H-pyrrolo[3,4-c]pyridin-6-yl][3-(piperazin-1-yl)phenyl]methanol

To a solution of tert-butyl 4-[3-[[2-[3-fluoro-4-(1,3-oxazol-2-yl)benzenesulfonyl]-1H,2H,3H-pyrrolo[3,4-c]pyridin-6-yl](hydroxy)methyl]phenyl}piperazine-1-carboxylate (40 mg, 0.06 mmol) in dichloromethane (4 mL) was added TFA (1 mL). The resulting solution was stirred for 2 h at room temperature and concentrated under vacuum. The resulting mixture was basified to pH 8 with saturated potassium carbonate solution and then extracted with dichloromethane (2×5 mL). The combined organic layers were dried over anhydrous sodium sulfate, filtered, and concentrated under vacuum. The resulting crude product was purified by Prep-HPLC (Column: XBridge Shield RP18 OBD Column, 5 μm, 19×150 mm; Mobile Phase, A: water (containing 10 mmol/L NH₄HCO₃) and B: CH₃CN (25% to 45% over 7 min); Flow rate: 20 mL/min; Detector: UV 254 nm). The product fractions were concentrated and lyophilized to afford [2-[3-fluoro-4-(1,3-oxazol-2-yl)benzenesulfonyl]-1H,2H,3H-pyrrolo[3,4-c]pyridin-6-yl][3-(piperazin-1-yl)phenyl]methanol (13.1 mg, 37%). ¹H-NMR (DMSO-d₆, 400 MHz) δ (ppm) 8.35 (d, J=16.8 Hz, 2H), 8.24-8.22 (m, 1H), 7.90-7.83 (m, 2H), 7.52 (s, 1H), 7.47 (s, 1H), 7.06-7.02 (m, 1H), 6.95 (s, 1H), 6.76-6.67 (m, 2H), 5.98 (d, J=4.0 Hz, 1H), 5.60 (d, J=4.0 Hz, 1H), 4.73-4.62 (m, 4H), 2.98-2.81 (m, 4H), 2.80-2.70 (m, 4H), 2.57-2.54 (m, 1H). LCMS (ES, m/z) 536 [M+H]⁺.

### Method C′

### [2-(2,3-dihydro-1,4-benzodioxine-6-sulfonyl)-11H,2H,3H-pyrrolo[3,4-c]pyridin-6-yl][3-(piperazin-1-yl)phenyl]methanol

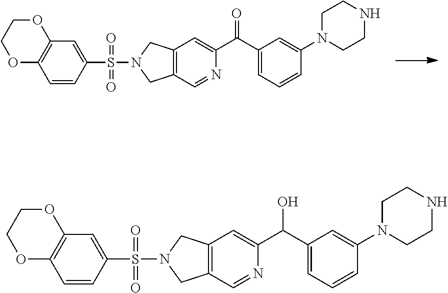 [SMILES: O=C(c1cccc(N2CCNCC2)c1)c1cc2c(cn1)CN(S(=O)(=O)c1ccc3c(c1)OCCO3)C2 ; O=S(=O)(c1ccc2c(c1)OCCO2)N1Cc2cnc(C(O)c3cccc(N4CCNCC4)c3)cc2C1]

To a solution of 1-(3-[[2-(2,3-dihydro-1,4-benzodioxine-6-sulfonyl)-1H,2H,3H-pyrrolo[3,4-c]pyridin-6-yl]carbonyl]phenyl)piperazine (30 mg, 0.05 mmol) in THF (0.5 mL) was added NaBH₄ (2 mg, 0.05 mmol) at 0° C. The resulting solution was stirred for 30 min at 0° C. The reaction mixture was poured into water (3 mL) and then extracted with DCM (3×3 mL). The combined organic layers were dried over anhydrous sodium sulfate, filtered and concentrated under vacuum. The resulting crude product was purified by silica gel chromatography (eluting with 1:10 MeOH/DCM), and further purified by Prep-HPLC (Column: XBridge Shield C18 OBD Column, 5 μm, 19×150 mm; Mobile Phase, A: water (containing 10 mmol/L NH₄HCO₃) and B: CH₃CN (20% to 42% over 7 min); Flow rate: 20 mL/min; Detector: UV 254/220 nm). The product fractions were concentrated and lyophilized to afford [2-(2,3-dihydro-1,4-benzodioxine-6-sulfonyl)-1H,2H,3H-pyrrolo[3,4-c]pyridin-6-yl][3-(piperazin-1-yl)phenyl]methanol as a white solid (3.6 mg, 13%). ¹H-NMR (Methanol-d₄, 400 MHz) δ (ppm): 8.30 (s, 1H), 7.49 (s, 1H), 7.40-7.27 (m, 2H), 7.21-7.10 (m, 1H), 7.08-7.01 (m, 1H), 6.99-6.94 (m, 1H), 6.88-6.79 (m, 2H), 5.71 (s, 1H), 4.59 (s, 4H), 4.36-4.20 (m, 4H), 3.16-3.07 (m, 4H), 3.01-2.91 (m, 4H). LCMS (ES, m/z) 509 [M+H]⁺.

### Method F′

### [2-[4-(5-fluoro-1H-pyrazol-1-yl)benzenesulfonyl]-1H,2H,3H-pyrrolo[3,4-c]pyridine-6-yl](phenyl) methanol

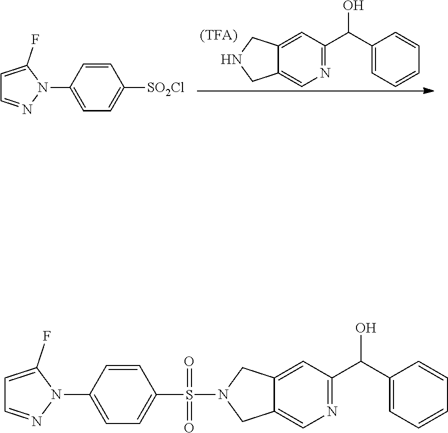 [SMILES: O=S(=O)(Cl)c1ccc(-n2nccc2F)cc1 ; OC(c1ccccc1)c1cc2c(cn1)CNC2 ; O=S(=O)(c1ccc(-n2nccc2F)cc1)N1Cc2cnc(C(O)c3ccccc3)cc2C1]

To a solution of phenyl([1H,2H,3H-pyrrolo[3,4-c]pyridine-6-yl])methanol (TFA salt) (100 mg, 0.29 mmol) in dichloromethane (10 mL) and N,N-dimethylformamide (2 mL) was added potassium carbonate (122 mg, 0.88 mmol). The resulting mixture was stirred for 30 min at room temperature. To this was added 4-(5-fluoro-1H-pyrazol-1-yl)benzene-1-sulfonyl chloride (77 mg, 0.30 mmol). The resulting solution was stirred for 1 h at room temperature. The reaction mixture was poured into water (3 mL) and then extracted with ethyl acetate (3×10 mL). The combined organic layers were dried over anhydrous sodium sulfate, filtered, and concentrated under vacuum. The resulting crude product was purified by prep-TLC (eluting with 99:1 ethyl acetate/petroleum ether), and further purified by prep-HPLC (Column: XBridge Shield RP18 OBD Column, 5 μm, 19×150 mm; Mobile Phase, A: water (containing 10 mmol/L NH₄HCO₃) and B: CH₃CN (30% B to 62% B over 7 min); Flow rate: 20 mL/min; Detector: UV 254 nm). The product fractions were concentrated and lyophilized to afford [2-[4-(5-fluoro-1H-pyrazol-1-yl)benzenesulfonyl]-1H,2H,3H-pyrrolo[3,4-c]pyridine-6-yl](phenyl)methanol (13.6 mg, 10%). ¹H-NMR (DMSO-d₆, 400 MHz) δ (ppm): 8.33 (s, 1H), 8.07-7.98 (m, 2H), 7.88-7.85 (m, 2H), 7.74-7.73 (m, 1H), 7.49 (s, 1H), 7.31 (d, J=6.8 Hz, 2H), 7.24-7.21 (m, 2H), 7.17-7.14 (m, 1H). 6.30-6.29 (m, 1H), 6.08-6.06 (m, 1H), 5.64 (d, J=4.0 Hz, 1H), 4.69-4.57 (m, 4H). LCMS (ES, m/z) 451 [M+H]⁺.

### Method J′

### Phenyl(2-{[1-(1,3-thiazol-2-yl)piperidin-4-yl]sulfonyl}-1H,2H,3H-pyrrolo[3,4-c]pyridine-6-yl)methanol

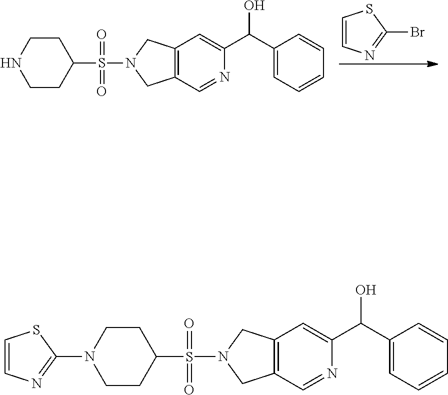

To a solution of phenyl[2-(piperidine-4-sulfonyl)-1H,2H,3H-pyrrolo[3,4-c]pyridin-6-yl]methanol (40 mg, 0.11 mmol) in 1,4-dioxane (1 mL), was added 2-bromo-1,3-thiazole (18 mg, 0.11 mmol), Cs₂CO₃ (105 mg, 0.32 mmol) and RuPhos 3G (10 mg, 0.01 mmol). The resulting mixture was stirred for 16 h at 100° C. and then cooled to room temperature. The reaction mixture was poured into water (2 mL) and then extracted with ethyl acetate (2×5 mL). The combined organic layers were dried over anhydrous sodium sulfate, filtered, and concentrated under vacuum. The resulting crude product was purified by silica gel chromatography (eluting with 0:100 to 10:90 methanol/dichloromethane) and further purified by Prep-HPLC (Column: XBridge Shield C18 OBD Column, 5 μm, 19×150 mm; Mobile Phase, A: water (containing 10 mmol/L NH₄HCO₃) and B: CH₃CN (30% to 65% over 7 min); Flow rate: 20 mL/min; Detector: UV 254/220 nm). The product fractions were concentrated and lyophilized to afford phenyl(2-[[1-(1,3-thiazol-2-yl)piperidin-4-yl]sulfonyl]-1H,2H,3H-pyrrolo[3,4-c]pyridin-6-yl)methanol as a white solid (2.0 mg, 4%). 1H-NMR (CDCl₃, 400 MHz) δ (ppm): 8.51 (s, 1H), 7.47-7.35 (m, 4H), 7.32-7.31 (m, 1H), 7.19 (s, 1H), 7.10 (s, 1H), 6.62-6.60 (s, 1H), 5.79 (s, 1H), 4.98 (s, 1H), 4.87-4.83 (m, 2H), 4.79-4.69 (m, 2H), 4.19-4.16 (m, 2H), 3.29-3.22 (m, 1H), 3.07-3.01 (m, 2H), 2.23-2.20 (m, 2H), 2.06-1.96 (m, 2H). LCMS (ES, m/z) 457 [M+H]+

### Method L′

### (S)-[2-[3-fluoro-4-(1,3-oxazol-2-yl)benzenesulfonyl]-1H,2H,3H-pyrrolo[3,4-c]pyridin-6-yl]([3-[(1-methylazetidin-3-yl)oxy]phenyl])methanol; (R)-[2-[3-fluoro-4-(1,3-oxazol-2-yl)benzenesulfonyl]-1H,2H,3H-pyrrolo[3,4-c]pyridin-6-yl]([3-[(1-methylazetidin-3-yl)oxy]phenyl])methanol

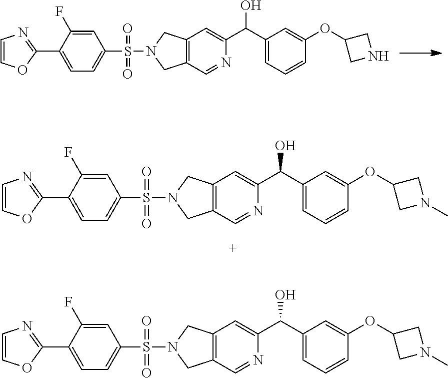 [SMILES: O=S(=O)(c1ccc(-c2ncco2)c(F)c1)N1Cc2cnc(C(O)c3cccc(OC4CNC4)c3)cc2C1 ; CN1CC(Oc2cccc([C@H](O)c3cc4c(cn3)CN(S(=O)(=O)c3ccc(-c5ncco5)c(F)c3)C4)c2)C1 ; CN1CC(Oc2cccc([C@@H](O)c3cc4c(cn3)CN(S(=O)(=O)c3ccc(-c5ncco5)c(F)c3)C4)c2)C1]

To a solution of [3-(azetidin-3-yloxy)phenyl]([2-[3-fluoro-4-(1,3-oxazol-2-yl)benzenesulfonyl]-1H,2H,3H-pyrrolo[3,4-c]pyridin-6-yl])methanol (TFA salt) (80 mg, 0.14 mmol) in MeOH (2 mL) was added formaldehyde (1 mL, 30% in water). The resulting solution was stirred for 30 min at room temperature. This was followed by the addition of STAB (92 mg, 0.41 mmol). The resulting solution was stirred for 12 h at room temperature. The reaction mixture was poured into water (10 mL) and then extracted with ethyl acetate (3×10 mL). The organic layers were combined, dried over anhydrous sodium sulfate, filtered, and concentrated under vacuum. The resulting crude product was purified by Prep-HPLC (Column: XBridge Shield RP18 OBD Column, 5 μm, 30×150 mm; Mobile Phase, A: water (containing 10 mmol/L NH₄HCO₃) and B: CH₃CN (25% to 50% in 7 min); Flow rate: 60 mL/min; Detector: UV 220 nm). The product fractions were concentrated under vacuum. The two enantiomers were further separated by Chiral Prep-HPLC (Column: CHIRALPAK IC, 5 μm, 20×250 mm; Mobile Phase, A: MeOH (containing 0.1% DEA) and B: DCM (keep 40% B in 15 min); Flow rate: 20 mL/min; Detector: UV 254/220 nm; Retention time: 1^{st} eluting isomer, 10.772 min; 2^{nd} eluting isomer, 13.314 min). The product fractions were concentrated and lyophilized to afford 1^{st} eluting isomer as a white solid (12.4 mg, 16%). ¹H-NMR (DMSO-d₆, 400 MHz) δ (ppm): 8.38 (s, 1H), 8.34 (s, 1H), 8.24-8.20 (m, 1H), 7.90-7.83 (m, 2H), 7.53 (s, 1H), 7.47 (s, 1H), 7.14-7.10 (m, 1H), 6.88 (d, J=7.6 Hz, 1H), 6.81 (s, 1H), 6.60-6.57 (m, 1H), 6.10 (d, J=4.0 Hz, 1H), 5.63 (d, J=4.4 Hz, 1H), 4.70-4.63 (m, 5H), 3.71-3.68 (m, 2H), 2.92-2.89 (m, 2H), 2.27 (s, 3H). LCMS (ES, m/z): 537 [M+H]⁺. The product fractions were concentrated and lyophilized to afford 2^{nd} eluting isomer as a white solid (13.3 mg, 18%). ¹H-NMR (DMSO-d₆, 400 MHz) δ (ppm): 8.38 (s, 1H), 8.34 (s, 1H), 8.24-8.20 (m, 1H), 7.90-7.83 (m, 2H), 7.53 (s, 1H), 7.47 (s, 1H), 7.14-7.10 (m, 1H), 6.88 (d, J=7.6 Hz, 1H), 6.81 (s, 1H), 6.60-6.58 (m, 1H), 6.10 (d, J=4.4 Hz, 1H), 5.63 (d, J=4.0 Hz, 1H), 4.69-4.65 (m, 5H), 3.74-3.71 (in 2H), 2.92-2.89 (m, 2H), 2.29 (s, 3H). LCMS (ES, m/z): 537 [M+H]⁺.

TABLE 20

| Cmpd. No. | Purification Methods |
|---|---|
| 7 | Prep-HPLC (Column: XBridge Shield C18 OBD Column, 5 μm, 19 × 150 mm; Mobile Phase, A: |
| | water (containing 10 mmol/L NH4HCO3) and B: CH3CN (20% to 42% over 7 min); Flow rate: |
| | 20 mL/min; Detector: UV 254/220 nm) |
| 35 | Prep-HPLC (Column: XBridge Shield RP18 OBD Column, 5 μm, 19 × 150 mm; Mobile Phase, |
| | A: water (containing 10 mmol/L NH4HCO3) and B: CH3CN (15% to 45% over 7 min) |
| 36 | Prep-HPLC (Column: XBridge Shield RP18 OBD Column, 5 μm, 30 × 150 mm; Mobile Phase, |
| | A: water (containing 10 mmol/L NH4HCO3) and B: CH3CN (25% to 55% over 7 min); Flow |
| | rate: 20 mL/min; Detector: UV 254/220 nm) |
| 38 and 39 | Chiral Prep-HPLC (Column: CHIRALPAK IE, 5 μm, 20 × 250 mm; Mobile Phase, A: MTBE |
| | (containing 0.1% TFA) and B: EtOH; Gradient: keep 5% B over 22 min; Flow rate: 20 mL/min; |
| | Detector: UV 254/220 nm; Retention time: 1^{st}, 15.705 min; 2^{nd}, 19.957 min) |
| 50 and 51 | Prep-HPLC (Column: XBridge Shield RP18 OBD Column, 5 μm, 30 × 150 mm; Mobile Phase, |
| | A: water (containing 10 mmol/L NH4HCO3) and B: CH3CN (25% to 40% over 8 min); Flow |
| | rate: 60 mL/min; Detector: UV 254 nm) |
| 54 | XBridge Shield RP18 OBD Column, 5 μm, 19 × 150 mm; Mobile Phase, A: water (containing 10 |
| | mmol/L NH4HCO3) and B: CH3CN (22% to 37% over 7 min); Flow rate: 20 mL/min; Detector: |
| | UV 254 nm). |
| 59 and 60 | Chiral Prep-HPLC (Column: CHIRALPAK IG, 5 μm, 20 × 250 mm; Mobile Phase, A: MTBE |
| | (0.2% IPA) and B: EtOH (keep 50% B over 25 min); Flow rate: 20 mL/min; Detector: UV |
| | 254/220 nm; Retention time: 1^{st}, 10.086 min; 2^{nd}, 17.541 min) |
| 71 and 72 | Chiral Prep-HPLC (Column: CHIRALPAK IC, 5 μm, 20 × 250 mm; Mobile Phase, A: MeOH |
| | (containing 0.1% DEA) and B: DCM (keep 40% B in 15 min); Flow rate: 20 mL/min; Detector: |
| | UV 254/220 nm; Retention time: 1^{st}, 10.772 min; 2^{nd}, 13.314 min) |
| 84 and 85 | Chiral Prep-HPLC (Column: CHIRALPAK IC, 5 μm, 20 × 250 mm; Mobile Phase, A: methanol |
| | (containing 0.1% DEA) and B: DCM (keep 50% B over 18 min); Flow rate: 20 mL/min; |
| | Detector: UV 254/220 nm; Retention time: 1^{st}, 10.692 min; 2^{nd}, 14.71 min) |
| 96 and 97 | Chiral Prep-HPLC (Column: CHIRALPAK IG, 5 μm, 20 × 250 mm; Mobile Phase, A: |
| | hexane:DCM = 3:1 (containing 0.1% IPA) and B: MeOH; Gradient: keep 40% B over 22 min; Flow rate: |
| | 20 mL/min; Detector: UV 254/220 nm; Retention time: 1^{st}, 15.522 min; 2^{nd}, 19.584 min) |

As set forth in Table 21, IC₅₀ values are defined as follows: ≤25 μM and >10 μM (+); ≤10 μM and >1 μM (++); ≤1 μM and >0.1 μM (+++); ≤0.1 μM and >0.001 μM (++++); based upon the Biochemical Assay of Example A.

In Tables 11 and 21, absolute stereochemistry has not been determined for some Examples. Accordingly, assignment of any Examples as the “R” or “S” stereoisomer is arbitrary, unless otherwise noted. In some cases, Examples are labeled with “1^{st} eluting isomer”, “2^{nd} eluting isomer”, etc. based on the purification method used to separate the stereoisomers (see Table 20).

TABLE 21

| Cmpd. | IC₅₀ | | Synth. | | MS m/z | |
|---|---|---|---|---|---|---|
| No. | (μM) | Intermediates | Method | Chemical Name | [M + H]⁺ | ¹H NMR |
| 7 | ++++ | 3-1, | C′ | [2-(2,3-dihydro-1,4- | 509.0 | (Methanol-d4, 400 MHz) δ |
| | | 4′-1, | | benzodioxine-6-sulfonyl)- | | (ppm): 8.30 (s, 1H), 7.49 (s, |
| | | 10′-1 | | 1H,2H,3H-pyrrolo[3,4- | | 1H), 7.40-7.27 (m, 2H), |
| | | | | c]pyridin-6-yl][3-(piperazin-1- | | 7.21-7.10 (m, 1H), 7.08-7.01 |
| | | | | yl)phenyl]methanol | | (m, 1H), 6.99-6.94 (m, 1H), |
| | | | | | | 6.88-6.79 (m, 2H), 5.71 (s, |
| | | | | | | 1H), 4.59 (s, 4H), 4.36-4.20 |
| | | | | | | (m, 4H), 3.16-3.07 (m, 4H), |
| | | | | | | 3.01-2.91 (m, 4H). |
| 35 | ++++ | 21′-1, | A′ | 2,3-dihydro-1H-isoindol-4- | 475.0 | (DMSO-d6, 400 MHz) δ |
| | | 2′-2, | | yl({2-[4-(1,3-oxazol-2- | | (ppm): 8.32 (d, J = 3.2 Hz, |
| | | 8′-15 | | yl)benzenesulfonyl]- | | 2H), 8.21-8.15 (m, 2H), |
| | | | | 1H,2H,3H-pyrrolo[3,4- | | 8.04-7.97 (m, 2H), 7.48 (d, J = |
| | | | | c]pyridin-6-yl})methanol | | 7.6 Hz, 2H), 7.26-7.03 (m, |
| | | | | | | 3H), 6.14-6.02 (m, 1H), |
| | | | | | | 5.68-5.65 (m, 1H), 4.71-4.56 |
| | | | | | | (m,5H), 4.15-3.95 (m, 3H). |
| 36 | ++++ | 2′-1, | F′ | {6-[3-fluoro-4-(1,3-oxazol-2- | 453.0 | (DMSO-d6, 400 MHz) δ |
| | | 20′-1 | | yl)benzenesulfonyl]- | | (ppm): 8.65 (s, 1H), 8.39 (s, |
| | | | | 5H,6H,7H-pyrrolo[3,4- | | 1H), 8.21 (t, J = 8.0 Hz, 1H), |
| | | | | d]pyrimidin-2- | | 7.96-7.93 (m, 1H), 7.88-7.85 |
| | | | | yl}(phenyl)methanol | | (s, 1H), 7.53 (s, 1H), 7.44- |
| | | | | | | 7.36 (m, 2H), 7.26-7.22 (m, |
| | | | | | | 2H), 7.20-7.15 (m, 1H), |
| | | | | | | 5.98-5.95 (m, 1H), 5.74-5.72 |
| | | | | | | (m, 1H), 4.71-4.66 (m, 4H). |
| 38 | ++++ | 20′-2, | F′ | (S)-{6-[3-fluoro-4-(1,3-oxazol- | 483.0 | (CDCl₃, 400 MHz) δ (ppm): |
| (1^{st} | | 2′-1 | | 2-yl)benzenesulfonyl]- | | 8.63 (br s, 1H), 8.30-8.21 |
| eluting | | | | 5H,6H,7H-pyrrolo[3,4- | | (m, 1H), 7.86 (s, 1H), 7.79- |
| isomer) | | | | d]pyrimidin-2-yl}(3- | | 7.75 (m, 2H), 7.39 (s, 1H), |
| 39 | ++++ | | | methoxyphenyl)methanol; | | 7.29-7.22 (m, 1H), 7.04-7.00 |
| (2^{nd} | | | | (R)-{6-[3-fluoro-4-(1,3-oxazol- | | (m, 2H), 6.81-6.80 (m, 1H), |
| eluting | | | | 2-yl)benzenesulfonyl]- | | 5.96-5.86 (m, 1H), 4.83-4.67 |
| isomer) | | | | 5H,6H,7H-pyrrolo[3,4- | | (m, 4H), 3.79 (s, 3H). |
| | | | | d]pyrimidin-2-yl}(3- | | |
| | | | | methoxyphenyl)methanol | | |
| 50 | ++++ | 27′-1, | F′ | (S)-{3-[(3S)-3,4- | 547.0 | 1^{st}: (CDCl₃, 400 MHz) δ |
| (1^{st} | | 25′-3, | | dimethylpiperazin-1- | | (ppm): 8.57 (s, 1H), 8.28- |
| eluting | | 3′-5, | | yl]phenyl}({6-[4-(1,3-oxazol- | | 8.23 (m, 2H), 8.00 (d, J = 8.0 |
| isomer) | | 4′-11, | | 2-yl)benzenesulfonyl]- | | Hz, 2H), 7.80 (s, 1H), 7.32 |
| | | 33′-1 | | 5H,6H,7H-pyrrolo[3,4- | | (s, 1H), 7.29-7.18 (m, 1H), |
| | | | | d]pyrimidin-2-yl})methanol; | | 7.02 (s, 1H), 6.93 (s, 1H), |
| | | | | (R)-{3-[(3S)-3,4- | | 6.80 (d, J = 8.4 Hz, 1H), 5.81 |
| | | | | dimethylpiperazin-1- | | (s, 1H), 4.77-4.62 (m, 4H), |
| | | | | yl]phenyl}({6-[4-(1,3-oxazol- | | 4.53 (s, 1H), 3.56-3.47 (m, |
| | | | | 2-yl)benzenesulfonyl]- | | 2H), 3.02 (s, 2H), 2.42 (s, |
| | | | | 5H,6H,7H-pyrrolo[3,4- | | 6H), 1.21 (s, 3H). |
| 51 | ++++ | | | d]pyrimidin-2-yl})methanol; | | 2^{nd}: (CDCl₃, 400 MHz) δ |
| (2^{nd} | | | | (S)-{3-[(3R)-3,4- | | (ppm): 8.58 (s, 1H), 8.25- |
| eluting | | | | dimethylpiperazin-1- | | 8.23 (m, 2H), 8.00 (d, J = 8.0 |
| isomer) | | | | yl]phenyl}({6-[4-(1,3-oxazol- | | Hz, 2H), 7.80 (s, 1H), 7.32 |
| | | | | 2-yl)benzenesulfonyl]- | | (s, 1H), 7.28-7.19 (m, 1H), |
| | | | | 5H,6H,7H-pyrrolo[3,4- | | 7.04 (s, 1H), 6.96 (s, 1H), |
| | | | | d]pyrimidin-2-yl})methanol; | | 6.80 (d, J = 8.4 Hz, 1H), 5.82 |
| | | | | (R)-{3-[(3R)-3,4- | | (s, 1H), 4.73-4.62 (m, 4H), |
| | | | | dimethylpiperazin-1- | | 4.55 (s, 1H), 3.55-3.52 (m, |
| | | | | yl]phenyl}({6-[4-(1,3-oxazol- | | 2H), 3.02 (s, 2H), 2.42 (s, |
| | | | | 2-yl)benzenesulfonyl]- | | 6H), 1.21 (s, 3H). |
| | | | | 5H,6H,7H-pyrrolo[3,4- | | |
| | | | | d]pyrimidin-2-yl})methanol | | |
| 54 | ++++ | | | 2-[3-(4-methylpiperazin-1- | 541.0 | (DMSO-d6, 400 MHz) δ |
| | | | | yl)phenyl]-2-{2-[4-(1,3- | | (ppm): 8.47 (s, 1H), 8.32 (s, |
| | | | | oxazol-2-yl)benzenesulfonyl]- | | 1H), 8.15 (d, J = 8.4 Hz, |
| | | | | 1H,2H,3H-pyrrolo[3,4- | | 2H), 8.00 (d, J = 8.4 Hz, |
| | | | | c]pyridin-6-yl}acetonitrile | | 2H), 7.47 (s, 1H), 7.35 (s, |
| | | | | | | 1H), 7.17-7.15 (m, 1H), 6.97 |
| | | | | | | (s, 1H), 6.85-6.83 (m, 1H), |
| | | | | | | 6.72-6.70 (m, 1H), 5.75 (s, |
| | | | | | | 1H), 4.66 (s, 4H), 3.10-3.08 |
| | | | | | | (m, 4H), 2.43-2.40 (m, 4H), |
| | | | | | | 2.21 (s, 3H). |
| 59 | ++++ | 2′-1, | A′ | (S)-{6-[3-fluoro-4-(1,3-oxazol- | 524.0 | (DMSO-d6, 400 MHz) δ |
| (1^{st} | | 27′-1, | | 2-yl)benzenesulfonyl]- | | (ppm): 8.63 (s, 1H), 8.39 (s, |
| eluting | | 33′-2, | | 5H,6H,7H-pyrrolo[3,4- | | 1H), 8.24-8.21 (m, 1H), |
| isomer) | | 32′-2 | | d]pyrimidin-2-yl}(2,3,4,5- | | 7.98-7.95 (m, 1H), 7.89-7.87 |
| 60 | +++ | | | tetrahydro-1,4-benzoxazepin-9- | | (m, 1H), 7.53 (s, 1H), 7.39- |
| (2^{nd} | | | | yl)methanol; | | 7.36 (m, 1H), 7.06-7.04 (m, |
| eluting | | | | (R)-{6-[3-fluoro-4-(1,3-oxazol- | | 1H), 6.96-6.92 (m, 1H), 5.98 |
| isomer) | | | | 2-yl)benzenesulfonyl]- | | (d, J = 5.6 Hz, 1H), 5.85 (d, J = |
| | | | | 5H,6H,7H-pyrrolo[3,4- | | 6.0 Hz, 1H), 4.72-4.63 (m, |
| | | | | d]pyrimidin-2-yl}(2,3,4,5- | | 4H), 3.71 (s, 2H), 3.52-3.45 |
| | | | | tetrahydro-1,4-benzoxazepin-9- | | (m, 1H), 3.34-3.25 (m, 2H), |
| | | | | yl)methanol | | 2.90-2.81 (m, 2H). |
| 71 | ++++ | 2′-1, | L′ | (S)-{2-[3-fluoro-4-(1,3-oxazol- | 537.0 | (DMSO-d6, 400 MHz) δ |
| (1^{st} | | 4′-15, | | 2-yl)benzenesulfonyl]- | | (ppm): 8.38 (s, 1H), 8.34 (s, |
| eluting | | 37′-1, | | 1H,2H,3H-pyrrolo[3,4- | | 1H), 8.24-8.20 (m, 1H), |
| isomer) | | 8′-22 | | c]pyridin-6-yl}({3-[(1- | | 7.90-7.83 (m, 2H), 7.53 (s, |
| 72 | ++++ | | | methylazetidin-3- | | 1H), 7.47 (s, 1H), 7.14-7.10 |
| (2^{nd} | | | | yl)oxy]phenyl})methanol; | | (m, 1H), 6.88 (d, J = 7.6 Hz, |
| eluting | | | | (R)-{2-[3-fluoro-4-(1,3-oxazol- | | 1H), 6.81 (s, 1H), 6.60-6.57 |
| isomer) | | | | 2-yl)benzenesulfonyl]- | | (m, 1H), 6.10 (d, J = 4.0 Hz, |
| 83 | +++ | | | 1H,2H,3H-pyrrolo[3,4- | | 1H), 5.63 (d, J = 4.4 Hz, 1H), |
| (2^{nd} | | | | c]pyridin-6-yl}({3-[(1- | | 4.70-4.63 (m, 5H), 3.71-3.68 |
| eluting | | | | methylazetidin-3- | | (m, 2H), 2.92-2.89 (m, 2H), |
| isomer) | | | | yl)oxy]phenyl})methanol | | 2.27 (s, 3H). |
| 84 | ++++ | 8′-11 | J′ | (S)-(3-{6-methyl-2,6- | 544.0 | (DMSO-d6, 400 MHz) δ |
| (1^{st} | | | | diazaspiro[3.3]heptan-2- | | (ppm): 8.32 (s, 2H), 8.16 (d, |
| eluting | | | | yl}phenyl)({2-[4-(1,3-oxazol- | | J = 8.8 Hz, 2H), 8.01 (d, J = |
| isomer) | | | | 2-yl)benzenesulfonyl]- | | 8.4 Hz, 2H), 7.47-7.44 (m, |
| 85 | ++++ | | | 1H,2H,3H-pyrrolo[3,4- | | 2H), 7.00-6.96 (m, 1H), 6.58 |
| (2^{nd} | | | | c]pyridin-6-yl})methanol; | | (d, J = 8.0 Hz, 1H), 6.44 (s, |
| eluting | | | | (R)-(3-{6-methyl-2,6- | | 1H), 6.20 (d, J = 8.4 Hz, |
| isomer) | | | | diazaspiro[3.3]heptan-2- | | 1H), 5.96 (d, J = 4.0 Hz, |
| | | | | yl}phenyl)({2-[4-(1,3-oxazol- | | 1H), 5.56 (d, J = 3.6 Hz, |
| | | | | 2-yl)benzenesulfonyl]- | | 1H), 4.64-4.58 (m, 4H), 3.78 |
| | | | | 1H,2H,3H-pyrrolo[3,4- | | (s, 4H), 3.33-3.27 (m, 4H), |
| | | | | c]pyridin-6-yl})methanol | | 2.20 (s, 3H). |
| 96 | ++++ | 4′-13, | B′ | (S)-(3-{3,6- | 530.0 | (CD₃OD, 400 MHz) δ |
| (1^{st} | | 8′-11 | | diazabicyclo[3.1.1]heptan-3- | | (ppm): 8.31 (s, 1H), 8.21 (d, |
| eluting | | | | yl}phenyl)({2-[4-(1,3-oxazol- | | J = 8.8 Hz, 2H), 8.07-8.03 |
| isomer) | | | | 2-yl)benzenesulfonyl]- | | (m, 3H), 7.53 (s, 1H), 7.38 |
| 97 | ++++ | | | 1H,2H,3H-pyrrolo[3,4- | | (s, 1H), 7.16-7.12 (m, 1H), |
| (2^{nd} | | | | c]pyridin-6-yl})methanol; | | 6.84 (s, 1H), 6.70-6.63 (m, |
| eluting | | | | (R)-(3-{3,6- | | 2H), 5.74 (s, 1H), 4.72 (s, |
| isomer) | | | | diazabicyclo[3.1.1]heptan-3- | | 4H), 4.02-3.99 (m, 2H), |
| | | | | yl}phenyl)({2-[4-(1,3-oxazol- | | 3.65-3.55 (m, 4H), 2.82-2.80 |
| | | | | 2-yl)benzenesulfonyl]- | | (m, 1H), 1.75-1.73 (m, 1H). |
| | | | | 1H,2H,3H-pyrrolo[3,4- | | |
| | | | | c]pyridin-6-yl})methanol | | |
| 108 | ++++ | | | (S)-(3-(6-methyl-2,6- | | |
| (1^{st} | | | | diazaspiro[3.3]heptan-2- | | |
| eluting | | | | yl)phenyl)(2-(naphthalen-2- | | |
| isomer) | | | | ylsulfonyl)-2,3-dihydro-1H- | | |
| 109 | ++++ | | | pyrrolo[3,4-c]pyridin-6- | | |
| (2^{nd} | | | | yl)methanol; | | |
| eluting | | | | (R)-(3-(6-methyl-2,6- | | |
| isomer) | | | | diazaspiro[3.3]heptan-2- | | |
| | | | | yl)phenyl)(2-(naphthalen-2- | | |
| | | | | ylsulfonyl)-2,3-dihydro-1H- | | |
| | | | | pyrrolo[3,4-c]pyridin-6- | | |
| | | | | yl)methanol | | |

### Example A. Biochemical Assay for USP9X Inhibitory Activity

The assay was performed in a final volume of 6 μL assay buffer containing 20 mM Tris-HCl (pH 8.0, (1M Tris-HCl, pH 8.0 solution; Corning 46-031-CM)), L-Glutathione (GSH) reducing agent (1 mM, Sigma-Aldrich, G4251-100G), 0.03% Bovine Gamma Globulin (BGG) (0.22 μM filtered, Sigma, G7516-25G), and 0.01% Triton X-100 (Sigma, T9284-10L). DMSO solutions of the compounds in nanoliter quantities (10-point, 3-fold serial dilutions) were dispensed into 1536 assay plates (Corning, #3724BC) for final test concentrations of 25 μM to 1.3 nM, top to lowest dose, respectively. Concentration and incubation times were optimized for the maximal signal-to-background while maintaining initial velocity conditions at a fixed substrate concentration (<<Kₘ). The final concentration of USP9X (Enzyme, E) was 0.025 nM, and the final concentration of Ubiquitin-Rhodamine 110 (Ub-Rh110, UbiQ-126) (Substrate, S) was 25 nM. To assay plates (pre-stamped with compound) was added 3 μL 2x Enzyme. The enzyme was preincubated for 30 minutes and then treated with 3 μL of 2x Substrate. Plates were incubated for 11 min (continuous kinetic read) at room temperature before the fluorescence was read on the Envision plate reader (Perkin Elmer) or PheraSTAR plate reader (BMG), with excitation at 485 nm and emission at 535 nm. The slope (best fit linear regression) of the five reads was used to normalize for inhibition. For all assays, data are reported as percent inhibition compared with control wells based on the following equation: % inh=100*((FLU−AveLow)/(AveHigh−AveLow)), wherein FLU is measured Fluorescence, AveLow is average Fluorescence of no enzyme control (n=64), and AveHigh is average Fluorescence of DMSO control (n=64). IC₅₀ values are determined by curve fitting of the standard 4 parameter logistic fitting algorithm included in the Activity Base software package: IDBS XE Designer Model205. Data are fitted using the Levenburg Marquardt algorithm.

### EQUIVALENTS

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description and drawings are by way of example to illustrate the discoveries provided herein.

1. A method of treating cancer in a patient in need thereof, comprising administering to the patient a USP9X Inhibitor.

2. A method of treating cancer in a patient in need thereof, comprising administering to the patient an antineoplastic therapy consisting of the administration of a USP9X Inhibitor.

3. A method of treating cancer in a patient in need thereof, comprising administering to the patient a USP9X Inhibitor, wherein the patient is receiving or has received an immune checkpoint pathway inhibitor.

4. A method of treating cancer in a patient in need thereof, comprising administering to the patient an immune checkpoint pathway inhibitor, wherein the patient is receiving or has received a USP9X Inhibitor.

5. A method of treating cancer in a patient in need thereof, comprising administering to the patient an antineoplastic therapy consisting of the administration of a USP9X Inhibitor and the administration of an immune checkpoint pathway inhibitor.

6. A method of treating a patient diagnosed with a cancer, comprising administering a USP9X Inhibitor to the patient, wherein the patient is already being treated for the cancer with an immune checkpoint pathway inhibitor.

7. A method of treating a patient diagnosed with a cancer, comprising administering a USP9X Inhibitor to the patient, wherein the cancer has progressed while receiving an immune checkpoint pathway inhibitor.

8. A method of treating a patient diagnosed with a cancer, wherein the patient i) has been diagnosed with cancer that has progressed, or ii) has relapsed after previously being administered an immune checkpoint pathway inhibitor for the cancer.

9. A method of treating a patient diagnosed with a cancer, wherein the method comprises administering a USP9X Inhibitor to the patient while the patient continues to receive an immune checkpoint pathway inhibitor after being diagnosed with a cancer that is refractory to an immune checkpoint pathway inhibitor.

10. The method of any one of the preceding embodiments, wherein the cancer comprises a tumor that expresses PD-L1.

11. The method of any one of the preceding embodiments, wherein the cancer comprises a tumor that expresses PD-L1 and the PD-L1 can be detected using PD-L1 IHC 22C3 pharmDx.

12. The method of any one of embodiments 1-9, wherein the cancer comprises a tumor that expresses CTLA-4.

13. The method of any one of the preceding embodiments, wherein the cancer is selected from unresectable or metastatic melanoma, cutaneous melanoma, advanced renal cell carcinoma, microsatellite instability-high (MSI-H) or mismatch repair deficient (dMMR) metastatic colorectal cancer, metastatic squamous non-small cell lung cancer, head and neck squamous cell cancer, classical Hodgkin lymphoma, primary mediastinal large B-cell lymphoma, urothelial carcinoma, microsatellite instability-high cancer, gastric cancer, cervical cancer, hepatocellular carcinoma, or Merkel cell carcinoma.

14. The method of any one of the preceding embodiments, wherein the patient has received one or more prior lines of chemotherapy.

15. The method of any one of the preceding embodiments, wherein the patient has received two or more prior lines of chemotherapy.

16. The method of any one of the preceding embodiments, wherein the patient has received three or more prior lines of chemotherapy.

17. The method of any one of the preceding embodiments, wherein the patient has not responded to a prior line of chemotherapy.

18. The method of any one of the preceding embodiments, wherein the patient has relapsed after receiving a prior line of chemotherapy.

19. The method of any one of embodiments 14-18, wherein the prior line of chemotherapy is selected from platinum-based chemotherapy, fluoropyrimidine therapy, irinotecan therapy, paclitaxel therapy, nab-paclitaxel therapy, HER2/neu-targeted therapy, or sorafenib therapy.

20. The method of any one of the preceding embodiments, wherein the patient has not responded to prior therapy with an immune checkpoint pathway inhibitor.

21. The method of any one of the preceding embodiments, wherein the cancer is refractory or resistant to treatment with an immune checkpoint pathway inhibitor.

22. The method of any one of the preceding embodiments, wherein the cancer is unresectable or metastatic melanoma.

23. The method of any one of the preceding embodiments, wherein the cancer is cutaneous melanoma with pathologic involvement of regional lymph nodes.

24. The method of claim 23, wherein the patient has undergone complete resection and/or a total lymphadenectomy.

25. The method of any one of the preceding embodiments, wherein the cancer is previously untreated advanced renal cell carcinoma.

26. The method of any one of the preceding embodiments, wherein the cancer is microsatellite instability-high (MSI-H) or mismatch repair deficient (dMMR) metastatic colorectal cancer.

27. The method of claim 26, wherein the cancer has progressed following treatment with a fluoropyrimidine, oxaliplatin, and irinotecan.

28. The method of any one of the preceding embodiments, wherein the cancer is unresectable or metastatic melanoma that has progressed following treatment with ipilimumab.

29. The method of any one of the preceding embodiments, wherein the cancer is metastatic squamous non-small cell lung cancer.

30. The method of claim 29, wherein the cancer has progressed on or after platinum-based chemotherapy.

31. The method of any one of the preceding embodiments, wherein the cancer is melanoma with involvement of lymph node(s) following complete resection.

32. The method of any one of the preceding embodiments, wherein the cancer is metastatic nonsquamous non-small cell lung cancer with no EGFR or ALK genomic tumor aberrations.

33. The method of any one of the preceding embodiments, wherein the cancer is metastatic non-small cell lung cancer, wherein the cancer comprises a tumor with high PD-L1 expression with no EGFR or ALK genomic tumor aberrations.

34. The method of claim 33, wherein high PD-L1 expression is a Tumor Proportion Score (TPS)≥50% as determined by an FDA-approved test.

35. The method of any one of the preceding embodiments, wherein the cancer is metastatic non-small cell lung cancer, wherein the cancer comprises a tumor with PD-L1 expression of TPS≥1% as determined by an FDA-approved test, with disease progression on or after platinum-containing chemotherapy.

36. The method of any one of the preceding embodiments, wherein the cancer is recurrent or metastatic head and neck squamous cell cancer (HNSCC) with disease progression on or after platinum-containing chemotherapy.

37. The method of any one of the preceding embodiments, wherein the cancer is refractory classical Hodgkin lymphoma.

38. The method of any one of the preceding embodiments, wherein the cancer is refractory primary mediastinal large B-cell lymphoma.

39. The method of any one of the preceding embodiments, wherein the cancer is locally advanced or metastatic urothelial carcinoma, wherein the cancer comprises a tumor with PD-L1 expression of Combined Positive Score (CPS)≥10 as determined by an FDA-approved test.

40. The method of any one of the preceding embodiments, wherein the cancer is locally advanced or metastatic urothelial carcinoma, and wherein the patient is not eligible for platinum-containing chemotherapy.

41. The method of any one of the preceding embodiments, wherein the cancer is locally advanced or metastatic urothelial carcinoma.

42. The method of any one of the preceding embodiments, wherein the cancer is unresectable or metastatic, microsatellite instability-high (MSI-H) or mismatch repair deficient solid tumors that have progressed following prior treatment.

43. The method of any one of the preceding embodiments, wherein the cancer is locally advanced or metastatic gastric or gastroesophageal junction adenocarcinoma, wherein the cancer comprises a tumor with PD-L1 expression of Combined Positive Score (CPS)≥1 as determined by an FDA-approved test.

44. The method of claim 43, wherein the cancer has progressed on or after prior lines of therapy.

45. The method of any one of the preceding embodiments, wherein the cancer is recurrent or metastatic cervical cancer with disease progression on or after chemotherapy whose tumors express PD-L1 (CPS≥1) as determined by an FDA-approved test.

46. The method of any one of the preceding embodiments, wherein the cancer is hepatocellular carcinoma (HCC), and wherein the patient has previously been treated with sorafenib.

47. The method of any one of the preceding embodiments, wherein the cancer is recurrent locally advanced or metastatic Merkel cell carcinoma.

48. The method of any one of the preceding embodiments, wherein the immune checkpoint pathway inhibitor is selected from ipilimumab, nivolumab, or pembrolizumab.

49. The method of any one of the preceding embodiments, comprising administering two or more immune checkpoint pathway inhibitors.

50. The method of any one of the preceding embodiments, wherein the immune checkpoint pathway inhibitor is ipilimumab.

51. The method of any one of the preceding embodiments, wherein the patient is receiving or has received ipilimumab in a dose of any one of the following:
(i) 3 mg/kg over 90 minutes every 3 weeks for a total of 4 doses; and/or
(ii) 10 mg/kg over 90 minutes every 3 weeks for a total of 4 doses; and/or
(iii) 10 mg/kg every 12 weeks for up to 3 years; and/or
(iv) 1 mg/kg over 30 minutes every 3 weeks for a total of 4 doses.

52. The method of any one of the preceding embodiments, comprising administering to the patient ipilimumab in a dose of any one of the following:
(i) 3 mg/kg over 90 minutes every 3 weeks for a total of 4 doses; and/or
(ii) 10 mg/kg over 90 minutes every 3 weeks for a total of 4 doses; and/or
(iii) 10 mg/kg every 12 weeks for up to 3 years; and/or
(iv) 1 mg/kg over 30 minutes every 3 weeks for a total of 4 doses.

53. The method of any one of the preceding embodiments, wherein the immune checkpoint pathway inhibitor is nivolumab.

54. The method of any one of the preceding embodiments, wherein the patient is receiving or has received nivolumab in a dose of any one of the following:
(i) 3 mg/kg over 60 minutes every 2 weeks; and/or
(ii) 3 mg/kg over 30 minutes.

55. The method of any one of the preceding embodiments, comprising administering to the patient nivolumab in a dose of any one of the following:
(i) 3 mg/kg over 60 minutes every 2 weeks; and/or
(ii) 3 mg/kg over 30 minutes.

56. The method of any one of the preceding embodiments, wherein the immune checkpoint pathway inhibitor is ipilimumab and nivolumab.

57. The method of any one of the preceding embodiments, wherein the patient is receiving or has received the immune checkpoint pathway inhibitor in a dose of any one of the following:
(i) 3 mg/kg nivolumab over 30 minutes followed 1 mg/kg ipilimumab over 30 minutes on the same day, every 3 weeks for a total of 4 doses; and/or
(ii) 3 mg/kg nivolumab over 30 minutes, followed by 1 mg/kg ipilimumab over 30 minutes on the same day, every 3 weeks for a total of 4 doses, followed by 240 mg nivolumab every 2 weeks over 30 minutes; and/or
(iii) 3 mg/kg nivolumab over 30 minutes, followed by 1 mg/kg ipilimumab over 30 minutes on the same day, every 3 weeks for a total of 4 doses, followed by 480 mg nivolumab every 2 weeks over 30 minutes.

58. The method of any one of the preceding embodiments, comprising administering to the patient the immune checkpoint pathway inhibitor in a dose of any one of the following:
(i) 3 mg/kg nivolumab over 30 minutes followed 1 mg/kg ipilimumab over 30 minutes on the same day, every 3 weeks for a total of 4 doses; and/or
(ii) 3 mg/kg nivolumab over 30 minutes, followed by 1 mg/kg ipilimumab over 30 minutes on the same day, every 3 weeks for a total of 4 doses, followed by 240 mg nivolumab every 2 weeks over 30 minutes; and/or
(iii) 3 mg/kg nivolumab over 30 minutes, followed by 1 mg/kg ipilimumab over 30 minutes on the same day, every 3 weeks for a total of 4 doses, followed by 480 mg nivolumab every 2 weeks over 30 minutes.

59. The method of any one of the preceding embodiments, wherein the immune checkpoint pathway inhibitor is pembrolizumab.

60. The method of any one of the preceding embodiments, wherein the patient is receiving or has received pembrolizumab in a dose of any one of the following:
(i) 200 mg every 3 weeks; and/or
(ii) 2 mg/kg over 30 minutes every 3 weeks.

61. The method of any one of the preceding embodiments, comprising administering to the patient pembrolizumab in a dose of any one of the following:
(i) 200 mg every 3 weeks; and/or
(ii) 2 mg/kg over 30 minutes every 3 weeks.

62. The method of any one of the preceding embodiments, wherein the USP9X Inhibitor has an IC₅₀ value of ≤10 μM in the Biochemical Assay of Example A.

63. The method of any one of the preceding embodiments, wherein the USP9X Inhibitor has an IC₅₀ value of ≤2 μM in the Biochemical Assay of Example A.

64. The method of any one of the preceding embodiments, wherein the USP9X Inhibitor has an IC₅₀ value of ≤1 μM in the Biochemical Assay of Example A.

65. The method of any one of the preceding embodiments, wherein the USP9X Inhibitor has an IC₅₀ value of ≤0.2 μM in the Biochemical Assay of Example A.

66. The method of any one of the preceding embodiments, wherein the USP9X Inhibitor has an IC₅₀ value of ≤0.1 μM in the Biochemical Assay of Example A.

67. The method of any one of the preceding embodiments, wherein the USP9X Inhibitor has an IC₅₀ value of ≤0.05 μM in the Biochemical Assay of Example A.

68. The method of any one of the preceding embodiments, wherein the USP9X Inhibitor is a compound of Formula I:

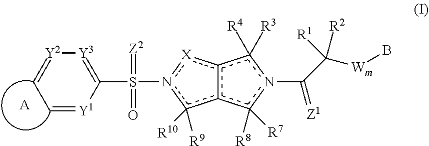

or a pharmaceutically acceptable salt thereof, wherein:
X is CR⁵R⁶, CR⁵, NR⁵, or N, as valency permits;
dashed bonds are each independently a single or a double bond, as valency permits;
Y¹, Y², and Y³ are each independently N or CR^{a};
each R^{a} is independently —H, halogen, or —CN;
Ring A is a 5- to 6-membered aryl, 5- to 6-membered heteroaryl containing 1-4 heteroatoms independently selected from the group consisting of O, N, and S, 5- to 7-membered heterocyclyl containing 1-4 heteroatoms independently selected from the group consisting of O, N, and S, or 5- to 7-membered cycloalkyl,
   wherein each aryl, heteroaryl, heterocyclyl, or cycloalkyl is optionally substituted with one or more halogen, —C₁-C₆alkyl, —C₂-C₆alkenyl, —C₂-C₆alkynyl, oxo, or —C(O)R′;
Z¹ is O, S, or NR;
Z² is O or NR;
W is CR^{1′}R^{2′}, O, S, or NR;
m is 0 or 1;
R¹ and R² are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each cycloalkyl, cycloalkenyl, heterocyclyl, aryl, or heteroaryl is optionally substituted with one or more R^{e},
   wherein each alkyl, alkenyl, or alkynyl is optionally substituted with one or more halogen,
   wherein each heterocyclyl is 3- to 14-membered and contains 1-4 heteroatoms independently selected from the group consisting of O, N, and S, and wherein the heterocyclyl does not contain an O in the γ-position relative to C(═Z¹), and
   wherein each heteroaryl is 5- to 14-membered and contains 1-4 heteroatoms independently selected from the group consisting of O, N, and S;
or R¹ and R² combine with the carbon to which they are attached to form oxo, a C₃-C₈cycloalkyl, or a 3- to 8-membered heterocyclyl containing 1-4 heteroatoms independently selected from the group consisting of O, N, and S, and wherein the heterocyclyl does not contain an O in the γ-position relative to C(═Z¹), and
   wherein each cycloalkyl or heterocyclyl is optionally substituted with one or more R^{e}.
R^{1′} and R^{2′} are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —(CR^{b}R^{c})ₙNR², —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
   wherein each alkyl, alkenyl, alkynyl, cycloalkyl, cycloalkenyl, heterocyclyl, aryl, or heteroaryl is optionally substituted with one or more R^{e},
   wherein each heterocyclyl is 3- to 14-membered and contains 1-4 heteroatoms independently selected from the group consisting of O, N, and S, and
   wherein each heteroaryl is 5- to 14-membered and contains 1-4 heteroatoms independently selected from the group consisting of O, N, and S;
or R^{1′} and R^{2′} combine with the carbon to which they are attached to form oxo, a C₃-C₈cycloalkyl, or a 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e};
or R¹ and R^{1′} combine with the carbons to which they are attached to form a C₃-C₈cycloalkyl or 3- to 8-membered heterocyclyl containing 1-4 heteroatoms independently selected from the group consisting of O, N and S,
   wherein each heterocyclyl does not contain an O in the γ-position relative to C(═Z¹), and
   wherein each cycloalkyl or heterocyclyl is optionally substituted with one or more R^{e};
R^{b} and R^{c} are each independently selected from the group consisting of —H, halogen, and —C₁-C₆alkyl;
each n is independently 0, 1, 2, 3, or 4;
each R^{e} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S, and wherein —OR of R^{e} does not result in an O in the γ-position relative to C(═Z¹),
   wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
B is a monocyclic or bicyclic 3- to 14-membered ring,
   wherein the ring is saturated, fully or partially unsaturated, or aromatic, and
   wherein the ring contains 0-4 heteroatoms independently selected from the group consisting of O, N, and S,
   wherein the ring is optionally substituted with one or more R^{d}, and
   when m is 0 and the ring is saturated or partially unsaturated, then the ring does not contain an O in the γ-position relative to C(═Z¹);
each R^{d} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
each R³, R⁴, R⁵, R⁶, R⁷, R⁸, R⁹, and R¹⁰ is independently —H, —C₁-C₆alkyl, —C₃-C₈cycloalkyl, or 3- to 8-membered heterocyclyl containing 1-4 heteroatoms independently selected from the group consisting of O, N, and S,
   wherein each alkyl, cycloalkyl, or heterocyclyl is optionally substituted with one or more halogen, oxo, —C₁-C₆alkyl, —C₂-C₆alkenyl, —C₂-C₆alkynyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₃-C₈cycloalkyl, or 3- to 8-membered heterocyclyl containing 1-4 heteroatoms independently selected from the group consisting of O, N, and S, and
   wherein R³, R⁷, and R⁹ are each independently present or absent, as valency permits;
or R³ and R⁴, R⁵ and R⁶, R⁷ and R⁸, R⁹ and R¹⁰, or combinations thereof, combine with the carbon to which they are attached to form an oxo, C₃-C₈cycloalkyl, or 3- to 8-membered heterocyclyl containing 1-4 heteroatoms independently selected from the group consisting of O, N, and S;
each R is independently selected from the group consisting of —H, —OH, —O(C₁-C₆alkyl), —NH₂, —NH(C₁-C₆alkyl), —N(C₁-C₆alkyl)₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —O—C₁-C₆alkyl, —NH(C₁-C₆alkyl), —N(C₁-C₆alkyl)₂, —C₁-C₆alkyl optionally substituted with one or more oxo or —OH, —C₂-C₆alkenyl, —C₂-C₆alkynyl, —C₃-C₁₂cycloalkyl, or 3- to 8-membered heterocyclyl containing 1-4 heteroatoms independently selected from the group consisting of O, N, and S; and
each R′ is independently selected from the group consisting of —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, aryl, and 5- to 14-membered heteroaryl containing 1-4 heteroatoms independently selected from the group consisting of O, N, and S,
   wherein each alkyl, alkenyl, alkynyl, cycloalkyl, cycloalkenyl, heterocyclyl, aryl, or heteroaryl is optionally substituted with one or more halogen, oxo, —C₁-C₆alkyl optionally substituted with one or more oxo or —OH, —C₂-C₆alkenyl, —C₂-C₆alkynyl, —O—C₁-C₆alkyl, —NH(C₁-C₆alkyl), or —N(C₁-C₆alkyl)₂.

69. The method of any one of the preceding embodiments, wherein the USP9X Inhibitor is a compound of Formula I-a:

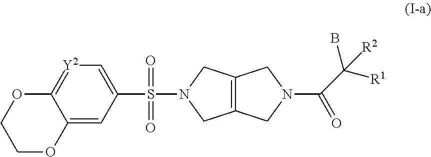

or a pharmaceutically acceptable salt thereof
wherein Y² is CH or N.

70. The method of any one of the preceding embodiments, wherein the USP9X Inhibitor is a compound of Formula I-b:

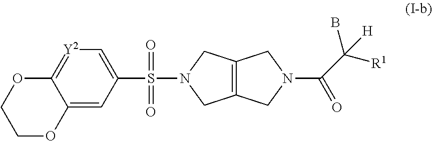

or a pharmaceutically acceptable salt thereof, wherein:
Y² is CH or N;
R¹ is —OH or —(CH₂)NHMe;
B is a phenyl ring or a bicyclic ring,
   wherein at least one of the rings in the bicyclic ring is a phenyl ring,
   wherein the phenyl ring or bicyclic ring contains 0-4 heteroatoms independently selected from the group consisting of O, N, and S, and
   wherein the phenyl ring or bicyclic ring is optionally substituted with one or more R^{d};
each R^{d} is independently selected from the group consisting of halogen, —C₁-C₆alkyl, and —OR; and
each R is independently —H, —C₁-C₆alkyl, or 3- to 8-membered heterocyclyl optionally substituted with —C₁-C₆alkyl.

71. The method of any one of the preceding embodiments, wherein the USP9X Inhibitor is a compound Formula I-c:

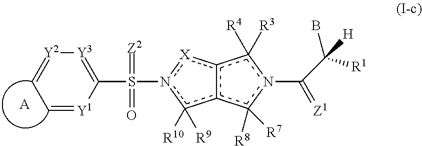

or a pharmaceutically acceptable salt thereof.

72. The method of any one of the preceding embodiments, wherein the USP9X Inhibitor is a compound of Formula I-d:

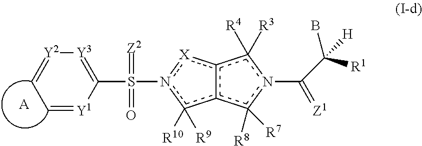

or a pharmaceutically acceptable salt thereof.

73. The method of any one of the preceding embodiments, wherein the USP9X Inhibitor is a compound of Formula I-e:

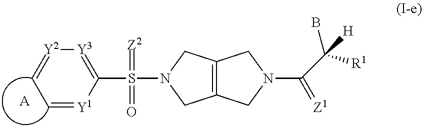

or a pharmaceutically acceptable salt thereof.

74. The method of any one of the preceding embodiments, wherein the USP9X Inhibitor is a compound of Formula I-f:

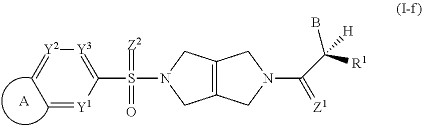

or a pharmaceutically acceptable salt thereof.

75. The method of any one of the preceding embodiments, wherein the USP9X Inhibitor is a compound of Formula II:

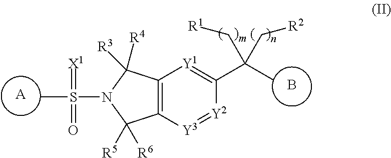

or a pharmaceutically acceptable salt thereof, wherein:
X¹ is NR or O;
Y¹ is CR⁷ or N;
Y² is CR⁸ or N;
Y³ is CR⁹ or N;
   wherein the heteroaryl formed when at least one of Y¹, Y², or Y³ is N may comprise an N-oxide;
Ring A is a monocyclic or bicyclic 3- to 12-membered ring,
   wherein the ring is saturated, fully or partially unsaturated, or aromatic, and
   wherein the ring contains 0-4 heteroatoms independently selected from the group consisting of N, O, and S, and
   wherein Ring A is optionally substituted with one or more R^{a};
each R^{a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R^{a} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
Ring B is a monocyclic or bicyclic 3- to 12-membered ring,
   wherein the ring is saturated, fully or partially unsaturated, or aromatic, and
   wherein the ring contains 0-4 heteroatoms independently selected from the group consisting of N, O, and S, and
   wherein Ring B is optionally substituted with one or more R^{b};
each R^{b} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, optionally substituted C₁-C₆ aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R^{b} group may be substituted with one or more substituents selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R¹ and R² are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
or R¹ and R² combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
   wherein an optionally substituted R¹ and R² group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
R³, R⁴, R⁵, and R⁶ are each independently selected from the group consisting of —H, optionally substituted C₁-C₆aliphatic, optionally substituted C₃-C₈cycloalkyl, and optionally substituted 3- to 8-membered heterocyclyl containing 1-4 heteroatoms independently selected from the group consisting of N, O, and S,
or R³ and R⁴, or R⁵ and R⁶, or a combination thereof, combine with the carbon to which they are attached to form an optionally substituted C₃-C₈cycloalkyl or an optionally substituted 3- to 8-membered heterocyclyl containing 1-4 heteroatoms independently selected from the group consisting of N, O, and S,
   wherein an optionally substituted R³, R⁴, R⁵, and R⁶ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆ aliphatic;
R⁷, R⁸, and R⁹ are each independently selected from the group consisting of —H, halogen, —OR, —OC(O)R′, —OS(O)₂R′, —OS(O)₂NR₂, —OC(O)NR₂, —OC(O)OR, —NR₂, —NRC(O)R′, —NRS(O)₂R′, —NRC(O)NR₂, —NRC(O)OR, —CN, —NO₂, —SR, —C(O)R′, —C(O)OR, —C(O)NR₂, —S(O)₂R′, —SO₂NR₂, —S(O)₂OR, and optionally substituted C₁-C₆aliphatic,
   wherein an optionally substituted R⁷, R⁸, and R⁹ group may be substituted with one or more of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, and C₁-C₆aliphatic;
each R is independently selected from the group consisting of —H, optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R group may be optionally substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
each R′ is independently selected from the group consisting of optionally substituted C₁-C₆aliphatic, optionally substituted C₃-C₁₀cycloalkyl, optionally substituted 3- to 10-membered heterocyclyl containing 1-4 heteroatoms independently selected from N, O, and S, optionally substituted phenyl, and optionally substituted 5- to 10-membered heteroaryl containing 1-4 heteroatoms independently selected from N, O, and S,
   wherein an optionally substituted R′ group may be substituted with one or more of halogen, oxo, —OH, —O(C₁-C₆aliphatic), —NH₂, —NH(C₁-C₆aliphatic), —N(C₁-C₆aliphatic)₂, —CN, and C₁-C₆aliphatic;
m is 0, 1, or 2; and
n is 0, 1, or 2.

76. The method of any one of the preceding embodiments, wherein the USP9X Inhibitor is a compound of Formula II-a:

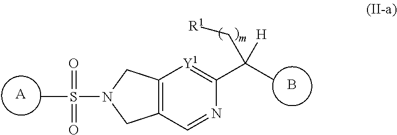

or a pharmaceutically acceptable salt thereof.

77. The method of any one of the preceding embodiments, wherein the USP9X Inhibitor is a compound of Formula II-b:

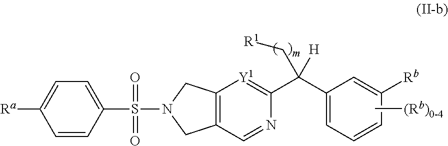

or a pharmaceutically acceptable salt thereof.

78. The method of any one of the preceding embodiments, wherein the USP9X Inhibitor is a compound of Formula II-c:

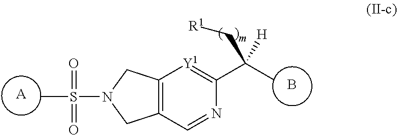

or a pharmaceutically acceptable salt thereof.

79. The method of any one of the preceding embodiments, wherein the USP9X Inhibitor is a compound of Formula II-d:

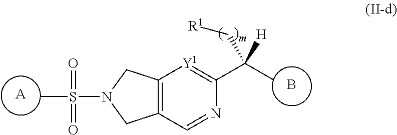

or a pharmaceutically acceptable salt thereof.

80. The method of any one of the preceding embodiments, wherein the USP9X Inhibitor is a compound of Formula II-e:

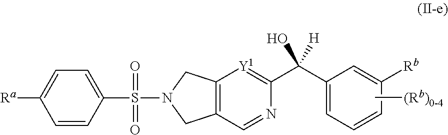

or a pharmaceutically acceptable salt thereof.

81. The method of any one of the preceding embodiments, wherein the USP9X Inhibitor is a compound of Formula II-f:

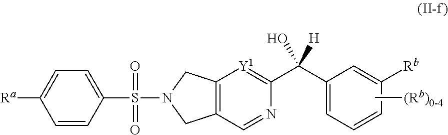

or a pharmaceutically acceptable salt thereof.

82. The method of any one of the preceding embodiments, wherein the USP9X Inhibitor is selected from:

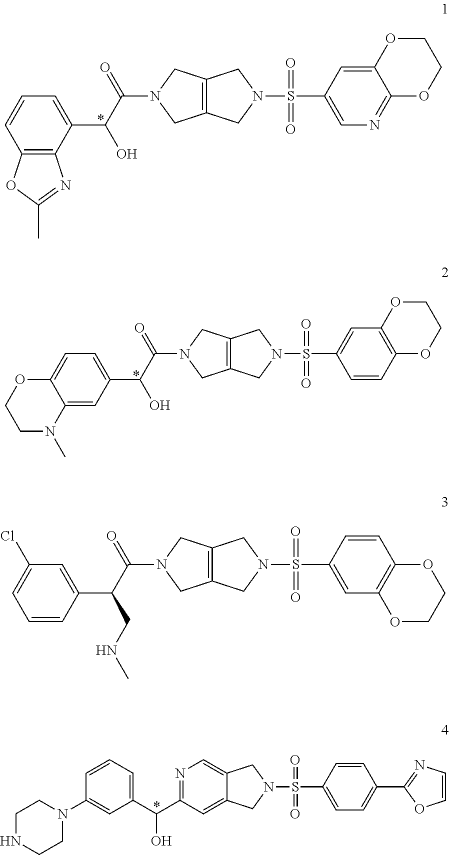 [SMILES: Cc1nc2c(C(O)C(=O)N3CC4=C(C3)CN(S(=O)(=O)c3cnc5c(c3)OCCO5)C4)cccc2o1 ; CN1CCOc2ccc(C(O)C(=O)N3CC4=C(C3)CN(S(=O)(=O)c3ccc5c(c3)OCCO5)C4)cc21 ; CNC[C@H](C(=O)N1CC2=C(C1)CN(S(=O)(=O)c1ccc3c(c1)OCCO3)C2)c1cccc(Cl)c1 ; O=S(=O)(c1ccc(-c2ncco2)cc1)N1Cc2cnc(C(O)c3cccc(N4CCNCC4)c3)cc2C1]

or a pharmaceutically acceptable salt thereof.

83. The method of any one of the preceding embodiments, wherein the USP9X Inhibitor is administered in a therapeutically effective amount.

1. A method of treating cancer in a patient in need thereof, comprising administering to the patient a USP9X Inhibitor of Formula I: 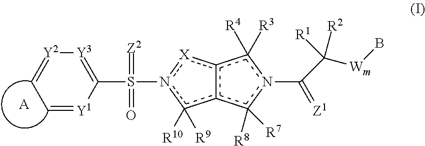
or a pharmaceutically acceptable salt thereof, wherein:
X is CR⁵R⁶, CR⁵, NR⁵, or N, as valency permits;
dashed bonds are each independently a single or a double bond, as valency permits;
Y¹, Y², and Y³ are each independently N or CR^{a};
each R^{a} is independently —H, halogen, or —CN;
Ring A is a 5- to 6-membered aryl, 5- to 6-membered heteroaryl containing 1-4 heteroatoms independently selected from the group consisting of O, N, and S, 5- to 7-membered heterocyclyl containing 1-4 heteroatoms independently selected from the group consisting of O, N, and S, or 5- to 7-membered cycloalkyl,
wherein each aryl, heteroaryl, heterocyclyl, or cycloalkyl is optionally substituted with one or more halogen, —C₁-C₆alkyl, —C₂-C₆alkenyl, —C₂-C₆alkynyl, oxo, or —C(O)R′;
Z¹ is O, S, or NR;
Z² is O or NR;
W is CR^{1′}R^{2′}, O, S, or NR;
m is 0 or 1;
R¹ and R² are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —OR, —OC(O)R′, —OS(O)₂R′, —OS(O)₂NR₂, —OC(O)NR₂, —OC(O)OR, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
wherein each cycloalkyl, cycloalkenyl, heterocyclyl, aryl, or heteroaryl is optionally substituted with one or more R^{e},
wherein each alkyl, alkenyl, or alkynyl is optionally substituted with one or more halogen,
wherein each heterocyclyl is 3- to 14-membered and contains 1-4 heteroatoms independently selected from the group consisting of O, N, and S, and wherein the heterocyclyl does not contain an O in the γ-position relative to C(═Z¹), and
wherein each heteroaryl is 5- to 14-membered and contains 1-4 heteroatoms independently selected from the group consisting of O, N, and S;
or R¹ and R² combine with the carbon to which they are attached to form oxo, a C₃-C₈cycloalkyl, or a 3- to 8-membered heterocyclyl containing 1-4 heteroatoms independently selected from the group consisting of O, N, and S, and wherein the heterocyclyl does not contain an O in the γ-position relative to C(═Z¹), and
wherein each cycloalkyl or heterocyclyl is optionally substituted with one or more R^{e};
R^{1′} and R^{2′} are each independently —H, halogen, —C₁-C₆alkyl, —C₂-C₆alkenyl, —C₂-C₆alkynyl, —(CR^{b}R^{c})ₙC₃-C₁₂cycloalkyl, —(CR^{b}R^{c})ₙC₄-C₁₂cycloalkenyl, —(CR^{b}R^{c})ₙheterocyclyl, —(CR^{b}R^{c})ₙC₆-C₁₄aryl, —(CR^{b}R^{c})ₙheteroaryl, —(CR^{b}R^{c})ₙNR₂, —(CR^{b}R^{c})ₙNRC(O)R′, —(CR^{b}R^{c})ₙNRS(O)₂R′, —(CR^{b}R^{c})ₙNRC(O)NR₂, —(CR^{b}R^{c})ₙNRC(O)OR, —(CR^{b}R^{c})ₙCN, —(CR^{b}R^{c})ₙNO₂, —(CR^{b}R^{c})ₙSR, —(CR^{b}R^{c})ₙC(O)R′, —(CR^{b}R^{c})ₙC(O)OR, —(CR^{b}R^{c})ₙC(O)NR₂, —(CR^{b}R^{c})ₙSO₂R′, —(CR^{b}R^{c})ₙSO₂NR₂, or —(CR^{b}R^{c})ₙSO₂OR,
wherein each alkyl, alkenyl, alkynyl, cycloalkyl, cycloalkenyl, heterocyclyl, aryl, or heteroaryl is optionally substituted with one or more R^{e},
wherein each heterocyclyl is 3- to 14-membered and contains 1-4 heteroatoms independently selected from the group consisting of O, N, and S, and
wherein each heteroaryl is 5- to 14-membered and contains 1-4 heteroatoms independently selected from the group consisting of O, N, and S;
or R^{1′} and R^{2′} combine with the carbon to which they are attached to form oxo, a C₃-C₈cycloalkyl, or a 3- to 8-membered heterocyclyl containing 1-4 heteroatoms independently selected from the group consisting of O, N and S,
wherein each heterocyclyl does not contain an O in the γ-position relative to C(═Z¹), and
wherein each cycloalkyl or heterocyclyl is optionally substituted with one or more R^{e};
or R¹ and R^{1′} combine with the carbons to which they are attached to form a C₃-C₈cycloalkyl or 3- to 8-membered heterocyclyl containing 1-4 heteroatoms independently selected from the group consisting of O, N and S,
wherein each heterocyclyl does not contain an O in the γ-position relative to C(═Z¹), and
wherein each cycloalkyl or heterocyclyl is optionally substituted with one or more R^{e};
R^{b} and R^{c} are each independently selected from the group consisting of —H, halogen, and —C₁-C₆alkyl;
each n is independently 0, 1, 2, 3, or 4;
each R^{e} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S, and wherein —OR of R^{e} does not result in an O in the γ-position relative to C(═Z¹),
wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
B is a monocyclic or bicyclic 3- to 14-membered ring,
wherein the ring is saturated, fully or partially unsaturated, or aromatic, and
wherein the ring contains 0-4 heteroatoms independently selected from the group consisting of O, N, and S,
wherein the ring is optionally substituted with one or more R^{d}, and
when m is 0 and the ring is saturated or partially unsaturated, then the ring does not contain an O in the γ-position relative to C(═Z¹);
each R^{d} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
wherein each alkyl, alkenyl, alkynyl, cycloalkyl, cycloalkenyl, heterocyclyl, aryl, or heteroaryl is optionally substituted with one or more substituents selected from the group consisting of halogen, oxo, —C₁-C₆alkyl optionally substituted with one or more halogen, —C₂-C₆alkenyl, —C₂-C₆alkynyl, —OR, —C₃-C₁₂cycloalkyl, or 3- to 8-membered heterocyclyl containing 1-4 heteroatoms independently selected from the group consisting of O, N, and S;
each R³, R⁴, R⁵, R⁶, R⁷, R⁸, R⁹, and R¹⁰ is independently —H, —C₁-C₆alkyl, —C₃-C₈cycloalkyl, or 3- to 8-membered heterocyclyl containing 1-4 heteroatoms independently selected from the group consisting of O, N, and S,
wherein each alkyl, cycloalkyl, or heterocyclyl is optionally substituted with one or more halogen, oxo, —C₁-C₆alkyl, —C₂-C₆alkenyl, —C₂-C₆alkynyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₃-C₈cycloalkyl, or 3- to 8-membered heterocyclyl containing 1-4 heteroatoms independently selected from the group consisting of O, N, and S, and
wherein R³, R⁷, and R⁹ are each independently present or absent, as valency permits;
or R³ and R⁴, R⁵ and R⁶, R⁷ and R⁸, R⁹ and R¹⁰, or combinations thereof, combine with the carbon to which they are attached to form an oxo, C₃-C₈cycloalkyl, or 3- to 8-membered heterocyclyl containing 1-4 heteroatoms independently selected from the group consisting of O, N, and S;
each R is independently selected from the group consisting of —H, —OH, —O(C₁-C₆alkyl), —NH₂, —NH(C₁-C₆alkyl), —N(C₁-C₆alkyl)₂, —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, C₆-C₁₄aryl, and 5- to 14-membered heteroaryl containing 1-4 heteroatoms independently selected from the group consisting of O, N, and S,
wherein each alkyl, alkenyl, alkynyl, cycloalkyl, cycloalkenyl, heterocyclyl, aryl, or heteroaryl is optionally substituted with one or more halogen, oxo, —O—C₁-C₆alkyl, —NH(C₁-C₆alkyl), —N(C₁-C₆alkyl)₂, —C₁-C₆alkyl optionally substituted with one or more oxo or —OH, —C₂-C₆alkenyl, —C₂-C₆alkynyl, —C₃-C₁₂cycloalkyl, or 3- to 8-membered heterocyclyl containing 1-4 heteroatoms independently selected from the group consisting of O, N, and S; and
each R′ is independently selected from the group consisting of —C₁-C₆alkyl, —C₂-C₆alkenyl, —C₂-C₆alkynyl, —C₃-C₁₂cycloalkyl, —C₄-C₁₂cycloalkenyl, 3- to 14-membered heterocyclyl containing 1-4 heteroatoms independently selected from the group consisting of O, N, and S, aryl, and 5- to 14-membered heteroaryl containing 1-4 heteroatoms independently selected from the group consisting of O, N, and S,
wherein each alkyl, alkenyl, alkynyl, cycloalkyl, cycloalkenyl, heterocyclyl, aryl, or heteroaryl is optionally substituted with one or more halogen, oxo, —C₁-C₆alkyl optionally substituted with one or more oxo or —OH, —C₂-C₆alkenyl, —C₂-C₆alkynyl, —O—C₁-C₆alkyl, —NH(C₁-C₆alkyl), or —N(C₁-C₆alkyl)₂.

2. The method of claim 1, comprising administering to the patient an antineoplastic therapy consisting of the administration of the USP9X Inhibitor.

3. The method of claim 1, comprising administering to the patient the USP9X Inhibitor, wherein the patient is receiving or has received an immune checkpoint pathway inhibitor.

4. The method of claim 1, comprising administering to the patient an immune checkpoint pathway inhibitor, wherein the patient is receiving or has received the USP9X Inhibitor.

5. The method of claim 1, comprising administering to the patient an antineoplastic therapy consisting of the administration of the USP9X Inhibitor and the administration of an immune checkpoint pathway inhibitor.

6. The method of claim 1, comprising administering the USP9X Inhibitor to the patient, wherein the patient is already being treated for the cancer with an immune checkpoint pathway inhibitor.

7. The method of claim 1, comprising administering the USP9X Inhibitor to the patient, wherein the cancer has progressed while receiving an immune checkpoint pathway inhibitor.

8. The method of claim 1, wherein the patient i) has been diagnosed with cancer that has progressed, or ii) has relapsed after previously being administered an immune checkpoint pathway inhibitor for the cancer.

9. The method of claim 1, wherein the method comprises administering the USP9X Inhibitor to the patient while the patient continues to receive an immune checkpoint pathway inhibitor after being diagnosed with a cancer that is refractory to an immune checkpoint pathway inhibitor.

10. The method of claim 1, wherein the cancer comprises a tumor that expresses PD-L1.

11. The method of claim 1, wherein the cancer comprises a tumor that expresses PD-L1 and the PD-L1 can be detected using PD-L1 IHC 22C3 pharmDx.

12. The method of claim 1, wherein the USP9X Inhibitor has an IC₅₀ value of ≤2 μM in the Biochemical Assay of Example A.

13. The method of claim 1, wherein the USP9X Inhibitor has an IC₅₀ value of ≤1 μM in the Biochemical Assay of Example A.

14. The method of claim 1, wherein the USP9X Inhibitor has an IC₅₀ value of ≤0.1 μM in the Biochemical Assay of Example A.

15. The method of claim 1, wherein the USP9X Inhibitor is administered in a therapeutically effective amount.
